(19) United States
(12) Patent Application Publication
(10) Pub. No.: US 2009/0029958 A1
(43) Pub. Date: Jan. 29, 2009

(54) PHENETHANOLAMINE DERIVATIVES AS BETA2 ADRENORECEPTOR AGONISTS

(76) Inventors: Lilian Alcaraz, Leicestershire (GB); Andrew Bailey, Leicestershire (GB); Rhona Cox, Leicestershire (GB); Premji Meghani, Leicestershire (GB); Garry Pairaudeau, Leicestershire (GB); Michael Stocks, Leicestershire (GB)

Correspondence Address: MORGAN LEWIS & BOCKIUS LLP, 1111 PENNSYLVANIA AVENUE NW, WASHINGTON, DC, 20004, US

(21) Appl. No.: 12/282,019

(22) PCT Filed: Mar. 6, 2007
(86) PCT No.: PCT/SE07/00217

§ 371 Date: Sep. 8, 2008

(30) Foreign Application Priority Data
Mar. 8, 2006 (US) 60/780251
Apr. 5, 2006 (US) 60/789516
Jul. 14, 2006 (US) 60/821091

(51) Int. Cl. A61K 31/4709; C07D 215/227; A61K 31/58; A61P 11/00

(52) U.S. Cl. 514/171; 546/157; 514/312

(57) ABSTRACT

The present invention relates to compounds according to formula (I), a process for preparing them, the intermediate compounds of the process and the use of the compounds in the manufacture of a medicament for use in treating diseases such as ARDS, pulmonary emphysema, bronchitis, bronchiectasis, COPD, asthma and rhinitis. The compounds are beta2 adrenoreceptor agonists.

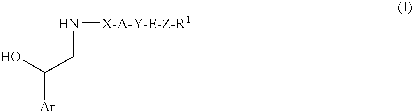

The present invention relates to phenethanolamine derivatives, processes for their preparation, pharmaceutical compositions containing them and their use in therapy.

Adreneoceptors are a group of G-protein coupled receptors divided into two major sub-families, α and β. These sub-families are further divided into sub-types of which the D sub-family has at least 3 members: β1, β2 and β3. β2 adrenoceptors (henceforth referred to as β2 receptors) are mainly expressed on smooth muscle cells.

Agonism of the β2 receptor on airway smooth muscle produces relaxation and therefore bronchodilatation. Through this mechanism, β2 agonists act as functional antagonists to all bronchoconstrictor substances such as the naturally-occurring histamine and acetylcholine as well as the experimental substances methacholine and carbachol. β2 agonists are widely used to treat airway diseases including asthma and chronic obstructive pulmonary disease (COPD), and this has been extensively reviewed in the literature and incorporated into national guidelines for the treatment of these diseases (British Guideline on the Management of Asthma, NICE guideline No. 12 on the Management of COPD).

β2 agonists are classed either as short-acting or long-acting. Short-acting β2 agonists (SABAs) such as salbutamol have a duration of action of 2-4 hours. They are suitable for rescue medication during a period of acute bronchoconstriction but are not suitable for continuous medication because the beneficial effect of these drugs wears off during the night. Long-acting 132 agonists (LABAs) currently have a duration of action of about 12 hours and are administered twice daily to provide continuous bronchodilatation. They are particularly effective when administered in combination with inhaled corticosteroids. This benefit is not seen when inhaled corticosteroids are combined with SABAs (Kips and Pauwels, Am. J. Respir. Crit. Care Med., 2001, 164, 923-932). LABAs are recommended as add-on therapy to patients already receiving inhaled corticosteroids for asthma to reduce nocturnal awakening and reduce the incidence of exacerbations of the disease.

Corticosteroids and LABAs are conveniently co-administered in a single inhaler to improve patient compliance.

There are shortcomings to existing LABAs and there is a need for a new drug in this class. Salmeterol, a commonly used LABA, has a narrow safety margin and side effects related to systemic agonism of β2 receptors (such as tremor, hypokalaemia, tachycardia and hypertension) are common. Salmeterol also has a long onset of action which precludes its use as both a rescue and a maintenance therapy. All current LABAs are administered twice daily and there is a medical need for once daily treatments to improve treatment and patient compliance. Such once daily compounds, co-administered with corticosteroids, will become the mainstay of asthma treatment (Barnes, Nature Reviews, 2004, 3, 831-844). The advantages of once-daily bronchodilator treatment in COPD has been demonstrated with tiotropium, a non-selective muscarinic antagonist (Koumis and Samuel, Clin. Ther. 2005, 27(4), 377-92). There is, however, a need for a once-daily LABA for the treatment of COPD to avoid the side effects of anti-muscarinics such as tiotropium.

Benzothiazolone derivatives having β2 adrenoreceptor agonist properties are known from WO 2004/016601.

In accordance with the present invention, there is provided a compound of formula (I):

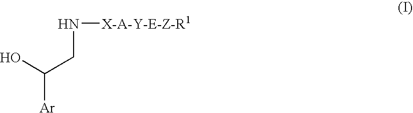

wherein Ar is

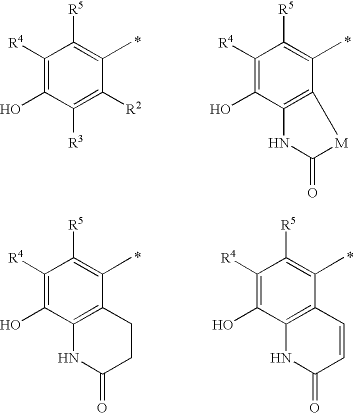

### M is C(O), NR⁶, S or CR⁷R⁸;

R², R³, R⁴ and R⁵ are, independently, hydrogen, halogen, trifluoromethyl, cyano, carboxy, hydroxy, nitro, S(O)₂R⁹, NR¹⁰S(O)₂R¹¹, C(O)NR¹²R¹³, NR¹⁴C(O)R¹⁵, C₁₋₆ alkyl, C₁₋₆ alkoxy, C(O)(C₁₋₆ alkyl) or C(O)₂(C₁₋₆ alkyl);
R³ can also be CH₂OH or NHS(O)₂NR¹⁷R¹⁸;
X is a bond, CR²⁷R²⁸ or CR²⁹R³⁰CR³¹R³²;
Y is CR³³R³⁴CR³⁵R³⁶, CR³⁷R³⁸CR³⁹R⁴⁰CR⁴¹R⁴² or CR⁴³R⁴⁴CR⁴⁵R⁴⁶CR⁴⁷R⁴⁸CR⁴⁹R⁵⁰;
or Y is CR⁵¹R⁵² provided that E is C(O)O—;
Z is a bond, CR⁵¹R⁵², CR⁵³R⁵⁴CR⁵⁵R⁵⁶, CR⁵⁷R⁵⁸CR⁵⁹R⁶⁰CR⁶¹R⁶² or CR⁶³R⁶⁴CR⁶⁵R⁶⁶CR⁶⁷R⁶⁸CR⁶⁹R⁷⁰;
A is a cycloalkyl-amino group selected from

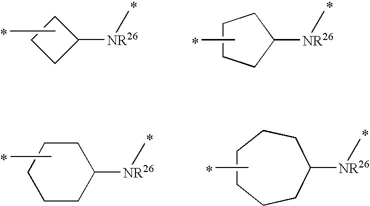

wherein said cycloalkyl ring is unsubstituted or substituted by 1 or 2 substituents independently selected from halogen, C₁₋₄ alkyl (optionally substituted by OR¹¹⁶, NR¹¹⁷R¹¹⁸ or NR¹¹⁹C(O)R¹²⁰), OR¹⁹, NR²⁰R²¹, C(O)NR²²R²³, NR²⁴C(O)R²⁵, CN, S(O)₂R¹⁶, or S(O)₂NR¹¹⁴R¹¹⁵;
when A is a cycloalkyl-amino group A is linked to X through a ring carbon atom and to Y through NR²⁶; or
when A is a cycloalkyl-amino group and X is CR²⁹R³⁰CR³¹R³² A can be linked to X through NR²⁶ and to Y through a ring carbon atom;
when X is a bond A is not connected to X through the ring-carbon atom carrying NR²⁶;
OR A is a heterocyclyl ring selected from

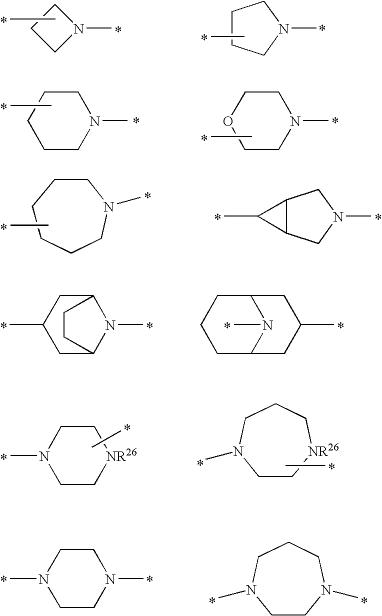

wherein the heterocyclyl ring is unsubstituted or substituted by 1 or 2 substituents (for example a substituent is on the same ring carbon atom as that joining A to either X or Y) independently selected from halogen, C₁₋₄ alkyl (optionally substituted by OR¹²¹, NR¹²²R¹²³ or NR¹²⁴C(O)R¹²⁵), OR¹⁹, NR²⁰R²¹, C(O)NR²²R²³, NR²⁴C(O)R²⁵, CN, S(O)₂R¹²⁶ or S(O)₂NR¹¹⁴R¹¹⁵;
when A is a heterocyclyl ring A is linked to Y through a ring nitrogen atom;
when A is a heterocyclyl ring A can be linked to X through a ring carbon atom; or, when A is heterocyclyl having 2 ring-nitrogen atoms and X is CR²⁹R³⁰CR³¹R³², A can be linked to X through the second ring nitrogen atom;

### E is O, S, S(O)₂, NR⁷¹, C(O)NR⁷², NR⁷³C(O), C(O)O, S(O)₂NR⁷⁴ or NR⁷⁵S(O)₂;

R¹ is aryl, aryloxy, NR⁷⁶aryl, S(O)₂aryl, heteroaryl or C₃₋₁₀ cycloalkyl (optionally substituted by C₁₋₆ alkyl, halogen or phenyl); wherein the aryl and heteroaryl rings are optionally substituted by halogen, cyano, trifluoromethyl, phenyl, OCF₃, O(CF₂)ₙO, O(CH₂)ₘO, OR⁷⁸, SR⁷⁹, NR⁸⁰R⁸¹, C(O)NR⁸²R⁸³, NR⁸⁴S(O)₂R⁸⁵, C(O)R⁸⁶, S(O)₂R⁸⁷, S(O)₂NR⁸⁸R⁸⁹, NR⁹⁰C(O)R⁹¹, C(O)OR⁹², C₁₋₆ alkyl (optionally substituted by fluoro, trifluoromethyl, phenyl, heteroaryl, OR⁹³, NR⁹⁴R⁹⁵, C(O)NR⁹⁶R⁹⁷, NR⁹⁸S(O)₂R⁹⁹, S(O)₂R¹⁰⁰ or S(O)₂NR¹¹¹R¹¹²) or C₁₋₆ alkoxy (optionally substituted by fluoro, trifluoromethyl, phenyl, heteroaryl, OR¹⁰³, NR¹⁰⁴R¹⁰⁵, C(O)NR¹⁰⁶R¹⁰⁷, NR¹⁰⁸S(O)₂R¹⁰⁹, S(O)₂R¹¹⁰ or S(O)₂NR¹¹¹R¹¹²); wherein 2 substituents on the aryl or heteroaryl ring which is R¹ can join together to form a 4- to 8-membered ring which is carbocyclic or heterocyclic (for example containing 1, 2, 3 or 4 heteroatoms independently selected from O, N and S), said 4- to 8-membered ring is fused and is optionally substituted by halogen, C₁₋₄ alkyl, CF₃ or C₁₋₄ alkoxy;
when Z is a bond E can also be C(O) provided R¹ is a group selected from:

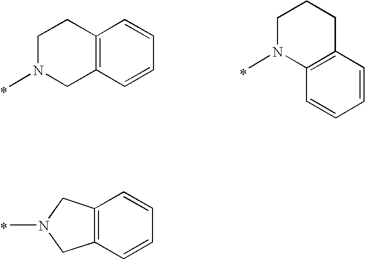

that is optionally substituted as for R¹ above;
n and m are, independently, 1 or 2;
R⁶, R⁷, R⁸, R¹⁰, R¹², R¹³, R¹⁴, R¹⁵, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, R⁷⁴, R⁷⁵, R⁷⁶, R⁷⁷, R⁷⁸, R⁷⁹, R⁸⁰, R⁸¹, R⁸², R⁸³, R⁸⁴, R⁸⁵, R⁸⁶, R⁸⁷, R⁸⁸, R⁸⁹, R⁹⁰, R⁹¹, R⁹², R⁹³, R⁹⁴, R⁹⁵, R⁹⁶, R⁹⁷, R⁹⁸, R¹⁰¹, R¹⁰², R¹⁰³, R¹⁰⁴, R¹⁰⁵, R¹⁰⁶, R¹⁰⁷, R¹⁰⁸, R¹¹¹, R¹¹², R¹¹³, R¹¹⁴, R¹¹⁵, R¹¹⁶, R¹¹⁷, R¹¹⁸, R¹¹⁹, R¹²⁰, R¹²¹, R¹²², R¹²³, R¹²⁴ and R¹²⁵ are,
independently, hydrogen or C₁₋₆ alkyl;
R⁵² can also be phenyl;
R⁷² can also be phenyl(C₁₋₄ alkyl) (for example benzyl);
R⁹, R¹¹, R¹⁶, R⁸⁵, R⁸⁷, R⁹⁹, R¹⁰⁰, R¹⁰⁹, R¹¹⁰ and R¹²⁶ are, independently, C₁₋₆ alkyl;
provided that when R¹ is aryloxy, NR⁷⁶aryl or S(O)₂aryl; and E is O, S, S(O)₂, NR⁷¹, C(O)NR⁷², S(O)₂NR⁷⁴ or NR⁷⁵S(O)₂, then Z is CR⁵³R⁵⁴CR⁵⁵R⁵⁶, CR⁵⁷R⁵⁸CR⁵⁹R⁶⁰CR⁶¹R⁶² or CR⁶³R⁶⁴CR⁶⁵R⁶⁶CR⁶⁷R⁶⁸CR⁶⁹R⁷⁰;
or a pharmaceutically acceptable salt thereof.

In one particular aspect the present invention provides a compound of formula (I) wherein:

### Ar is:

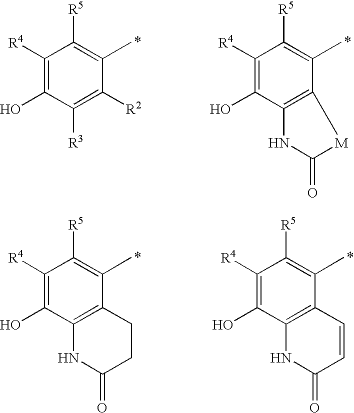

### M is C(O), NR⁶, S or CR⁷R⁸;

R², R³, R⁴ and R⁵ are, independently, hydrogen, halogen, trifluoromethyl, cyano, carboxy, hydroxy, nitro, S(O)₂R⁹, NR¹⁰S(O)₂R¹¹, C(O)NR¹²R¹³, NR¹⁴C(O)R¹⁵, C₁₋₆ alkyl, C₁₋₆ alkoxy, C(O)(C₁₋₆ alkyl) or C(O)₂(C₁₋₆ alkyl);
R³ can also be CH₂OH or NHS(O)₂NR¹⁷R¹⁸;
X is a bond, CR²⁷R²⁸ or CR²⁹R³⁰CR³¹R³²;
Y is CR³³R³⁴CR³⁵R³⁶, CR³⁷R³⁸CR³⁹R⁴⁰CR⁴¹R⁴² or CR⁴³R⁴⁴CR⁴⁵R⁴⁶CR⁴⁷R⁴⁸CR⁴⁹R⁵⁰;
or Y is CR⁵¹R⁵² provided that E is C(O)O—;
Z is a bond, CR⁵¹R⁵², CR⁵³R⁵⁴CR⁵⁵R⁵⁶, CR⁵⁷R⁵⁸CR⁵⁹R⁶⁰CR⁶¹R⁶² or CR⁶³R⁶⁴CR⁶⁵R⁶⁶CR⁶⁷R⁶⁸CR⁶⁹R⁷⁰;
A is a cycloalkyl-amino group selected from

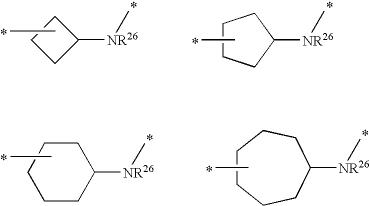

wherein said cycloalkyl ring is unsubstituted or substituted by 1 or 2 substituents independently selected from halogen, C₁₋₄ alkyl (optionally substituted by OR¹¹⁶, NR¹¹⁷R¹¹⁸ or NR¹¹⁹C(O)R¹²⁰), OR¹⁹, NR²⁰R²¹, C(O)NR²²R²³, NR²⁴C(O)R²⁵, CN, S(O)₂R¹⁶, or S(O)₂NR¹¹⁴R¹¹⁵;
when A is a cycloalkyl-amino group A can be linked to X through a ring carbon atom and to Y through NR²⁶; or
when A is a cycloalkyl-amino group and X is CR²⁹R³⁰CR³¹R³² A can be linked to X through NR²⁶ and to Y through a ring carbon atom;
when X is a bond A is not connected to X through the ring-carbon atom carrying NR²⁶;
OR A is a heterocyclyl ring selected from

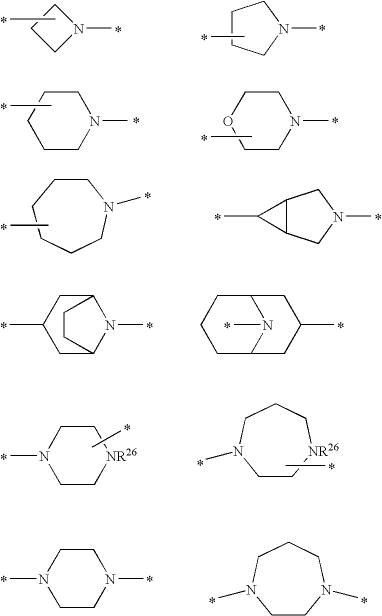

wherein the heterocyclyl ring is unsubstituted or substituted by 1 or 2 substituents (for example a substituent is on the same ring carbon atom as that joining A to either X or Y) independently selected from halogen, C₁₋₄ alkyl (optionally substituted by OR¹²¹, NR¹²²R¹²³ or NR¹²⁴C(O)R¹²⁵), OR¹⁹, NR²⁰R²¹, C(O)NR²²R²³, NR²⁴C(O)R²⁵, CN, S(O)₂R¹²⁶ or S(O)₂NR¹¹⁴R¹¹⁵;
when A is a heterocyclyl ring A is linked to Y through a ring nitrogen atom;
when A is a heterocyclyl ring A can be linked to X through a ring carbon atom; or, when A is heterocyclyl having 2 ring-nitrogen atoms and X is CR²⁹R³⁰CR³¹R³², A can be linked to X through the second ring nitrogen atom;

### E is O, S, S(O)₂, NR⁷¹, C(O)NR⁷², NR⁷³C(O), C(O)O, S(O)₂NR⁷⁴ or NR⁷⁵S(O)₂;

R¹ is aryl, aryloxy, NR⁷⁶aryl, S(O)₂aryl, heteroaryl or C₃₋₁₀ cycloalkyl (optionally substituted by C₁₋₆ alkyl, halogen or phenyl); wherein the aryl and heteroaryl rings are optionally substituted by halogen, cyano, trifluoromethyl, phenyl, OCF₃, O(CF₂)ₙO, O(CH₂)ₘO, OR⁷⁸, SR⁷⁹, NR⁸⁰R⁸¹, C(O)NR⁸²R⁸³, NR⁸⁴S(O)₂R⁸⁵, C(O)R⁸⁶, S(O)₂R⁸⁷, S(O)₂NR⁸⁸R⁸⁹, NR⁹⁰C(O)R⁹¹, C(O)OR⁹², C₁₋₆ alkyl (optionally substituted by fluoro, trifluoromethyl, phenyl, heteroaryl, OR⁹³, NR⁹⁴R⁹⁵, C(O)NR⁹⁶R⁹⁷, NR⁹⁸S(O)₂R⁹⁹, S(O)₂R¹⁰⁰ or S(O)₂NR¹⁰¹R¹⁰²) or C₁₋₆ alkoxy (optionally substituted by fluoro, trifluoromethyl, phenyl, heteroaryl, OR¹⁰³, NR¹⁰⁴R¹⁰⁵, C(O)NR¹⁰⁶R¹⁰⁷, NR¹⁰⁸S(O)₂R¹⁰⁹, S(O)₂R¹¹⁰ or S(O)₂NR¹¹¹R¹¹²); wherein 2 substituents on the aryl or heteroaryl ring which is R¹ can join together to form a 4- to 8-membered ring which is carbocyclic or heterocyclic (for example containing 1, 2, 3 or 4 heteroatoms independently selected from O, N and S), said 4- to 8-membered ring is fused and is optionally substituted by halogen, C₁₋₄ alkyl, CF₃ or C₁₋₄ alkoxy;
when Z is a bond E can also be C(O) provided R¹ is a group selected from:

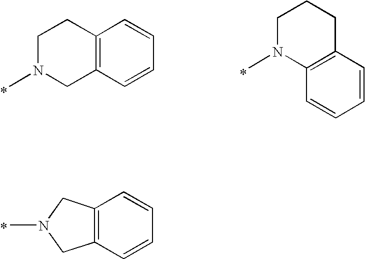

that is optionally substituted as for R¹ above;
n and m are, independently, 1 or 2;
R⁶, R⁷, R⁸, R¹⁰, R¹², R¹³, R¹⁴, R¹⁵, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, R⁷⁴, R⁷⁵, R⁷⁶, R⁷⁷, R⁷⁸, R⁷⁹, R⁸⁰, R⁸¹, R⁸², R⁸³, R⁸⁴, R⁸⁶, R⁸⁸, R⁸⁹, R⁹⁰, R⁹¹, R⁹², R⁹³, R⁹⁴, R⁹⁵, R⁹⁶, R⁹⁷, R⁹⁸, R¹⁰¹, R¹⁰², R¹⁰³, R¹⁰⁴, R¹⁰⁵, R¹⁰⁷, R¹⁰⁸, R¹¹¹, R¹¹², R¹¹³, R¹¹⁴, R¹¹⁵, R¹¹⁶, R¹¹⁷, R¹¹⁸, R¹¹⁹, R¹²⁰, R¹²¹, R¹²², R¹²³, R¹²⁴ and R¹²⁵ are, independently, hydrogen or C₁₋₆ alkyl;
R⁹, R¹¹, R¹⁶, R⁸⁵, R⁸⁷, R⁹⁹, R¹⁰⁰, R¹⁰⁹, R¹¹⁰ and R¹²⁶ are, independently, C₁₋₆ alkyl;
provided that when R¹ is aryloxy, NR⁷⁶aryl or S(O)₂aryl; and E is O, S, S(O)₂, NR⁷¹, C(O)NR⁷², S(O)₂NR⁷⁴ or NR⁷⁵S(O)₂, then Z is CR⁵³R⁵⁴CR⁵⁵R⁵⁶, CR⁵⁷R⁵⁸CR⁵⁹R⁶⁰CR⁶¹R⁶² or CR⁶³R⁶⁴CR⁶⁵R⁶⁶CR⁶⁷R⁶⁸CR⁶⁹R⁷⁰;
or a pharmaceutically acceptable salt thereof.

In another aspect the present invention provides a compound of formula (I) wherein Ar is

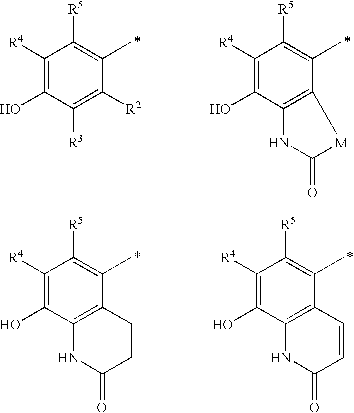

### M is C(O), NR⁶ or CR⁷R⁸;

R², R³, R⁴ and R⁵ are, independently, hydrogen, halogen, trifluoromethyl, cyano, carboxy, hydroxy, nitro, S(O)₂R⁹, NR¹⁰S(O)₂R¹¹, C(O)NR¹²R¹³, NR¹⁴C(O)R¹⁵, C₁₋₆ alkyl, C₁₋₆ alkoxy, C(O)(C₁₋₆ alkyl) or C(O)₂(C₁₋₆ alkyl);
R³ can also be CH₂OH or NHS(O)₂NR¹⁷R¹⁸;
X is a bond, CR²⁷R²⁸ or CR²⁹R³⁰CR³¹R³²;
Y is CR³³R³⁴CR³⁵R³⁶, CR³⁷R³⁸CR³⁹R⁴⁰CR⁴¹R⁴² or CR⁴³R⁴⁴CR⁴⁵R⁴⁶CR⁴⁷R⁴⁸CR⁴⁹R⁵⁰;
Z is a bond, CR⁵¹R⁵², CR⁵³R⁵⁴CR⁵⁵R⁵⁶, CR⁵⁷R⁵⁸CR⁵⁹R⁶¹CR⁶¹R⁶² or CR⁶³R⁶⁴CR⁶⁵R⁶⁶CR⁶⁷R⁶⁸CR⁶⁹R⁷⁰;
A is a cycloalkyl ring selected from

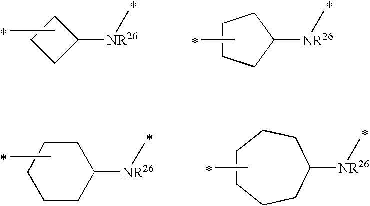

wherein said cycloalkyl ring is unsubstituted or substituted by 1 or 2 substituents independently selected from halogen, C₁₋₄ alkyl (optionally substituted by OR¹¹⁶, NR¹¹⁷R¹¹⁸ or NR¹¹⁹C(O)R¹²⁰)OR¹⁹, NR²⁰R²¹, C(O)NR²²R²³, NR²⁴C(O)R²⁵, CN, S(O)₂R¹⁶, or S(O)₂NR¹¹⁴R¹¹⁵;
when A is a cycloalkyl ring it is linked to Y through NR²⁶;
when A is a cycloalkyl ring it is linked to X either through a ring carbon atom, or through NR²⁶ provided that X is CR²⁹R³⁰CR³¹R³²;
when X is a bond A is not connected to X through the ring-carbon atom carrying NR²⁶;
OR A is a heterocyclyl ring selected from

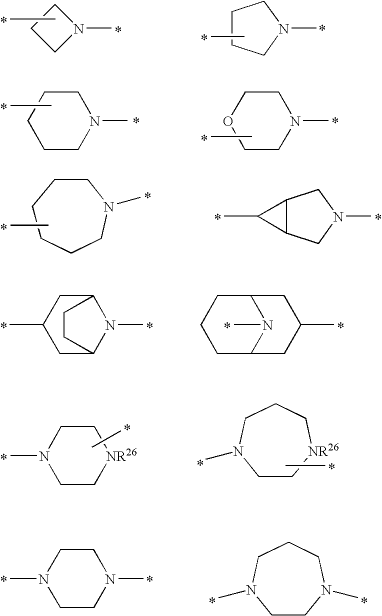

wherein the heterocyclyl ring is unsubstituted or substituted by 1 or 2 substituents independently selected from halogen, C₁₋₄ allyl (optionally substituted by OR¹²¹, NR¹²²R¹²³ or NR²⁴C(O)R¹²⁵), OR¹⁹, NR²⁰R²¹, C(O)NR²²R²³, NR²⁴C(O)R²⁵, CN, S(O)₂R¹²⁶ or S(O)₂NR¹¹⁴R¹¹⁵;
when A is a heterocyclyl ring it is linked to Y through a ring nitrogen atom;
when A is a heterocyclyl ring it is linked to X either through a ring carbon atom or through a second ring nitrogen atom provided that X is CR²⁹R³⁰CR³¹R³²;

### E is O, S, S(O)₂, NR⁷¹, C(O)NR⁷², NR⁷³C(O)S(O)₂NR⁷⁴ or NR⁷⁵S(O)₂;

R¹ is aryl, aryloxy, NR⁷⁶aryl, S(O)₂aryl, heteroaryl or C₃₋₇ cycloalkyl; wherein the aryl and heteroaryl rings are optionally substituted by halogen, cyano, trifluoromethyl, phenyl, O(CF₂)ₙO, O(CH₂)ₘO, OR⁷⁸, SR⁷⁹, NR⁸⁰R⁸¹, C(O)NR⁸²R⁸³, NR⁸⁴S(O)₂R⁸⁵, C(O)R⁸⁶, S(O)₂R⁸⁷, S(O)₂NR⁸⁸R⁸⁹, NR⁹⁰C(O)R⁹¹, C(O)OR⁹², C₁₋₆ alkyl (optionally substituted by fluoro, trifluoromethyl, phenyl, heteroaryl, OR⁹³, NR⁹⁴R⁹⁵, C(O)NR⁹⁶R⁹⁷, NR⁹⁸S(O)₂R⁹⁹, S(O)₂R¹⁰⁰ or S(O)₂NR¹⁰¹R¹⁰²) or C₁₋₆ alkoxy (optionally substituted by fluoro, trifluoromethyl, phenyl, heteroaryl, OR¹⁰³, NR¹⁰⁴R¹⁰⁵, C(O)NR¹⁰⁶R¹⁰⁷, NR¹⁰⁸S(O)₂R¹⁰⁹, S(O)₂R¹¹⁰ or S(O)₂NR¹¹¹R¹¹²); wherein 2 substituents on the aryl or heteroaryl ring which is R¹ can join together to form a 4- to 8-membered ring which is carbocyclic or heterocyclic (for example containing 1, 2, 3 or 4 heteroatoms independently selected from O, N and S), said 4- to 8-membered ring is fused and is optionally substituted by halogen, C₁₋₄ alkyl, CF₃ or C₁₋₄ alkoxy;
when Z is a bond E can also be C(O) provided R¹ is a group selected from:

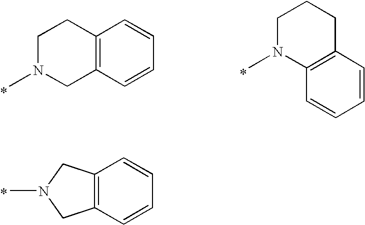

that is optionally substituted as for R¹ above;
n and m are, independently, 1 or 2;
R⁶, R⁷, R⁸, R¹⁰, R¹², R¹³, R¹⁴, R¹⁵, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, R⁷⁴, R⁷⁵, R⁷⁶, R⁷⁷, R⁷⁸, R⁷⁹, R⁸⁰, R⁸¹, R⁸², R⁸³, R⁸⁴, R⁸⁵, R⁸⁶, R⁸⁵, R⁸⁹, R⁹⁰, R⁹¹, R⁹², R⁹³, R⁹⁴, R⁹⁵, R⁹⁶, R⁹⁷, R⁹⁸, R¹⁰¹, R¹⁰², R¹⁰³, R¹⁰⁴, R¹⁰⁵, R¹⁰⁶, R¹⁰⁷, R¹⁰⁸, R¹¹¹, R¹¹², R¹¹³, R¹¹⁴, R¹¹⁵, R¹¹⁶, R¹¹⁷, R¹¹⁸, R¹¹⁹, R¹²⁰, R¹²¹, R¹²², R¹²³, R¹²⁴ and R¹²⁵ are, independently, hydrogen or C₁₋₆ alkyl;
R⁹, R¹¹, R¹⁶, R⁸⁵, R⁸⁷, R⁹⁹, R¹⁰⁰, R¹⁰⁹, R¹¹⁰ and R¹²⁶ are, independently, C₁₋₆ alkyl;
provided that when R¹ is aryloxy, NR⁷⁶aryl or S(O)₂aryl; and E is O, S, S(O)₂, NR⁷¹, C(O)NR⁷², S(O)₂NR⁷⁴ or NR⁷⁵S(O)₂, then Z is CR⁵³R⁵⁴CR⁵⁵R⁵⁶, CR⁵⁷R⁵⁸CR⁵⁹R⁶⁰CR⁶¹R⁶² or CR⁶³R⁶⁴CR⁶⁵R⁶⁶CR⁶⁷R⁶⁸CR⁶⁹R⁷⁰;
or a pharmaceutically acceptable salt thereof.

The compounds of the invention are selective β2 receptor agonists and possess properties that make them more suitable for once-a-day administration. Compounds have been optimised to have a predicted appropriate duration in an in vitro guinea pig trachea model, or mammalian model such as a histamine-challenged guinea pig. The compounds also have advantageous pharmokinetic half lives in a rat system. In particular, certain compounds of the invention are at least 10-fold more potent at the β2 receptor compared to the α1, β1, or dopamine (D2) receptors. Certain compounds are also notable for having a fast onset of action that is the time interval between administration of a compound of the invention to a patient and the compound providing symptomatic relief. Onset can be predicted in vitro using isolated trachea from guinea pig or human.

A suitable pharmaceutically acceptable salt is, for example, an acid addition salt, such as a hydrochloride, hydrobromide, trifluoroacetate, sulphate, phosphate, acetate, fumarate, maleate, tartrate, lactate, citrate, pyruvate, succinate, oxalate, methanesulphonate or p-toluenesulphonate. Further examples of acid addition salts are: bisulphate, benzenesulphonate, ethanesulphonate, malonate, xinafoate, ascorbate, oleate, nicotinate, saccharinate, adipate, formate, glycolate, L-lactate, D-lactate, aspartate, malate, L-tartrate, D-tartrate, stearate, 2-furoate, 3-furoate, napadisylate (naphthalene-1,5-disulfonate or naphthalene-1-(sulfonic acid)-5-sulfonate), edisylate (ethane-1,2-disulfonate or ethane-1-(sulfonic acid)-2-sulfonate), isethionate (2-hydroxyethylsulfonate), 2-mesitylenesulphonate and 2-naphthalenesulphonate.

The present invention covers all permissible ratios of compound of formula (I) to pharmaceutically acceptable salt, for example mono-hydrobromide, dihydrobromide or a hemi-salt (such as a hemi-fumarate).

Compounds of formula (I) are capable of existing in stereoisomeric forms. It will be understood that the invention encompasses the use of all geometric and optical isomers (including atropisomers) of the compounds of formula (I) and mixtures thereof including racemates. The use of tautomers and mixtures thereof also form an aspect of the present invention. Enantiomerically pure forms are particularly desired.

In the context of the present specification, unless otherwise stated, an alkyl substituent group or an alkyl moiety in a substituent group may be linear or branched. Examples of C₁₋₆ alkyl groups/moieties include methyl, ethyl, n-propyl, iso-propyl, n-butyl, isobutyl, tert-butyl, n-pentyl and n-hexyl.

Aryl is, for example, phenyl or naphthyl.

C₃₋₁₀ Cycloalkyl is optionally bridged by 1, 2, 3 or 4 carbon atoms. Examples include, cyclopropyl, cyclobutyl, cyclopentyl, cyclohexyl, cycloheptyl and adamantyl. In one aspect of the invention C₃₋₁₀ cycloalkyl is, for example, cyclohexyl or adamantyl.

Heteroaryl is an aromatic monocyclic or bicyclic ring, containing 5 to 10 ring atoms of which 1, 2, 3 or 4 ring atoms are chosen from nitrogen, sulphur or oxygen. Examples of heteroaryl include pyrrolyl, furanyl, thienyl, thiazolyl, isothiazolyl, oxazolyl, isoxazolyl, imidazolyl, pyrazolyl, triazolyl, tetrazolyl, isoxadiazolyl, oxadiazolyl, isothiadiazolyl, thiadiazolyl, pyridyl, pyrimidinyl, pyrazinyl, pyridazinyl, triazinyl, pyridinonyl, pyrimidindionyl, benzfuranyl, benzthienyl, indolyl, benzimidazolyl, benzoxazolyl, benzthiazolyl, indazolyl, benzisoxazolyl, benzisothiazolyl, benztriazolyl, quinolinyl, isoquinolinyl, 4H-chromen-4-onyl, tetrahydroquinolinyl, tetrahydroisoquinolinyl, indolinyl, isoindolinyl and naphthiridinyl.

When 2 substituents on the aryl or heteroaryl ring which is R¹ join together to form a 4- to 8-membered ring which is carbocyclic or heterocyclic (for example containing 1, 2, 3 or 4 heteroatoms independently selected from O, N and S), said 4- to 8-membered ring is fused and the resulting ring system of R¹ is, for example, 2,3-dihydro-1,4-benzodioxine, 2,2,3,3-tetrafluoro-2,3-dihydro-1,4-benzodioxine, 1,2,3,4-tetrahydronaphthalene, indane, 1,3-benzodioxole, 2,2-dimethyl-1,3-benzodioxole, 2,3-dihydro-1-benzofuran, 2,2-dimethyl-2,3-dihydro-1-benzofuran, indoline, 2,3-dihydro-1-benzothiophene, thiochromane, chromane, 1,2,3,4-tetrahydroquinoline, 6,7,8,9-tetrahydro-5H-benzo[7]annulene, 3,4-dihydro-2H-1,5-benzodioxepine, bicyclo[4.2.0]octa-1,3,5-triene, spiro[1,3-benzodioxole-2,1′-cyclohexane], spiro[1,3-benzodioxole-2,1′-cyclopentane], 4,5,6,7-tetrahydro-1,3-benzothiazole, 5,6-dihydro-4H-cyclopenta[d][1,3]thiazole, 5,6-dihydro-4H-cyclopenta[d][1,3]oxazole, 4,5,6,7-tetrahydro-1,3-benzoxazole, 4,5,6,7-tetrahydro-1H-benzimidazole, 1,4,5,6-tetrahydrocyclopenta[d]imidazole, 4,5,6,7-tetrahydro-1-benzofuran, 4,5,6,7-tetrahydro-1-benzothiophene, 5,6,7,8-tetrahydroquinoline, 5,6,7,8-tetrahydroisoquinoline, 5,6,7,8-tetrahydroquinazoline, indolinyl or isoindolinyl.

In a further aspect the present invention provides compounds of formula (I) wherein Ar is:

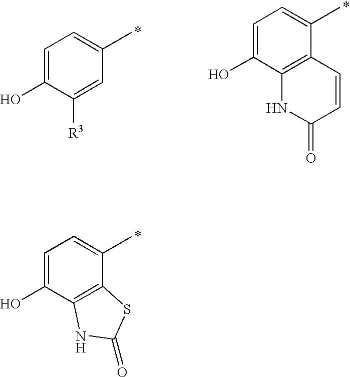

### and R³ is CH₂OH or NHC(O)H.

In another aspect the invention provides a compound of formula (I) wherein Ar is:

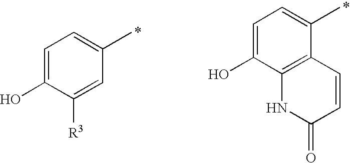

In yet another aspect Ar is:

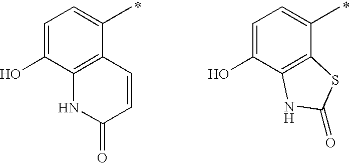

In another aspect Ar is:

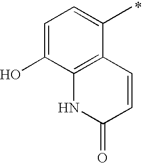

In yet another aspect Ar is:

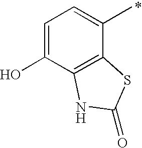

In a further aspect the present invention provides a compound of formula (I) wherein X is a bond or CR²⁷R²⁸ or CR²⁹R³⁰CR³¹R³² (for example X is a bond or CR²⁷R²⁸). In another aspect R²⁷, R²⁸, R²⁹, R³⁰, R³¹ and R³² are, independently, hydrogen or C₁₋₄ alkyl (for example methyl). In yet another aspect R²⁷, R²⁸, R²⁹, R³⁰, R³¹ and R³² are all hydrogen.

In a further aspect the present invention provides a compound of formula (I) wherein X is a bond, CH₂ or C(CH₃)₂ or (CH₂)₂ (for example X is a bond, CH₂ or C(CH₃)₂).

In another aspect the present invention provides a compound of formula (I) wherein X is a bond or CH₂.

In a still further aspect the present invention provides a compound of formula (I) wherein Y is CR³³R³⁴CR³⁵R³⁶, CR³⁷R³⁸CR³⁹R⁴⁰CR⁴¹R⁴² or CR⁴³R⁴⁴CR⁴⁵R⁴⁶CR⁴⁷R⁴⁸CR⁴⁹R⁵⁰, or Y is CR⁵¹R⁵² provided E is C(O)O. In another aspect the present invention provides a compound of formula (I) wherein Y is CR³³R³⁴CR³⁵R³⁶ or CR³⁷R³⁸CR³⁹R⁴⁰CR⁴¹R⁴², or Y is CR⁵¹R⁵² provided E is C(O)O. In yet another aspect Y is CR³³R³⁴CR³⁵R³⁶ or CR³⁷R³⁸CR³⁹R⁴⁰CR⁴¹R⁴². In a further aspect R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹ and R⁵⁰ are, independently, hydrogen or C₁₋₄ alkyl (for example methyl). In a still further aspect R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹ and R⁵⁰ are all hydrogen.

In a still further aspect the present invention provides a compound of formula (I) wherein Y is (CH₂)₂, (CH₂)₃ or CH₂C(CH₃)₂CH₂.

In a further aspect the present invention provides a compound of formula (I) wherein Y is (CH₂)₂.

In another aspect the present invention provides a compound of formula (I) wherein Z is a bond, CR⁵¹R⁵², CR⁵³R⁵⁴CR⁵⁵R⁵⁶ or CR⁵⁷R⁵⁸CR⁵⁹R⁶⁰CR⁶⁰R⁶².

In yet another aspect the present invention provides a compound of formula (I) wherein Z is CR⁵¹R⁵², CR⁵³R⁵⁴CR⁵⁵R⁵⁶ or CR⁵⁷R⁵⁸CR⁵⁹R⁶⁰CR⁶¹R⁶²R⁶².

In a further aspect the present invention provides a compound of formula (I) wherein Z is a bond, CR⁵¹R⁵² or CR⁵³R⁵⁴CR⁵⁵R⁵⁶.

In a still further aspect R⁵¹, R⁵², R⁵³, R⁵⁴, R⁵⁵, R⁵⁶, R⁵⁷, R⁵⁸, R⁵⁹, R⁶⁰, R⁶¹ and R⁶² are independently, hydrogen or C₁₋₄ alkyl (for example methyl). In a still further aspect R⁵¹, R⁵², R⁵³, R⁵⁴, R⁵⁵, R⁵⁶, R⁵⁷, R⁵⁸, R⁵⁹, R⁶⁰, R⁶¹ and R⁶² are all hydrogen

In another aspect the present invention provides a compound of formula (I) wherein Z is a bond, CH₂, (CH₂)₂, (CH₂)₃ or CH₂C(CH₃)₂CH₂.

In yet another aspect the present invention provides a compound of formula (I) wherein Z is CH₂, (CH₂)₂, (CH₂)₃ or CH₂C(CH₃)₂CH₂.

In another aspect the present invention provides a compound of formula (I) wherein Z is a bond, CH₂ or (CH₂)₂ (for example Z is CH₂ or (CH₂)₂).

In a further aspect the present invention provides a compound of formula (I) wherein A is an azetidine, pyrrolidine, piperidine, morpholine, piperazine, azepane, 1,4-diazepane, 3,8-diazabicyclo[3.2.1]octane, 3-azabicyclo[3.1.0]hexane, 8-azabicyclo[3.2.1]octane, 9-azabicyclo[3.3.1]nonane, cyclobutane, cyclopentane, cyclohexane or cycloheptane ring.

In another aspect A is:

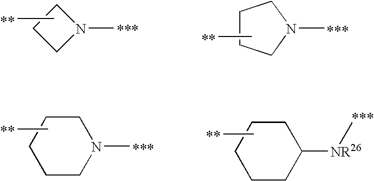

wherein ** is linked to X and *** I linked to Y; the ring A being optionally substituted by hydroxy, C₁₋₄ alkyl (such as methyl) or hydroxy(C₁₋₄ alkyl) (such as HOCH₂); R²⁶ is hydrogen or C₁₋₄ alkyl (for example methyl). In one aspect R²⁶ is hydrogen.

In yet another aspect A is:

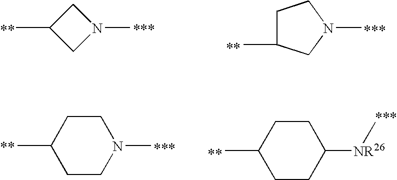

wherein ** is linked to X and *** I linked to Y; A is optionally substituted as recited herein; and R²⁶ is as defined herein.

In a further aspect A is:

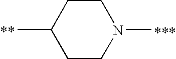

wherein ** is linked to X and *** I linked to Y; A is optionally substituted as recited herein. For example A is unsubstituted or substituted at the ring-carbon linked to X by hydroxy, C₁₋₄ alkyl (such as methyl) or hydroxy(C₁₋₄ alkyl) (such as HOCH₂).

In another aspect of the invention A is unsubstituted.

In still a further aspect of the invention provides compound of formula (I) wherein A is substituted by 1 or 2 substituents independently selected from halogen, C₁₋₄ alkyl (optionally substituted by OR¹²¹, NR¹²²R¹²³ or NR¹²⁴C(O)R¹²⁵), OR¹⁹, NR²⁰R²¹, C(O)NR²²R²³, NR²⁴C(O)R²⁵, CN, S(O)₂R¹²⁶ or S(O)₂NR¹¹⁴R¹¹⁵. R¹⁹, R²⁰, R²¹, R²², R²³, R²⁴, R²⁵, R¹¹⁴, R¹¹⁵, R¹²¹, R¹²², R¹²³, R¹²⁴ and R¹²⁵, are, for example, hydrogen or C₁₋₄ alkyl; and R¹²⁶ is, for example, C₁₋₄ alkyl.

In yet still a further aspect of the invention provides compound of formula (I) wherein A is substituted (for example on the same ring carbon atom as that joining A to X or Y) by 1 substituent independently selected from halogen, C₁₋₄ alkyl (optionally substituted by OR¹²¹) or OR¹⁹ (for example OR¹⁹ is hydroxy or C₁₋₄ alkoxy), R¹⁹ and R¹²¹ are, independently, hydrogen or C₁₋₄ alkyl.

In another aspect of the invention provides compound of formula (I) wherein A is substituted (for example on the same ring carbon atom as that joining A to X or Y) by hydroxyl, C₁₋₄ alkyl (optionally substituted by hydroxy) or C₁₋₄ alkoxy.

In a still further aspect the present invention provides a compound of formula (I) wherein E is O, S(O)₂, NR⁷¹, C(O)NR⁷² or NR⁷³C(O).

In another aspect the present invention provides a compound of formula (I) wherein E is O, S(O)₂, C(O)NR⁷² or NR⁷³C(O).

In a further aspect the present invention provides a compound of formula (I) wherein E is O or C(O)NR⁷²; wherein R⁷² is hydrogen or C₁₋₄ alkyl (such as methyl or ethyl) (for example R⁷² is hydrogen).

In yet another aspect the present invention provides a compound of formula (I) wherein E is C(O)NR⁷², wherein R⁷² is hydrogen or C₁₋₄ alkyl (such as methyl or ethyl). For example R⁷² is hydrogen.

In a further aspect the present invention provides a compound of formula (I) wherein E is O.

In a still further aspect the present invention provides a compound of formula (I) wherein E is O and Z is CH₂CH₂.

In another aspect the present invention provides a compound of formula (I) wherein E is C(O)NR⁷² (for example R⁷² is hydrogen); and Z is CH₂.

In yet another aspect the present invention provides a compound of formula (I) wherein R¹ is aryl, aryloxy or heteroaryl wherein these aryl and heteroaryl rings are optionally substituted by halogen, cyano, trifluoromethyl, phenyl, OR⁷⁸, C(O)NR⁸²R⁸³, S(O)₂R⁸⁷, S(O)₂NR⁸⁸R⁸⁹, NR⁹⁰C(O)R⁹¹, C₁₋₃ alkyl or C₁₋₃ alkoxy; wherein 2 substituents on the aryl or heteroaryl ring which is R¹ can join together to form a 4- to 8-membered which is carbocyclic or heterocyclic (for example containing 1, 2, 3 or 4 heteroatoms independently selected from O, N and S), the fused ring being optionally substituted halogen, C₁₋₄ alkyl, CF₃ or C₁₋₄ alkoxy. The variables R⁷⁸, R⁸², R⁸³, R⁸⁷, R⁸⁸, R⁸⁹, R⁹⁰ and R⁹¹ are as herein defined.

In a further aspect the present invention provides a compound of formula (I) wherein R¹ is unsubstituted phenyl or phenyl substituted by (for example by 1, 2 or 3) the same or different: halogen (such as fluoro or chloro), C₁₋₄ alkyl (such as methyl), C₁₋₄ alkoxy (such a methoxy), cyano, OH, CF₃, OCF₃ or phenyl.

In another aspect the present invention provides a compound of formula (I) wherein R¹ is unsubstituted phenyl or phenyl substituted by (for example by 1 or 2) the same or different: halogen (such as fluoro or chloro) or C₁₋₄ alkyl (such as methyl).

In a still further aspect the present invention provides a compound of formula (I) wherein R¹ is C₃₋₁₀ cycloalkyl (for example cyclopropyl, cyclohexyl or adamantyl) optionally substituted by phenyl.

In another aspect the present invention provides a compound of formula (I) wherein Z is a bond, E is C(O) and R¹ is a group selected from:

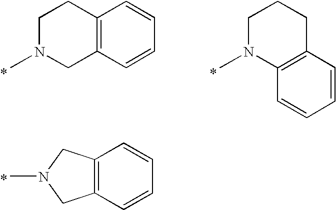

In yet another aspect the present invention provides a compound of formula (I) wherein Z is a bond, E is C(O) and R¹ is:

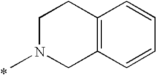

In a further aspect the present invention provides a compound of formula (I) wherein Ar is

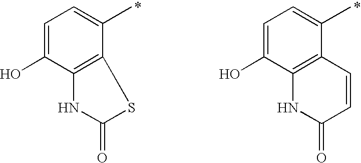

X is a bond, CR²⁷R²⁸ or CR²⁹R³⁰CR³¹R³²;
Y is CR³³R³⁴CR³⁵R³⁶, CR³⁷R³⁸CR³⁹R⁴⁰CR⁴¹R⁴² or CR⁴³R⁴⁴CR⁴⁵R⁴⁶CR⁴⁷R⁴⁸CR⁴⁹R⁵⁰; or Y is CR⁵¹R⁵² provided that E is C(O)O—;
Z is a bond, CR⁵¹R⁵² or CR⁵³R⁵⁴CR⁵⁵R⁵⁶;
A is a heterocyclyl ring selected from

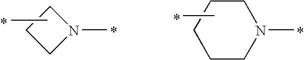

and is linked to Y through the ring nitrogen atom, wherein the heterocyclyl ring is unsubstituted or substituted by 1 substituent (for example the substituent is on the same ring carbon atom as that joining A to Y) independently selected from C₁₋₄ alkyl (optionally substituted by OR¹²¹) or OR¹⁹;

### E is O or C(O)NR⁷²;

R¹ is aryl or C₃₋₁₀ cycloalkyl (optionally substituted by C₁₋₆ alkyl, halogen or phenyl);
wherein aryl is optionally substituted by halogen, cyano, trifluoromethyl, phenyl, OCF₃, OR⁷⁸, C₁₋₆ alkyl (optionally substituted by fluoro, trifluoromethyl, OR⁹³ or NR⁹⁴R⁹⁵) or C₁₋₆ alkoxy (optionally substituted by fluoro, trifluoromethyl, OR¹⁰³ or NR¹⁰⁴R¹⁰⁵); {for example R¹ is phenyl (optionally substituted by halogen, C₁₋₄ alkyl or phenyl) or C₃₋₁₀ cycloalkyl (optionally substituted by phenyl)};
when Z is a bond E can also be C(O) provided R¹ is:

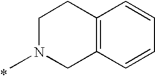

that is optionally substituted as for R¹ above;
R¹⁹, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁷², R⁷⁸, R⁹³, R⁹⁴, R⁹⁵, R¹⁰³, R¹⁰⁴, R¹⁰⁵ and R¹²¹ are, independently, hydrogen or C₁₋₆ alkyl;
or a pharmaceutically acceptable salt thereof.

Examples of compounds of the invention are now listed. Each compound, or a pharmaceutically acceptable salt thereof, represents a single embodiment of the invention. Thus, the invention further provides:
8-Hydroxy-5-{(1R)-1-hydroxy-2-[({1-[2-(2-phenylethoxy)ethyl]piperidin-4-yl}methyl)amino]ethyl}quinolin-2(1H)-one;
8-Hydroxy-5-{(1R)-1-hydroxy-2-[(trans-4-{[2-(2-phenylethoxy)ethyl]amino}cyclohexyl)amino]ethyl}quinolin-2(1H)-one;
8-Hydroxy-4-[(1R)-1-hydroxy-2-({1-[3-(2-phenylethoxy)propyl]piperidin-4-yl}amino)ethyl]quinolin-2(1H)-one;
8-Hydroxy-4-[(1R)-1-hydroxy-2-({1-[2-(2-phenylethoxy)ethyl]piperidin-4-yl}amino)ethyl]quinolin-2(1H)-one;
8-Hydroxy-5-{(1R)-1-hydroxy-2-[({4-hydroxy-1-[2-(2-phenylethoxy)ethyl]piperidin-4-yl}methyl)amino]ethyl}quinolin-2(1H)-one;
N-Benzyl-3-(4-{[(2R)-2-hydroxy-2-(8-hydroxy-2-oxo-1,2-dihydroquinolin-5-yl)ethyl]amino}piperidin-1-yl)propanamide;
N-Benzyl-3-[4-({[(2R)-2-hydroxy-2-(8-hydroxy-2-oxo-1,2-dihydroquinolin-5-yl)ethyl]amino}methyl)piperidin-1-yl]propanamide;
5-[(1R)-2-({1-[3-(3,4-Dihydroisoquinolin-2(1H)-yl)-3-oxopropyl]piperidin-4-yl}amino)-1-hydroxyethyl]-8-hydroxyquinolin-2(1H)-one;
3-(4-{[(2R)-2-Hydroxy-2-(8-hydroxy-2-oxo-1,2-dihydroquinolin-5-yl)ethyl]amino}piperidin-1-yl)-N-(2-phenylethyl)propanamide;
N-(2-Chlorobenzyl)-3-(4-{[(2R)-2-hydroxy-2-(8-hydroxy-2-oxo-1,2-dihydroquinolin-5-yl)ethyl]amino}piperidin-1-yl)propanamide;
3-(4-{[(2R)-2-Hydroxy-2-(8-hydroxy-2-oxo-1,2-dihydroquinolin-5-yl)ethyl]amino}piperidin-1-yl)-N-(2-methoxybenzyl)propanamide;
N-(4-Cyanobenzyl)-3-(4-{[(2R)-2-hydroxy-2-(8-hydroxy-2-oxo-1,2-dihydroquinolin-5-yl)ethyl]amino}piperidin-1-yl)propanamide;
N-(2-Hydroxybenzyl)-3-(4-{[(2R)-2-hydroxy-2-(8-hydroxy-2-oxo-1,2-dihydroquinolin-5-yl)ethyl]amino}piperidin-1-yl)propanamide;
8-Hydroxy-5-{(1R)-1-hydroxy-2-[({4-hydroxy-1-[3-(2-phenylethoxy)propyl]piperidin-4-yl}methyl)amino]ethyl}quinolin-2(1H)-one;
4-Hydroxy-7-{(1R)-1-hydroxy-2-[({1-[2-(2-phenylethoxy)ethyl]piperidin-4-yl}methyl)amino]ethyl}-1,3-benzothiazol-2(3H)-one;
4-Hydroxy-7-{(1R)-1-hydroxy-2-[({1-[3-(2-phenylethoxy)propyl]azetidin-3-yl}methyl)amino]ethyl}-1,3-benzothiazol-2(3H)-one;
4-Hydroxy-7-{(1R)-1-hydroxy-2-[(2-{1-[2-(2-phenylethoxy)ethyl]piperidin-4-yl}ethyl)amino]ethyl}-1,3-benzothiazol-2(3H)-one;
4-Hydroxy-7-{1-hydroxy-2-[1-(2-phenethyloxy-ethyl)-piperidin-4-ylamino]-ethyl}-3H-benzothiazol-2-one;
4-Hydroxy-7-((1R)-1-hydroxy-2-{[(3R)-1-(2-phenethyloxy-ethyl)-piperidin-3-ylmethyl]-amino}-ethyl)-3H-benzothiazol-2-one;
4-Hydroxy-7-((1R)-1-hydroxy-2-{[(3R)-1-(2-phenethyloxy-ethyl)-piperidin-3-ylmethyl]-amino}-ethyl)-3H-benzothiazol-2-one;
5-{(1R)-2-[({1-[3-(benzyloxy)propyl]-4-hydroxypiperidin-4-yl}methyl)amino]-1-hydroxyethyl}-8-hydroxyquinolin-2(1H)-one;
5-{(1R)-2-[({1-[2-(benzyloxy)ethyl]-4-hydroxypiperidin-4-yl}methyl)amino]-1-hydroxyethyl}-8-hydroxyquinolin-2(1H)-one;
N-(2,5-dichlorobenzyl)-3-(4-{[(2R)-2-hydroxy-2-(8-hydroxy-2-oxo-1,2-dihydroquinolin-5-yl)ethyl]amino}piperidin-1-yl)propanamide;
N-(biphenyl-2-ylmethyl)-3-(4-{[(2R)-2-hydroxy-2-(8-hydroxy-2-oxo-1,2-dihydroquinolin-5-yl)ethyl]amino}piperidin-1-yl)propanamide;
N-(2,6-dichlorobenzyl)-3-(4-{[(2R)-2-hydroxy-2-(8-hydroxy-2-oxo-1,2-dihydroquinolin-5-yl)ethyl]amino}piperidin-1-yl)propanamide;
N-(cyclohexylmethyl)-3-(4-{[(2R)-2-hydroxy-2-(8-hydroxy-2-oxo-1,2-dihydroquinolin-5-yl)ethyl]amino}piperidin-1-yl)propanamide;
N-(2-chloro-6-methylbenzyl)-3-(4-{[(2R)-2-hydroxy-2-(8-hydroxy-2-oxo-1,2-dihydroquinolin-5-yl)ethyl]amino}piperidin-1-yl)propanamide;
3-(4-{[(2R)-2-hydroxy-2-(8-hydroxy-2-oxo-1,2-dihydroquinolin-5-yl)ethyl]amino}piperidin-1-yl)-N-[(1R,2S)-2-phenylcyclopropyl]propanamide;
N-(4-chlorobenzyl)-3-(4-{[(2R)-2-hydroxy-2-(8-hydroxy-2-oxo-1,2-dihydroquinolin-5-yl)ethyl]amino}piperidin-1-yl)propanamide;
N-(3-chlorobenzyl)-3-(4-{[(2R)-2-hydroxy-2-(8-hydroxy-2-oxo-1,2-dihydroquinolin-5-yl)ethyl]amino}piperidin-1-yl)propanamide;
N-(2-chloro-6-fluorobenzyl)-3-(4-{[(2R)-2-hydroxy-2-(8-hydroxy-2-oxo-1,2-dihydroquinolin-5-yl)ethyl]amino}piperidin-1-yl)propanamide;
N-(2,3-dichlorobenzyl)-3-(4-{[(2R)-2-hydroxy-2-(8-hydroxy-2-oxo-1,2-dihydroquinolin-5-yl)ethyl]amino}piperidin-1-yl)propanamide;
N-(2-chlorobenzyl)-3-(4-{[(2R)-2-hydroxy-2-(8-hydroxy-2-oxo-1,2-dihydroquinolin-5-yl)ethyl]amino}piperidin-1-yl)-N-methylpropanamide;
5-((1R)-2-{[(1-{2-[2-(3-chlorophenyl)ethoxy]ethyl}-4-hydroxypiperidin-4-yl)methyl]amino}-1-hydroxyethyl)-8-hydroxyquinolin-2(1H)-one;
benzyl (4-{[(2R)-2-hydroxy-2-(8-hydroxy-2-oxo-1,2-dihydroquinolin-5-yl)ethyl]amino}piperidin-1-yl)acetate;
8-Hydroxy-5-[(1R)-1-hydroxy-2-({[4-hydroxy-1-(4-phenoxybutyl)piperidin-4-yl]methyl}amino)ethyl]quinolin-2(1H)-one;
N-1-Adamantyl-3-(4-{[(2R)-2-hydroxy-2-(8-hydroxy-2-oxo-1,2-dihydroquinolin-5-yl)ethyl]amino}piperidin-1-yl)propanamide;
N-(3,5-Dichlorobenzyl-3-(4-{[(2R)-2-hydroxy-2-(8-hydroxy-2-oxo-1,2-dihydroquinolin-5-yl)ethyl]amino}piperidin-1-yl)propanamide;
8-Hydroxy-5-{(1R)-1-hydroxy-2-[({4-(hydroxymethyl)-1-[2-(2-phenylethoxy)ethyl]piperidin-4-yl}methyl)amino]ethyl}quinolin-2(1H)-one;
2,6-Dichloro-N-[2-(4-{[(2R)-2-hydroxy-2-(8-hydroxy-2-oxo-1,2-dihydroquinolin-5-yl)ethyl]amino}piperidin-1-yl)ethyl]benzamide;
8-Hydroxy-5-[(1R)-1-hydroxy-2-({[1-(2-{[(2S)-2-phenylpropyl]oxy}ethyl)piperidin-4-yl]methyl}amino)ethyl]quinolin-2(1H)-one;
5-((1R)-2-{[(1-{2-[2-(2-chlorophenyl)ethoxy]ethyl}-4-hydroxypiperidin-4-yl)methyl]amino}-1-hydroxyethyl)-8-hydroxyquinolin-2(1H)-one;
N-(2,5-Dimethylbenzyl-3-(4-{[(2R)-2-hydroxy-2-(8-hydroxy-2-oxo-1,2-dihydroquinolin-5-yl)ethyl]amino}piperidin-1-yl)propanamide;
N-(Adamant-1-ylmethyl)-3-(4-{[(2R)-2-hydroxy-2-(8-hydroxy-2-oxo-1,2-dihydroquinolin-5-yl)ethyl]amino}piperidin-1-yl)propanamide;
N-(3-Chloro-2-methylbenzyl)-3-(4-{[(2R)-2-hydroxy-2-(8-hydroxy-2-oxo-1,2-dihydroquinolin-5-yl)ethyl]amino}piperidin-1-yl)propanamide;
3-(4-{[(2R)-2-Hydroxy-2-(8-hydroxy-2-oxo-1,2-dihydroquinolin-5-yl)ethyl]amino}piperidin-1-yl)-N-(2-trifluoromethoxybenzyl)-propanamide;
N-((3-Fluoro-5-trifluoromethyl)benzyl)-3-(4-{[(2R)-2-hydroxy-2-(8-hydroxy-2-oxo-1,2-dihydroquinolin-5-yl)ethyl]amino}piperidin-1-yl)propanamide;
N-[2-Fluoro-3-(trifluoromethyl)benzyl]-3-(4-{[(2R)-2-hydroxy-2-(8-hydroxy-2-oxo-1,2-dihydroquinolin-5-yl)ethyl]amino}piperidin-1-yl)propanamide;
N-((2-Chloro-5-trifluoromethyl)benzyl)-3-(4-{[(2R)-2-hydroxy-2-(8-hydroxy-2-oxo-1,2-dihydroquinolin-5-yl)ethyl]amino}piperidin-1-yl)propanamide;
N-((5-Fluoro-2-trifluoromethyl)benzyl)-3-(4-{[(2R)-2-hydroxy-2-(8-hydroxy-2-oxo-1,2-dihydroquinolin-5-yl)ethyl]amino}piperidin-1-yl)propanamide;
3-(4-{[(2R)-2-Hydroxy-2-(8-hydroxy-2-oxo-1,2-dihydroquinolin-5-yl)ethyl]amino}piperidin-1-yl)-N-[(2-trifluoromethyl)benzyl]propanamide;
N-(5-Chloro-2-methylbenzyl)-3-(4-{[(2R)-2-hydroxy-2-(8-hydroxy-2-oxo-1,2-dihydroquinolin-5-yl)ethyl]amino}piperidin-1-yl)propanamide;
N-(3,5-Dimethylbenzyl)-3-(4-{[(2R)-2-hydroxy-2-(8-hydroxy-2-oxo-1,2-dihydroquinolin-5-yl)ethyl]amino}piperidin-1-yl)propanamide;
3-(4-{[(2R)-2-hydroxy-2-(8-hydroxy-2-oxo-1,2-dihydroquinolin-5-yl)ethyl]amino}piperidin-1-yl)-N-(3-trifluoromethoxybenzyl)propanamide;
N-(3-Chloro-2-fluorobenzyl)-3-(4-{[(2R)-2-hydroxy-2-(8-hydroxy-2-oxo-1,2-dihydroquinolin-5-yl)ethyl]amino}piperidin-1-yl)propanamide;
N-[(2-Fluoro-5-trifluoromethyl)benzyl]-3-(4-{[(2R)-2-hydroxy-2-(8-hydroxy-2-oxo-1,2-dihydroquinolin-5-yl)ethyl]amino}piperidin-1-yl)propanamide;
N-[(5-Chloro-2-fluoro)benzyl]-3-(4-{[(2R)-2-hydroxy-2-(8-hydroxy-2-oxo-1,2-dihydroquinolin-5-yl)ethyl]amino}piperidin-1-yl)propanamide;
3-(4-{[(2R)-2-Hydroxy-2-(8-hydroxy-2-oxo-1,2-dihydroquinolin-5-yl)ethyl]amino}piperidin-1-yl)-N-(3-trifluoromethyl)benzylpropanamide;
N-Benzhydryl-3-(4-{[(2R)-2-hydroxy-2-(8-hydroxy-2-oxo-1,2-dihydroquinolin-5-yl)ethyl]amino}piperidin-1-yl)propanamide;
N,N-Dibenzyl-3-(4-{[(2R)-2-hydroxy-2-(8-hydroxy-2-oxo-1,2-dihydroquinolin-5-yl)ethyl]amino}piperidin-1-yl)propanamide;
N-[(3,5-Bistrifluoromethyl)benzyl]-3-(4-{[(2R)-2-hydroxy-2-(8-hydroxy-2-oxo-1,2-dihydroquinolin-5-yl)ethyl]amino}piperidin-1-yl)propanamide;
N-[(Biphenyl-3-yl)methyl]-3-(4-{[(2R)-2-hydroxy-2-(8-hydroxy-2-oxo-1,2-dihydroquinolin-5-yl)ethyl]amino}piperidin-1-yl)propanamide;
3-(4-{[(2R)-2-hydroxy-2-(8-hydroxy-2-oxo-1,2-dihydroquinolin-5-yl)ethyl]amino}piperidin-1-yl)-N-[(5,6,7,8-tetrahydronaphthalen-1-yl)methyl]propanamide;
5-((1R)-2-{[(1-{2-[2-(2,6-Dichlorophenyl)ethoxy]ethyl}-4-hydroxypiperidin-4-yl)methyl]amino}-1-hydroxyethyl)-8-hydroxyquinolin-2(1H)-one;
8-Hydroxy-5-{(1R)-1-hydroxy-2-[({4-hydroxy-1-[2-(2-methyl-2-phenylpropoxy)ethyl]piperidin-4-yl}methyl)amino]ethyl}quinolin-2(1H)-one;
5-{(1R)-2-[({1-[2-(1,1-Dimethyl-2-phenylethoxy)ethyl]-4-hydroxypiperidin-4-yl}methyl)amino]-1-hydroxyethyl}-8-hydroxyquinolin-2(1H)-one;
5-((1R)-2-{[(1-{2-[2-(2,3-Dichlorophenyl)ethoxy]ethyl}-4-hydroxypiperidin-4-yl)methyl]amino}-1-hydroxyethyl)-8-hydroxyquinolin-2(1H)-one;
5-{(1R)-2-[({1-[2-(1,1-Dimethyl-2-phenylethoxy)ethyl]piperidin-4-yl}methyl)amino]-1-hydroxyethyl}-8-hydroxyquinolin-2(1H)-one;
8-Hydroxy-5-{(1R)-1-hydroxy-2-[({1-[2-(2-methyl-2-phenylpropoxy)ethyl]piperidin-4-yl}methyl)amino]ethyl}quinolin-2(1H)-one;
8-Hydroxy-5-{(2R)-1-hydroxy-2-[(4-hydroxy-1-{2-[2-(5,6,7,8-tetrahydronaphthalen-1-yl)ethoxy]ethyl}piperidin-4-ylmethyl)amino]ethyl}-1H-quinolin-2-one;
8-Hydroxy-5-{(1R)-1-hydroxy-2-[({(2S)-1-[3-(2-phenylethoxy)propyl]pyrrolidin-2-yl}methyl)amino]ethyl}quinolin-2(1H)-one; or,
N-[2-(4-{[(2R)-2-hydroxy-2-(8-hydroxy-2-oxo-1,2-dihydroquinolin-5-yl)ethyl]amino}piperidin-1-yl)ethyl]benzenesulfonamide;
   or a pharmaceutically acceptable salt thereof.

The present invention further provides a process for the preparation of a compound of formula (I) or a pharmaceutically acceptable salt thereof as defined above. Suitable processes are described below.

### Process 1

Reacting a compound of formula (II), wherein Ar is as defined in formula (I), with or without a suitable protecting group (PG²) on the phenolic group (as shown below) and wherein PG¹ is a suitable protecting group which may be the same or different to PG², the variables being as defined in formula (I), with a compound of formula (III).

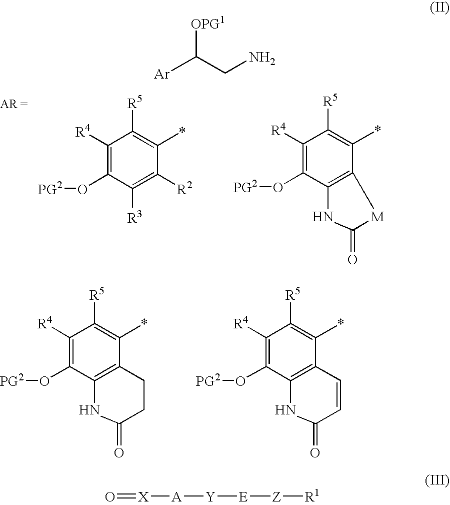

in the presence of a suitable reducing agent, organic acid and solvent under conditions known as “reductive amination” in organic synthesis. Where reference is made herein to protected functional groups or to protecting groups, the protecting groups may be chosen in accordance with the nature of the functional group, for example as described in “Protective Groups in Organic Synthesis”, T. W. Greene and P. G. M. Wuts, John Wiley & Sons Inc, 3^{rd} Edition, 1999, which references therein also describe procedures for replacement of the protecting groups by hydrogen.

Examples of suitable protecting groups for PG¹ include tert-butyldimethylsilyl, tert-butyl-diphenylsilyl, methyldiphenylsilyl, tetrahydropyranyl, trimethylsilylethoxymethyl, phenyloxymethyl, methyloxymethyl, benzyloxymethyl. For example tert-butyldimethyl-silyl is used. Examples of suitable protecting groups for PG² include benzyl, trimethylsilylethoxymethyl, phenyloxymethyl, methyloxymethyl, benzyloxymethyl. In one aspect of the invention PG² is benzyl.

Examples of suitable reducing agents include sodium cyanoborohydride or sodium triacetoxyborohydride or hydrogen in the presence of a suitable catalyst such as palladium on carbon or palladium oxide, in the absence or presence of a suitable organic acid such as a C₁₋₆ aliphatic carboxylic acid. For example sodium triacetoxyborohydride in the presence of acetic acid is used.

Examples of suitable solvents include N-methyl-2-pyrrolidinone, acetonitrile, butyronitrile and ethers such as tetrahydrofuran, 2-methyltetrahydrofuran, 1,4-dioxane, glyme and diglyme. The reaction can be performed at temperatures between 0° C. and 100° C. For example N-methyl-2-pyrrolidinone or tetrahydrofuran at ambient (10-30° C.) temperature is used.

b) Reacting a compound of formula (IV) wherein Ar is as defined in formula (I) with a compound of formula (III) the variables being as defined in formula (I)

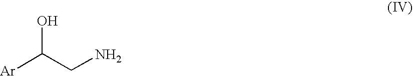

in the presence of a suitable reducing agent, organic acid and solvent.

Examples of suitable reducing agents include sodium cyanoborohydride or sodium triacetoxyborohydride in the presence of a suitable organic acid such as a C₁₋₆ aliphatic carboxylic acid. For example sodium triacetoxyborohydride in the presence of acetic acid is used.

Examples of suitable solvents include N-methyl-2-pyrrolidinone, acetonitrile, butyronitrile, and ethers such as tetrahydrofuran, 2-methyltetrahydrofuran, 1,4-dioxane, glyme and diglyme. The reaction can be performed at temperatures between 0° C. and 100° C. For example N-methyl-2-pyrrolidinone at ambient (10-30° C.) temperature is used.

c) Reacting a compound of formula (II) or (IV) wherein Ar is defined in formula (I), with or without a suitable protecting group (PG²) on the phenolic group (as shown above), with a compound of formula (XXV) in a suitable solvent to produce compounds of formula (I) wherein A is substituted by OR¹⁹ and wherein R¹⁹ is hydrogen.

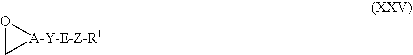

Examples of suitable solvents include dimethylsulphoxide, N,N-dimethylformamide, N-methyl-2-pyrrolidinone, butyronitrile, acetonitrile, and ethers such as tetrahydrofuran, 2-methyltetrahydrofuran, 1,4-dioxane, glym, diglyme, alcohols such methanol, ethanol, isopropanol, tert-butanol or iso-butanol. The process can be performed between 25° C. and 150° C. For example the process is conducted in methanol or ethanol at 50-90° C.

### Process 2

Reacting a compound of general formula (V) wherein Ar is as defined in formula (I) with a suitable protecting group (PG²) on the phenolic group (as shown above), with a compound of formula (VI), wherein PG¹ is a suitable protecting group which may be the same or different to PG² and wherein the variables are as defined in formula (I), and L is a halogen,

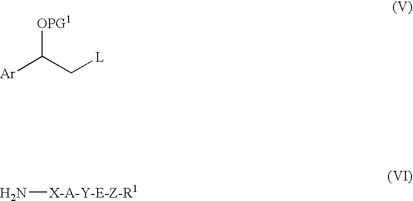

in the presence of a suitable base and solvent (optionally in the presence of a catalyst such as a alkali metal iodide or tri-alkonium iodide may be used) the variables being as defined in formula (I).

Examples of suitable catalysts include Li, Na, K iodides or tri-n-butylammonium iodide. For example potassium iodide is optionally used.

L is a halogen such as chlorine or bromine. For example bromine is used.

Examples of suitable protecting groups for PG¹ include tert-butyldimethylsilyl, tert-butyl-diphenylsilyl, methyldiphenylsilyl, tetrahydropyranyl, trimethylsilylethoxymethyl, phenyloxymethyl, methyloxymethyl or benzyloxymethyl. For example tert-butyldimethyl silyl is used.

Examples of suitable protecting groups for PG² include benzyl, trimethylsilylethoxymethyl, phenyloxymethyl, methyloxymethyl, benzyloxymethyl. For example benzyl is used.

Examples of suitable bases include trialkylamines, such as triethylamine or N,N-diisopropylethylamine, 2,6-lutidine, or pyridine (optionally in the presence of a catalyst such as 4-dimethylaminopyridine), or an alkali metal carbonate or bicarbonate (wherein alkali metal is, for example, Li, Na, K or Cs). For example sodium bicarbonate is used.

Examples of suitable solvents include dimethylsulphoxide, N,N-dimethylformamide, N-methyl-2-pyrrolidinone, butyronitrile, acetonitrile, and ethers such as tetrahydrofuran, 2-methyltetrahydrofuran, 1,4-dioxane, glyme or diglyme. The process can be performed between 25° C. and 150° C. For example the process is conducted in dimethylsulphoxide at 65-145° C.

### Process 3

Reacting a compound of formula (VII), wherein Ar is as defined in formula (I) with a suitable protecting group (PG²) on the phenolic group, with a compound of formula (VI) in the presence of a suitable base and suitable solvent.

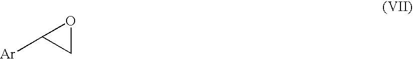

Examples of suitable protecting groups for PG² include benzyl, trimethylsilylethoxymethyl, phenyloxymethyl, methyloxymethyl, benzyloxymethyl. For example benzyl is used.

Examples of suitable bases include trialkylamines, such as triethylamine or N,N-diisopropylethylamine or pyridine (optionally in the presence of a catalyst such as 4-dimethylaminopyridine) or an alkali metal carbonate or bicarbonate (wherein alkali metal is, for example, Li, Na, K or Cs).

Examples of suitable solvents include dimethylsulphoxide, N,N-dimethylformamide, N-methyl-2-pyrrolidinone, butyronitrile, acetonitrile, and ethers such as tetrahydrofuran, 2-methyltetrahydrofuran, 1,4-dioxane, glyme or diglyme. The process can be performed between 25° C. and 150° C. For example the process is conducted in dimethylsulphoxide at 90-145° C.

### Process 4

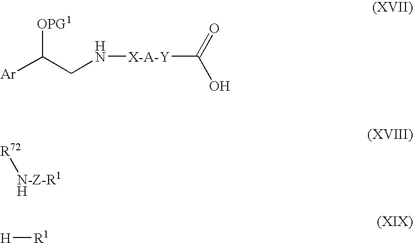

Reacting a compound of formula (XVII) wherein Ar is defined in formula (I), with or without a suitable protecting group (PG²) on the phenolic group (as shown above), either with a compound of formula (XVIII), to produce compounds of formula (I) wherein E is C(O)NR⁷², or with a compound of formula (XIX) to produce compounds of formula (I) wherein Z is a bond, E is C(O) and R¹ is a group selected from:

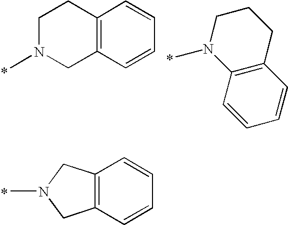

wherein PG¹ is a suitable protecting group or hydrogen which may be the same or different to PG² and wherein the variables are as defined in formula (I).

The reaction is carried out in the presence of a suitable activating coupling agent, a suitable base and a suitable solvent.

Examples of suitable activating coupling agents include carbonyldiimidazole or O-(7-azabenzotriazol-1-yl) N,N,N′,N′-tetramethyluroniumhexafluorophosphate (HATU), or a mixture of N-(3-dimethylaminopropyl)-N′-ethylcarbodiimide hydrochloride and 1-hydroxybenzotriazole. For example, O-(7-azabenzotriazol-1-yl) N,N,N′,N′-tetramethyluroniumhexafluorophosphate (HATU) is used.

Examples of suitable bases include trialkylamines, such as triethylamine or N,N-diisopropylethylamine or pyridine (optionally in the presence of a catalyst such as 4-dimethylaminopyridine). For example triethylamine is used.

Examples of suitable solvents include N,N-dimethylformamide, N-methyl-2-pyrrolidinone, butyronitrile, acetonitrile, and ethers such as tetrahydrofuran, 2-methyltetrahydrofuran, 1,4-dioxane, glyme or diglyme or chlorinated solvents such as dichloromethane or chloroform. The process can be performed between 0° C. and 60° C. For example the process is conducted in N,N-dimethylformamide at ambient (10 to 30° C.) temperature.

Examples of suitable protecting groups for PG¹ include tert-butyldimethylsilyl, tert-butyl-diphenylsilyl, methyldiphenylsilyl, tetrahydropyranyl, trimethylsilylethoxymethyl, phenyloxymethyl, methyloxymethyl or benzyloxymethyl. For example tert-butyldimethyl silyl is used.

Examples of suitable protecting groups for PG² include benzyl, trimethylsilylethoxymethyl, phenyloxymethyl, methyloxymethyl, benzyloxymethyl. For example benzyl is used.

### Preparation of the Intermediates (II), (III), (IV), (V), (VI), (VII), (XVII), (XX), (XXI), (XXII) and (XXV):

Compounds of formula (II) can be prepared by reaction of compounds of formula (V) with a suitable nitrogen nucleophile in the presence (or absence) of a suitable base and solvent followed by reduction. Wherein PG¹ and PG² are suitable protecting groups as described above.

Examples of suitable nucleophiles include metal azides of Li, Na or K.

Examples of suitable bases include trialkylamines, such as triethylamine or N,N-diisopropylethylamine or pyridine (optionally in the presence of a catalyst such as 4-dimethylaminopyridine) or an alkali metal carbonate or bicarbonate (alkali metal is, for example, Li, Na, K or Cs).

Examples of suitable solvents include dimethylsulphoxide, N,N-dimethylamides, N-methyl-2-pyrrolidinone, butyronitrile, acetonitrile, and ethers such as tetrahydrofuran, 2-methyltetrahydrofuran, 1,4-dioxane, glyme and diglyme. The reaction can be performed between 25° C. and 100° C. For example the process is conducted using sodium azide as nucleophile, in N,N-dimethylformamide as solvent and at 40-60° C.

Examples of suitable reducing agents include hydrogen gas in the presence of a suitable catalyst and solvent or triphenylphosphine in the presence of water. For example hydrogen gas in the presence of 10% palladium on charcoal is used in a mixture of tetrahydrofuran and ethanol at ambient (10-30° C.) temperature.

Compounds of formula (III) can be prepared by oxidation of compounds of formula (VIII):

HO—X-A-Y-E-Z-R¹ (VIII)

wherein the variables are as defined in formula (I), by use of a suitable oxidising agent. For example pyridinium chloroctromate or Dess-Martin periodinane in an organic solvent (for example dichloromethane) at ambient (10-30° C.) temperature. Other oxidative procedures may also be employed as known to persons skilled in the art, for example, the Swern oxidation which is outlined in Synthesis, 1981, 3, 165.

Alternatively, compounds of formula (III) can be prepared by reacting compounds of formula (IX) through a ring or exocyclic nitrogen atom with compounds of formula (X) followed by deprotection of the carbonyl protecting group (PG³), wherein the variables are as defined in formula (I)

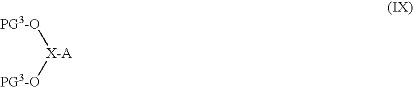

O═Y-E-Z-R¹ (X)

in the presence of a suitable reducing agent, organic acid and solvent.

Examples of suitable protecting group (PG³) include alkyl/cyclic acetals or ketals. For example the ketal derived from ethylene glycol is used.

Examples of suitable reducing agents include sodium cyanoborohydride or sodium triacetoxyborohydride in the presence of a suitable organic acid such as a C₁₋₆ aliphatic carboxylic acid. For example sodium triacetoxyborohydride in the presence of acetic acid is used.

Examples of suitable solvents include N-methyl-2-pyrrolidinone, acetonitrile, butyronitrile, and ethers such as tetrahydrofuran, 2-methyltetrahydrofuran, 1,4-dioxane, glyme and diglyme. The reaction can be performed between 0° C. and 100° C. For example tetrahydrofuran at ambient (10-30° C.) temperature is used.

Alternatively, compounds of formula (III) can be prepared by reaction of compounds of formula (IX) with compounds of formula (XII) followed by subsequent reduction and deprotection

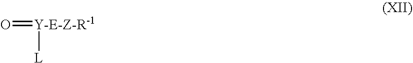

wherein L is a leaving group (such as hydroxyl or halogen, for example chlorine) and the variables are as defined in formula (I). When L is hydroxyl, the reaction is conveniently carried out in the presence of an activating reagent, for example, carbonyldiimidazole or O-(7-azabenzotriazol-1-yl) N,N,N′,N′-tetramethyluroniumhexafluorophosphate (HATU), in organic solvent, for example, N,N-dimethylformamide or dichloromethane, at a temperature, for example in the range 0° C. to 60° C. When L is chlorine, the reaction is conveniently carried out in the presence of a base, for example triethylamine or N,N-diisopropylethylamine in an organic solvent, for example, dichloromethane or tetrahydrofuran at a temperature, for example, in the range 0° C. to 25° C. Examples of subsequent reducing agents include borane-THF complex, lithium aluminium hydride or diisobutylaluminium hydride in a suitable solvent such as tetrahydrofuran, 2-methyltetrahydrofuran, 1,4-dioxane, glyme and diglyme at a temperature, for example, in the range 0° C. to 60° C.

Compounds of formula (VIII) can be prepared by reacting compounds of formula (XI):

HO—X-A (XI)

through a ring or exocyclic nitrogen atom with compounds of formula (X), in the presence of a suitable reducing agent, organic acid and solvent, followed by deprotection of the carbonyl protecting group, wherein X and A are as defined in formula (I).

Examples of suitable reducing agents include sodium cyanoborohydride or sodium triacetoxyborohydride in the presence of a suitable organic acid such as a C₁₋₆ aliphatic carboxylic acid. For example sodium triacetoxyborohydride in the presence of acetic acid is used.

Examples of suitable solvents include N-methyl-2-pyrrolidinone, acetonitrile, butyronitrile, and ethers such as tetrahydrofuran, 2-methyltetrahydrofuran, 1,4-dioxane, glyme and diglyme. The reaction can be performed between 0° C. and 100° C. For example tetrahydrofuran at ambient (10-30° C.) temperature is used.

Alternatively, compounds of formula (VIII) can be prepared by reaction of compounds of formula (XI) with compounds of formula (XII) followed by subsequent reduction and deprotection wherein L is a leaving group (such as hydroxyl or halogen, for example chlorine) and the variables are as defined in formula (I). When L is hydroxyl, the reaction is conveniently carried out in the presence of an activating reagent, for example, carbonyldiimidazole or O-(7-azabenzotriazol-1-yl) N,N,N′,N′-tetramethyluroniumhexafluorophosphate (HATU), in organic solvent, for example, N,N-dimethylformamide or dichloromethane, at a temperature, for example in the range 0° C. to 60° C. When L is chlorine, the reaction is conveniently carried out in the presence of a base, for example triethylamine or N,N-diisopropylethylamine in an organic solvent, for example, dichloromethane or tetrahydrofuran at a temperature, for example, in the range 0° C. to 25° C. Examples of subsequent reducing agents include borane-THF complex, lithium aluminium hydride or diisobutylaluminium hydride in a suitable solvent such as tetrahydrofuran, 2-methyltetrahydrofuran, 1,4-dioxane, glyme and diglyme at a temperature, for example, in the range 0° C. to 60° C.

Compounds of formula (VI) can be prepared from compounds of formula (III) by reaction with suitable sources of ammonia, for example, ammonium chloride in the presence of a suitable reducing agent, organic acid and solvent. Examples of suitable reducing agents include sodium cyanoborohydride or sodium triacetoxyborohydride in the presence of a suitable organic acid such as a C₁₋₆ aliphatic carboxylic acid. For example sodium cyanoborohydride in the presence of acetic acid is used. Examples of suitable solvents include N-methyl-2-pyrrolidinone, acetonitrile, butyronitrile, and ethers such as tetrahydrofuran, 2-methyltetrahydrofuran, 1,4-dioxane, glyme and diglyme. The temperature of the reaction can be performed between 0° C. and 100° C. For example tetrahydrofuran at ambient (0° C. to 30° C.) temperature is used.

Alternatively, compounds of formula (VI) can be prepared by reacting compounds of formula (XIII):

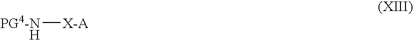

through a ring or exocylic nitrogen atom with compounds of formula (X) followed by removal of the amine protecting group, wherein the variables are as defined in formula (I) in the presence of a suitable reducing agent, organic acid and solvent.

Examples of suitable amine protecting groups (PG⁴) include tert-butyloxycarbonyl, benzyloxycarbonyl, trifluoromethylcarbonyl or phthalimido. For example tert-butyloxycarbonyl is used.

Examples of suitable reducing agents include sodium cyanoborohydride or sodium triacetoxyborohydride in the presence of a suitable organic acid such as a C₁₋₆ aliphatic carboxylic acid. For example sodium triacetoxyborohydride in the presence of acetic acid is used. Examples of suitable solvents include N-methyl-2-pyrrolidinone, acetonitrile, butyronitrile, and ethers such as tetrahydrofuran, 2-methyltetrahydrofuran, 1,4-dioxane, glyme and diglyme. The temperature of the reaction can be performed between 0° C. and 100° C. For example tetrahydrofuran at ambient (0° C. to 30° C.) temperature is used.

Alternatively, compounds of formula (VI) can be prepared by reaction of compounds of formula (XIII) with compounds of formula (XII) followed by subsequent reduction and deprotection wherein L is a leaving group (such as hydroxyl or halogen, eg chlorine) and the variables are as defined in formula (I). When L is hydroxyl, the reaction is conveniently carried out in the presence of an activating reagent, for example, carbonyldiimidazole or O-(7-azabenzotriazol-1-yl) N,N,N′,N′-tetramethyluroniumhexafluorophosphate (HATU), in organic solvent, for example, N,N-dimethylformamide or dichloromethane, at a temperature, for example in the range 0° C. to 60° C. When L is chlorine, the reaction is conveniently carried out in the presence of a base, for example triethylamine or N,N-diisopropylethylamine in an organic solvent, for example, dichloromethane or tetrahydrofuran at a temperature, for example, in the range 0° C. to 25° C. Examples of subsequent reducing agents include borane-THF complex, lithium aluminium hydride or diisobutylaluminium hydride in a suitable solvent such as tetrahydrofuran, 2-methyltetrahydrofuran, 1,4-dioxane, glyme and diglyme at a temperature, for example, in the range 0° C. to 60° C.

Alternatively compounds of formula (III), (VIII), and (VI) may be prepared by reacting the corresponding compounds (IX), (XI) and (XIII) with compounds of formula (XIV):

L-Y-E-Z-R¹ (XIV)

wherein L is a leaving group (such as mesylate, tosylate, triflate or halogen, eg chlorine) and the variables are as defined in formula (I). For example, where L is tosylate is used.

The reaction is conveniently carried out in the presence of a base, for example trialkylamines, such as triethylamine or N,N-diisopropylethylamine or pyridine (optionally in the presence of a catalyst such as 4-dimethylaminopyridine) or an alkali metal carbonate or bicarbonate (alkali metal is, for example, Li, Na, K or Cs) and a suitable solvent.

Examples of suitable solvents include N-methyl-2-pyrrolidinone, acetonitrile, butyronitrile, and ethers such as tetrahydrofuran, 2-methyltetrahydrofuran, 1,4-dioxane, glyme and diglyme. The temperature of the reaction can be performed between 0° C. and 100° C. For example triethylamine in N-methyl-2-pyrrolidinone at 85° C. is used.

Compounds of formula (IV), wherein Ar is as defined in formula (I) with a suitable protecting group (PG²) on the phenolic group, can be prepared by reaction of compounds of formula (XV):

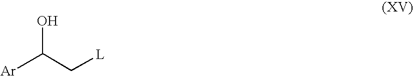

with a suitable nitrogen nucleophile in the presence of a suitable base and solvent followed by reduction. Wherein PG² is a suitable protecting group as described above.

Examples of suitable nucleophiles include metal azides of Li, Na or K. Examples of suitable bases include trialkylamines, such as triethylamine or N,N-diisopropylethylamine or pyridine (optionally in the presence of a catalyst such as 4-dimethylaminopyridine) or an alkali metal carbonate or bicarbonate (wherein alkali metal is, for example Li, Na, K or Cs).

Examples of suitable solvents include dimethylsulphoxide, N,N-dimethylformamide, N-methyl-2-pyrrolidinone, butyronitrile, acetonitrile, and ethers such as tetrahydrofuran, 2-methyltetrahydrofuran, 1,4-dioxane, glyme and diglyme. The temperature of the reaction can be performed between 25° C. and 100° C. For example the nucleophile is sodium azide in N,N-dimethylformamide as solvent at 50° C. (for example 40° C. to 60° C.) is used.

Examples of suitable reducing agents include hydrogen gas in the presence of a suitable catalyst and solvent or triphenylphosphine in the presence of water. For example, hydrogen gas in the presence of 10% palladium on charcoal in a mixture of tetrahydrofuran and ethanol at ambient temperature (10° C. to 30° C.) is used

Compounds of formula (V), wherein Ar is as defined in formula (I) with a suitable protecting group (PG²) on the phenolic group, can be prepared by reaction of compounds of formula (XV) by reacting with a reagent capable of acting as a suitable protecting group (PG¹) such as tert-butyldimethylsilyl chloride or tert-butyldimethylsilyl triflate in the presence of a suitable base and solvent. For example tert-butyldimethylsilyl triflate is used.

Example of suitable bases include trialkylamines, such as triethylamine, N,N-diisopropylethylamine, 2,6-lutidine or pyridine (optionally in the presence of a catalyst such as imidazole or 4-dimethylaminopyridine). For example 2,6-lutidine is used.

Examples of suitable solvents include N,N-dimethylformamide, N-methyl-2-pyrrolidinone, butyronitrile, acetonitrile, and ethers such as tetrahydrofuran, 2-methyltetrahydrofuran, 1,4-dioxane, glyme and diglyme. The reaction can be performed at temperatures between 25° C. and 100° C. For example N,N-dimethylformamide at ambient temperature (10° C. to 30° C.) is used.

Compounds of formula (XV), wherein Ar is as defined in formula (I) with a suitable protecting group (PG²) on the phenolic group, can be prepared by reaction of compounds of formula (XVI), wherein L is a halogen, with a suitable reducing agent such as borane-THF complex in a solvent to produce achiral compounds or with a suitable chiral reducing agent in a suitable solvent to produce single enantiomeric compounds.

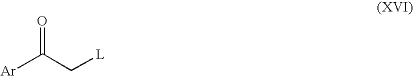

Examples of suitable chiral reducing agents include either (1R,2S)-(+)-cis-1-amino-2-indanol or (R)-(+)-2-methyl-CBS-oxazaborolidine in the presence of a reducing agent such as borane-tetrahydrofuran complex. Suitable solvents include ethers such as tetrahydrofuran, 2-methyltetrahydrofuran, 1,4-dioxane, glyme and diglyme. The reaction can be performed at temperatures between −30° C. to +30° C. For example (R)-(+)-2-methyl-CBS-oxazaborolidine as catalyst with reducing agent borane-THF complex in tetrahydrofuran at −10° C. is used.

Compounds of formula (VII) can be prepared from compounds of formula (XV) in the presence of a suitable base and solvent, wherein L is a halogen and PG² is a suitable protecting group as described above. Examples of suitable bases include an alkali metal carbonate or bicarbonate, such as a carbonate of Li, Na, K or Cs. Examples of suitable solvents include dimethylsulphoxide, N,N-dimethylamides, N-methyl-2-pyrrolidinone, butyronitrile, 2-butanone, water, acetonitrile, and ethers such as tetrahydrofuran, 2-methyltetrahydrofuran, 1,4-dioxane, glyme and diglyme. The reaction can be performed at temperatures between 25° C. and 125° C. For example potassium carbonate in butanone and is water as solvent at reflux is used.

Compounds of formula (XXV) can be prepared by reacting a compound of formula (III) with a sulphur ylide for example timethylsulphonium- or trimethylsulphoxonium-methylide (prepared from timethylsulphonium- or trimethylsulphoxonium iodide and a base such as sodium hydride or potassium- or sodium-tert-butoxide) in a solvent such as dimethylsulfoxide and/or ethers such as tetrahydrofuran, 2-methyltetrahydrofuran, 1,4-dioxane, glyme and diglyme. The reaction can be performed at temperatures in the range from −10 to 100° C. For example trimethylsulphoxnium iodide with sodium hydride in mixtures of dimethylsulphoxide and tetrahydrofuran are used at ambient temperature.

Compounds of formula (XVII), wherein Ar is as defined in formula (I) with or without a suitable protecting group (PG²), on the phenolic group (as shown above) can be prepared from compounds of formula (XX) in the presence of a suitable acid or base and a suitable solvent, wherein PG⁵ and PG¹ are suitable protecting groups or hydrogen as described above.

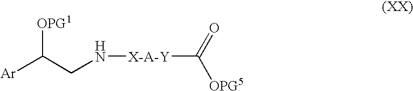

Examples of suitable protecting groups for PG¹ include tert-butyldimethylsilyl, tert-butyl-diphenylsilyl, methyldiphenylsilyl, tetrahydropyranyl, trimethylsilylethoxymethyl, phenyloxymethyl, methyloxymethyl or benzyloxymethyl. For example tert-butyldimethyl silyl is used.

Examples of suitable protecting groups for PG⁵ include, methyl, ethyl, propyl, isopropyl, butyl, isobutyl, tert-butyl or benzyl. For example tert-butyl is used.

Examples of suitable acids and bases include trifluoroacetic acid, hydrochloric acid, alkali metal hydroxides. Examples of suitable solvents include dichloromethane, methanol, trifluoroacetic acid, tetrahydrofuran, water. The reaction can be performed at temperatures between 0° C. and 60° C. For example, when PG⁵ is tert-butyl, trifluoroacetic acid in trifluoroacetic acid as solvent at ambient (10° C. to 30° C.) temperature is used.

Compounds of formula (XX) can be prepared from the reaction of compounds of formula (XXI) with compounds of formula (II) or (IV) in the presence of a suitable reducing agent, acid and solvent.

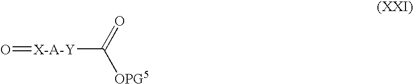

Examples of suitable reducing agents include sodium cyanoborohydride or sodium triacetoxyborohydride or hydrogen in the presence of a suitable catalyst such as palladium on carbon or palladium oxide, in the absence or presence of a suitable organic acid such as a C₁₋₆ aliphatic carboxylic acid. For example sodium triacetoxyborohydride in the presence of acetic acid is used.

Examples of suitable solvents include N-methyl-2-pyrrolidinone, acetonitrile, butyronitrile and ethers such as tetrahydrofuran, 2-methyltetrahydrofuran, 1,4-dioxane, glyme and diglyme. The reaction can be performed at temperatures between 0° C. and 100° C. For example N-methyl-2-pyrrolidinone or tetrahydrofuran at ambient (10-30° C.) temperature is used.

Alternatively, compounds of formula (XX) can be prepared from the reaction of compounds of formula (XXII) with compounds of formula (V) in the presence of a suitable base and solvent (optionally in the presence of a catalyst such as a alkali metal iodide or tri-alkonium iodide may be used).

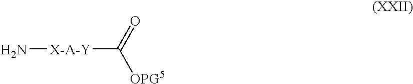

Examples of suitable catalysts include Li, Na, K iodides or tri-n-butylammonium iodide. For example potassium iodide is used.

Examples of suitable bases include trialkylamines, such as triethylamine or N,N-diisopropylethylamine, 2,6-lutidine, or pyridine (optionally in the presence of a catalyst such as 4-dimethylaminopyridine), or an alkali metal carbonate or bicarbonate (wherein alkali metal is, for example, Li, Na, K or Cs). For example sodium bicarbonate is used.

Examples of suitable solvents include dimethylsulphoxide, N,N-dimethylformamide, N-methyl-2-pyrrolidinone, butyronitrile, acetonitrile, and ethers such as tetrahydrofuran, 2-methyltetrahydrofuran, 1,4-dioxane, glyme or diglyme. The process can be performed between 25° C. and 150° C. For example the process is conducted in dimethylsulphoxide at 65-145° C.

Compounds of formula (XXI) can be prepared from the reaction of compounds of formula (XXIII) with compounds of formula (XXIV) in the presence of a suitable base and solvent wherein L is a suitable leaving group (such as a halogen, eg Cl, Br, I). For example where L is chlorine and bromine is used.

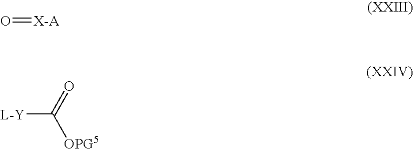

Examples of suitable bases include trialkylamines, such as triethylamine or N,N-diisopropylethylamine or pyridine or 1,5-diazabicyclo[5.4.0]undec-5-ene (optionally in the presence of a catalyst such as 4-dimethylaminopyridine) or an alkali metal carbonate or bicarbonate (wherein alkali metal is, for example, Li, Na, K or Cs). For example, triethylamine is used.

Examples of suitable solvents include dimethylsulphoxide, N,N-dimethylformamide, N-methyl-2-pyrrolidinone, butyronitrile, acetonitrile, chloroform, dichloromethane and ethers such as tetrahydrofuran, 2-methyltetrahydrofuran, 1,4-dioxane, glyme or diglyme. The is process can be performed between 25° C. and 150° C. For example the process is conducted in chloroform at reflux.

Compounds of formula (XXII) can be prepared from the reaction of compounds of formula (XIII) with compounds of formula (XXIV) in the presence of a suitable base and solvent, followed by removal of the amine protecting group.

Examples of suitable bases include trialkylamines, such as triethylamine or N,N-diisopropylethylamine or pyridine or 1,5-diazabicyclo[5.4.0]undec-5-ene (optionally in the presence of a catalyst such as 4-dimethylaminopyridine) or an alkali metal carbonate or bicarbonate (wherein alkali metal is, for example, Li, Na, K or Cs). For example, triethylamine is used.

Examples of suitable solvents include dimethylsulphoxide, N,N-dimethylformamide, N-methyl-2-pyrrolidinone, butyronitrile, acetonitrile, chloroform, dichloromethane and ethers such as tetrahydrofuran, 2-methyltetrahydrofuran, 1,4-dioxane, glyme or diglyme. The process can be performed between 25° C. and 150° C. For example the process is conducted in chloroform at reflux.

Alternatively, compounds of formula (XXII) can be prepared from compounds of formula (XXI) by reaction with a suitable source of ammonia, in the presence of a suitable reducing agent, acid and solvent.

Examples of suitable sources of ammonia include ammonia gas, ammonium chloride and ammonium acetate.

Examples of suitable reducing agents include sodium cyanoborohydride or sodium triacetoxyborohydride or hydrogen in the presence of a suitable catalyst such as palladium on carbon or palladium oxide, in the absence or presence of a suitable organic acid such as a C₁₋₆ aliphatic carboxylic acid. For example sodium triacetoxyborohydride in the presence of acetic acid is used.

Examples of suitable solvents include N-methyl-2-pyrrolidinone, acetonitrile, butyronitrile and ethers such as tetrahydrofuran, 2-methyltetrahydrofuran, 1,4-dioxane, glyme and diglyme. The reaction can be performed at temperatures between 0° C. and 100° C. For example N-methyl-2-pyrrolidinone or tetrahydrofuran at ambient (10-30° C.) temperature is used.

Compounds of formula (II), wherein PG¹ and PG² are suitable protecting groups, (IX), (X), (XI), (XII), (XIII), (XIV), (XVI), (XVIII), (XIX), (XXIII) and (XXIV) can be prepared by processes known in the literature or using known methods in the literature or are available commercially.

The present invention further relates to novel chiral intermediate compounds, for example compounds of formula (IV)

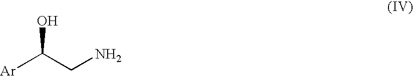

wherein Ar is:

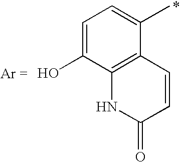

such as (5-[(1R)-2-amino-1-hydroxyethyl]-8-hydroxyquinolin-2(1H)-one) prepared as described above.

The present invention also further relates to an intermediate compound of formula (XX):

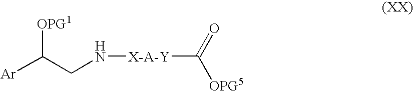

wherein
Ar is as defined in formula (I);
PG¹ is suitable protecting group;
X is a bond;
A is piperidinyl linked to X through the 4-position and N-linked to Y;

### Y is (CH₂)₂; and

PG⁵ is either hydrogen or a suitable protecting group.

PG¹ is, for example, tert-butyldimethylsilyl, tert-butyl-diphenylsilyl or methyldiphenylsilyl. In an embodiment of the invention PG¹ is, for example, tert-butyldimethyl silyl.

PG⁵ is, for example, methyl, ethyl, propyl, isopropyl, butyl, iso-butyl, tert-butyl or benzyl. In an embodiment of the invention PG⁵ is, for example, tert-butyl.

Compounds of formula (I) can be converted into further compounds of formula (I) using standard procedures.

It will be appreciated by those skilled in the art that in the processes of the present invention certain functional groups such as hydroxyl or amino groups in the reagents may need to be protected by protecting groups. Thus, the preparation of the compounds of formula (I) may involve, at an appropriate stage, the removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 3^{rd} edition, T. W. Greene and P. G. M. Wuts, Wiley-Interscience (1999).

The compounds of formula (I) and their pharmaceutically acceptable salts can be used in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) or adenovirus; or eosinophilic esophagitis;
2. bone and joints: arthritides associated with or including osteoarthritis/osteoarthrosis, both primary and secondary to, for example, congenital hip dysplasia; cervical and lumbar spondylitis, and low back and neck pain; osteoarthritis; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;
3. pain and connective tissue remodelling of musculoskeletal disorders due to injury [for example sports injury] or disease: arthritides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,
15. gastrointestinal tract: Coeliac disease, proctitis, eosinopilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema.

Thus, the present invention provides a compound of formula (I) or a pharmaceutically-acceptable salt thereof as hereinbefore defined for use in therapy.

In a further aspect, the present invention provides the use of a compound of formula (I) or a pharmaceutically acceptable salt thereof as hereinbefore defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

The invention still further provides a method of treating, or reducing the risk of, an inflammatory disease or condition (including a reversible obstructive airways disease or condition) which comprises administering to a patient in need thereof a therapeutically effective amount of a compound of formula (I) or a pharmaceutically acceptable salt thereof as hereinbefore defined.

In particular, the compounds of this invention may be used in the treatment of adult respiratory distress syndrome (ARDS), pulmonary emphysema, bronchitis, bronchiectasis, chronic obstructive pulmonary disease (COPD), asthma and rhinitis.

For the above-mentioned therapeutic uses the dosage administered will, of course, vary with the compound employed, the mode of administration, the treatment desired and the disorder indicated. For example, the daily dosage of the compound of the invention, if inhaled, may be in the range from 0.05 micrograms per kilogram body weight (μg/kg) to 100 micrograms per kilogram body weight (μg/kg). Alternatively, if the compound is administered orally, then the daily dosage of the compound of the invention may be in the range from 0.01 micrograms per kilogram body weight (μg/kg) to 100 milligrams per kilogram body weight (mg/kg).

The compounds of formula (I) and pharmaceutically acceptable salts thereof may be used on their own but will generally be administered in the form of a pharmaceutical composition in which the formula (I) compound/salt (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition will for example comprise from 0.05 to 99% w (percent by weight), such as from 0.05 to 80% w, for example from 0.10 to 70% w, and such as from 0.10 to 50% w, of active ingredient, all percentages by weight being based on total composition.

The present invention also provides a pharmaceutical composition comprising a compound of formula (I) or a pharmaceutically acceptable salt thereof as hereinbefore defined, in association with a pharmaceutically acceptable adjuvant, diluent or carrier.

The invention further provides a process for the preparation of a pharmaceutical composition of the invention which comprises mixing a compound of formula (I) or a pharmaceutically acceptable salt thereof as hereinbefore defined with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane (HFA) aerosols and dry powder formulations, for example, formulations in the inhaler device known as the Turbuhaler®; or systemically, e.g. by oral administration in the form of tablets, capsules, syrups, powders or granules; or by parenteral administration in the form of solutions or suspensions; or by subcutaneous administration; or by rectal administration in the form of suppositories; or transdermally.

Dry powder formulations and pressurized HFA aerosols of the compounds of the invention may be administered by oral or nasal inhalation. For inhalation, the compound is desirably finely divided. The finely divided compound has, for example, a mass median diameter of less than 10 μm, and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

The compounds of the invention may also be administered by means of a dry powder inhaler. The inhaler may be a single or a multi dose inhaler, and may be a breath actuated dry powder inhaler.

One possibility is to mix the finely divided compound of the invention with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

For oral administration the compound of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compound of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The compounds of the invention may also be administered in conjunction with other compounds used for the treatment of the above conditions.

The invention therefore further relates to combination therapies wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with the following agents: non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The present invention still further relates to the combination of a compound of the invention, or a pharmaceutically acceptable salt thereof, together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline.

In addition the invention relates to a combination of a compound of the invention, or a pharmaceutically acceptable salt thereof, with a monoclonal antibody targeting B-Lymphocytes (such as CD20 (rituximab), MRA-aIL16R and T-Lymphocytes, CTLA4-Ig, HuMax Il-15).

The present invention still further relates to the combination of a compound of the invention, or a pharmaceutically acceptable salt thereof, with a modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C-C family); CXCR1, CXCR2, CXCR3, CXCR4 and CXCR5 (for the C-X-C family) and CX₃CR1 for the C-X₃-C family.

The present invention further relates to the combination of a compound of the invention, or a pharmaceutically acceptable salt thereof, with an inhibitor of matrix metalloprotease (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention, or a pharmaceutically acceptable salt thereof, and a leulcotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY x 1005.

The present invention further relates to the combination of a compound of the invention, or a pharmaceutically acceptable salt thereof, and a receptor antagonist for leukotrienes (LT) B4, LTC4, LTD4, and LTE4. selected from the group consisting of the phenothiazin-3-1s such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention, or a pharmaceutically acceptable salt thereof, and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention, or a pharmaceutically acceptable salt thereof, and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention, or a pharmaceutically acceptable salt thereof, and a proton pump inhibitor (such as omeprazole) or a gastroprotective histamine type 2 receptor antagonist.

The present invention further relates to the combination of a compound of the invention, or a pharmaceutically acceptable salt thereof, and an antagonist of the histamine type 4 receptor.

The present invention still further relates to the combination of a compound of the invention, or a pharmaceutically acceptable salt thereof, and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention, or a pharmaceutically acceptable salt thereof, and an anticholinergic agents including muscarinic receptor (M1, M2, and M3) antagonist such as atropine, hyoscine, glycopyrrrolate, ipratropium bromide, tiotropium bromide, oxitropium bromide, pirenzepine or telenzepine.

The present invention further relates to the combination of a compound of the invention, or a pharmaceutically acceptable salt thereof, and a chromone, such as sodium cromoglycate or nedocromil sodium.

The present invention still further relates to the combination of a compound of the invention, or a pharmaceutically acceptable salt thereof, with a glucocorticoid, such as flunisolide, triamcinolone acetonide, beclomethasone dipropionate, budesonide, fluticasone propionate, ciclesonide or mometasone furoate.

The present invention further relates to the combination of a compound of the invention, or a pharmaceutically acceptable salt thereof, with an agent that modulates a nuclear hormone receptor such as PPARs.

The present invention still further relates to the combination of a compound of the invention, or a pharmaceutically acceptable salt thereof, together with an immunoglobulin (Ig) or Ig preparation or an antagonist or antibody modulating Ig function such as anti-IgE (for example omalizumab).

The present invention further relates to the combination of a compound of the invention, or a pharmaceutically acceptable salt thereof, and another systemic or topically-applied anti-inflammatory agent, such as thalidomide or a derivative thereof, a retinoid, dithranol or calcipotriol.

The present invention still further relates to the combination of a compound of the invention, or a pharmaceutically acceptable salt thereof, and combinations of aminosalicylates and sulfapyridine such as sulfasalazine, mesalazine, balsalazide, and olsalazine; and immunomodulatory agents such as the thiopurines, and corticosteroids such as budesonide.

The present invention further relates to the combination of a compound of the invention, or a pharmaceutically acceptable salt thereof, together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention, or a pharmaceutically acceptable salt thereof, and a cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

The present invention further relates to the combination of a compound of the invention, or a pharmaceutically acceptable salt thereof, and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a compound of the invention, or a pharmaceutically acceptable salt thereof, and an agent for the treatment of acute or chronic pain, such as a centrally or peripherally-acting analgesic (for example an opioid or derivative thereof), carbamazepine, phenyloin, sodium valproate, amitryptiline or other anti-depressant agents, paracetamol, or a non-steroidal anti-inflammatory agent.

The present invention further relates to the combination of a compound of the invention, or a pharmaceutically acceptable salt thereof, together with a parenterally or topically-applied (including inhaled) local anaesthetic agent such as lignocaine or a derivative thereof. A compound of the present invention, or a pharmaceutically acceptable salt thereof, can also be used in combination with an anti-osteoporosis agent including a hormonal agent such as raloxifene, or a biphosphonate such as alendronate.

The present invention still further relates to the combination of a compound of the invention, or a pharmaceutically acceptable salt thereof, together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B.sub1.- or B.sub2.-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK.sub1. or NK.sub3. receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of P38; (xxv) agent modulating the function of Toll-like receptors (TLR), (xxvi) agent modulating the activity of purinergic receptors such as P2X7; (xxvii) inhibitor of transcription factor activation such as NFkB, API, or STATS; or, (xxviii) a glucocorticoid receptor agonist.

In a further aspect the present invention provides a combination (for example for the treatment of COPD, asthma or allergic rhinitis) of a compound of formula (I) and one or more agents is selected from the list comprising:
a non-steroidal glucocorticoid receptor (GR-receptor) agonist;
a steriod (such as budesonide or fluticasone)
PDE4 inhibitor including an inhibitor of the isoform PDE4D;
a muscarinic receptor antagonist (for example a M1, M2 or M3 antagonist, such as a selective M3 antagonist) such as ipratropium bromide, tiotropium bromide, oxitropium bromide, pirenzepine or telenzepine;
a modulator of chemokine receptor function (such as a CCR1 receptor antagonist); or,
an inhibitor of p38 kinase function.

A compound of the invention, or a pharmaceutically acceptable salt thereof, can also be used in combination with an existing therapeutic agent for the treatment of cancer, for example suitable agents include:

(i) an antiproliferative/antineoplastic drug or a combination thereof, as used in medical oncology, such as an alkylating agent (for example cisplatin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine, or vinorelbine, or a taxoid such as taxol or taxotere); or a topoisomerase inhibitor (for example an epipodophyllotoxin such as etoposide, teniposide, amsacrine, topotecan or a camptothecin);
(ii) a cytostatic agent such as an antioestrogen (for example tamoxifen, toremifene, raloxifene, droloxifene or iodoxyfene), an estrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;
(iii) an agent which inhibits cancer cell invasion (for example a metalloproteinase inhibitor like marimastat or an inhibitor of urokinase plasminogen activator receptor function);
(iv) an inhibitor of growth factor function, for example: a growth factor antibody (for example the anti-erbb2 antibody trastuzumab, or the anti-erbb1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;
(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ3 function or an angiostatin);
(vi) a vascular damaging agent such as combretastatin A4, or a compound disclosed in WO 99/02166, WO 00/40529, WO 00/41669, WO 01/92224, WO 02/04434 or WO 02/08213;
(vii) an agent used in antisense therapy, for example one directed to one of the targets listed above, such as ISIS 2503, an anti-ras antisense;
(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or is a bacterial nitroreductase enzyme and approaches to increase patient tolerance to chemotherapy or radiotherapy such as multi-drug resistance gene therapy; or
(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The present invention will now be further explained by reference to the following illustrative Examples.

### General Methods

### General Methods

¹H NMR spectra were recorded on a Varian Inova 400 MHz or a Varian Mercuty-VX 300 MHz instrument. The central peaks of chloroform-d (δ_{H} 7.27 ppm), dimethylsulfoxide-d₆ (δ_{H} 2.50 ppm), acetonitrile-d₃ (δ_{H} 1.95 ppm) or methanol-d₄ (δ_{H} 3.31 ppm) were used as internal references.

Compounds in the Examples were named using the ACD LABS v8.0 naming software.

Column chromatography was carried out using silica gel (0.040-0.063 mm, Merdo). Unless stated otherwise, starting materials were commercially available. All solvents and commercial reagents were of laboratory grade and were used as received.

Reverse phase High Pressure Liquid Chromatography (HPLC) purification was performed using either a Waters Micromass LCZ with a Waters 600 pump controller, Waters 2487 detector and Gilson FC024 fraction collector or a Waters Delta Prep 4000 or a Gilson Auto Purification System, using a ACE®, Symmetry®, NovaPak® or Xterra® reverse phase silica column.

The following method was used for LC/MS analysis:

Instrument Agilent 1100; Column Waters Symmetry 2.1×30 mm; Mass APCI; Flow rate 0.7 ml/min; Wavelength 254 nm; Solvent A: water+0.1% TFA; Solvent B: acetonitrile+0.1% TFA; Gradient 15-95%/B 8 min, 95% B1 min.

Analytical chromatography was run on a Symmetry C₁₈-column, 2.1×30 mm with 3.5 μm particle size, with acetonitrile/water/0.1% trifluoroacetic acid as mobile phase in a gradient from 5% to 95% acetonitrile over 8 minutes at a flow of 0.7 ml/min.

The abbreviations or terms used in the examples have the following meanings:

HPLC high performance liquid chromatography
AcOH acetic acid
CHCl₃ chloroform
DCM dichloromethane

### DMF N,N-dimethylformamide

DMSO dimethylsulfoxide
Et₂O diethyl ether
EtOAc ethyl acetate
MgSO₄ magnesium sulfate
Na₂SO₄ sodium sulphate
NMP N-methyl-2-pyrrolidinone
THF tetrahydrofuran
H₂O water
NH₃ ammonia
TFA trifluoroacetic acid
TsOH tosic acid
NaCl sodium chloride
HATU O-(7-azabenzotriazol-1-yl)-N,N,N′,N′-tetramethyluronium hexafluorophosphate
PyBROP Benzotriazol-1-yl-oxytripyrrolidinephosphonium Hexafluorophosphate

### NMO N-methylmorpholine N-oxide

TPAP terapropylammonium perruthenate

### EXAMPLE 1

### 8-Hydroxy-5-{(1R)-1-hydroxy-2-[{1-[2-(2-phenylethoxy)ethyl]piperidin-4-yl}methyl)amino]ethyl}quinolin-2(1H)-one

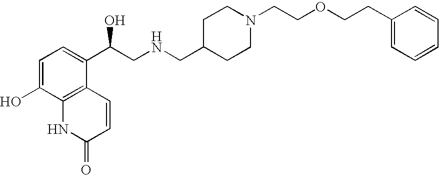

### i) [1-(2-Phenethyloxy-ethyl)-piperidin-4-yl]-methanol

A solution of 2-phenethyloxyacetaldehyde (WO 94/27601) (0.72 g) and 4-piperidine-methanol (0.5 g) in methanol (20 mL) was treated with AcOH (20 mg) and stirred at room temperature for 30 min. At the end of this time, sodium cyanoborohydride (103 mg) was added and the mixture was stirred for 18 h at room temperature. The reaction mixture was basified by addition of concentrated aqueous NH₃ (1 mL) and the solvent was removed under reduced pressure. The crude product was purified by column chromatography eluting with 1% concentrated aqueous NH₃ and 5% ethanol in DCM to give the sub-title compound. Yield: 0.1 g

¹H NMR (CDCl₃) δ 7.30-7.18 (m, 5H), 3.67-3.64 (m, 2H), 3.60-3.57 (m, 2H), 3.49-3.47 (m, 2H), 2.95-2.87 (m, 4H), 2.58-2.55 (m, 2H), 2.02-1.96 (m, 2H), 1.72-1.69 (m, 2H), 1.54-1.43 (m, 1H), 1.33-1.23 (m, 2H).

### ii) 1-(2-Phenethyloxy-ethyl)-piperidine-4-carbaldehyde

A solution of DMSO (0.136 mL) in DCM (0.5 mL) was added to oxalyl chloride (0.130 mL) in DCM (5 mL) at −78° C. The reaction was stirred for 15 min at −78° C., treated with the product of step (i) [1-(2-phenethyloxy-ethyl)-piperidin-4-yl]-methanol) (0.23 g) in DCM (3 mL), and stirred for a further 15 min at −78° C. Triethylamine (0.53 mL) was added and the reaction was allowed to warm to room temperature over 1 h. The mixture was subsequently quenched with concentrated aqueous NH₃ solution and concentrated in vacuo. The crude product was azeotroped twice with toluene and used without further purification in the next step (iii).

MS APCI+ 262 [M+H]⁺

### iii) (R)-8-Hydroxy-5-(1-hydroxy-2-{[1-(2-phenethyloxy-ethyl)-piperidin-4-ylmethyl]-amino}-ethyl)-1H-quinolin-2-one

The crude product from step (ii) was dissolved in methanol (5 mL). The product of example 5 step (ii) [5-((1R)-2-amino-1-{[tert-butyl(dimethyl)silyl]oxy}ethyl)-8-hydroxyquinolin-2(1H)-one (WO 2004/106333)] (0.25 g), was then added along with AcOH (0.043 mL). After stirring at room temperature for 2 h, sodium cyanoborohydride (57 mg) was added and the reaction mixture was stirred for 18 h at room temperature. The reaction was quenched with concentrated aqueous NH₃ solution and concentrated in vacuo. The crude material was redissolved in DCM, filtered and purified by column chromatography eluting with 1% NH₃/9% methanol in DCM. The product fractions were concentrated in vacuo. The product was redissolved in THF (5 mL) and treated with triethylamine trihydrofluoride (1.24 mL) and stirred for 18 h at room temperature. The reaction was concentrated in vacuo and the residue applied to Argonaut Technologies MP-TsOH(65) resin column (1 g). The resin was washed with methanol and the product eluted with 3M methanolic NH₃. The methanolic NH₃ fraction was concentrated in vacuo, the residue redissolved in methanol, filtered, then purified by reverse phase HPLC using an Xterra® C8 5 micron 19×50 mm column eluting with a gradient of acetonitrile in 0.2% aqueous 0.880 NH₃ over 6 min. at 20 ml/min to give the title compound. Yield: 55 mg.

MS APCI+ 466 [M+H]⁺

¹H NMR (CDCl₃, 50° C.) δ 7.84 (s, 1H), 7.27-7.22 (m, 2H), 7.21-7.14 (m, 3H), 6.94-6.88 (m, 1H), 6.75-6.67 (m, 1H), 6.41-6.32 (m, 1H), 4.88 (s, 1H), 3.66-3.60 (m, 2H), 3.57-3.50 (m, 2H), 2.89-2.76 (m, 4H), 2.75-2.58 (m, 2H), 2.56-2.31 (m, 4H), 1.96-1.84 (m, 3H), 1.66-1.51 (m, 2H), 1.42-1.30 (m, 1H), 1.24-1.09 (m, 2H).

### EXAMPLE 2

### 8-Hydroxy-5-{(1R)-1-hydroxy-2-[(trans-4-{[2-(2-phenylethoxy)ethyl]amino}cyclohexyl)amino]ethyl}quinolin-2(1H)-one

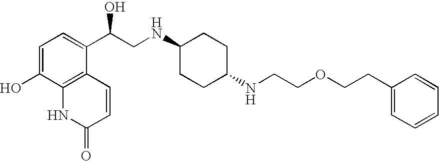

### i) 8-(Benzyloxy)-5-(bromoacetyl)quinolin-2(1H)-one

To a solution of 5-acetyl-8-(benzyloxy)quinolin-2(1H)-one (WO 2005/123684) (18.05 g) in DCM (200 mL) at 0° C. was added boron trifluoride etherate complex (9.2 mL) dropwise over 15 min and then the mixture allowed to warm to room temperature forming a thick yellow suspension. The mixture was heated at 40° C. and a solution of bromine (3.4 mL) in DCM (100 mL) added slowly over 40 min. After a further 15 min the mixture was allowed to reach room temperature before removing the volatiles on a rotary evaporator. The residue was triturated with excess 10% aqueous sodium carbonate for 1 h. The gummy solid collected by filtration and further washed with H₂O and drying the solid in vacuo at 40° C. overnight. Purification was by further washing the solid with 1:1 DCM/methanol solutions and filtration followed by drying in vacuo at 40° C. to give the sub-title compound as a off white solid. Yield: 14.5 g

MS APCI+ 372/374 [M+H]⁺

### ii) 8-(Benzyloxy)-5-[(1R)-2-bromo-1-hydroxyethyl]quinolin-2(1H)-one

The product of step (i) (8-(benzyloxy)-5-(bromoacetyl)quinolin-2(1H)-one) (5.0 g) was placed in an oven-dried flask and dried for 2 d under vacuum at 40° C., then to it was added dry THF (100 mL). (R)-(+)-2-Methyl-CBS-oxazaborolidine (2.20 mL, 1.0M in toluene) was added and the mixture cooled to −10° C. Borane-THF complex (16.2 mL, 1.0M) was added over 3.5 h using a syringe pump. The reaction mixture was stirred at −10 to 0° C. for 1 h. Methanol (100 mL) was added and the volatiles removed in vacuo, followed by azeotroping with methanol (×3). The residue was dissolved in boiling acetonitrile (140 mL), allowed to cool slowly and filtered to afford the sub-title compound as a pale yellow solid. Yield: 3.8 g

MS APCI+ 374/376[M+H]⁺

### iii) 8-(Benzyloxy)-5-((1R)-2-bromo-1-{[tert-butyl(dimethyl)silyl]oxy}ethyl)quinolin-2(1H)-one

A solution of the product of step (ii) (8-(benzyloxy)-5-[(1R)-2-bromo-1-hydroxyethyl]quinolin-2(1H)-one) (1.00 g) in DMF (4 mL) was stirred and cooled to 0° C. and to it was added dropwise 2,6-lutidine (0.622 mL) followed by tert-butyldimethylsilyl triflate (1.23 mL). The reaction mixture was stirred at room temperature overnight then the volatiles partially evaporated. The residue was dissolved in EtOAc and washed with H₂O, 2M aqueous HCl, H₂O and saturated aqueous NaCl. The organics collected, dried (Na₂SO₄) and the volatiles evaporated. The crude material was purified using a Biotage 40S column, eluting with 1:1 EtOAc:isohexane to afford the sub-title compound as a white solid. Yield: 1.3 g

MS APCI+ 488/490[M+H]⁺

### iv) Tert-Butyl (trans-4-{[2-(2-phenylethoxy)ethyl]amino}cyclohexyl)carbamate

To a solution of (2-phenylethoxy)acetaldehyde (WO 1994/27601) (0.47 g) in methanol (30 mL) was added tert-butyl (trains-4-aminocyclohexyl)carbamate (Bioorg. Med. Chem. Lett. 2004, 14(20), 5223-5226) (0.61 g) followed by H₂O (0.05 mL) and AcOH (0.05 mL) then sodium cyanoborohydride (0.18 g) and the mixture stirred at room temperature for 18 h. The volatiles were evaporated in vacuo and the residue partitioned between EtOAc and H₂O. The organic layer was further washed with saturated aqueous sodium bicarbonate solution and saturated aqueous NaCl then collected, dried (MgSO₄) and evaporated to leave a yellow gum. Purification was by column chromatography eluting with EtOAc with 0.5% triethylamine stepping to 1% triethylamine mixtures to give the sub-title compound as a white solid. Yield: 0.31 g

MS APCI+ 363 [M+H]⁺

### (v) Trans-N-[2-(2-phenylethoxy)ethyl]cyclohexane-1,4-diamine dihydrochloride

To a solution of the product from step (iv) (0.13 g) was stirred in 4 M HCl in dioxan (5 mL) for 2 h. The volatiles were evaporated in vacuo to give the sub-title compound as a white solid. Yield: 0.29 g.

MS APCI+ 263 [M+H]⁺

Used in the next step (vi) without any further purification.

### (vi) 8-(Benzyloxy)-5-{(1R)-1-{[tert-butyl(dimethyl)silyl]oxy}-2-[(trans-4-{[2-(2-phenylethoxy)ethyl]amino}cyclohexyl)amino]ethyl}quinolin-2(1H)-one

A mixture of the product from step (v) (0.1 g), the product from step (iii) (0.15 g), sodium bicarbonate (0.1 g) and sodium iodide (90 mg) in DMSO (0.3 mL) was heated at 140° C. for 1 h then allowed to cool to room temperature and further stirred for 48 h. The mixture was then partitioned between EtOAc and H₂O. The organics further washed with brine, collected, dried (MgSO₄) and evaporated to leave brown gum. Purification was by column chromatography eluting with EtOAc:triethylamine (9:1) mixtures followed by EtOAc:methanol (9:1) to give the sub-title compound as a yellow gum Yield: 70 mg

MS APCI+ 670 [M+H]⁺

### (vii) 8-Hydroxy-5-{(1R)-1-hydroxy-2-[(trans-4-{[2-(2-phenylethoxy)ethyl]amino}cyclohexyl)amino]ethyl}quinolin-2(1H)-one

A solution of the product from step (vii) (7 mg) in dry THF (1 mL) was treated with triethylamine trihydrofluoride (0.025 mL) and stirred at room temperature overnight. The volatiles evaporated and the residue re-dissolved in ethanol (10 mL) and 2M aqueous HCl (2 mL) followed by addition of 10% palladium on charcoal (0.05 g) and then stirred under 5 bar pressure of hydrogen gas overnight. The mixture was filtered through glass fibre paper and the solvent evaporated in vacuo to leave orange solid. Purification was by silica gel preparatory plate chromatography eluting with EtOAc/methanol/aq 0.880 NH₃ (65:30:5) to give the title compound as a yellow solid.

Yield: 21 mg

MS APCI+ 466 [M+H]⁺

¹H NMR (DMSO) δ 8.20 (d, 1H), 7.20 (m, 5H), 7.10 (d, 1H), 6.94 (d, 1H), 6.53 (d, 1H), 5.13 (m, 1H), 3.61 (t, 2H), 3.51 (m, 2H), 2.82 (m, 5H), 1.91 (m, 5H), 1.11 (m, 4H)

### EXAMPLE 3

### 8-Hydroxy-5-[(1R)-1-hydroxy-2-({1-[3-(2-phenylethoxy)propyl]piperidin-4-yl}amino)ethyl]quinolin-2(1H)-one

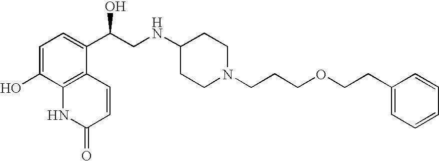

### i) 4-[(1R)-2-azido-1-hydroxyethyl]-8-(benzyloxy)quinolin-2(1H)-one

Sodium iodide (0.47 g) and sodium azide (0.74 g) were added to a solution of the product of example 2 step (ii) (8-(benzyloxy)-5-[(1R)-2-bromo-1-hydroxyethyl]quinolin-2(1H)-one) (1.07 g) in dry DMSO (10 mL). The reaction mixture was heated at 65° C. for 2 h. The mixture was allowed to cool to room temperature them diluted with EtOAc and H₂O and the layers separated. The aqueous material was further extracted with EtOAc (×4) then the combined organic extracts washed with saturated aqueous NaCl. The organics collected, dried (Na₂SO₄) and the volatiles removed in vacuo to leave a yellow solid. The solid residue was purified by trituration with 1:1 diethyl ether/EtOAc to give the sub-title compound as a white solid. Yield: 0.71 g

MS APCI+ 337 [M+H]⁺

### ii) 4-[(1R)-2-Amino-1-hydroxyethyl]-8-(benzyloxy)quinolin-2(1H)-one

The product from step (i) (4-[(1R)-2-azido-1-hydroxyethyl]-8-(benzyloxy)quinolin-2(1H)-one) (0.71 g) was dissolved in ethanol (10 mL) and THF (2 mL) and to it was added a suspension of 10% palladium on charcoal (71 mg) in ethanol (5 mL). The reaction mixture was hydrogenated at 5 bar pressure for 14 h then filtered through glass microfibre paper and concentrated in vacuo to give the sub-title compound as a dull yellow solid.

Yield: 0.64 g

MS APCI+ 311 [M+H]⁺

Used without further purification in the next step (iii)

### iii) 4-[(1R)-2-Amino-1-hydroxyethyl]-8-hydroxyquinolin-2(1H)-one

The product from step (ii) (4-[(1R)-2-amino-1-hydroxyethyl]-8-(benzyloxy)quinolin-2(1H)-one) (0.64 g) was dissolved in ethanol (20 mL), methanol (2 mL) and concentrated hydrochloric acid (1.5 mL) and to it was added a suspension of 10% palladium on charcoal (0.21 g) in ethanol (5 mL). The reaction mixture was hydrogenated at 5 bar pressure for 4 h. A further aliquot of 10% palladium on charcoal (50 mg) was added and the mixture further hydrogenated at 4 bar pressure for 14 h then filtered through glass microfibre paper and further washing with methanol/water mixtures followed by concentration in vacuo to leave a dull yellow solid. This was purified by trituration in ethanol and filtration to give the sub-title compound as a yellow solid. Yield: 0.3 g

MS APCI+ 221 [M+H]⁺

¹H NMR (DMSO) δ 10.45 (m, 2H), 8.15 (d, 1H), 7.98 (s, 2H), 7.14 (d, 1H), 6.98 (d, 1H), 6.58 (d, 1H), 6.05 (m, 1H), 5.20 (m, 1H), 3.0 (m, 2H)

### iv) 8-[3-(2-Phenylethoxy)propyl]-1,4-dioxa-8-azaspiro[4.5]decane

A solution of 3-(2-phenylethoxy)propanal (Sciences Chimiques; 1968, 266(18), 1379-1380) (2.5 g) and 1,4-dioxa-8-azaspiro[4.5]decane (2 g) in THF (40 ml) were stirred at room temperature for 10 min. At the end of this time, sodium triacetoxyborohydride (3 g) was added and the mixture was further stirred for 18 h at room temperature. The reaction mixture was quenched with saturated aqueous sodium bicarbonate (30 mL) and then extracted with EtOAc (×2). The organic layers collected, dried (MgSO₄), solvents removed under reduced pressure to leave a colourless oil. Purification was by column chromatography eluting with 800:150:5 EtOAc/methanol/triethylamine to give the sub-title compound as a colourless oil. Yield: 3.2 g

¹H NMR (CDCl₃) δ 7.20 (m, 5H), 3.95 (s, 4H), 3.60 (t, 2H), 3.50 (t, 2H), 2.95 (m, 4H), 2.40 (t, 2H), 1.80 (m, 6H)

### v) 1-[3-(2-phenylethoxy)propyl]piperidin-4-one

A solution of the product from step (iv) (8-[3-(2-phenylethoxy)propyl]-1,4-dioxa-8-azaspiro[4.5]decane) (1.88 g) in THF (25 mL) was treated with aqueous 2M HCl (15 ml) and the whole stirred and set at 50° C. for 48 h. The cooled mixture was partitioned between EtOAc and saturated aqueous sodium bicarbonate. The organic layer collected and the aqueous layer further extracted with EtOAc (×3). The organic layers collected, dried (MgSO₄), solvents removed under reduced pressure to leave a colourless oil. Purification was by applying a solution of the crude oil dissolved in acetonitrile to a Argonaut Technologies MP-TsOH(65) resin column (2 g) followed by eluting with acetonitrile. The pure product was obtained by eluting with triethylamine/acetonitrile mixtures. The volatiles evaporated in vacuo to give the sub-title compound as a colourless oil. Yield: 1.65 g

¹H NMR (CDCl₃) δ 7.20 (m, 5H), 3.65 (t, 2H), 3.45 (t, 2H), 2.95 (t, 2H), 2.80 (t, 4H), 2.50 (m, 6H), 1.90 (m, 2H)

### vi) 8-Hydroxy-4-[(1R)-1-hydroxy-2-({1-[3-(2-phenylethoxy)propyl]piperidin-4-yl}amino)ethyl]quinolin-2 (1H)-one

A solution of the product from step (iii) (4-[(1R)-2-amino-1-hydroxyethyl]-8-hydroxyquinolin-2(1H)-one) (0.3 g) in N-methyl-2-pyrrolidinone (25 mL) was treated with the product from step (v) (1-[3-(2-phenylethoxy)propyl]piperidin-4-one) (0.36 g) followed by AcOH (0.08 mL) and stirred at room temperature for 3 h. The mixture was then treated with sodium triacetoxyborohydride (0.58 g) and the whole further stirred for 24 h. The volatiles evaporated in vacuo to near dryness and then the solution loaded onto a SCX cartridge eluting with 1:1 isopropanol/acetonitrile. The product was eluted with 10% 0.880 NH₃ in 1:1 isopropanol/acetonitrile to give the crude product as a yellow oil. Purification was by reverse phase HPLC using an Xterra® C8 5 micron 19×50 mm column eluting with a gradient of 95% 0.880 NH₃ in acetonitrile to 50% 0.880 NH₃ in acetonitrile to give the product as a yellow oil. This was re-dissolved in acetonitrile/water mixtures and then the pH adjusted to pH 1 with trifluoroacetic acid. Purification was continued by using a Symmetry® C8 5 micron 19×50 mm column eluting with a gradient of 95% aqueous trifluoroacetic acid in acetonitrile to 50% aqueous trifluoroacetic acid in acetonitrile followed by trituration with ether to give the title compound as a off white solid.

Yield: 0.1 g.

MS APCI+ 466[M+H]⁺

1H NMR: (DMSO) δ10.54-10.46 (m, 2H), 8.15 (d, 1H), 7.32-7.15 (m, 5H), 6.99 (d, 1H), 6.59 (dd, 1H), 6.25-6.21 (m, 1H), 5.35-5.28 (m, 1H), 3.59 (t, 4H), 3.48-3.29 (m, 4H), 3.16-2.86 (m, 4H), 2.81 (t, 2H), 2.34-2.20 (m, 2H), 1.92-1.69 (m, 4H)

### EXAMPLE 4

### 8-Hydroxy-5-[(1R)-1-hydroxy-2-({1-[2-(2-phenylethoxy)ethyl]piperidin-4-yl}amino)ethyl]quinolin-2(1H)-one

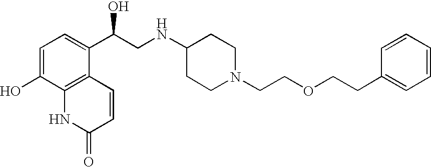

### i) 2-(2-Phenylethoxy)ethyl 4-methylbenzenesulfonate

To a solution of 2-(2-phenylethoxy)ethanol (J. Med. Chem. 1983, 26(11), 1570-1576) (1.13 g) in pyridine (5 mL) was added tosyl chloride (1.4 g) and N,N-dimethylaminopyridine (20 mg) at room temperature with further stirring for 14 h. The volatiles were evaporated in vacuo and the residue partitioned between EtOAc and aqueous 2M HCl. The organic layer collected and further washed with saturated aqueous sodium bicabonate solution and saturated aqueous NaCl followed by H₂O. The organic layer collected, dried (MgSO₄) and solvents evaporated to give the sub-title compound as a pink oil. Yield: 1.4 g

¹H NMR (CDCl₃) δ 7.80 (d, 2H), 7.20 (m, 7H), 4.10 (m, 2H), 3.60 (m, 4H), 2.81 (t, 2H), 2.44 (s, 3H)

Used without further purification in the next step (ii)

### ii) 1-[2-(2-phenylethoxy)ethyl]piperidin-4-one

A solution of the product from step (i) (2-(2-phenylethoxy)ethyl 4-methylbenzenesulfonate) (1.9 g) in N-methyl-2-pyrrolidinone (15 mL) and triethylamine (4 mL) was treated with piperidin-4-one hydrochloride (1.1 g). The whole was set at 85° C. for 2 h. The cooled mixture partitioned between saturated aqueous sodium bicarbonate solution and EtOAc. The organic layer collected and further washed with saturated aqueous NaCl followed by H₂O. The organic layer collected, dried (MgSO₄) and solvents evaporated to give the sub-title compound as a brown oil. Yield: 1.6 g

¹H NMR (CDCl₃) δ 7.30 (m, 5H), 3.70 (m, 8H), 2.90 (m, 3H), 2.80 (t, 2H), 2.72 (t, 1H), 2.42 (t, 2H).

Used without further purification in the next step (iii)

### iii) 5-[(1R)-1-{[tert-butyl(dimethyl)silyl]oxy}-2-({1-[2-(2-phenylethoxy)ethyl]piperidin-4-yl}amino)ethyl]-8-hydroxyquinolin-2(1H)-one

A solution of the product from step (ii) (1-[2-(2-phenylethoxy)ethyl]piperidin-4-one) (0.22 g) in N-methyl-2-pyrrolidinone (10 mL) was treated with the product from example 5 step (ii) (5-((1R)-2-amino-1-{[tert-butyl(dimethyl)silyl]oxy}ethyl)-8-hydroxyquinolin-2(1H)-one) (0.28 g) followed by AcOH (0.05 mL) and stirred at room temperature for 2 h. The mixture was then treated with sodium triacetoxyborohydride (0.36 g) and the whole further stirred for 24 h. The reaction mixture was then treated with 0.880 NH₃ (10 mL) and the volatiles removed in vacuo. The residue was diluted with isopropanol (5 mL) and loaded onto a Argonaut Technologies MP-TsOH(65) resin column (4.6 g), which had previously been washed with isopropanol. The column was eluted with ˜50 ml isopropanol, followed by 1:3 0.880 NH₃:isopropanol. The product-containing fraction was concentrated in vacuo to leave a dark yellow oil. This was purified using a Biotage 40S column, eluting with 1% 7M NH₃ in methanol in DCM, increasing the amount of methanol to 10%. Followed by flushing with 2% 7M NH₃ in methanol/18% methanol/80% EtOAc then 2% 7M NH₃ in methanol/48% methanol/50% EtOAc to give the sub-title compound as a dark yellow oil. Yield: 0.14 g

MS APCI+ 566 [M+H]⁺

Used without further purification in the next step (iv)

### iv) 8-Hydroxy-4-[(1R)-1-hydroxy-2-({1-[2-(2-phenylethoxy)ethyl]piperidin-4-yl}amino)ethyl]quinolin-2 (1H)-one

The product from step (iii) (5-[(1R)-1-{[tert-butyl(dimethyl)silyl]oxy}-2-({1-[2-(2-phenylethoxy)ethyl]piperidin-4-yl}amino)ethyl]-8-hydroxyquinolin-2(1H)-one) (0.114 g) was suspended in THF (5 mL) and to it was added triethylamine trihydrofluoride (0.2 mL). The reaction mixture was stirred at room temperature for 3 d, then diluted with methanol and the volatiles evaporated. The residue was dissolved in methanol and loaded onto an Argonaut Technologies MP-TsOH(65) resin column (1 g), which had previously been washed with isopropanol. The column was eluted with 30 ml isopropanol, followed by 1:3 aqueous. NH₃: isopropanol. The product-containing fractions were concentrated in vacuo to afford the crude product as a yellow gum. Purification was by reverse phase HPLC using a Symmetry® C8 5 micron 19×50 mm column, eluting with gradient 5-50% acetonitrile in 0.2% aqueous trifluoroacetic acid give the title compound as a off white solid. Yield: 26 mg

MS APCI(+ve) 452 [M+H]⁺

¹H NMR (DMSO) δ 10.57 (s, 1H), 10.50 (s, 1H), 9.87 (bs, 1H), 9.26 (bs, 1H), 8.94 (bs, 1H), 8.18 (d, 1H), 7.25 (m, 6H), 7.00 (d, 1H), 6.58 (d, 1H), 6.26 (bs, 1H), 5.35 (d, 1H), 3.70 (m, 4H), 3.30 (m, 5H), 3.10 (m, 2H), 2.85 (m, 4H), 2.0 (m, 4H)

### EXAMPLE 5

### 8-Hydroxy-5-{(1R)-1-hydroxy-2-[({4-hydroxy-1-[2-(2-phenylethoxy)ethyl]piperidin-4-yl}methyl)amino]ethyl}quinolin-2(1H)-one

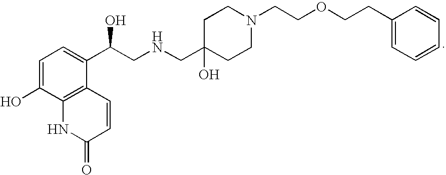

### i) 5-((1R)-2-azido-1-{[tert-butyl(dimethyl)silyl]oxy}ethyl)-8-(benzyloxy)quinolin-2(1H)-one

Sodium iodide (0.38 g) and sodium azide (0.60 g) were added to a solution of the product of example 2 step (iii) (8-(benzyloxy)-5-((1R)-2-bromo-1-{[tert-butyl(dimethyl)silyl]oxy}ethyl)quinolin-2(1H)-one) (1.12 g) in dry DMSO (20 mL). The reaction mixture was heated at 65° C. for 3 h. The mixture was allowed to cool to room temperature them diluted with EtOAc and H₂O and the layers separated. The aqueous material was extracted with further EtOAc (×4) then the combined organic extracts washed with saturated aqueous NaCl, collected, dried (Na₂SO₄) and the volatiles removed in vacuo. The residue was purified by Biotage on a 40S column, eluting with 40% EtOAc in isohexane to afford the sub-title compound as a white solid. Yield: 0.94 g

MS APCI+ 451 [M+H]⁺

### ii) 5-((1R)-2-Amino-1-{[tert-butyl(dimethyl)silyl]oxy}ethyl)-8-hydroxyquinolin-2(1H)-one

The product from step (i) (5-((1R)-2-azido-1-{[tert-butyl(dimethyl)silyl]oxy}ethyl)-8-(benzyloxy)quinolin-2(1H)-one) (0.94 g) was dissolved in ethanol (33 mL) and to it was added a suspension of 10% palladium on charcoal (0.49 g) in ethanol (5 mL). The reaction mixture was hydrogenated at 5 bar for 4 h, then filtered through glass microfibre paper and concentrated in vacuo to afford the sub-title compound as a yellow oil. Yield: 0.65 g

MS APCI+ 335[M+H]⁺

### iii) 6-[2-(2-phenylethoxy)ethyl]-1-oxa-6-azaspiro[2.5]octane

To dry DMSO (0.27 g) was added 60% sodium hydride (65 mg) at room temperature followed by trimethylsulfoxonium iodide (0.27 g) and the resulting solution further stirred for 2.5 h. A solution of the product from example 4 step (ii) 1-[2-(2-phenylethoxy)ethyl]piperidin-4-one (0.2 g) in dry THF (2 mL) was added to the above mixture dropwise followed by further stirring at ambient temperature for 1.5 h. The mixture was partitioned between EtOAc and H₂O. The organic layer further washed with saturated aqueous NaCl, collected, dried (MgSO₄) and evaporated in vacuo to leave a yellow oil. Yield: 0.14 g

MS APCI+ 262[M+H]⁺

### iv) 5-{(1R)-1-{[tert-Butyl(dimethyl)silyl]oxy}-2-[({4-hydroxy-1-[2-(2-phenylethoxy)ethyl]piperidin-4-yl}methyl)amino]ethyl}-8-hydroxyquinolin-2(1H)-one

A mixture of the product from step (ii) (5-((1R)-2-amino-1-{[tert-butyl(dimethyl)silyl]oxy}ethyl)-8-hydroxyquinolin-2(1H)-one) (0.26 g) and the product from step (iii) (0.54 g) in methanol (2 mL) and N,N-diisopropylethyamine (0.092 mL) was heated at reflux for 3 d. The volatiles were evaporated in vacuo to leave a black gum. Purification was by reverse phase HPLC using an Xterra® C8 5 micron 19×50 mm column eluting with a gradient of 95% 0.880 NH₃ in acetonitrile to 5% 0.880 NH₃ in acetonitrile give the sub-title compound as a yellow foam. Yield: 79 mg

MS APCI+ 596 [M+H]⁺

### v) 8-Hydroxy-5-{(1R)-1-hydroxy-2-[({4-hydroxy-1-[2-(2-phenylethoxy)ethyl]piperidin-4-yl}methyl)amino]ethyl}quinolin-2(1H)-one

The product from step (iv) 5-{(1R)-1-{[tert-butyl(dimethyl)silyl]oxy}-2-[({4-hydroxy-1-[2-(2-phenylethoxy)ethyl]piperidin-4-yl}methyl)amino]ethyl}-8-hydroxyquinolin-2(1H)-one) (80 mg) in THF (10 mL) was treated with triethylamine trihydrofluoride (0.033 mL) and further stirred at room temperature for 18 h. The volatiles were evaporated in vacuo to leave a yellow foam. Purification was by reverse phase HPLC using an Xterra® C8 5 micron 19×50 mm column eluting with a gradient of 95% 0.880 NH₃ in acetonitrile to 5% 0.880 NH₃ in acetonitrile give the title compound as a yellow foam. Yield: 62 mg

MS APCI+ 482[M+H]⁺

¹H NMR (DMSO) δ 8.20 (d, 1H), 7.20 (m, 5H), 7.10 (d, 1H), 6.90 (d, 1H), 6.50 (d, 1H), 5.00 (m, 1H), 3.55 (t, 2H), 3.50 (m, 2H), 2.70 (m, 2H), 2.40 (m, 2H), 2.40 (m, 9H), 1.50 (m, 4H)

### EXAMPLE 6

### N-Benzyl-3-(4-{[(2R)-2-hydroxy-2-(8-hydroxy-2-oxo-1,2-dihydroquinolin-5-yl)ethyl]amino}piperidin-1-yl)propanamide

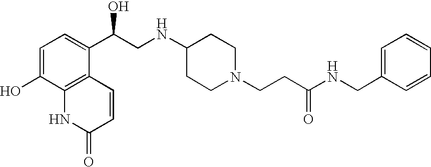

### i) tert-Butyl 3-(4-oxopiperidin-1-yl)propanoate

To a solution of piperidin-4-one (1 g) in CHCl₃ (30 mL) was added tert-butyl 3-bromopropanoate (1.2 mL) followed by triethylamine (1.8 mL). The reaction mixture was heated at reflux for 18 h. The mixture was allowed to cool to room temperature then diluted with EtOAc and H₂O and the layers separated. The aqueous material was extracted with further EtOAc (×2) then the combined organic extracts washed with water followed by saturated aqueous NaCl, collected and dried (MgSO₄). The volatiles were removed in vacuo to afford the sub-title compound as an orange oil. Yield: 0.92 g

¹H NMR (CDCl₃) δ 2.81-2.75 (m, 6H), 2.47-2.42 (m, 6H), 1.46 (s, 9H).

### ii) tert-Butyl 3-(4-{[(2R)-2-{[tert-butyl(dimethyl)silyl]oxy}-2-(8-hydroxy-2-oxo-1,2-dihydroquinolin-5-yl)ethyl]amino}piperidin-1-yl)propanoate

A solution of the product from step (i) (tert-butyl 3-(4-oxopiperidin-1-yl)propanoate) (2.09 g) in NMP (15 mL) was treated with the product from example 3 step (ii) (4-[(1R)-2-amino-1-hydroxyethyl]-8-(benzyloxy)quinolin-2(1H)-one) (1.53 g) followed by AcOH (0.28 mL) then powdered molecular sieves and stirred at room temperature for 7 h. The mixture was then treated with sodium triacetoxyborohydride (1.95 g) and the whole further stirred for 18 h. The reaction mixture was filtered and the filtrate was concentrated in vacuo. The resulting oil was loaded onto a Varian Bond Elut SCX resin column (50 g). The column was eluted with 50 mL 1:1 isopropanol:acetonitrile, followed by 1:2:2 0.880 NH₃:isopropanol:acetonitrile. The product-containing fraction was concentrated in vacuo to leave an oil which was purified using a Biotage column eluting with 8% methanol in DCM, increasing the amount of methanol to 20% to give the sub-title compound as a dark orange oil. Yield: 1.66 g

MS APCI+ 546 [M+H]⁺

¹H NMR (DMSO) δ 10.38 (s, 1H), 8.24 (d, 1H), 7.03 (d, 1H), 6.91 (d, 1H), 6.50 (d, 1H), 3.46-1.08 (m, 16H), 1.39-1.38 (m, 9H), 0.84 (s, 9H), 0.05 (s, 3H), −0.18 (s, 3H).

### iii) 3-(4-{[(2R)-2-{[tert-Butyl(dimethyl)silyl]oxy}-2-(8-hydroxy-2-oxo-1,2-dihydroquinolin-5-yl)ethyl]amino}piperidin-1-yl)propanoic acid

A solution of the product from step (ii) (tert-butyl 3-(4-{[(2R)-2-{[tert-butyl(dimethyl)silyl]oxy}-2-(8-hydroxy-2-oxo-1,2-dihydroquinolin-5-yl)ethyl]amino}piperidin-1-yl)propanoate) in TFA (10 mL) was stirred at room temperature for 2 h. then concentrated in vacuo and azeotroped with methanol (×2). The resulting residue was loaded onto a Varian Bond Elut SCX resin column (50 g). The column was eluted with 50 mL 1:1 isopropanol:acetonitrile, followed by 1:2:2 0.880 NH₃:isopropanol:acetonitrile. The product-containing fraction was concentrated in vacuo to give the sub-title compound as a dark yellow oil. Yield: 1.3 g

MS APCI+ 490 [M+H]⁺

### iv) N-Benzyl-3-(4-{[(2R)-2-{[tert-butyl(dimethyl)silyl]oxy}-2-(8-hydroxy-2-oxo-1,2-dihydroquinolin-5-yl)ethyl]amino}piperidin-1-yl)propanamide

A mixture of the product from step (iii) (3-(4-{[(2R)-2-{[tert-butyl(dimethyl)silyl]oxy}-2-(8-hydroxy-2-oxo-1,2-dihydroquinolin-5-yl)ethyl]amino}piperidin-1-yl)propanoic acid) (0.25 g), benzylamine (0.56 mL), triethylamine (0.21 mL) and HATU (0.35 g) in DMF (8 mL) was stirred at room temperature for 48 h then the volatiles were removed in vacuo. The resulting residue was loaded onto a Varian Bond Elut SCX resin column (10 g). The column was eluted with 50 mL 1:1 isopropanol:acetonitrile, followed by 1:2:2 0.880 NH₃:isopropanol:acetonitrile. The product-containing fraction was concentrated in vacuo then further purified using a Biotage column, eluting with 5% 7M NH₃ in methanol in 1:9 methanol:DCM, to give the sub-title compound as a dark orange oil. Yield: 0.2 g

MS APCI+ 579 [M+H]⁺

### v) N-Benzyl-3-(4-{[(2R)-2-hydroxy-2-(8-hydroxy-2-oxo-1,2-dihydroquinolin-5-yl)ethyl]amino}piperidin-1-yl)propanamide

The product from step (iv) (N-benzyl-3-(4-{[(2R)-2-{[tert-butyl(dimethyl)silyl]oxy}-2-(8-hydroxy-2-oxo-1,2-dihydroquinolin-5-yl)ethyl]amino}piperidin-1-yl)propanamide) (0.20 g) in THF (5 mL) was treated with triethylamine trihydrofluoride (0.28 mL) and stirred at room temperature for 18 h. The volatiles were evaporated in vacuo and the resulting residue loaded onto a Varian Bond Elut SCX resin column (10 g). The column was eluted with 50 mL 1:1 isopropanol:acetonitrile, followed by 1:2:2 0.880 NH₃:isopropanol:acetonitrile. The product-containing fraction was concentrated in vacuo then further purified using reverse phase HPLC using a Symmetry® C8 5 micron 19×50 mm column, eluting with gradient 0-50% acetonitrile in 0.2% aqueous TFA to give the title compound as a off white solid. Yield: 0.11 g

MS APCI+ 465 [M+H]⁺

¹H NMR (DMSO) δ 10.55-10.46 (m, 2H), 8.16 (d, 1H), 7.36-7.23 (m, 5H), 7.18 (d, 1H), 6.99 (d, 1H), 6.59 (d, 1H), 6.30-6.20 (m, 1H), 5.36-5.29 (m, 1H), 4.30 (d, 2H), 3.79-3.47 (m, 8H), 3.16-3.07 (m, 2H), 3.02-2.91 (m, 1H), 2.70-2.62 (m, 2H), 2.35-2.20 (m, 1H), 1.93-1.71 (m, 1H).

### EXAMPLE 7

### N-Benzyl-3-[4-({[(2R)-2-hydroxy-2-(8-hydroxy-2-oxo-1,2-dihydroquinolin-5-yl)ethyl]amino}methyl)piperidin-1-yl]propanamide

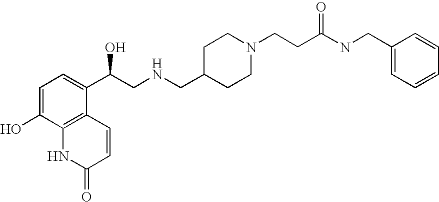

### i) tert-Butyl 4-[4-(hydroxymethyl)piperidin-1-yl]butanoate

The sub-titled compound was prepared according to the procedure outlined in example 6 step (i) using piperidin-4-ylmethanol (2 g) in CHCl₃ (40 mL), tert-butyl 3-bromopropanoate (3.9 mL), triethylamine (2.8 mL) at reflux for 18 h to give the sub-title compound as an orange oil. Yield: 3 g

¹H NMR (CDCl₃) δ 3.57-3.47 (m, 2H), 3.03-2.92 (m, 2H), 2.77-2.66 (m, 2H), 2.53-2.42 (m, 2H), 2.13-1.99 (m, 2H), 1.83-1.71 (m, 2H), 1.57-1.24 (m, 12H).

### ii) tert-Butyl 3-[4-({[(2R)-2-{[tert-butyl(dimethyl)silyl]oxy}-2-(8-hydroxy-2-oxo-1,2-dihydroquinolin-5-yl)ethyl]amino}methyl)piperidin-1-yl]propanoate

Oxalyl chloride (0.67 mL) was added dropwise to a cooled (−78° C.) solution of DMSO (0.57 mL) in DCM (30 mL) and stirred for 30 min. A solution of the product from step (i) (tert-butyl 4-[4-(hydroxymethyl)piperidin-1-yl]butanoate) (1.5 g) in DCM was then added dropwise over 10 min and stirred for a further 1 h at −78° C. After the addition of triethylamine (1.9 mL) the reaction mixture was allowed to warm to room temperature and stirred for a further 18 h then diluted with DCM and H₂O and the layers separated. The aqueous material was extracted with further DCM then the combined organic extracts washed with saturated aqueous NaCl, collected and dried (MgSO₄). The volatiles removed in vacuo to afford the intermediate compound, tert-butyl 4-(4-formylpiperidin-1-yl)butanoate, as an orange oil. Yield: 1.5 g

The sub-titled compound was prepared according to the procedure outlined in example 6 step (ii) using the intermediate compound detailed above (tert-butyl 4-(4-formylpiperidin-1-yl)butanoate) (0.48 g), the product from example 3 step (ii) (4-[(1R)-2-amino-1-hydroxyethyl]-8-(benzyloxy)quinolin-2(1H)-one) (0.34 g) followed by AcOH (0.06 mL) in NMP (15 mL) at room temperature for 2 h. Sodium triacetoxyborohydride (0.42 g) was subsequently added and stirred for a further 2 h to afford the sub-title compound as a yellow oil. Yield: 0.53 g

MS APCI+ 560 [M+H]⁺

¹H NMR (DMSO) δ 8.24 (d, 1H), 7.02 (d, 1H), 6.90 (d, 1H), 6.50 (d, 1H), 5.13-5.07 (m, 1H), 2.82-0.99 (m, 17H), 1.40 (s, 9H), 0.83 (s, 9H), 0.04 (s, 3H), −0.18 (s, 3H).

### iii) 3-[4-({[(2R)-2-{[tert-Butyl(dimethyl)silyl]oxy}-2-(8-hydroxy-2-oxo-1,2-dihydroquinolin-5-yl)ethyl]amino}methyl)piperidin-1-yl]propanoic acid

The sub-titled compound was prepared according to the procedure outlined in example 6 step (iii) using the product from step (ii) (tert-butyl 3-[4-({[(2R)-2-{[tert-butyl(dimethyl)silyl]oxy}-2-(8-hydroxy-2-oxo-1,2-dihydroquinolin-5-yl)ethyl]amino}methyl)piperidin-1-yl]propanoate) (0.53 g) in TFA (20 mL) at room temperature for 1 h to afford the sub-title compound as a dark yellow oil. Yield: 0.24 g

MS APCI+ 504 [M+H]⁺

### iv) N-Benzyl-3-[4-({[(2R)-2-{[tert-butyl(dimethyl)silyl]oxy}-2-(8-hydroxy-2-oxo-1,2-dihydroquinolin-5-yl)ethyl]amino}methyl)piperidin-1-yl]propanamide

The sub-titled compound was prepared according to the procedure outlined in example 6 step (iv) using a solution of the product from step (iii) (3-[4-({[(2R)-2-{[tert-butyl(dimethyl)silyl]oxy}-2-(8-hydroxy-2-oxo-1,2-dihydroquinolin-5-yl)ethyl]amino}methyl)piperidin-1-yl]propanoic acid) (0.17 g), benzylamine (0.37 mL), triethylamine (0.14 mL) and HATU (0.34 g) in DMF (6 mL) and acetonitrile (3 mL) at room temperature for 30 min to afford the sub-title compound as a dark yellow oil. Yield: 0.2 g

MS APCI+ 593 [M+H]⁺

### v) N-Benzyl-3-[4-({[(2R)-2-hydroxy-2-(8-hydroxy-2-oxo-1,2-dihydroquinolin-5-yl)ethyl]amino}methyl)piperidin-1-yl]propanamide

The sub-titled compound was prepared according to the procedure outlined in example 6-step (v) using a solution of the product from step (iv) (N-benzyl-3-[4-({[(2R)-2-{[tert-butyl(dimethyl)silyl]oxy}-2-(8-hydroxy-2-oxo-1,2-dihydroquinolin-5-yl)ethyl]amino}methyl)piperidin-1-yl]propanamide) (0.2 g), with triethylamine trihydrofluoride (0.30 mL) in THF (5 mL) at room temperature for 3 h to afford the title compound as a off white solid. Yield: 70 mg

MS APCI+ 479 [M+H]⁺

¹H NMR (DMSO) δ 8.17 (d, 1H), 7.37-7.22 (m, 5H), 7.15 (d, 1H), 6.99 (d, 1H), 6.59 (d, 1H), 5.34 (d, 1H), 4.31 (d, 2H), 3.58-2.85 (m, 1H), 2.71-2.63 (m, 2H), 2.04-1.91 (m, 2H), 1.48-1.34 (m, 2H).

### EXAMPLE 8

### 5-[(1R)-2-({1-[3-(3,4-Dihydroisoquinolin-2(1H)-yl)-3-oxopropyl]piperidin-4-yl}amino)-1-hydroxyethyl]-8-hydroxyquinolin-2(1H)-one

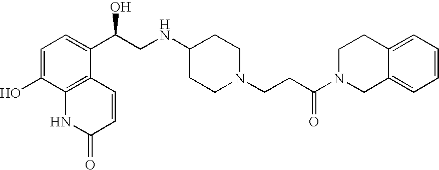

### i) 5-[(1R)-1-{[tert-Butyl(dimethyl)silyl]oxy}-2-({1-[3-(3,4-dihydroisoquinolin-2(1H)-yl)-3-oxopropyl]piperidin-4-yl}amino)ethyl]-8-hydroxyquinolin-2(1H)-one

The sub-titled compound was prepared according to the procedure outlined in Example 6 step (iv) using a solution of the product from example 6 step (iii) (3-(4-{[(2R)-2-{[tert-butyl(dimethyl)silyl]oxy}-2-(8-hydroxy-2-oxo-1,2-dihydroquinolin-5-yl)ethyl]amino}piperidin-1-yl)propanoic acid) (0.22 g), 1,2,3,4-tetrahydro-isoquinoline (0.12 g), triethylamine (0.17 mL) and HATU (0.31 g) in DMF (5 mL) at room temperature for 10 min to afford the sub-title compound as a dark yellow oil. Yield: 0.27 g

MS APCI+ 605 [M+H]⁺

### ii) 5-[(1R)-2-({1-[3-(3,4-Dihydroisoquinoln-2(1H)-yl)-3-oxopropyl]piperidin-4-yl}amino)-1-hydroxyethyl]-8-hydroxyquinolin-2(1H)-one

The sub-titled compound was prepared according to the procedure outlined in Example 6 step (v) using a solution of the product from step (i) (5-[(1R)-1-{[tert-butyl(dimethyl)silyl]oxy}-2-({1-[3-(3,4-dihydroisoquinolin-2(1H)-yl)-3-oxopropyl]piperidin-4-yl}amino)ethyl]-8-hydroxyquinolin-2(1H)-one) (0.27 g), with triethylamine trihydrofluoride (0.36 mL) in THF (5 mL) at room temperature for 18 h to afford the title compound as an off-white solid. Yield: 0.14 g

MS APCI+ 491 [M+H]⁺

¹H NMR (DMSO) δ 8.16 (1H, d), 7.22-7.15 (5H, m), 6.99 (1H, d), 6.59 (1H, d), 5.36-5.30 (1H, m), 4.64 (2H, d), 3.99-1.73 (19H, m).

### EXAMPLE 9

### 3-(4-{[(2R)-2-Hydroxy-2-(8-hydroxy-2-oxo-1,2-dihydroquinolin-5-yl)ethyl]amino}piperidin-1-yl)-N-(2-phenylethyl)propanamide

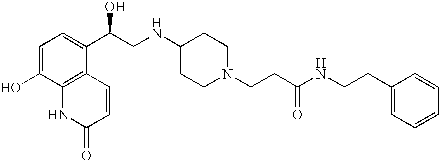

### i) 3-(4-{[(2R)-2-{[tert-Butyl(dimethyl)silyl]oxy}-2-(8-hydroxy-2-oxo-1,2-dihydroquinolin-5-yl)ethyl]amino}piperidin-1-yl)-N-(2-phenylethyl)propanamide

The sub-titled compound was prepared according to the procedure outlined in Example 6 step (iv) using a solution of the product from Example 6 step (iii) (3-(4-{[(2R)-2-{[tert-butyl(dimethyl)silyl]oxy}-2-(8-hydroxy-2-oxo-1,2-dihydroquinolin-5-yl)ethyl]amino}piperidin-1-yl)propanoic acid) (0.22 g), 2-phenylethanamine (0.11 g), triethylamine (0.17 mL) and HATU (0.31 g) in DMF (5 mL) at room temperature for 10 min to afford the sub-title compound as a dark yellow oil. Yield: 0.27 g.

MS APCI+ 593 [M+H]⁺

### ii) 3-(4-{[(2R)-2-Hydroxy-2-(8-hydroxy-2-oxo-1,2-dihydroquinolin-5-yl)ethyl]amino}piperidin-1-yl)-N-(2-phenylethyl)propanamide

The sub-titled compound was prepared according to the procedure outlined in Example 6 step (v) using a solution of the product from step (i) (3-(4-{[(2R)-2-{[tert-butyl(dimethyl)silyl]oxy}-2-(8-hydroxy-2-oxo-1,2-dihydroquinolin-5-yl)ethyl]amino}piperidin-1-yl)-N-(2-phenylethyl)propanamide) (0.27 g), with triethylamine trihydrofluoride (0.36 mL) in THF (5 mL) at room temperature for 18 h to afford the title compound as an off-white solid. Yield: 90 mg

MS APCI+ 479 [M+H]⁺

¹H NMR (DMSO) δ 8.16 (d, 1H), 7.35-7.15 (m, 6H), 6.99 (d, 1H), 6.59 (d, 1H), 5.37-5.29 (m, 1H), 3.59-1.68 (m, 19H).

### EXAMPLE 10

### N-(3-Chlorobenzyl)-3-(4-{[(2R)-2-hydroxy-2-(8-hydroxy-2-oxo-1,2-dihydroquinolin-5-yl)ethyl]amino}piperidin-1-yl)propanamide

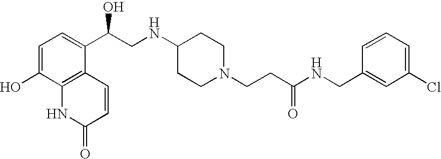

### i) 3-(4-{[(2R)-2-{[tert-Butyl(dimethyl)silyl]oxy}-2-(8-hydroxy-2-oxo-1,2-dihydroquinolin-5-yl)ethyl]amino}piperidin-1-yl)-N-(2-chlorobenzyl)propanamide

The sub-titled compound was prepared according to the procedure outlined in Example 6 step (iv) using a solution of the product from Example 6 step (iii) (3-(4-{[(2R)-2-{[tert-butyl(dimethyl)silyl]oxy}-2-(8-hydroxy-2-oxo-1,2-dihydroquinolin-5-yl)ethyl]amino}piperidin-1-yl)propanoic acid) (0.22 g), 1-(2-chlorophenyl)methanamine (0.13 g), triethylamine (0.17 mL) and HATU (0.31 g) in DMF (5 mL) at room temperature for 18 h to afford the sub-title compound as a dark yellow oil. Yield: 0.28 g.

MS APCI+ 613 [M+H]⁺

### ii) N-(2-Chlorobenzyl)-3-(4-{[(2R)-2-hydroxy-2-(8-hydroxy-2-oxo-1,2-dihydroquinolin-5-yl)ethyl]amino}piperidin-1-yl)propanamide

The sub-titled compound was prepared according to the procedure outlined in Example 6 step (v) using a solution of the product from step (i) (3-(4-{[(2R)-2-{[tert-butyl(dimethyl)silyl]oxy}-2-(8-hydroxy-2-oxo-1,2-dihydroquinolin-5-yl)ethyl]amino}piperidin-1-yl)-N-(2-chlorobenzyl)propanamide) (0.28 g), with triethylamine trihydrofluoride (0.36 mL) in THF (5 mL) at room temperature for 18 h to afford the title compound as an off-white solid. Yield: 0.18 g

MS APCI+ 499 [M+H]⁺

¹H NMR (DMSO) δ 8.16 (d, 1H), 7.47-7.43 (m, 1H), 7.39-7.28 (m, 3H), 7.17 (d, 1H), 6.99 (d, 1H), 6.58 (d, 1H), 5.36-5.30 (m, 1H), 4.40-1.70 (m, 17H).

### EXAMPLE 11

### 3-(4-{[(2R)-2-Hydroxy-2-(8-hydroxy-2-oxo-1,2-dihydroquinolin-5-yl)ethyl]amino}piperidin-1-yl)-N-(2-methoxybenzyl)propanamide

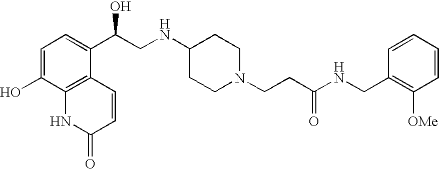

### i) 3-(4-{[(2R)-2-{[tert-Butyl(dimethyl)silyl]oxy}-2-(8-hydroxy-2-oxo-1,2-dihydroquinolin-5-yl)ethyl]amino}piperidin-1-yl)-N-(2-methoxybenzyl)propanamide

The sub-titled compound was prepared according to the procedure outlined in Example 6 step (iv) using a solution of the product from Example 6 step (iii) (3-(4-{[(2R)-2-{[tert-butyl(dimethyl)silyl]oxy}-2-(8-hydroxy-2-oxo-1,2-dihydroquinolin-5-yl)ethyl]amino}piperidin-1-yl)propanoic acid) (0.22 g), 1-(2-methoxyphenyl)methanamine (0.12 g), triethylamine (0.17 mL) and HATU (0.31 g) in DMF (5 mL) at room temperature for 18 h to afford the sub-title compound as a dark yellow oil. Yield: 0.27 g.

MS APCI+ 609 [M+H]⁺

### ii) 3-(4-{[(2R)-2-Hydroxy-2-(8-hydroxy-2-oxo-1,2-dihydroquinolin-5-yl)ethyl]amino}piperidin-1-yl)-N-(2-methoxybenzyl)propanamide

The sub-titled compound was prepared according to the procedure outlined in Example 6 step (v) using a solution of the product from step (i) (3-(4-{[(2R)-2-{[tert-butyl(dimethyl)silyl]oxy}-2-(8-hydroxy-2-oxo-1,2-dihydroquinolin-5-yl)ethyl]amino}piperidin-1-yl)-N-(2-methoxybenzyl)propanamide) (0.27 g), with triethylamine trihydrofluoride (0.36 mL) in THF (5 mL) at room temperature for 18 h to afford the title compound as an off-white solid. Yield: 0.13 g

MS APCI+ 495 [M+H]⁺

¹H NMR (DMSO) δ 8.16 (d, 1H), 7.25 (t, 1H), 7.17 (d, 2H), 6.99 (d, 2H), 6.91 (t, 1H), 6.59 (d, 1H), 5.36-5.30 (m, 1H), 4.26 (d, 2H), 3.81 (s, 3H), 3.73-1.70 (m, 15H).

### EXAMPLE 12

### N-(4-Cyanobenzyl)-3-(4-{[(2R)-2-hydroxy-2-(8-hydroxy-2-oxo-1,2-dihydroquinolin-5-yl)ethyl]amino}piperidin-1-yl)propanamide

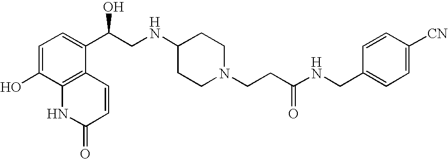

### i) 3-(4-{[(2R)-2-{[tert-Butyl(dimethyl)silyl]oxy}-2-(8-hydroxy-2-oxo-1,2-dihydroquinolin-5-yl)ethyl]amino}piperidin-1-yl)-N-(4-cyanobenzyl)propanamide

The sub-titled compound was prepared according to the procedure outlined in Example 6 step (iv) using a solution of the product from Example 6 step (iii) (3-(4-{[(2R)-2-{[tert-butyl(dimethyl)silyl]oxy}-2-(8-hydroxy-2-oxo-1,2-dihydroquinolin-5-yl)ethyl]amino}piperidin-1-yl)propanoic acid) (0.22 g), 4-(aminomethyl)benzonitrile (0.12 g), triethylamine (0.17 mL) and HATU (0.31 g) in DMF (5 mL) at room temperature for 18 h to afford the sub-title compound as a dark yellow oil. Yield: 0.27 g.

MS APCI+ 604 [M+H]⁺

### ii) N-(4-Cyanobenzyl)-3-(4-{[(2R)-2-hydroxy-2-(8-hydroxy-2-oxo-1,2-dihydroquinolin-5-yl)ethyl]amino}piperidin-1-yl)propanamide

The sub-titled compound was prepared according to the procedure outlined in Example 6 step (v) using a solution of the product from step (i) (3-(4-f{[(2R)-2-{[tert-butyl(dimethyl)silyl]oxy}-2-(8-hydroxy-2-oxo-1,2-dihydroquinolin-5-yl)ethyl]amino}piperidin-1-yl)-N-(4-cyanobenzyl)propanamide) (0.27 g), with triethylamine trihydrofluoride (0.36 mL) in THF (5 mL) at room temperature for 18 h to afford the title compound as an off-white solid. Yield: 0.1 g

MS APCI+ 490 [M+H]⁺

¹H NMR (DMSO) δ 8.17 (d, 1H), 7.80 (d, 2H), 7.46 (d, 2H), 7.45 (d, 2H), 6.59 (d, 1H), 5.37-5.30 (m, 1H), 4.38 (d, 2H), 3.71-1.70 (m, 15H).

### EXAMPLE 13

### N-(2-Hydroxybenzyl)-3-(4-{[(2R)-2-hydroxy-2-(8-hydroxy-2-oxo-1,2-dihydroquinolin-5-yl)ethyl]amino}piperidin-1-yl)propanamide

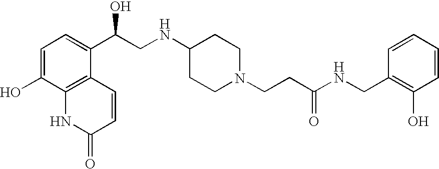

### i) 3-(4-{[(2R)-2-{[tert-Butyl(dimethyl)silyl]oxy}-2-(8-hydroxy-2-oxo-1,2-dihydroquinolin-5-yl)ethyl]amino}piperidin-1-yl)-N-(2-hydroxybenzyl)propanamide

The sub-titled compound was prepared according to the procedure outlined in Example 6 step (iv) using a solution of the product from Example 6 step (iii) (3-(4-{[(2R)-2-{[tert-butyl(dimethyl)silyl]oxy}-2-(8-hydroxy-2-oxo-1,2-dihydroquinolin-5-yl)ethyl]amino}piperidin-1-yl)propanoic acid) (0.22 g), 2-(aminomethyl)phenol (0.12 g), triethylamine (0.17 mL) and HATU (0.31 g) in DMF (5 mL) at room temperature for 18 h to afford the sub-title compound as a dark yellow oil. Yield: 0.27 g.

MS APCI+ 595 [M+H]⁺

### ii) N-(2-Hydroxybenzyl)-3-(4-{[(2R)-2-hydroxy-2-(8-hydroxy-2-oxo-1,2-dihydroquinolin-5-yl)ethyl]amino}piperidin-1-yl)propanamide

The sub-titled compound was prepared according to the procedure outlined in Example 6 step (v) using a solution of the product from step (i) (3-(4-{[(2R)-2-{[tert-butyl(dimethyl)silyl]oxy}-2-(8-hydroxy-2-oxo-1,2-dihydroquinolin-5-yl)ethyl]amino}piperidin-1-yl)-N-(2-hydroxybenzyl)propanamide) (0.27 g), with triethylamine trihydrofluoride (0.36 mL) in THF (5 mL) at room temperature for 18 h to afford the title compound as an off-white solid. Yield: 0.14 g

MS APCI+ 481 [M+H]⁺

¹H NMR (DMSO) δ 8.15 (d, 1H), 7.17 (d, 1H), 7.13-7.05 (m, 2H), 6.99 (d, 1H), 6.81 (d, 1H), 6.75 (t, 1H), 6.60 (d, 1H), 5.35-5.29 (m, 1H), 4.23 (d, 2H), 3.63-1.68 (m, 15H).

### EXAMPLE 14

### 8-Hydroxy-5-{(1R)-1-hydroxy-2-[({(3R)-1-[2-(2-phenylethoxy)ethyl]piperidin-3-yl}methyl)amino]ethyl}quinolin-2(1H)-one

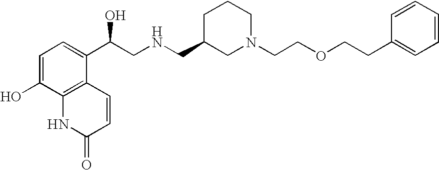

### i) Ethyl (3S)-1-[2-(2-phenylethoxy)ethyl]piperidine-3-carboxylate

To a solution of (2-phenylethoxy)acetic acid (0.6 g) (J. Med. Chem. 1983, 26 (11), 1570) in DCM (12 mL) was added oxalyl chloride (1.6 mL) followed by 1 drop of DMF and the whole stirred at room temperature for 3 h. The volatiles were then evaporated in vacuo and the residue azeotroped with DCM (×3). Dry THF (5 mL) was added and this suspension then added batchwise to a mixture of ethyl (3S)-piperidine-3-carboxylate tartrate salt (1.0 g) in dry THF (12 mL) and triethylamine (2.25 mL) at 3° C. The mixture was further stirred for 10 min before allowing the temperature to rise to room temperature and then further stirring for 2.5 h. The mixture was partioned between EtOAc and H₂O. The organic phase was further washed with saturated sodium bicarbonate solution, then saturated aqueous NaCl, collected, dried (MgSO₄) and the solvent evaporated to leave an orange-yellow oil. Purification was by silica gel column chromatography eluting with 4:6 EtOAc/isohexane to give the sub-title compound as a clear oil. Yield: 0.36 g

MS APCI+ 320 [M+H]⁺

### ii) {(3S)-1-[2-(2-Phenylethoxy)ethyl]piperidin-3-yl}methanol

A solution of the product from step (i) (ethyl (3S)-1-[2-(2-phenylethoxy)ethyl]piperidine-3-carboxylate) (0.36 g) in dry THF (1.4 mL) was added dropwise to a solution of lithium aluminum hydride (1.8 mL, 1.0M in THF) in THF (5.6 mL) under nitrogen. After stirring for 2.5 h EtOAc (0.86 mL) was added dropwise followed by water (1.43 mL). The mixture was partitioned between EtOAc and saturated aqueous NaCl. The organic phase was collected and the product then extracted into 1N HCl followed by basification of the aqueous layer with saturated aqueous sodium bicarbonate solution and the product extracted into DCM. The organic phase was collected, dried (MgSO₄) and the solvent evaporated leaving the sub-title compound as a yellow gum. Yield: 0.23 g

MS APCI+ 264 [M+H]⁺

### iii) (3S)-1-[2-(2-Phenylethoxy)ethyl]piperidine-3-carbaldehyde

To oxalyl chloride (0.086 mL) in DCM (1.5 mL) at −78° C. was added DMSO (0.07 mL) dropwise. After 35 min a solution of the product from step (ii) ({(3S)-1-[2-(2-phenylethoxy)ethyl]piperidin-3-yl}methanol) (0.23 g) in DCM (1.5 mL) was added dropwise at −65° C. The mixture was further stirred at −70° C. for 3 h before addition of triethylamine (0.27 mL) and the mixture allowed to reach room temperature with further stirring for 18 h. The volatiles were evaporated in vacuo and the residue azeotroped with toluene (×2) to leave the crude sub-title compound as a clear gum. This was used in step (iv) without further purification. Yield: 0.2 g

### iv) 5-{(1R)-1-{[tert-Butyl(dimethyl)silyl]oxy}-2-[({(3R)-1-[2-(2-phenylethoxy)ethyl]piperidin-3-yl}methyl)amino]ethyl}-8-hydroxyquinolin-2(1H)-one

The crude product from step (iii) was dissolved in methanol (7 mL). The product of Example 5 step (ii) (5-((1R)-2-amino-1-{[tert-butyl(dimethyl)silyl]oxy}ethyl)-8-hydroxyquinolin-2(1H)-one) (0.2 g), was then added along with AcOH (0.037 mL). After stirring at room temperature for 2 h, sodium cyanoborohydride (41 mg) was added and the reaction mixture was stirred for 18 h at room temperature. The solvent was evaporated in vacuo and the residue partitioned between EtOAc and water. The organic phase washed with saturated sodium bicarbonate solution, collected, dried (MgSO₄) and the solvent evaporated to leave a yellow foam. Purification was by silica gel chromatography eluting with EtOAc/methanol/ammonia mixtures to give the sub-title compound as a yellow foam.

Yield: 0.27 g

MS APCI+ 580 [M+H]⁺

### v) 8-Hydroxy-5-{(1R)-1-hydroxy-2-[({(3R)-1-[2-(2-phenylethoxy)ethyl]piperidin-3-yl}methyl)amino]ethyl}quinolin-2(1H)-one

A solution of the product from step (iv) (5-{(1R)-1-{[tert-butyl(dimethyl)silyl]oxy}-2-[({(3R)-1-[2-(2-phenylethoxy)ethyl]piperidin-3-yl}methyl)amino]ethyl}-8-hydroxyquinolin-2(1H)-one) (0.27 g) in dry THF (10 mL) was treated with triethylamine trihydrofluoride (0.76 mL) and the whole further stirred at room temperature for 18 h. The solvent was evaporated in vacuo and the residue purified by reverse phase HPLC eluting with gradient 0-50% acetonitrile in 0.2% aqueous TFA on an ACE® column to give the titled compound after trituration with Et₂O as an off-white solid. Yield: 0.19 g

MS APCI+ 466 [M+H]⁺

¹H NMR (DMSO) δ 10.50 (bs, 1H), 9.75 (bs, 1H), 8.17 (d, 1H), 7.24 (m, 6H), 6.99 (d, 1H), 6.57 (d, 1H), 6.23 (bs, 1H), 5.34 (bd, 1H), 3.75 (bs, 2H), 3.69 (t, 2H), 3.40 (m, 2H), 3.25 (bs, 2H), 3.10 (bs, 1H), 2.80 (m, 5H), 2.27 (bs, 1H), 1.80 (m, 2H), 1.67 (m, 1H), 1.10 (m, 1H).

### EXAMPLE 15

### 8-Hydroxy-5-{(1R)-1-hydroxy-2-[({4-hydroxy-1-[3-(2-phenylethoxy)propyl]piperidin-4-yl}methyl)amino]ethyl}quinolin-2(1H)-one

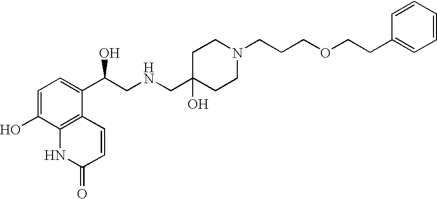

### i) 6-[3-(2-Phenylethoxy)propyl]-1-oxa-6-azaspiro[2.5]octane

The sub-titled compound was prepared according to the procedure outlined in Example 5 step (iii) using a solution of the product from Example 3 step (v) (1-[3-(2-phenylethoxy)propyl]piperidin-4-one) (0.5 g), trimethylsulfoxonium iodide (0.63 g), 60% sodium hydride (0.15 g) and DMSO (4 mL) to afford the sub-title compound as a colourless oil. Yield: 0.5 g

¹H NMR (DMSO) δ 7.30 (m, 5H), 3.56 (t, 2H), 3.41 (t, 2H), 2.79 (t, 2H), 2.42 (t, 4H), 2.31 (t, 2H), 1.60 (m, 4H), 1.40 (m, 2H).

### ii) 5-{(1R)-1-{[tert-Butyl(dimethyl)silyl]oxy}-2-[({4-hydroxy-1-[3-(2-phenylethoxy)propyl]piperidin-4-yl}methyl)amino]ethyl}-8-hydroxyquinolin-2(1H)-one

The sub-titled compound was prepared according to the procedure outlined in Example 5 step (iv) using a solution of the product from step (i) (6-[3-(2-phenylethoxy)propyl]-1-oxa-6-azaspiro[2.5]octane) (0.26 g) and the product from Example 5 step (ii) (5-((1R)-2-amino-1-{[tert-butyl(dimethyl)silyl]oxy}ethyl)-8-hydroxyquinolin-2(1H)-one) (0.32 g) in methanol (10 mL) and N,N-diisopropylethyamine (0.092 mL) which was heated at reflux for 24 h. The volatiles were evaporated in vacuo to leave a brown gum. Purification was by silica gel chromatography eluting with 10% methanol in DCM to give the sub-title compound as a yellow foam. Yield: 0.17 g

MS APCI+ 610 [M+H]⁺

### iii) 8-Hydroxy-5-{(1R)-1-hydroxy-2-[({4-hydroxy-1-[3-(2-phenylethoxy)propyl]piperidin-4-yl}methyl)amino]ethyl}quinolin-2(1H)-one

The title compound was prepared according to the procedure outlined in Example 5 step (v) using a solution of the product from step (ii) (5-{(1R)-1-{[tert-butyl(dimethyl)silyl]oxy}-2-[({4-hydroxy-1-[3-(2-phenylethoxy)propyl]piperidin-4-yl}methyl)amino]ethyl}-8-hydroxyquinolin-2(1H)-one) (0.17 g) in THF (5 mL), treated with triethylamine trihydrofluoride (0.23 mL). Purification was by reverse phase HPLC using an Xterra® C8 5 micron 19×50 mm column eluting with a gradient of 95% 0.880 NH₃ in acetonitrile to 5% 0.880 NH₃ in acetonitrile followed by trituration with Et₂O to give the title compound as an off-white solid. Yield: 56 mg

MS APCI+ 496[M+H]⁺

¹H NMR (DMSO) δ 10.55 (s, 2H), 8.20 (d, 1H), 7.20 (m, 6H), 7.00 (d, 1H), 6.59 (d, 1H), 5.40 (m, 1H), 3.60 (t, 4H), 3.40 (m, 4H), 3.10 (m, 6H), 2.82 (t, 2H), 1.80 (m, 6H).

### EXAMPLE 16

### 8-Hydroxy-5-{(1R)-1-hydroxy-2-[({4-methyl-1-[2-(2-phenylethoxy)ethyl]piperidin-4-yl}methyl)amino]ethyl}quinolin-2(1H)-one

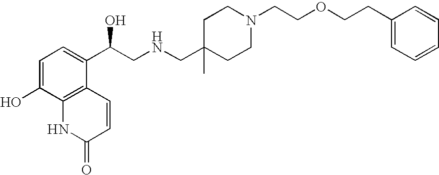

### i) 4-Cyano-4-methylpiperidine hydrochloride

4M HCl in dioxane (9 mL) was added to tert-butyl 4-cyano-4-methylpiperidine-1-carboxylate (WO 03/042174(A1)) (0.794 g). After 1.5 h a thick white precipitate had formed and the reaction mixture was concentrated in vacuo to afford the sub-title compound as a cream fluffy solid. Yield: 0.569 g

¹H NMR (DMSO) δ 9.03 (s, 2H), 3.36-3.30 (m, 2H), 2.96-2.87 (m, 2H), 2.10 (d, 2H), 1.82-1.72 (m, 2H), 1.39 (s, 3H).

### ii) 4-Methyl-1-[2-(2-phenylethoxy)ethyl]piperidine-4-carbonitrile

A mixture of the product from example 4 step (i) (2-(2-phenylethoxy)ethyl 4-methylbenzenesulfonate) (1.344 g) and the product from step (i) (4-cyano-4-methylpiperidine hydrochloride) (0.569 g) was dissolved in NMP (12 mL) and triethylamine (3 mL) added. The reaction mixture was heated at 85° C. for 3 h then allowed to cool and partitioned between EtOAc and water. The layers were separated and the aqueous layer extracted with further EtOAc. The combined organic extracts were washed with saturated aqueous NaHCO₃, water, saturated aqueous NaCl, dried (Na₂SO₄) and concentrated. The residue was diluted with isopropanol then loaded onto a Varian SCX column (50 g). The column was washed with isopropanol then eluted with 1:3 0.880 NH₃/isopropanol to afford the sub-title compound as a brown oil which contains 1 mole equivalent of NMP. Yield: 0.691 g

¹H NMR (CDCl₃) δ 7.30-7.19 (m, 5H), 3.66 (t, 2H), 3.57 (t, 2H), 2.91-2.87 (m, 4H), 2.61 (t, 2H), 2.35-2.30 (m, 2H), 1.89-1.85 (m, 2H), 1.58 (td, 2H), 1.37 (s, 3H).

### iii) 8-(Benzyloxy)-5-{(1R)-1-{[tert-butyl(dimethyl)silyl]oxy}-2-[({4-methyl-1-[2-(2-phenylethoxy)ethyl]piperidin-4-yl}methyl)amino]ethyl}quinolin-2(1H)-one

Lithium aluminium hydride (15 mL, 1M in THF) was added slowly to a cooled (−78° C.) solution of the product from step (ii) (4-methyl-1-[2-(2-phenylethoxy)ethyl]piperidine-4-carbonitrile) (0.691 g) in dry THF (10 mL). The reaction mixture was allowed to warm to room temperature gradually and stirred overnight. It was cooled in ice then EtOAc (5 mL) added cautiously, followed by isopropanol. When no further effervescence occurred, the reaction mixture was poured onto ice and extracted with EtOAc (×10). The combined organic extracts were washed with 10% aqueous sodium potassium tartrate followed by water, saturated aqueous NaCl, dried (Na₂SO₄) and the volatiles removed in vacuo to afford the intermediate compound 1-{4-methyl-1-[2-(2-phenylethoxy)ethyl]piperidin-4-yl}methanamine as a pale yellow oil (0.455 g) which was used without further purification.

A mixture of the product from Example 2 step (iii) (8-(benzyloxy)-5-((1R)-2-bromo-1-{[tert-butyl(dimethyl)silyl]oxy}ethyl)quinolin-2(1H)-one) (0.403 g), the intermediate compound prepared as above (1-{4-methyl-1-[2-(2-phenylethoxy)ethyl]piperidin-4-yl}methanamine) (0.228 g), sodium iodide (0.247 g) and sodium bicarbonate (0.277 g) in DMSO (1 mL) was heated at 100° C. for 8 h then diluted with EtOAc and water, and the aqueous material extracted with further EtOAc (×3). The combined organic extracts were washed with water, saturated aqueous NaCl, dried (Na₂SO₄) and concentrated. Purification was by Biotage chromatography (gradient 1:4:95 to 3:12:85 7M NH₃ in methanol:methanol:EtOAc) to afford the sub-title compound as a yellow oil. Yield: 0.311 g

MS APCI+ 684 [M+H]⁺

### iv) 8-(Benzyloxy)-5-{(1R)-1-hydroxy-2-[({4-methyl-1-[2-(2-phenylethoxy)ethyl]piperidin-4-yl}methyl)amino]ethyl}quinolin-2(1H)-one

The product from step (iii) (8-(benzyloxy)-5-{(1R)-1-{[tert-butyl(dimethyl)silyl]oxy}-2-[({4-methyl-1-[2-(2-phenylethoxy)ethyl]piperidin-4-yl}methyl)amino]ethyl}quinolin-2(1H)-one) (0.311 g) was dissolved in THF (5 mL) and treated with triethylamine trihydrofluoride (0.25 mL). The reaction mixture was stirred at room temperature overnight then the volatiles evaporated to afford the sub-title compound as a yellow oil, which was used without further purification. Yield: 0.259 g

MS APCI+ 570 [M+H]⁺

### v) 8-Hydroxy-5-{(1R)-1-hydroxy-2-[({4-methyl-1-[2-(2-phenylethoxy)ethyl]piperidin-4-yl}methyl)amino]ethyl}quinolin-2(1H)-one

A suspension of palladium hydroxide (20 wt % on carbon, wet, 70 mg) in 1:1 DCM:methanol (5 mL) was added to a solution of the product from step (iv) (8-(benzyloxy)-5-{(1R)-1-hydroxy-2-[({4-methyl-1-[2-(2-phenylethoxy)ethyl]piperidin-4-yl}methyl)amino]ethyl}quinolin-2(1H)-one) (0.259 g) and hydrogenated at 4 bar for 3.5 h. The catalyst was removed and the solvent evaporated, then the crude material was purified by reverse phase HPLC using a Symmetry® column eluting with a gradient of 10-40% acetonitrile in 0.2% aqueous TFA) to afford the title compound ditrifluoroacetate salt as a pale yellow solid. Yield: 0.150 g

MS APCI+ 480 [M+H]⁺

¹H NMR (90° C., DMSO) δ 8.22 (d, 1H), 7.31-7.18 (m, 5H), 7.14 (d, 1H), 7.01 (d, 1H), 6.54 (d, 1H), 5.46-5.42 (m, 1H), 3.77-3.74 (m, 2H), 3.71 (t, 2H), 3.29-3.26 (m, 4H), 3.20-3.17 (m, 4H), 3.12-3.10 (m, 2H), 2.85 (t, 2H), 1.86-1.65 (m, 4H), 1.16 (s, 3H).

### EXAMPLE 17

### N-Benzyl-4-(4-{[(2R)-2-hydroxy-2-(8-hydroxy-2-oxo-1,2-dihydroquinolin-5-yl)ethyl]amino}piperidin-1-yl)butanamide

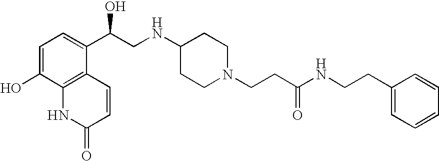

### i) tert-Butyl 4-(4-oxopiperidin-1-yl)butanoate

Sodium bicarbonate (1.31 g) and potassium iodide (87 mg) were added to a solution of piperidin-4-one hydrochloride monohydrate (0.80 g) and tert-butyl 4-bromobutanoate (1.0 g) in methyl isobutylketone (25 mL). The reaction mixture was heated at reflux for 4 h then allowed to cool and the solvent partially evaporated. The residue was dissolved in DCM and water, the layers separated and the organic layer washed with 10% aqueous sodium thiosulfate, water, saturated aqueous NaCl, dried (Na₂SO₄) and the volatiles removed in vacuo. Purification was by Biotage chromatography (gradient 0-10% methanol in DCM) to afford the sub-title compound as an orange oil. Yield: 0.697 g

¹H NMR (CDCl₃) δ 2.75 (t, 4H), 2.43-2.51 (m, 6H), 2.31 (t, 2H), 1.82 (quintet, 2H), 1.46 (s, 9H).

### ii) tert-Butyl 4-(4-{[(2R)-2-hydroxy-2-(8-hydroxy-2-oxo-1,2-dihydroquinolin-5-yl)ethyl]amino}piperidin-1-yl)butanoate

The product from Example 3 step (iii) (4-[(1R)-2-amino-1-hydroxyethyl]-8-hydroxyquinolin-2(1H)-on)e (0.154 g) and the product from step (i) (tert-butyl 4-(4-oxopiperidin-1-yl)butanoate) (0.188 g) were partially dissolved in NMP (5 mL) and AcOH (40 μl) added. The reaction mixture was stirred at room temperature for 1 h, then further NMP (5 mL) was added. After a further 2 h sodium triacetoxyborohydride (0.254 g) was added. The reaction mixture was stirred at room temperature overnight, then 0.880 aqueous NH₃ (10 mL) was added and the volatiles removed in vacuo. The residue was diluted with isopropanol and loaded onto a Varian SCX column (10 g) which was eluted with 50 mL isopropanol followed by 1:3 aqueous NH₃:isopropanol. The basic wash was concentrated in vacuo to afford to afford the sub-title compound as a dark yellow oil. Yield: 0.27 g

MS APCI+ 446 [M+H]⁺

### iii) 4-(4-{[(2R)-2-Hydroxy-2-(8-hydroxy-2-oxo-1,2-dihydroquinolin-5-yl)ethyl]amino}piperidin-1-yl)butanoic acid

TFA (1.5 mL) was added to the product from step (ii) (tert-butyl 4-(4-{[(2R)-2-hydroxy-2-(8-hydroxy-2-oxo-1,2-dihydroquinolin-5-yl)ethyl]amino}piperidin-1-yl)butanoate) (0.27 g) and the mixture was stirred for 2 h. The mixture was concentrated in vacuo then diluted with isopropanol, neutralised with 0.880 aqueous NH₃ and concentrated before loading onto a Varian SCX column (10 g). The column was eluted with isopropanol then 1:3 0.880 aqueous NH₃:isopropanol to afford the sub-title compound as a yellow solid. Yield; 0.267 g

MS APCI+ 390 [M+H]⁺

### iv) N-Benzyl-4-(4-{[(2R)-2-hydroxy-2-(8-hydroxy-2-oxo-1,2-dihydroquinolin-5-yl)ethyl]amino}piperidin-1-yl)butanamide

The product from step (iii) (4-(4-{[(2R)-2-hydroxy-2-(8-hydroxy-2-oxo-1,2-dihydroquinolin-5-yl)ethyl]amino}piperidin-1-yl)butanoic acid) (0.267 g) was dissolved in DMF (3 mL) then benzylamine (0.65 mL), triethylamine (0.25 mL) and HATU (0.46 g) were added. After 2 h more HATU (0.51 g) was added. The reaction mixture was stirred at room temperature overnight then the DMF partially evaporated and the residue dissolved in 1:1 isopropanol:water, neutralised with 2M aqueous HCl and filtered. The filtrate was loaded onto a Varian SCX cartidge (10 g), washed with isopropanol then eluted with 1:3 0.880 aqueous NH₃:isopropanol to afford crude product which was purified by reverse phase HPLC using a Symmetry® column eluting with a gradient of 10-50% acetonitrile in 0.2% aqueous TFA to afford the title compound ditrifluoroacetate salt as a pale tan solid

Yield: 0.110 g

MS APCI+ 479 [M+H]⁺

¹H NMR (DMSO) δ 10.50 (s, 2H), 9.75 (s, 1H), 9.21 (s, 1H), 8.94 (s, 1H), 8.49 (t, 1H), 8.17 (d, 1H), 7.34-7.24 (m, 5H), 7.17 (d, 1H), 6.99 (d, 1H), 6.58 (d, 1H), 5.33 (d, 1H), 4.28 (d, 2H), 3.62-3.59 (m, 1H), 3.40-3.34 (m, 2H), 3.15-2.91 (m, 6H), 2.33-2.25 (m, 4H), 1.92-1.78 (m, 4H).

### EXAMPLE 18

### 8-Hydroxy-5-{(1R)-1-hydroxy-2-[({4-methoxy-1-[2-(2-phenylethoxy)ethyl]piperidin-4-yl}methyl)amino]ethyl}quinolin-2(1H)-one

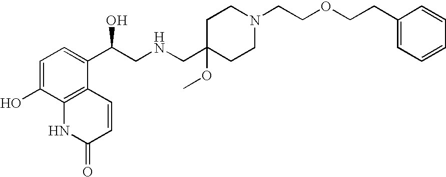

### i) 4-(Azidomethyl)-1-[2-(2-phenylethoxy)ethyl]piperidin-4-ol

A solution of the product from example 5 step (iii) (2.0 g) (6-[2-(2-phenylethoxy)ethyl]-1-oxa-6-azaspiro[2.5]octane) in dioxane (10 mL) and water (3 mL) was treated with sodium azide (1.7 g) and then heated at 85° C. for 14 h. The cooled mixture was partitioned between water and Et₂O. The organic phase was further washed with saturated aqueous NaCl, collected, dried (MgSO₄) and the solvent evaporated in vacuo to leave the sub-title compound as a yellow oil. Yield: 2.2 g

¹H NMR (CDCl₃) δ 7.24 (m, 5H), 3.67 (m, 2H), 3.58 (m, 2H), 3.27 (s, 2H), 2.89 (t, 2H), 2.60 (m, 6H), 2.36 (m, 2H).

### ii) 4-(Azidomethyl)-4-methoxy-1-[2-(2-phenylethoxy)ethyl]piperidine

A solution of the product from step (i) (4-(azidomethyl)-1-[2-(2-phenylethoxy)ethyl]piperidin-4-ol) (0.5 g) in dry DMF (2 mL) was treated with 60% sodium hydride (70 g) under nitrogen. After stirring for 15 min, iodomethane (0.13 mL) was added and the mixture further stirred for 18 h. The mixture was partitioned between water and EtOAc. The organic phase was further washed with water, collected, dried (MgSO₄) and the solvent evaporated in vacuo to leave the sub-title compound as a yellow oil. Yield: 0.25 g

MS APCI+ 319 [M+H]⁺

### iii) 1-{4-Methoxy-1-[2-(2-phenylethoxy)ethyl]piperidin-4-yl}methanamine

A solution of the product from step (ii) (4-(azidomethyl)-4-methoxy-1-[2-(2-phenylethoxy)ethyl]piperidine) (0.25 g) in ethanol (10 mL) was treated with 10% palladium on charcoal (20 mg) and then stirred under 4 bar pressure of hydrogen gas for 3 h. The mixture was filtered through glass fibre paper and the filtate evaporated in vacuo to leave the sub-title compound as a yellow gum. Yield: 0.18 g

MS APCI+ 293[M+H]⁺

### iv) 8-(Benzyloxy)-5-{(1R)-1-{[tert-butyl(dimethyl)silyl]oxy}-2-[({4-methoxy-1-[2-(2-phenylethoxy)ethyl]piperidin-4-yl}methyl)amino]ethyl}quinolin-2(1H)-one

The sub-title compound was prepared according to the procedure outlined in Example 2 step (vi) using a solution of the product from step (iii) (1-{4-methoxy-1-[2-(2-phenylethoxy)ethyl]piperidin-4-yl}methanamine) (0.18 g) and the product of Example 2 step (iii) (8-(benzyloxy)-5-((1R)-2-bromo-1-{[tert-butyl(dimethyl)silyl]oxy}ethyl)quinolin-2(1H)-one) (0.195 g) in DMSO (2 mL) in the presence of sodium iodide (88 mg) and potassium carbonate (0.166 g) and heated at 90° C. for 6 h. Purification was by column chromatography eluting with EtOAc/isohexane (8:4) to give the sub-title compound as a yellow gum.

Yield: 60 mg

MS APCI+ 701 [M+H]⁺

### v) 8-Hydroxy-5-{(1R)-1-hydroxy-2-[({4-methoxy-1-[2-(2-phenylethoxy)ethyl]piperidin-4-yl}methyl)amino]ethyl}quinolin-2(1H)-one

The title compound was prepared according to the procedure outlined in Example 2 step (vii) using the product of step (iv) (8-(benzyloxy)-5-{(1R)-1-{[tert-butyl(dimethyl)silyl]oxy}-2-[({4-methoxy-1-[2-(2-phenylethoxy)ethyl]piperidin-4-yl}methyl)amino]ethyl}quinolin-2(1H)-one) (60 mg) in THF (2 mL) treated with triethylamine trihydrofluoride (0.14 mL) and stirred at room temperature for 5 h. The volatiles were evaporated and the residue azeoptroped with toluene (×2). The residue was dissolved in ethanol (10 mL) followed by addition of 10% palladium on charcoal (20 mg) and then stirred under 5 bar pressure of hydrogen gas overnight. The mixture was filtered and the solvent evaporated in vacuo to leave a yellow gum. Purification was by reverse phase HPLC using a Symmetry® C8 5 micron 19×50 mm column, eluting with gradient 0-50% acetonitrile in 0.2% aqueous TFA to give the title compound as an off-white solid.

Yield: 20 mg

MS APCI+ 496 [M+H]⁺

¹H NMR (DMSO) δ 10.54 (bs, 1H), 10.50 (bs, 1H), 8.77 (bs, 1H), 8.23 (d, 1H), 7.20 (m, 5H), 7.0 (d, 1H), 6.60 (d, 1H), 6.20 (bs, 1H), 5.40 (m, 1H), 3.60 (m, 4H), 2.90 (m, 4H), 2.0 (m, 4H), 1.80 (m, 2H), 1.20 (m, 2H).

### EXAMPLE 19

### 4-Hydroxy-7-{(1R)-1-hydroxy-2-[({1-[2-(2-phenylethoxy)ethyl]piperidin-4-yl}methyl)amino]ethyl}-1,3-benzothiazol-2(3H)-one

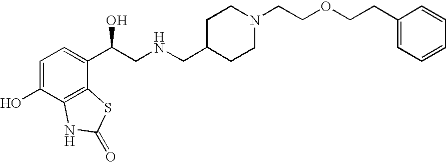

### i) [1-(2-Phenethyloxy-ethyl)-piperidin-4-yl]-methanol

A solution of 2-phenethyloxyacetaldehyde (WO 94/27601) (0.72 g) and 4-piperidine-methanol (0.5 g) in methanol (20 mL) was treated with acetic acid (20 mg) and stirred at room temperature for 30 minutes. At the end of this time, sodium cyanoborohydride (103 mg) was added and the mixture was stirred for 18 h at room temperature. The reaction mixture was basified by addition of concentrated aqueous ammonia (1 mL) and the solvent was removed under reduced pressure. The crude product was purified by flash chromatography on a silica column, eluting with 1% concentrated aqueous ammonia and 5% ethanol in dichloromethane to yield the sub-titled compound. Yield: 0.1 g

MS APCI+ 264 (M+H⁺, 100%)

¹H NMR (400 MHz, CDCl₃) δ 7.30-7.18 (m, 5H), 3.67-3.64 (m, 2H), 3.60-3.57 (m, 2H), 3.49-3.47 (m, 2H), 2.95-2.87 (m, 4H), 2.58-2.55 (m, 2H), 2.02-1.96 (m, 2H), 1.72-1.69 (m, 2H), 1.54-1.43 (m, 1H), 1.33-1.23 (m, 2H).

### ii) 1-(2-Phenethyloxy-ethyl)-piperidine-4-carbaldehyde

A solution of dimethyl sulphoxide (32 mg) in dichloromethane (3 mL) was cooled to −78° C. and treated dropwise with a solution of oxalyl chloride (53 mg) in dichloromethane (1 mL). The reaction mixture was stirred at −78° C. for 15 minutes and then treated dropwise with a solution of the product of step (i) ([1-(2-phenethyloxy-ethyl)-piperidin-4-yl]-methanol) (100 mg) in dichloromethane (1 mL). The reaction mixture was stirred at −78° C. for 1 hour and then treated dropwise with triethylamine (77 mg), after which the cooling bath was removed and the mixture was allowed to warm to room temperature. The reaction mixture was added to aqueous phosphate buffer (pH 7.2) (10 mL) and extracted with dichloromethane. The organic layer was dried with anhydrous magnesium sulphate and the solvent was evaporated under reduced pressure to give the sub-titled compound.

Yield: 99 mg

MS APCI+ 262 (M+H)⁺

### iii) 7-[(1R)-2-Azido-1-hydroxyethyl]-4-(benzyloxy)-1,3-benzothiazol-2(3H)-one

To a solution of 4-(benzyloxy)-7-[(1R)-2-bromo-1-hydroxyethyl]-1,3-benzothiazol-2(3H)-one (WO 2004/016578) (340 mg) in dimethyl sulfoxide (8 mL) was added sodium azide (231 mg) and sodium iodide (147 mg). The reaction mixture was heated at 65° C. for 5 hours. At the end of this time the mixture was partitioned between ethyl acetate and water, the organic phase was washed with water, dried with anhydrous magnesium sulphate, filtered and concentrated under reduced pressure to give a crude product. The residue was purified by flash chromatography on a silica column, eluting with 20% ethyl acetate in toluene to yield the sub-titled compound. Yield: 195 mg

¹H NMR (400 MHz, d₆-DMSO) δ 11.89 (s, 1H), 7.54 (d, 2H), 7.38 (t, 2H), 7.33-7.29 (m, 1H), 7.02 (s, 2H), 6.13 (d, 1H), 5.25 (s, 2H), 4.81-4.77 (m, 1H), 3.40-3.27 (m, 2H).

### iv) 7-[(1R)-2-Amino-1-hydroxyethyl]-4-(benzyloxy)-1,3-benzothiazol-2(3H)-one hydrochloride

A solution of the product of step (iii) (7-((1R)-2-azido-1-hydroxyethyl)-4-benzyloxy-3H-benzothiazol-2-one) (195 mg) in a mixture of ethanol (8 mL) and tetrahydrofuran (4 mL) was treated with 10% palladium on carbon catalyst (20 mg) and the resultant mixture was stirred vigorously under 3 atmospheres pressure of hydrogen gas for 20 hours. The catalyst was filtered off and the solvent was removed under reduced pressure. The residue was purified by flash chromatography on a silica column, eluting with 1% concentrated aqueous ammonia and 12% methanol in dichloromethane. The resultant product was dissolved in 1,4-dioxane and treated dropwise with a 4 molar solution of hydrogen chloride in 1,4 dioxane (0.5 mL). Evaporation of the solvent under reduced pressure gave the sub-titled compound. Yield: 160 mg

MS APCI+ 315 (M−H)⁺

¹H NMR (400 MHz, d₆-DMSO) δ 8.01 (s, 2H), 7.55 (d, 2H), 7.39 (t, 2H), 7.31 (t, 1H), 7.04 (q, 2H), 6.39 (d, 1H), 5.26 (s, 2H), 4.83 (dt, 1H), 2.97-2.83 (m, 2H).

### v) 4-(Benzyloxy)-7-{(1R)-1-hydroxy-2-[({1-[2-(2-phenylethoxy)ethyl]piperidin-4-yl}methyl)amino]ethyl}-1,3-benzothiazol-2(3H)-one

A solution of the product of step (iv) (7-[(1R)-2-amino-1-hydroxyethyl]-4-(benzyloxy)-1,3-benzothiazol-2(3H)-one hydrochloride) (133 mg) and the product of step (ii) (1-(2-phenethyloxy-ethyl)-piperidine-4-carbaldehyde) (99 mg) in methanol (20 mL) was treated with acetic acid (20 mg) and stirred at room temperature for 2 hours. At the end of this time sodium cyanoborohydride (9 mg) was added and the mixture was stirred for 18 hours at room temperature. The reaction mixture was basified by addition of concentrated aqueous ammonia and the solvent was removed under reduced pressure. The residue was purified by flash chromatography on a silica column, eluting with 1% concentrated aqueous ammonia and 6% methanol in dichloromethane to give the sub-titled compound.

Yield: 28 mg

MS APCI+ 560 (M−H)⁺

### vi) 4-Hydroxy-7-{(1R)-1-hydroxy-2-[({1-[2-(2-phenylethoxy)ethyl]piperidin-4-yl}methyl)amino]ethyl}-1,3-benzothiazol-2(3H)-one

A mixture of the product of step (v) (4-(benzyloxy)-7-{(1R)-1-hydroxy-2-[({1-[2-(2-phenylethoxy)ethyl]piperidin-4-yl}methyl)amino]ethyl}-1,3-benzothiazol-2(3H)-one) (28 mg) and 10% palladium on carbon (25 mg) in ethanol (10 mL) and concentrated hydrochloric acid (0.1 mL) was stirred vigorously under 4 atmospheres pressure of hydrogen gas for 7 hours. At the end of this time a further portion of 10% palladium on carbon (25 mg) and concentrated hydrochloric acid (0.1 mL) were added and stirring under hydrogen continued for 16 hours. The catalyst was filtered off and the solvent was evaporated under reduced pressure. The residue was purified by reverse phase HPLC using a gradient elution of 5% to 50% acetonitrile in 0.2% aqueous ammonia to give the titled compound. Yield: 6 mg

MS APCI+ 472 (M+H)⁺

¹H NMR (400 MHz, d₆-DMSO) δ 7.29-7.16 (m, 5H), 6.85 (d, 1H), 6.68 (d, 1H), 4.58-4.55 (m, 1H), 3.57 (t, 2H), 3.47 (t, 2H), 2.79 (t, 4H), 2.67-2.54 (m, 2H), 2.42-2.33 (m, 4H), 1.85 (t, 2H), 1.58 (d, 2H), 1.28-1.25 (m, 1H), 1.10-1.00 (m, 2H).

### EXAMPLE 20

### 4-Hydroxy-7-{(1R)-1-hydroxy-2-[({1-[3-(2-phenylethoxy)propyl]azetidin-3-yl}methyl)amino]ethyl}-1,3-benzothiazol-2(3H)-one hydrochloride

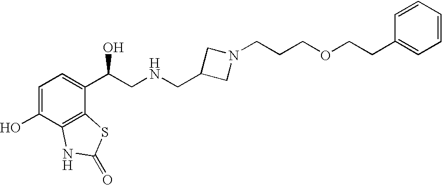

### i) {1-[3-(2-Phenylethoxy)propanoyl]azetidin-3-yl}methanol

A solution of 3-(2-phenylethoxy)-propanoic acid (718 mg) in dichloromethane (10 mL) was treated with oxalyl chloride (940 mg) and stirred for 4 hours at room temperature before evaporating the solvent under reduced pressure. The residue was dissolved in dichloromethane (5 mL) and the resultant solution was added dropwise to a solution of 3-azetidinemethanol (460 mg) and triethylamine (747 mg) in a mixture of dichloromethane (10 mL) and 1-methyl-2-pyrrolidinone (3 mL). The reaction mixture was stirred for 2 hours and then partitioned between dichloromethane and brine, the aqueous layer was re-extracted with further dichloromethane and the combined organic layers were dried with anhydrous magnesium sulphate, filtered and concentrated under reduced pressure. The residue was purified by flash chromatography on a silica column, eluting with 6% methanol in dichloromethane to give the sub-titled compound. Yield: 560 mg

MS APCI+ 264 (M+H)⁺

### ii) {1-[3-(2-Phenylethoxy)propyl]azetidin-3-yl}methanol

A solution of the product of step (i) ({1-[3-(2-phenylethoxy)propanoyl]azetidin-3-yl}methanol) (560 mg) in tetrahydrofuran (10 mL) was treated with a 1 molar solution of borane-tetrahydrofuran complex in tetrahydrofuran (6.3 mL). The mixture was heated at 50° C. for 30 minutes and then cooled to room temperature, after which methanol (1 mL) was added and the solution was stirred for 15 minutes. The solvents were removed under reduced pressure and the residue was dissolved in a mixture of methanol (24 mL) and concentrated hydrochloric acid (0.2 mL) and refluxed for 1 hour. At the end of this time the solvent was evaporated under reduced pressure and the residue was partitioned between ethyl acetate and brine. The aqueous layer was treated with excess solid sodium bicarbonate and the mixture was extracted six times with dichloromethane. The combined dichloromethane washings were dried with anhydrous magnesium sulphate, filtered and concentrated under reduced pressure to give the sub-titled compound. Yield: 520 mg

MS APCI+ 250 (M+H)⁺

### iii) 1-[3-(2-Phenylethoxy)propyl]azetidine-3-carbaldehyde

The sub-titled compound was prepared from the product of step (ii) ({1-[3-(2-phenylethoxy)propyl]azetidin-3-yl}methanol (350 mg) using the method of example 19 step (ii) to give the sub-titled compound. Yield: 280 mg

MS APCI+ 248 (M+H)⁺

### iv) 7-[(1R)-2-Amino-1-hydroxyethyl]-4-hydroxy-1,3-benzothiazol-2(3H)-one hydrochloride

A solution of the product of example 19 step (iv) (7-((1R)-2-amino-1-hydroxyethyl)-4-benzyloxy-3H-benzothiazol-2-one hydrochloride) (1.8 g) in methanol (60 mL) and concentrated hydrochloric acid (4 mL) was stirred vigorously in the presence of 10% palladium on carbon catalyst (0.36 g) and under 4 atmospheres pressure of hydrogen gas for 2 hours. Further 10% palladium on carbon catalyst (0.24 g) was added and stirring was continued under hydrogen for 1 hour. The catalyst was filtered off and the solvent was evaporated under reduced pressure to give the sub-titled compound. Yield: 1.3 g

MS APCI+ 227 (M+H)⁺

¹H NMR (400 MHz, d₆-DMSO) δ 11.70 (s, 1H), 10.21 (s, 1H), 8.04 (s, 3H), 6.92 (d, 1H), 6.79 (d, 1H), 6.32 (d, 1H), 4.81-4.79 (m, 1H), 2.90-2.81 (m, 2H).

### v) 4-Hydroxy-7-{(1R)-1-hydroxy-2-[({1-[3-(2-phenylethoxy)propyl]azetidin-3-yl}methyl)amino]ethyl}-1,3-benzothiazol-2(3H)-one hydrochloride

A solution of the product of step (iv) (7-[(1R)-2-amino-1-hydroxyethyl]-4-hydroxy-1,3-benzothiazol-2(3H)-one hydrochloride) (193 mg) and the product of step (iii) (1-(3-phenethyloxy-propyl)-azetidine-3-carbaldehyde) (140 mg) in methanol (5 mL) was treated with acetic acid (140 mg) and stirred for 40 minutes at room temperature. Sodium cyanoborohydride (29 mg) was added and stirring was continued for 18 hours. The solvent was removed under reduced pressure and the residue was partitioned between ethyl acetate (50 mL) and water (50 mL) containing concentrated aqueous ammonia (1 mL). The organic layer was dried with anhydrous magnesium sulphate, filtered and concentrated under reduced pressure to yield the crude product. Purification was by flash chromatography on a silica column, eluting with 1% concentrated aqueous ammonia and 20% methanol in dichloromethane. The resultant product was converted to the hydrochloride salt by dissolving in methanol and adding an excess of hydrogen chloride (4 molar in 1,4-dioxane). The solvent was then removed under reduced pressure to give the titled compound. Yield: 280 mg

MS APCI+ 458 (M+H)⁺

¹H NMR (400 MHz, d₆-DMSO) δ 11.70 (s, 1H), 10.95-10.80 (m, 1H), 10.23 (s, 1H), 9.43 (s, 1H), 8.95 (s, 1H), 7.32-7.17 (m, 5H), 6.95-6.91 (m, 1H), 6.79 (d, 1H), 6.45 (s, 1H), 4.99-4.97 (m, 1H), 4.14-4.11 (m, 1H), 4.03-3.99 (m, 2H), 3.83-3.80 (m, 1H), 3.61-3.57 (m, 4H), 3.44 (t, 2H), 3.22-3.20 (m, 2H), 3.17-3.13 (m, 2H), 3.00-2.97 (m, 2H), 2.82 (t, 2H), 1.73-1.67 (m, 2H).

### EXAMPLE 21

### 4-Hydroxy-7-{(1R)-1-hydroxy-2-[(2-{1-[2-(2-phenylethoxy)ethyl]piperidin-4-yl}ethyl)amino]ethyl}-1,3-benzothiazol-2(3H)-one

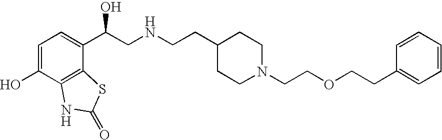

### i) 1-[4-(2-Hydroxy-ethyl)-piperidin-1-yl]-2-phenethyloxy-ethanone

A solution of (2-phenylethoxy)-acetyl chloride (WO 2002/059098) (1.1 g) in tetrahydrofuran (5 mL) was added dropwise to a solution of 4-piperidine-ethanol (0.72 g) and triethylamine (1.7 g) in tetrahydrofuran (20 mL) at 0° C. The reaction mixture was stirred at 0° C. for 10 minutes and then at room temperature for 2 hours. The mixture was partitioned between ethyl acetate and water, the organic layer was washed with dilute aqueous hydrochloric acid and then with aqueous brine. The solvent was removed under reduced pressure to give the sub-titled compound. Yield: 1.23 g

MS APCI+ 292 (M+H⁺, 100%)

### ii) 2-[1-(2-Phenethyloxy-ethyl)-piperidin-4-yl]-ethanol

A solution of the product of step (i) (1-[4-(2-hydroxyethyl)-piperidin-1-yl]-2-phenethyloxy-ethanone) (1.23 g) in tetrahydrofuran (20 mL) was treated with a 1 molar solution of borane-tetrahydrofuran complex in tetrahydrofuran (12.7 mL). The mixture was heated at 50° C. for 2 hours and then cooled to room temperature. Methanol (10 mL) was added and the solution was stirred for 15 minutes. The solvents were removed under reduced pressure and the residue was dissolved in a mixture of methanol (30 mL) and concentrated hydrochloric acid (0.5 mL) and refluxed for 1 hour. At the end of this time the solvent was evaporated under reduced pressure and the residue was partitioned between ethyl acetate and brine. The aqueous layer was treated with excess solid sodium bicarbonate and then extracted three times with dichloromethane. The combined dichloromethane washings were dried with anhydrous magnesium sulphate, filtered and concentrated under reduced pressure. The residue was purified by flash chromatography on silica gel eluting with 1% triethylamine and 2% methanol in dichloromethane to give the sub-titled compound. Yield: 0.5 g

MS APCI+ 278 (M+H⁺, 100%)

### iii) [1-(2-Phenethyloxy-ethyl)-piperidin-4-yl]-acetaldehyde

To a solution of the product of step (ii) (2-[1-(2-phenethyloxy-ethyl)-piperidin-4-yl]-ethanol) (250 mg) and triethylamine (273 mg) in dichloromethane (3 mL), cooled to −10° C., was added in one portion a solution of sulphur trioxide-pyridine complex (431 mg) in dimethyl sulphoxide (3 mL). The cooling bath was removed and the mixture was stirred vigorously for 10 minutes. The reaction mixture was partitioned between ethyl acetate (50 mL) and aqueous phosphate buffer (50 mL, pH 7.2), the organic layer was washed with brine and the solvent was evaporated under reduced pressure to give the sub-titled compound. Yield: 170 mg

MS APCI+ 276 (M+H⁺, 100%)

### iv) 4-Hydroxy-7-{(1R)-1-hydroxy-2-[(2-{1-[2-(2-phenylethoxy)ethyl]piperidin-4-yl}ethyl)amino]ethyl}-1,3-benzothiazol-2(3H)-one

The titled compound was prepared from the product of example 20 step (iv) (7-[(1R)-2-amino-1-hydroxyethyl]-4-hydroxy-1,3-benzothiazol-2(3H)-one hydrochloride) (80 mg) and the product of step (iii) ([1-(2-phenethyloxy-ethyl)-piperidin-4-yl]-acetaldehyde) (88 mg) using the method of example 20 step (v). Purification was by flash chromatography on silica gel eluting with 1% concentrated aqueous ammonia and 16% methanol in dichloromethane to give the titled compound. Yield: 16 mg

MS APCI+ 486 (M+H⁺, 100%)

¹H NMR (400 MHz, d₆-DMSO) δ 7.29-7.16 (m, 5H), 6.85 (d, 1H), 6.68 (d, 1H), 4.57 (q, 1H), 3.57 (t, 2H), 3.46 (t, 2H), 2.80-2.76 (m, 4H), 2.68-2.53 (m, 2H), 2.39 (t, 2H), 1.84 (t, 2H), 1.52 (d, 2H), 1.31-1.26 (m, 2H), 1.25-1.15 (m, 1H), 1.10-1.03 (m, 2H).

### EXAMPLE 22

### 4-Hydroxy-7-[(1R)-1-hydroxy-2-({1-[2-(2-phenylethoxy)ethyl]piperidin-4-yl}amino)ethyl]-1,3-benzothiazol-2(3H)-one

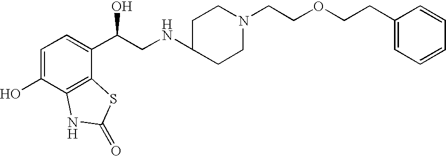

### i) Phenethyloxy-acetic acid

Sodium Hydride (60% in mineral oil) (3.3 g) was added portionwise to a solution of 2-phenyl-ethanol (10.0 g) in dimethylformamide (100 ml), under an atmosphere of nitrogen. After 30 minutes sodium 2-chloroacetate (9.6 g) was added, stir at ambient temperature for 30 minutes then warmed to 60° C. for 1 hour. The reaction mixture was then cooled and poured onto water (500 ml) and extracted with ether (500 ml). The aqueous phase was acidified with concentrated hydrochloric acid to pH 1 and extracted with ether (2×500 ml). The new ether extracts were combined, dried over magnesium sulfate and evaporated to give the sub-titled compound as an oil. Yield: 12.0 g

¹H NMR (300 MHz, CDCl₃) δ 7.38-7.16 (m, 5H), 4.12 (s, 2H), 3.80 (t, 2H), 2.96 (t, 2H).

### ii) 2-Phenethyloxy-ethanol

1M Borane tetrahydrofuran complex (130 ml) was added over 30 minutes to a solution of the product of step (i) (phenethyloxy-acetic acid) (12.0 g) in tetrahydrofuran (200 ml) at 0° C., under an atmosphere of nitrogen. After complete addition the reaction mixture was warmed to ambient temperature for 1.5 h, then methanol (100 ml) added dropwise [care, gas evolved]. The reaction mixture was evaporated to dryness and the residue redissolved in ether (400 ml), extracted with saturated sodium bicarbonate solution twice, once with brine, dried over magnesium sulfate and evaporated to give an oil (8.5 g). Purification by bulb to bulb distillation on a kugelruhr apparatus under high vacuum gave the sub-titled compound as an oil. Yield: 5.0 g

GC/MS 165 (71%), 104 (100%).

¹H NMR (400 MHz, CDCl₃) δ 7.33-7.27 (m, 2H), 7.24-7.19 (m, 3H), 3.74-3.68 (m, 4H), 3.57-3.55 (m, 2H), 2.91 (t, 2H), 1.88 (t, 1H).

### iii) Toluene-4-sulfonic acid 2-phenethyloxy-ethyl ester/2-phenethyloxyethylchloride

Toluenesulfonylchloride (1.40 g) was added at ambient temperature to a solution of the product of step (ii) (2-Phenethyloxy-ethanol) (1.13 g) and 4-dimethylaminopyridine (20 mg) in pyridine (5 ml) to give a purple solution. After 18 h, the reaction mixture was concentrated by rotary evaporation. The residue was dissolved in ethyl acetate and extracted sequentially twice with 2M hydrochloric acid, twice with saturated sodium bicarbonate solution, once with brine, dried over magnesium sulfate and evaporated to give a mixture of the sub-titled compound and 2-phenethyloxyethylchloride (2:1 by NMR) as an oil. Yield: 1.4 g

¹H NMR (300 MHz, CDCl₃) δ 7.79 (d, 2H), 7.35-7.14 (m, 7H), 4.17-4.12 (m, 2H), 3.66-3.57 (m, 4H), 2.81 (t, 2H), 2.44 (s, 3H). (+2-Phenethyloxyethylchloride signals): δ 7.34-7.14 (m, 7H), 4.16-4.10 (m, 2H), 3.74-3.68 (m, 4H), 2.91 (t, 2H).

This was used crude in the next step (iv) without further purification.

### iv) 1-(2-Phenethyloxy-ethyl)-piperidin-4-one

The crude mixture from step (iii) (Toluene-4-sulfonic acid 2-phenethyloxy-ethyl ester/2-phenethyloxyethylchloride [2:1]) (1.9 g) in 1-methylpyrrolidin-2-one (15 ml) and triethylamine (4 ml) was treated with 4-piperidone hydrochloride (1.1 g) and heated to 85° C. for 2 hours. On cooling the reaction mixture was diluted with ethylacetate and extracted with saturated sodium bicarbonate solution twice, once with brine, dried over sodium sulfate and evaporated to give the sub-titled compound as an oil. Yield: 1.6 g

¹H NMR (300 MHz, CDCl₃) δ 7.35-7.17 (m, 5H), 3.76-3.58 (m, 8H), 2.95-2.86 (m, 3H), 2.77 (t, 2H), 2.69 (t, 1H), 2.42 (t, 2H).

### v) 4-Hydroxy-7-{1-hydroxy-2-[1-(2-phenethyloxy-ethyl)-piperidin-4-ylamino]-ethyl}-3H-benzothiazol-2-one The product of step (iv) (1-(2-Phenethyloxy-ethyl)-piperidin-4-one) (150 mg) in methanol (10 ml) was added to the product of example 20 step (iv) (7-(2-Amino-1-hydroxy-ethyl)-4-hydroxy-3H-benzothiazol-2-one hydrochloride) (100 mg) and stirred at ambient temperature for 30 minutes. Sodium cyanoborohydride (40 mg) was added and stirred at ambient temperature for 18 hours. 8:80 Ammonia solution (0.5 ml) was added and evaporated to dryness. Purification by mass directed reverse phase HPLC to give the titled product as a white solid. Yield: 20 mg

MS APCI+ 458 (M+H)⁺

¹H NMR (400 MHz, d₆DMSO)[90° C.] δ 7.32-7.17 (m, 5H), 6.91 (d, 1H), 6.73 (d, 1H), 4.56 (t, 1H), 3.64 (t, 2H), 3.51 (t, 2H), 2.83 (t, 3H), 2.80-2.67 (m, 4H), 2.46 (t, 2H), 2.44-2.35 (m, 1H), 2.03 (t, 2H), 1.72 (t, 2H), 1.26 (t, 1H).

### EXAMPLE 23

### 4-Hydroxy-7-{(1R)-1-hydroxy-2-[({(3R)-1-[2-(2-phenylethoxy)ethyl]piperidin-3-yl}methyl)amino]ethyl}-1,3-benzothiazol-2(3H)-one

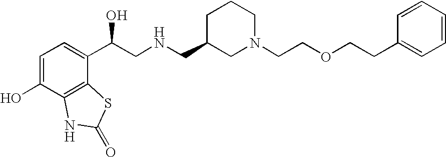

### i) (3R)-1-(2-Phenethyloxy-acetyl)-piperidine-3-carboxylic acid ethyl ester

A solution of ethyl (R)-nipecotate-L-tartrate (2.56 g) and triethylamine (4.2 g) in anhydrous tetrahydrofuran (30 mL) at 0° C. was treated dropwise with a solution of (2-phenylethoxy)-acetyl chloride (1.65 g) in anhydrous tetrahydrofuran (5 mL). The mixture was stirred at 0° C. for 10 minutes and then at room temperature for 2 hours. The reaction mixture was partitioned between water and ethyl acetate, the organic layer was washed with dilute aqueous hydrochloric acid followed by aqueous brine. The organic layer was separated, dried and the solvent removed under reduced pressure to give the sub-titled compound.

Yield: 1.27 g

MS APCI+ 320 (M+H)⁺

### ii) (3R)-[1-(2-Phenethyloxy-ethyl)-piperidin-3-yl]-methanol

A solution of 1 molar lithium aluminium hydride in tetrahydrofuran (6.3 mL) was added dropwise to anhydrous tetrahydrofuran (20 mL) stirred under nitrogen. To this mixture was then added dropwise a solution of the product of step (i) ((3R)-1-(2-phenethyloxy-acetyl)-piperidine-3-carboxylic acid ethyl ester) (1.27 g) in anhydrous tetrahydrofuran (5 mL). The mixture was stirred at room temperature for 2 hours. At the end of this time the reaction mixture was quenched by careful addition of ethyl acetate (3 mL) followed by water (5 mL). Most of the tetrahydrofuran was removed under reduced pressure and the residue was partitioned between aqueous brine and ethyl acetate, the aqueous layer was re-extracted with ethyl acetate and the combined organic layers were washed with excess dilute aqueous hydrochloric acid. The acidic layer was treated with excess solid sodium bicarbonate and the mixture extracted twice with dichloromethane. The combined dichloromethane layers were evaporated under reduced pressure and the residue purified by chromatography on silica gel eluting with 1% triethylamine and 4% methanol in dichloromethane to give the sub-titled compound. Yield: 0.42 g

MS APCI+ 264 (M+H)⁺

### iii) (3R)-1-(2-Phenethyloxy-ethyl)-piperidine-3-carbaldehyde

The sub-titled compound was prepared from the product of step (ii) ((3R)-[1-(2-phenethyloxy-ethyl)-piperidin-3-yl]-methanol) (420 mg) using the method of example 21 step (iii) to give the sub-titled compound. Yield: 250 mg

MS APCI+ 262 (M+H)⁺

### iv) 4-Hydroxy-7-((1R)-1-hydroxy-2-{[(3R)-1-(2-phenethyloxy-ethyl)-piperidin-3-ylmethyl]-amino}-ethyl)-3H-benzothiazol-2-one

A solution of the product of example 20 step (iv) (7-[(1R)-2-amino-1-hydroxyethyl]-4-hydroxy-1,3-benzothiazol-2(3H)-one hydrochloride) (70 mg) and the product of step (iii) ((3R)-1-(2-phenethyloxy-ethyl)-piperidine-3-carbaldehyde) (90 mg) in methanol (5 mL) was treated with acetic acid (32 mg). Sodium cyanoborohydride (10 mg) was added and stirring was continued for 18 hours. The solvent was removed under reduced pressure and the residue was partitioned between ethyl acetate (50 mL) and water (50 mL) containing concentrated aqueous ammonia (1 mL). The organic layer was dried with anhydrous magnesium sulphate, filtered and concentrated under reduced pressure to yield the crude product. Purification was by flash chromatography on a silica column, eluting with 1% concentrated aqueous ammonia and 11% methanol in dichloromethane to give the titled compound. Yield: 30 mg

MS APCI+ 472 (M+H)⁺

¹H NMR (400 MHz, d₆-DMSO) δ 7.28-7.15 (m, 5H), 6.85 (d, 1H), 6.69 (d, 1H), 4.57 (q, 1H), 3.57 (t, 2H), 3.47 (t, 2H), 2.78 (t, 3H), 2.71-2.54 (m, 3H), 2.41-2.34 (m, 4H), 1.87 (t, 1H), 1.64-1.51 (m, 4H), 1.42-1.35 (m, 1H), 0.85-0.78 (m, 1H).

### EXAMPLE 24

### 4-Hydroxy-7-{(1R)-1-hydroxy-2-[({(3S)-1-[2-(2-phenylethoxy)ethyl]piperidin-3-yl}methyl)amino]ethyl}-1,3-benzothiazol-2(3H)-one

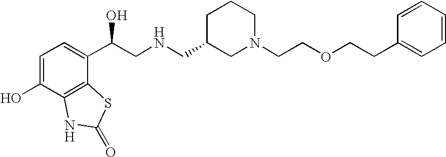

### i) (3R)-1-(2-Phenethyloxy-acetyl)-piperidine-3-carboxylic acid ethyl ester

The sub-titled compound was prepared from ethyl (S)-nipecotate-D-tartrate (2.56 g) using the method of example 23 step (i) to give the sub-titled compound. Yield: 1.37 g

MS APCI+ 320 (M+H)⁺

### ii) (3R)-[1-(2-Phenethyloxy-ethyl)-piperidin-3-yl]-methanol

The sub-titled compound was prepared from the product of step (i) ((3R)-1-(2-phenethyloxy-acetyl)-piperidine-3-carboxylic acid ethyl ester) (1.37 g) using the method of Example 23 step (ii) to give the sub-titled compound. Yield: 0.6 g

MS APCI+ 264 (M+H)⁺

### iii) (3R)-1-(2-Phenethyloxy-ethyl)-piperidine-3-carbaldehyde

The sub-titled compound was prepared from the product of step (ii) ((3R)-[1-(2-phenethyloxy-ethyl)-piperidin-3-yl]-methanol) (250 mg) using the method of Example 21 step (iii) to give the sub-titled compound. Yield: 162 mg

MS APCI+ 262 (M+H)⁺

### iv) 4-Hydroxy-7-((1R)-1-hydroxy-2-{[(3R)-1-(2-phenethyloxy-ethyl)-piperidin-3-ylmethyl]-amino}-ethyl)-3H-benzothiazol-2-one

The title compound was prepared from the product of step (iii) ((3R)-1-(2-phenethyloxy-ethyl)-piperidine-3-carbaldehyde) (70 mg) and the product of Example 20 step (iv) (7-[(1R)-2-amino-1-hydroxyethyl]-4-hydroxy-1,3-benzothiazol-2(3H)-one hydrochloride) (60 mg) using the method of example 23 step (iv) to give the titled compound. Yield: 22 mg

MS APCI+ 472 (M+H)⁺

¹H NMR (400 MHz, d₆-DMSO) δ 7.28-7.16 (m, 5H), 6.86 (d, 1H), 6.69 (d, 1H), 4.58-4.56 (m, 1H), 3.60-3.56 (m, 2H), 3.49-3.46 (m, 2H), 2.83-2.77 (m, 3H), 2.72-2.54 (m, 3H), 2.40-2.36 (m, 4H), 1.87 (t, 1H), 1.62-1.52 (m, 4H), 1.39-1.36 (m, 1H), 0.83-0.80 (m, 1H).

### EXAMPLE 25

### 5-{(1R)-2-[({1-[3-(Benzyloxy)propyl]-4-hydroxypiperidin-4-yl}methyl)amino]-1-hydroxyethyl}-8-hydroxyquinolin-2(1H)-one

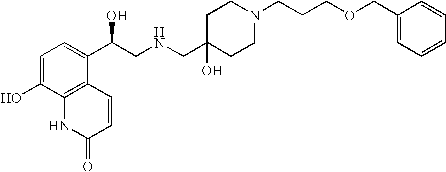

### i) 1-(3-Benzyloxy-propyl)-piperidin-4-one

A suspension of benzyl 3-bromo-propyl ether (2.29 g), 4-piperidinone hydrate hydrochloride (1.69 g), potassium carbonate (anhydrous) (3.45 g) and potassium iodide (830 mg) were stirred in dry DMF (40 ml) at 85° C. and continued for 24 h. The reaction was poured into water (200 mL plus brine 25 mL) and extracted with EtOAc. The organic extract was evaporated and the residue was chromatographed on silica gel eluting with 4:3 EtOAc/isohexane giving the sub-titled compound as a colourless oil. Yield: 1.8 g

¹H NMR (300 MHz, CDCl₃) δ 7.39-7.32 (4H, m), 7.31-7.25 (1H, m), 4.52 (2H, d), 3.60-3.53 (2H, m), 2.79-2.70 (4H, m), 2.61-2.54 (2H, m), 2.48-2.41 (4H, m), 1.89-1.80 (2H, m)

### ii) 4-Aminomethyl-1-(3-benzyloxy-propyl)-piperidin-4-ol

The sub-titled compound was prepared according to the method of Example 26 step (ii) using the product of step (i) (1-(3-benzyloxy-propyl)-piperidin-4-one) (1.8 g). Yield: 2.2 g

¹H NMR (300 MHz, CDCl₃) δ 7.38-7.31 (4H, m), 7.30-7.24 (1H, m), 4.50 (2H, s), 3.52 (2H, t), 2.71-2.63 (2H, m), 2.60 (2H, s), 2.47 (2H, dd), 2.35 (2H, td), 1.88-1.79 (2H, m), 1.61-1.48 (5H, m)

### iii) 8-Benzyloxy-5-[2-{[1-(3-benzyloxy-propyl)-4-hydroxy-piperidin-4-ylmethyl]-amino}-1-(tert-butyl-dimethyl-silanyloxy)-ethyl]-1H-quinolin-2-one

The sub-titled compound was prepared by the method of Example 26 step (iii) using the product of step (ii) (4-aminomethyl-1-(3-benzyloxy-propyl)-piperidin-4-ol) (310 mg) and the product of example 2 step (iii) (8-(Benzyloxy)-5-((1R)-2-bromo-1-{[tert-butyl(dimethyl)silyl]oxy}ethyl)quinolin-2(1H)-one) (122 mg) as a semi-solid.

Yield: 110 mg

¹H NMR (300 MHz, d₆DMSO) δ 0.00 (3H, s), 0.23 (3H, s), 1.00 (9H, s), 1.67-1.57 (4H, m), 1.85 (3H, t), 2.54-2.39 (4H, m), 2.64-2.53 (3H, m), 2.87-2.78 (1H, m), 3.03-2.93 (1H, m), 3.61 (2H, t), 4.14 (1H, s), 4.62 (2H, s), 5.35-5.29 (1H, m), 5.45 (2H, s), 6.72 (1H, d), 7.30 (1H, d), 7.37 (1H, d), 7.60-7.43 (8H, m), 7.75 (2H, d), 8.47 (1H, d), 10.73 (1H, s)

### iv) 5-[2-{[1-(3-Benzyloxy-propyl)-4-hydroxy-piperidin-4-ylmethyl]-amino}-1-(tert-butyl-dimethyl-silanyloxy)-ethyl]-8-hydroxy-1H-quinolin-2-one

The sub-titled compound was prepared by the method of Example 26 step (iv) using the product of step (iii) (8-benzyloxy-5-[2-{[1-(3-benzyloxy-propyl)-4-hydroxy-piperidin-4-ylmethyl]-amino}-1-(tert-butyl-dimethyl-silanyloxy)-ethyl]-1H-quinolin-2-one) (100 mg) as a solid. Yield: 19 mg

¹H NMR (300 MHz, CDCl₃) δ 8.47 (1H, d), 7.57-7.48 (5H, m), 7.21 (1H, d), 7.15 (1H, d), 6.78 (1H, d), 5.31-5.25 (1H, m), 4.68 (2H, s), 1.06 (9H, s), 0.24 (3H, s), 0.00 (3H, s)

### v) 5-(2-{[1-(3-Benzyloxy-propyl)-4-hydroxy-piperidin-4-ylmethyl]-amino}-1-hydroxy-ethyl)-8-hydroxy-1H-quinolin-2-one

The titled compound was prepared by the method of example 26 step (v), from the product of step (iv) (5-[2-{[1-(3-benzyloxy-propyl)-4-hydroxy-piperidin-4-ylmethyl]-amino}-1-(tert-butyl-dimethyl-silanyloxy)-ethyl]-8-hydroxy-1H-quinolin-2-one) (19 mg) as a solid.

Yield: 9 mg

MS APCI+ 482 (M+H)⁺

¹H NMR (d₆-DMSO) δ 8.19 (1H, d), 7.40-7.24 (5H, m), 7.07 (1H, d), 6.91 (1H, d), 6.51 (1H, d), 5.07 (1H, t), 4.45 (2H, s), 3.46-3.43 (10H, m), 2.77-2.45 (153H, m), 2.46-2.29 (68H, m), 1.76-1.68 (1H, m), 1.55-1.46 (4H, m), 1.08-0.96 (10H, m)

### EXAMPLE 26

### 5-{(1R)-2-[({1-[2-(Benzyloxy)ethyl]-4-hydroxypiperidin-4-yl}methyl)amino]-1-hydroxyethyl}-8-hydroxyquinolin-2(1H)-one

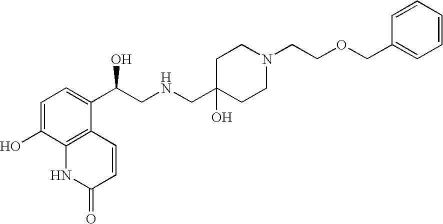

### i) 1-(2-Benzyloxy-ethyl)-piperidin-4-one

4-Piperidinone hydrate hydrochloride (0.714 g), potassium iodide (50 mg), benzyl 2-bromoethyl ether (0.8 g) were stirred and refluxed in MeCN (20 mL) in the presence of K₂CO₃ (1.28 g) for 24 h. The mixture was poured into water (150 mL) and extracted into EtOAc. Drying (MgSO₄) and evaporation left an oil (880 mg) which was chromatographed on silica gel eluting with 3:2 EtOAc-isohexane to give the sub-titled compound as a colourless oil. Yield: 550 mg.

¹H NMR (300 MHz, CDCl₃) δ 7.40-7.32 (4H, m), 7.33-7.28 (1H, m), 4.57 (2H, s), 3.63 (2H, t), 2.82 (4H, t), 2.75 (2H, t), 2.47 (4H, t)

### ii) 4-Aminomethyl-1-(2-benzyloxy-ethyl)-piperidin-4-ol

Trimethylsilylcyanide (0.351 g) was added dropwise to the product of step (i) (1-(2-benzyloxy-ethyl)-piperidin-4-one) (550 mg) plus zinc iodide (30 mg) cooled in ice. After 1 h at 0° C. the mixture was heated at 60° C. for a further 3 h then cooled to ambient temperature. All volatiles were evaporated in vacuo at 60° C. and the residue dissolved in ether (10 ml) plus dry THF (5 ml) and cooled in ice. Lithium aluminium hydride (1M solution in ether, 4 ml) was added and the mixture stirred for 2 h with in ice bath cooling. After 2 h the reaction was allowed to warm to ambient temperature. 1M NaOH (20 ml) was added. Excess ether and EtOAc was added and the resulting 2 phase mixture was separated. The aqueous solution was further extracted with EtOAc. The combined organic solutions were dried over Na₂SO₄, filtered and evaporated to give the sub-titled compound as an oil. Yield: 0.45 g

¹H NMR (300 MHz, CDCl₃) δ 7.38-7.31 (4H, m), 7.30-7.23 (1H, m), 4.54 (2H, s), 3.60 (2H, t), 2.74-2.67 (2H, m), 2.65 (2H, t), 2.60 (2H, s), 2.46-2.36 (2H, m), 1.77-1.35 (2H, m), 1.60-1.52 (3H, m)

### iii) Benzyloxy-5-[2-{[1-(2-benzyloxy-ethyl)-4-hydroxy-piperidin-4-ylmethyl]-amino}-1-(tert-butyl-dimethyl-silanyloxy)-ethyl]-1H-quinolin-2-one

The product of Example 2 step (iii) (8-(Benzyloxy)-5-((1R)-2-bromo-1-{[tert-butyl(dimethyl)silyl]oxy}ethyl)quinolin-2(1H)-one) (122 mg) and the product of step (ii) (4-aminomethyl-1-(2-benzyloxy-ethyl)-piperidin-4-ol) (290 mg) and N,N-diisopropylethylamine (129 mg) were dissolved in dry NMP (2 ml) and heated in a microwave oven for 90 min at 100° C. The reaction was poured into 10% brine (25 ml) and was then extracted with EtOAc. Drying (MgSO₄) and evaporation of the organic solution gave a brown oil. Purification was by chromatography on silica gel eluting with 17:3 EtOAc/MeOH+10% Et3N to give the sub-titled compound as an amber oil. Yield: 170 mg.

1H NMR (300 MHz, CDCl₃) δ 0.24 (3H, s), 1.06 (9H, s), 5.34 (2H, s), 6.85 (1H, d), 7.19 (1H, d), 7.30 (1H, d), 7.49-7.45 (11H, m), 7.54-7.49 (10H, m), 7.63-7.56 (9H, m), 8.43 (1H, d), 9.39 (1H, s), 5.29-5.24 (1H, m), 4.71-4.66 (4H, m)

### iv) 5-[2-{[1-(2-Benzyloxy-ethyl)-4-hydroxy-piperidin-4-ylmethyl]-amino}-1-(tert-butyl-dimethyl-silanyloxy)-ethyl]-8-hydroxy-1H-quinolin-2-one

The product of step (iii) (benzyloxy-5-[2-{[1-(2-benzyloxy-ethyl)-4-hydroxy-piperidin-4-ylmethyl]-amino}-1-(tert-butyl-dimethyl-silanyloxy)-ethyl]-1H-quinolin-2-one) (170 mg) in ethanol (3 ml) plus 2M HCl (0.25 ml) was hydrogenated at 5 bar pressure with 10% Pd/C catalyst (10 mg). After 3 hr more catalyst (5 mg) was added and the hydrogenation was continued for a further 6 h. Catalyst was filtered off and the solvent was evaporated. The residue was purified by reverse phase HPLC using an Xterra® C8 5 micron 19×50 mm column eluting with a gradient of 95% 0.880 NH₃ in acetonitrile to 5% 0.880 NH₃ in acetonitrile give to give the sub-titled compound. Yield: 57 mg

¹H NMR (300 MHz, d₆-DMSO) δ 8.44 (1H, d), 7.57-7.41 (5H, m), 7.20 (1H, d), 7.08 (1H, d), 6.67 (1H, d), 5.32-5.24 (1H, m), 4.64 (2H, s), 3.69 (3H, t), 3.04-2.93 (1H, m), 2.87-2.76 (2H, m), 1.00 (9H, s), 0.22 (3H, s), 0.18 (3H, s)

### v) 5-(2-{[1-(2-Benzyloxy-ethyl)-4-hydroxy-piperidin-4-ylmethyl]-amino}-1-hydroxy-ethyl)-8-hydroxy-1H-quinolin-2-one

The product from step (iv) (5-[2-{[1-(2-benzyloxy-ethyl)-4-hydroxy-piperidin-4-ylmethyl]-amino}-1-(tert-butyl-dimethyl-silanyloxy)-ethyl]-8-hydroxy-1H-quinolin-2-one) (50 mg) in dry THF (1 ml) was treated with triethylamine trihydrofluoride (154 mg) and stirred at ambient temperature overnight under nitrogen. The volatiles were evaporated in vacuo at 75° C. The residue was dissolved in MeOH and applied to a SCX cartridge, washed with further MeOH and finally eluted with 7N methanolic ammonia. Evaporation gave a gum which was dried in vacuo to give the titled compound as a semi-solid. Yield: 30 mg

MS APCI+ 468 (M+H)⁺

¹H NMR (300 MHz, d₆-DMSO) δ 8.19 (1H, d), 7.39-7.24 (5H, m), 7.07 (1H, d), 6.91 (1H, d), 6.49 (1H, d), 5.05 (1H, t), 4.47 (2H, s), 3.53 (2H, t), 2.75-2.70 (2H, m), 2.64-2.55 (7H, m), 2.55-2.46 (12H, m), 2.42-2.32 (2H, m), 1.53-1.43 (4H, m), 1.00 (14H, t)

### EXAMPLE 27

### N-(2,5-Dichlorobenzyl)-3-(4-{[(2R)-2-hydroxy-2-(8-hydroxy-2-oxo-1,2-dihydroquinolin-5-yl)ethyl]amino}piperidin-1-yl)propanamide

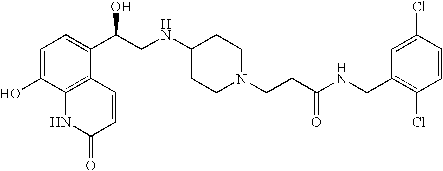

A solution of the product from Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.1 g), in dry DMF (2 ml) was treated with HATU (0.14 g), triethylamine (0.037 ml) and 1-(2,5-dichlorophenyl)methanamine (72 mg) at ambient temperature. After 1 h the volatiles were evaporated in vacuo and the residue treated with triethylamine trihydrofluoride (0.175 ml) with further stirring for 48 h. The volatiles were evaporated in vacuo and the residue re-dissolved in DMSO (2 ml). The residue was loaded onto a Varian SCX column (25 g) which was eluted with 1:1 isopropanol/acetonitrile (2×40 ml) and fractions discarded. The product fractions were eluted with 1:2:2 ammonium hydroxide/isopropanol/acetonitrile (40 ml). The volatiles were evaporated in vacuo to give the crude title product. Purification was by reverse phase HPLC using an Xterra® C8 5 micron 19×50 mm column eluting with a gradient of 95% 0.880 NH₃ in acetonitrile to 50% 0.880 NH₃ in acetonitrile to give the title compound as a pale yellow foam. Yield: 28 mg

MS APCI+ 533/535 [M+H]⁺

¹H NMR (300 MHz, d₆-DMSO) 8.51 (t, 1H), 8.17 (d, 1H), 7.48 (m, 1H), 7.34 (m, 2H), 7.07 (d, 1H), 6.91 (d, 1H), 6.50 (d, 1H), 4.98 (t, 1H), 4.30 (d, 2H), 2.79 (bd, 2H), 2.72 (d, 2H), 2.50 (m, 2H), 2.43 (m, 1H), 2.32 (t, 2H), 1.93 (m, 2H), 1.75 (t, 2H), 1.26 (m, 2H)

### EXAMPLE 28

### N-(Biphenyl-2-ylmethyl)-3-(4-{[(2R)-2-hydroxy-2-(8-hydroxy-2-oxo-1,2-dihydroquinolin-5-yl)ethyl]amino}piperidin-1-yl)propanamide

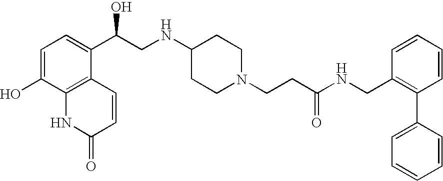

The title compound was prepared from the product of Example 6 step (iii) (3-[4-({[(2R)-1-{[tert-butyl(dimethyl)silyl]oxy}-2-(8-hydroxy-2-oxo-1,2-dihydroquinolin-5-yl)ethyl]amino}piperidin-1-yl]propanoic acid) (0.1 g), using HATU (0.14 g), triethylamine (0.037 ml) and 1-biphenyl-2-ylmethanamine (75 mg) in dry DMF (2 ml) according to the procedure described in Example 27. Purification was by reverse phase HPLC using an Xterra® C8 5 micron 19×50 mm column eluting with a gradient of 95% 0.880 NH₃ in acetonitrile to 5% 0.880 NH₃ in acetonitrile give the title compound as a pale yellow foam. Yield: 31 mg

MS APCI+ 541 [M+H]⁺

¹H NMR (300 MHz, d₆-DMSO) 8.32 (t, 1H), 8.17 (d, 1H), 7.38 (m, 8H), 7.20 (d, 1H), 7.07 (d, 1H), 6.91 (d, 1H), 6.50 (d, 1H), 4.98 (t, 1H), 4.17 (d, 2H), 2.76 (m, 2H), 2.71 (d, 2H), 2.50 (m, 2H), 2.40 (m, 1H), 2.24 (t, 2H), 1.90 (m, 2H), 1.73 (t, 2H), 1.20 (m, 2H).

### EXAMPLE 29

### N-(2,6-dichlorobenzyl)-3-(4-{[(2R)-2-hydroxy-2-(8-hydroxy-2-oxo-1,2-dihydroquinolin-5-yl)ethyl]amino}piperidin-1-yl)propanamide

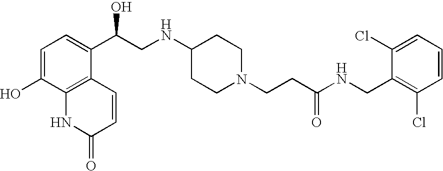

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.1 g), using HATU (0.14 g), triethylamine (0.037 ml) and 1-(2,6-dichlorophenyl)methanamine (72 mg) in dry DMF (2 ml) according to the procedure described in Example 27. Purification was by reverse phase HPLC using an Xterra® C8 5 micron 19×50 mm column eluting with a gradient of 95% 0.880 NH₃ in acetonitrile to 50% 0.880 NH₃ in acetonitrile give the title compound as a pale yellow foam. Yield: 32 mg

MS APCI+ 532/534/536 [M+H]⁺

¹H NMR (300 MHz, d₆-DMSO) 8.31 (m, 1H), 8.16 (d, 1H), 7.48 (m, 2H), 7.33 (t, 1H), 7.08 (d, 1H), 6.92 (d, 1H), 6.51 (d, 1H), 4.99 (t, 1H), 4.47 (d, 2H), 2.73 (m, 4H), 250 (m, 3H), 2.23 (t, 2H), 1.87 (t, 2H), 1.71 (t, 2H), 1.15 (m, 2H)

### EXAMPLE 30

### N-(Cyclohexylmethyl)-3-(4-{[(2R)-2-hydroxy-2-(8-hydroxy-2-oxo-1,2-dihydroquinolin-5-yl)ethyl]amino}piperidin-1-yl)propanamide

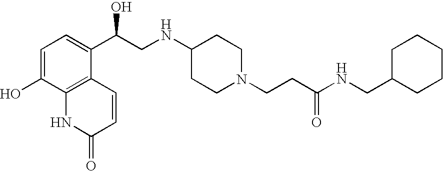

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.1 g), using HATU (0.14 g), triethylamine (0.037 ml) and 1-cyclohexylmethanamine (46 mg) in dry DMF (2 ml) according to the procedure described in Example 27. Purification was by reverse phase HPLC using an Xterra® C8 5 micron 19×50 mm column eluting with a gradient of 95% 0.880 NH₃ in acetonitrile to 50% 0.880 NH₃ in acetonitrile give the title compound as a pale yellow foam. Yield: 23 mg

MS APCI+ 471 [M+H]⁺

¹H NMR (300 MHz, d₆-DMSO) 8.17 (d, 1H), 7.92 (t, 1H), 7.06 (d, 1H), 6.90 (d, 1H), 6.49 (d, 1H), 4.96 (t, 1H), 2.87 (m, 2H), 2.75 (m, 2H), 2.70 (d, 2H), 2.46 (m, 2H), 2.38 (m, 1H), 2.19 (t, 2H), 1.90 (m, 2H), 1.68 (m, 7H), 1.33 (m, 1H), 1.14 (m, 5H), 0.84 (m, 2H)

### EXAMPLE 31

### N-(2-Chloro-6-methylbenzyl)-3-(4-{[(2R)-2-hydroxy-2-(8-hydroxy-2-oxo-1,2-dihydroquinolin-5-yl)ethyl]amino}piperidin-1-yl)propanamide

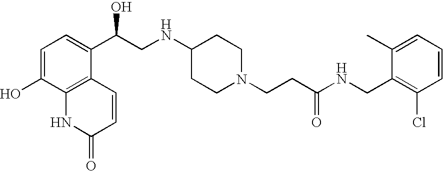

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.1 g), using HATU (0.14 g), triethylamine (0.037 ml) and 1-(2-chloro-6-methylphenyl)methanamine (64 mg) in dry DMF (2 ml) according to the procedure described in Example 27. Purification was by reverse phase HPLC using an Xterra® C8 5 micron 19×50 mm column eluting with a gradient of 95% 0.880 NH₃ in acetonitrile to 50% 0.880 NH₃ in acetonitrile give the title compound as a pale yellow foam. Yield: 30 mg

MS APCI+ 513/515 [M+H]⁺

¹H NMR (300 MHz, d₆-DMSO) 8.24 (t, 1H), 8.17 (d, 1H), 7.26 (m, 1H), 7.16 (m, 2H), 7.06 (d, 1H), 6.91 (d, 1H), 6.49 (d, 1H), 4.95 (t, 1H), 4.37 (d, 2H), 2.69 (m, 4H), 2.45 (t, 2H), 2.35 (m, 2H), 2.22 (t, 2H), 1.87 (m, 2H), 1.67 (t, 2H), 1.09 (m, 2H)

### EXAMPLE 32

### 3-(4-{[(2R)-2-hydroxy-2-(8-hydroxy-2-oxo-1,2-dihydroquinolin-5-yl)ethyl]amino}piperidin-1-yl)-N-[(1R,2S)-2-phenylcyclopropyl]propanamide

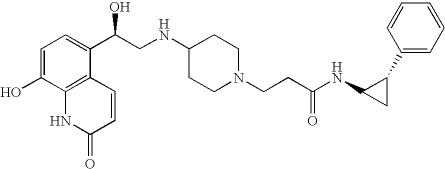

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.1 g), using HATU (0.14 g), triethylamine (0.037 ml) and (1R,2S)-2-phenylcyclopropanamine (55 mg) in dry DMF (2 ml) according to the procedure described in Example 27. Purification was by reverse phase HPLC using an Xterra® C8 5 micron 19×50 mm column eluting with a gradient of 95% 0.880 NH₃ in acetonitrile to 50% 0.880 NH₃ in acetonitrile give the title compound as a pale yellow foam. Yield: 50 mg

MS APCI+ 491 [M+H]⁺

¹H NMR (300 MHz, d₆-DMSO) 8.21 (m, 1H), 8.17 (d, 1H), 7.25 (t, 2H), 7.15 (t, 1H), 7.08 (m, 3H), 6.91 (d, 1H), 6.50 (d, 1H), 4.98 (t, 1H), 2.75 (m, 5H), 2.50 (m, 2H), 2.37 (m, 1H), 2.19 (t, 2H), 1.89 (m, 3H), 1.74 (t, 2H), 1.18 (m, 2H), 1.13 (m, 2H)

### EXAMPLE 33

### N-(4-chlorobenzyl)-3-(4-{[(2R)-2-hydroxy-2-(8-hydroxy-2-oxo-1,2-dihydroquinolin-5-yl)ethyl]amino}piperidin-1-yl)propanamide

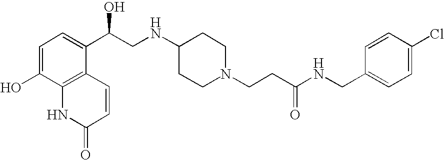

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.1 g), using HATU (0.14 g), triethylamine (0.037 ml) and 1-(4-chlorophenyl)methanamine (58 mg) in dry DMF (2 ml) according to the procedure described in Example 27. Purification was by reverse phase HPLC using an Xterra® C8 5 micron 19×50 mm column eluting with a gradient of 95% 0.880 NH₃ in acetonitrile to 50% 0.880 NH₃ in acetonitrile give the title compound as a pale yellow foam. Yield: 27 mg

MS APCI+ 499/501 [M+H]⁺

¹H NMR (300 MHz, d₆-DMSO) 8.43 (t, 1H), 8.17 (d, 1H), 7.36 (d, 2H), 7.29 (d, 2H), 7.07 (d, 1H), 6.91 (d, 1H), 6.50 (d, 1H), 4.99 (t, 1H), 4.25 (d, 2H), 2.75 (m, 4H), 2.50 (m, 2H), 2.43 (bs, 1H), 2.27 (t, 2H), 1.92 (m, 2H), 1.74 (t, 2H), 1.21 (m, 2H)

### EXAMPLE 34

### N-(3-Chlorobenzyl)-3-(4-{[(2R)-2-hydroxy-2-(8-hydroxy-2-oxo-1,2-dihydroquinolin-5-yl)ethyl]amino}piperidin-1-yl)propanamide

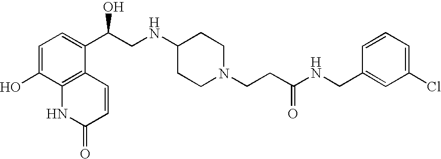

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.1 g), using HATU (0.14 g), triethylamine (0.037 ml) and 1-(3-chlorophenyl)methanamine (58 mg) in dry DMF (2 ml) according to the procedure described in Example 27. Purification was by reverse phase HPLC using an Xterra® C8 5 micron 19×50 mm column eluting with a gradient of 95% 0.880 NH₃ in acetonitrile to 50% 0.880 NH₃ in acetonitrile give the title compound as a pale yellow foam. Yield: 44 mg

MS APCI+ 499/501 [M+H]⁺

¹H NMR (300 MHz, d₆-DMSO) 8.45 (t, 1H), 8.17 (d, 1H), 7.28 (m, 4H), 7.07 (d, 1H), 6.91 (d, 1H), 6.60 (d, 1H), 4.97 (t, 1H), 4.27 (t, 1H), 2.76 (d, 2H), 2.71 (d, 2H), 2.50 (m, 2H), 2.38 (m 1H), 2.28 (t, 2H), 1.91 (m, 2H), 1.73 (t, 2H), 1.17 (m, 2H)

### EXAMPLE 35

### N-(2-chloro-6-fluorobenzyl)-3-(4-{[(2R)-2-hydroxy-2-(8-hydroxy-2-oxo-1,2-dihydroquinolin-5-yl)ethyl]amino}piperidin-1-yl)propanamide

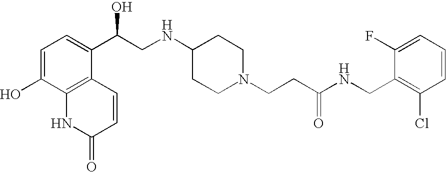

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.1 g), using HATU (0.14 g), triethylamine (0.037 ml) and 1-(2-chloro-6-fluorophenyl)methanamine (65 mg) in dry DMF (2 ml) according to the procedure described in Example 27. Purification was by reverse phase HPLC using an Xterra® C8 5 micron 19×50 mm column eluting with a gradient of 95% 0.880 NH₃ in acetonitrile to 50% 0.880 NH₃ in acetonitrile to give the title compound as a pale yellow foam. Yield: 56 mg

MS APCI+ 517/519 [M+H]⁺

¹H NMR (300 MHz, d₆-DMSO) 8.39 (t, 1H), 8.17 (d, 1H), 7.35 (m, 2H), 7.21 (m, 1H), 7.07 (d, 1H), 6.91 (d, 1H), 6.50 (d, 1H), 4.96 (t, 1H), 4.37 (m, 2H), 2.69 (m, 4H), 2.45 (t, 4H), 2.37 (m, 1H), 2.21 (t, 2H), 1.88 (m, 2H), 1.69 (t, 2H), 1.14 (m, 2H)

### EXAMPLE 36

### N-(2,3-Dichlorobenzyl)-3-(4-{[(2R)-2-hydroxy-2-(8-hydroxy-2-oxo-1,2-dihydroquinolin-5-yl)ethyl]amino}piperidin-1-yl)propanamide

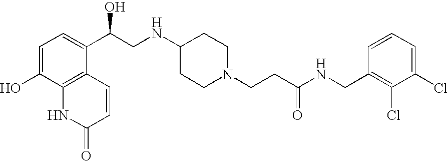

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.1 g), using HATU (0.14 g), triethylamine (0.037 ml) and 1-(2,3-dichlorophenyl)methanamine (72 mg) in dry DMF (2 ml) according to the procedure described in Example 27. Purification was by reverse phase HPLC using an Xterra® C8 5 micron 19×50 mm column eluting with a gradient of 95% 0.880 NH₃ in acetonitrile to 50% 0.880 NH₃ in acetonitrile give the title compound as a pale yellow foam. Yield: 29 mg

MS APCI+ 647/649/651 [M+H]⁺

¹H NMR (300 MHz, d₆-DMSO) 8.54 (t, 1H), 8.17 (d, 1H), 7.53 (dd, 1H), 7.40 (d, 1H), 7.31 (t, 1H), 7.08 (d, 1H), 6.92 (d, 1H), 6.50 (d, 1H), 4.99 (t, 1H), 4.34 (d, 2H), 2.79 (m, 2H), 2.73 (d, 2H), 2.50 (m, 2H), 2.44 (m, 1H), 2.31 (t, 2H), 1.91 (m, 2H), 1.75 (t, 2H), 1.21 (m, 2H)

### EXAMPLE 37

### N-(2-Chlorobenzyl)-3-(4-{[(2R)-2-hydroxy-2-(8-hydroxy-2-oxo-1,2-dihydroquinolin-5-yl)ethyl]amino}piperidin-1-yl)-N-methylpropanamide

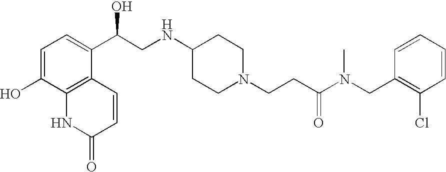

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.1 g), using HATU (0.14 g), triethylamine (0.037 ml) and 1-(2-chlorophenyl)-N-methylmethanamine (64 mg) in dry DMF (2 ml) according to the procedure described in Example 27. Purification was by reverse phase HPLC using an Xterra® C8 5 micron 19×50 mm column eluting with a gradient of 95% 0.880 NH₃ in acetonitrile to 50% 0.880 NH₃ in acetonitrile give the title compound as a pale yellow foam. Yield: 21 mg

MS APCI+ 513/515 [M+H]⁺

¹H NMR (300 MHz, d₆-DMSO 90° C.) 8.16 (d, 1H), 7.42 (d, 1H), 7.30 (bs, 2H), 7.21 (bs, 1H), 7.07 (d, 1H), 6.93 (d, 1H), 6.48 (d, 1H), 4.99 (t, 1H), 4.60 (s, 2H), 2.79 (d, 4H), 2.59 (bs, 2H), 2.02 (bs, 2H), 1.74 (m, 2H), 1.26 (m, 2H)

### EXAMPLE 38

### 5-((1R)-2-{[(1-{2-[2-(3-Chlorophenyl)ethoxy]ethyl}-4-hydroxypiperidin-4-yl)methyl]amino}-1-hydroxyethyl)-8-hydroxyquinolin-2(1H)-one

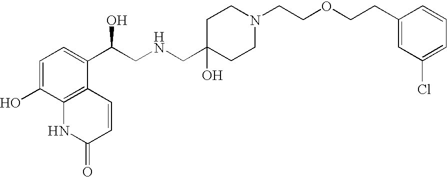

### i) [2-(3-chlorophenyl)ethoxy]acetic acid

2-(3-Chlorophenyl)ethanol (7.32 g, 6.2 ml) was dissolved in DMF (100 ml) and cooled in ice. Sodium hydride (1.72 g, 60% in mineral oil) was added batchwise over 15 min. The reaction mixture was stirred at ambient temperature. for 1.75 h after which time effervescence had ceased, then solid sodium chloroacetate (5.00 g) was added. The mixture was stirred at ambient temperature for 15 min then heated to 60° C. for 6 h. The mixture was left overnight then poured onto water and extracted with ether (×6), the ether extracts then being discarded. The aqueous material was acidified with conc. HCl from pH 9 to pH 2 then extracted with EtOAc (×7). The combined EtOAc extracts were washed with water, saturated aqueous NaCl, dried (Na₂SO₄) and concentrated to afford the sub-titled compound as a colourless oil. Yield: 7.65 g

¹H NMR (300 MHz, CDCl₃) δ 7.26-7.11 (m, 4H), 4.11 (s, 2H), 3.78 (t, 2H), 2.93 (t, 2H).

### ii) 2-[2-(3-Chlorophenyl)ethoxy]ethanol

A solution of the product of step (i) [2-(3-chloro-phenyl)-ethoxy]-acetic acid (7.65 g) in dry THF (180 mL) was cooled to 0° C. Borane-THF (˜1M, 120 mL) was added slowly keeping the internal temperature below 4° C., then the solution was stirred at ambient temperature for 3 h then MeOH (100 mL) was added dropwise keeping the internal temperature below 23° C. The mixture was stirred for 15 min then concentrated in vacuo and partitioned between ether and saturated aqueous NaHCO₃. The layers were separated and the aqueous layer extracted with further ether and EtOAc. The combined organic layers were washed with water, saturated aqueous NaCl, dried (Na₂SO₄) and the volatiles evaporated. Purification was by Biotage column eluting with 1:9 to 1:1 EtOAc:isohexane to afford the sub-titled compound as a colourless oil. Yield: 3.16 g

¹H NMR (300 MHz, CDCl₃) δ 7.25-7.09 (m, 4H), 3.74-3.68 (m, 4H), 3.57-3.54 (m, 2H), 2.88 (t, 2H).

### iii) 2-[2-(3-chlorophenyl)ethoxy]ethyl 4-methylbenzenesulfonate/1-chloro-3-[2-(2-chloroethoxy)ethyl]benzene

p-Toluenesulphonyl chloride (1.56 g) was added at ambient temperature to a solution of the product of step (ii) (2-[2-(3-chloro-phenyl)-ethoxy]-ethanol) (1.52 g) and 4-N,N-dimethylaminopyridine (22 mg) in pyridine (5.5 ml). The reaction mixture was stirred at ambient temperature overnight then diluted with ethyl acetate (120 ml) and washed with 2M aqueous HCl (3×10 ml), saturated aqueous NaHCO₃, water, saturated aqueous NaCl, dried (Na₂SO₄) and the volatiles evaporated to give the sub-titled compound and 1-chloro-3-[2-(2-chloroethoxy)ethyl]benzene (2:1 by NMR) as a pale yellow oil Yield: 1.63 g

¹H NMR (300 MHz, CDCl₃) δ 7.81-7.77 (m, 2H), 7.35-7.04 (m, 6H), 4.17-4.13 (m, 2H), 3.65-3.57 (m, 4H), 2.79 (td, 2H), 2.45 (s, 3H). (+1-chloro-3-[2-(2-chloroethoxy)ethyl]benzene signals): δ 7.35-7.04 (m, 4H), 4.17-4.13 (m, 2H), 3.74-3.69 (m, 4H), 2.89 (td, 2H).

This was used crude in the next step (iv) without further purification.

### iv) 1-{2-[2-(3-chlorophenyl)ethoxy]ethyl}piperidin-4-one

A mixture of the product from step (iii) (2-[2-(3-chlorophenyl)ethoxy]ethyl 4-methylbenzenesulfonate/1-chloro-3-[2-(2-chloroethoxy)ethyl]benzene) (2:1 by NMR) (1.6 g) and piperidin-4-one hydrate hydrochloride (1.102 g) was dissolved in NMP (25 ml) and triethylamine (5.3 ml) added. The reaction mixture was heated at 85° C. for 2.5 h then allowed to cool and partitioned between EtOAc and water. The layers were separated and the EtOAc layer washed with further water, then with saturated. aqueous NaHCO₃, water, saturated aqueous NaCl, dried (Na₂SO₄) and concentrated. The residue was diluted with iPrOH loaded onto a Varian SCX column (3×10 g). Neutral/acid material was washed off with iPrOH then the product was eluted with 1:3 aqueous ammonia/iPrOH then concentrated. The resulting gum partitioned between EtOAc and water, the layers separated, the aqueous layer extracted with further EtOAc and the organic extracts washed with saturated. aqueous NaCl, dried (Na₂SO₄) and concentrated to give the sub-titled compound as an orange oil.

Yield: 0.27 g

¹H NMR (400 MHz, CDCl₃) δ 7.23-7.18 (m, 3H), 7.11-7.09 (m, 1H), 3.67 (t, 2H), 3.60 (t, 2H), 2.87 (t, 2H), 2.77 (t, 4H), 2.69 (t, 2H), 2.43 (t, 4H).

### v) 6-{2-[2-(3-chlorophenyl)ethoxy]ethyl}-1-oxa-6-azaspiro[2.5]octane

The sub-titled compound was prepared from the product of step (iv) (1-{2-[2-(3-chlorophenyl)ethoxy]ethyl}piperidin-4-one) (0.27 g), using 60% sodium hydride (82 mg), trimethylsulphoxonium iodide (0.23 g) in DMSO (4 mL) by the method of example 5 step (iii) as a yellow oil. Yield: 0.28 g

¹H NMR (400 MHz, CDCl₃) δ 7.23-7.16 (m, 3H), 7.11-7.09 (m, 1H), 3.66 (t, 2H), 3.58 (t, 2H), 2.86 (t, 2H), 2.66-2.55 (m, 8H), 1.86-1.79 (m, 2H), 1.57-1.51 (m, 2H).

### vi) 5-((1R)-1-{[tert-butyl(dimethyl)silyl]oxy}-2-{[(1-{2-[2-(3-chlorophenyl)ethoxy]ethyl}-4-hydroxypiperidin-4-yl)methyl]amino}ethyl)-8-hydroxyquinolin-2(1H)-one

A solution of the product from Example 5 step (ii) (5-((1R)-2-amino-1-{[tert-butyl(dimethyl)silyl]oxy}ethyl)-8-hydroxyquinolin-2(1H)-one) (0.4 g) and the product of step (v) (6-{2-[2-(3-chloro-phenyl)-ethoxy]-ethyl}-1-oxa-6-aza-spiro[2.5]octane) (0.28 g) in methanol (4 mL) was heated at reflux for 19 h. The solvent was evaporated to give the crude material. Purification was by Isolute Flash silica-gel (20 g) eluting with 1:4:95 to 3:12:85 7M NH₃ in MeOH:MeOH:DCM to give the sub-titled compound as a yellow gum:

Yield: 0.39 g

MS APCI+ 630/632 [M+H]⁺

### vii) 5-((1R)-2-{[(1-{2-[2-(3-chlorophenyl)ethoxy]ethyl}-4-hydroxypiperidin-4 yl)methyl]amino}-1-hydroxyethyl)-8-hydroxyquinolin-2(1H)-one

The product from step (vi) (5-((1R)-1-{[tert-butyl(dimethyl)silyl]oxy}-2-{[(1-{2-[2-(3-chlorophenyl)ethoxy]ethyl}-4-hydroxypiperidin-4-yl)methyl]amino}ethyl)-8-hydroxyquinolin-2(1H)-one) (0.39 g) was dissolved in THF (5 ml) and to it was added triethylamine trihydrofluoride (0.6 mL). The reaction mixture was stirred at ambient temperature for 3d then the volatiles evaporated to afford a yellow oil. Purification was by reverse phase HPLC (Symmetry®, 100-50% 0.2% aqueous TFA in MeCN) to afford the title compound as a cream-coloured solid. Yield: 87 mg

MS APCI+ 516/518 [M+H]⁺

¹H NMR (300 MHz, DMSO) δ 10.50 (br s, 1H), 8.22 (d, 1H), 7.35-7.22 (m, 4H), 7.16 (d, 1H), 7.00 (d, 1H), 6.58 (d, 1H), 6.20 (br s, 1H), 5.63 (br s, 1H), 5.44-5.42 (m, 1H), 3.75-3.67 (m, 4H), 3.34-3.26 (m, 2H), 3.17-3.06 (m, 6H), 2.86 (t, 2H), 1.85-1.79 (m, 4H), 1.18 (t, 2H).

### EXAMPLE 39

### Benzyl (4-{[(2R)-2-hydroxy-2-(8-hydroxy-2-oxo-1,2-dihydroquinolin-5-yl)ethyl]amino}piperidin-1-yl)acetate

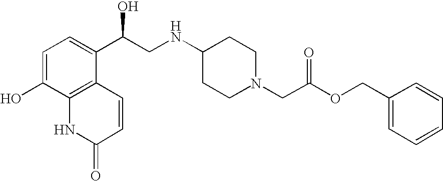

### i) Benzyl (4-oxopiperidin-1-yl)acetate

The sub-titled compound was prepared from 4-piperidinone monohydrate hydrochloride (01.5 g) dissolved in DMF (10 mL) followed by addition of anhydrous potassium carbonate (2.8 g) and benzyl bromoacetate (2.3 g). The mixture was stirred at room temperature overnight then poured into water and extracted with ethyl acetate (×3) and the combined extracts washed with water, collected, dried (Na₂SO₄) to give a colourless oil which solidified on standing. Yield: 2.6 g.

MS APCI+ 248 (M−18)+H+

¹H NMR (300 MHz, d6-DMSO) δ 7.42-7.31 (5H, m), 5.13 (2H, s), 3.50 (2H, s), 2.89-2.83 (4H, m), 2.34 (4H, t)

### ii) Benzyl (4-{[(2R)-2-{[tert-butyl(dimethyl)silyl]oxy}-2-(8-hydroxy-2-oxo-1,2-dihydroquinolin-5-yl)ethyl]amino}piperidin-1-yl)acetate

The sub-titled compound was prepared from the product of Example 5 step (ii) (5-((1R)-2-amino-1-{[tert-butyl(dimethyl)silyl]oxy}ethyl)-8-hydroxyquinolin-2(1H)-one) (100 mg) and the product from step (i) (benzyl (4-oxopiperidin-1-yl)acetate) (158 mg) dissolved in NMP (6 ml) followed by addition of acetic acid (1 drop) and activated 4 A molecular sieves (6 pellets). The mixture was stirred for 14 h before addition of sodium triacetoxyborohydride (253 mg) and further stirring for 24 h. The evaporated mixture was passed through an SCX cartridge using 1:1 isopropyl alcohol/acetonitrile then 10% 0.880 ammonia in 1:1 isopropyl alcohol/acetonitrile as eluent to give the crude product. Purification was by flash chromatography on silica get, eluting with 1% 0.880 ammonia in 9:1 dichloromethane/methanol to give the sub-titled compound. Yield: 160 mg

MS APCI+ 566 [M+H]⁺

### iii) Benzyl (4-{[(2R)-2-hydroxy-2-(8-hydroxy-2-oxo-1,2-dihydroquinolin-5-yl)ethyl]amino}piperidin-1-yl)acetate

The titled compound was prepared from the product of step (ii) (benzyl (4-{[(2R)-2-{[tert-butyl(dimethyl)silyl]oxy}-2-(8-hydroxy-2-oxo-1,2-dihydroquinolin-5-yl)ethyl]amino}piperidin-1-yl)acetate) (150 mg) dissolved in THF (4 ml) followed by addition of triethylaminetrihydrofluoride (0.3 ml) and the solution stirred at ambient temperature for 3 hours. The volatiles were removed in vacuo and the residue loaded onto an SCX cartridge using 1:1 isopropyl alcohol/acetonitrile then 10% 0.880 ammonia in 1:1 isopropyl alcohol/acetonitrile as eluent to give the crude product as a dark orange oil. Purification was by reverse phase HPLC using 0.1% aqueous TFA and acetonitrile to give the title compound as a cream solid. Yield: 22 mg.

MS APCI+ 452 [M+H]⁺

¹H NMR (300 MHz, d6-DMSO) δ 8.15 (1H, d), 7.37-7.28 (5H, m), 7.06 (1H, d), 6.91 (1H, d), 6.48 (1H, d), 5.09 (2H, s), 5.02 (1H, t), 3.15 (1H, s), 2.77 (4H, t), 2.52-2.47 (2H, m), 2.21-2.15 (2H, m), 1.75 (2H, t), 1.34-1.25 (2H, m).

### EXAMPLE 40

### 8-Hydroxy-5-[(1R)-1-hydroxy-2-({[4-hydroxy-1-(4-phenoxybutyl)piperidin-4-yl]methyl}amino)ethyl]quinolin-2(1H)-one

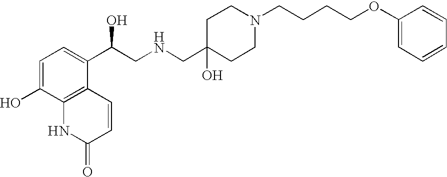

### i) 1-(4-Phenoxybutyl)piperidin-4-one

The subtitled compound was prepared from (4-bromobutoxy)benzene (3 g), 4-piperidinone hydrate hydrochloride (1.92 g), triethylamine (3.5 mL), in chloroform (50 ml) using the method of Example 7 step (i) as a red oil. Yield: 1.8 g

MS APCI+ 248 [M+H]⁺

### ii) 4-(Aminomethyl)-1-(4-phenoxybutyl)piperidin-4-ol

The subtitled compound was prepared from the product of step (i) (1-(4-phenoxybutyl)piperidin-4-one) (1.8 g), trimethylsilylcyanide (1.1 g) and zinc chloride (60 mg) in DMF (2 mL) followed by lithium aluminium hydride (12 mL, 1M in THF) using the method of Example 26 step (ii) as a yellow solid. Yield: 2.5 g

MS APCI+ 279 [M+H]⁺

### iii) 8-(Benzyloxy)-5-[(1R)-1-{[tert-butyl(dimethyl)silyl]oxy}-2-({[4-hydroxy-1-(4-phenoxybutyl)piperidin-4-yl]methyl}amino)ethyl]quinolin-2(1H)-one

The product of step (ii) (4-(aminomethyl)-1-(4-phenoxybutyl)piperidin-4-ol) (0.2 g) and the product of Example 2 step (iii) (8-(benzyloxy)-5-((1R)-2-bromo-1-{[tert-butyl(dimethyl)silyl]oxy}ethyl)quinolin-2(1H)-one) (0.23 g), potassium carbonate (0.19 g), sodium iodide (0.1 g) were heated in DMSO (2 mL) at 90° C. for 6 h. The cooled reaction mixture was partitioned between brine and ethyl acetate. The organic layer collected, dried (MgSO₄) and evaporated to leave a red gum. Purification was reverse phase HPLC using an Xterra® C8 5 micron 19×50 mm column eluting with a gradient of 95% 0.880 NH₃ in acetonitrile to 5% 0.880 NH₃ in acetonitrile give the subtitle compound as a pale yellow foam. Yield: 70 mg

MS APCI+ 686 [M+H]⁺

### iv) 8-Hydroxy-5-[(1R)-1-hydroxy-2-({[4-hydroxy-1-(4-phenoxybutyl)piperidin-4-yl]methyl}amino)ethyl]quinolin-2(1H)-one

The product of step (iii) (8-(benzyloxy)-5-[(1R)-1-{[tert-butyl(dimethyl)silyl]oxy}-2-({[4-hydroxy-1-(4-phenoxybutyl)piperidin-4-yl]methyl}amino)ethyl]quinolin-2(1H)-one) (70 mg) in THF (2 ml) was treated with triethylamine trihydroflouride (0.15 mL). After stirring for 18 h at ambient temperature the volatiles were evaporated in vacuo and the residue azeotroped with toluene (×2) to leave yellow gum. This was dissolved in ethanol (10 ml) and 2M HCl (2 ml), 10% palladium on charcoal (10 mg) and hydrogenated at 5 bar pressure for 4 h. The catalyst filtered and the residue purified by reverse phase using an Xterra® C8 5 micron 19×50 mm column eluting with a gradient of 95% 0.880 NH₃ in acetonitrile to 5% 0.880 NH₃ in acetonitrile give the title compound as a pale yellow solid. Yield: 21 mg

MS APCI+ 482 [M+H]⁺

¹H NMR (300 MHz, d6-DMSO) δ 8.19 (d, 1H), 7.27 (t, 2H), 7.07 (d, 1H), 6.91 (d, 4H), 6.50 (d, 1H), 5.03 (m, 1H), 3.96 (t, 2H), 2.72 (m, 2H), 2.46 (m, 2H), 2.33 (bs, 4H), 1.70 (t, 2H), 2.5 (m, 3H), 1.57 (m, 2H), 1.48 (bs, 3H)

### EXAMPLE 41

### N-1-Adamantyl-3-(4-{[(2R)-2-hydroxy-2-(8-hydroxy-2-oxo-1,2-dihydroquinolin-5-yl)ethyl]amino}piperidin-1-yl)propanamide

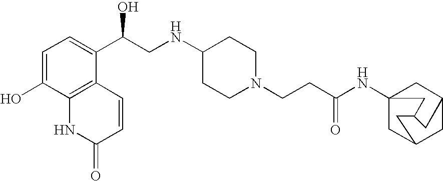

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.18 g), using HATU (0.29 g), triethylamine (0.19 mL) and adamantan-1-amine (0.13 g) in dry DMF (4.1 mL) according to the procedure described in Example 27 step. Purification was by reverse phase HPLC using an Xterra® C8 5 micron 19×50 mm column eluting with a gradient of 95% 0.880 NH₃ in acetonitrile to 60% 0.880 NH₃ in acetonitrile give the title compound as a yellow foam.

Yield: 50 mg

MS APCI+ 509 [M+H]⁺

¹H NMR (300 MHz, d₆-DMSO) δ 8.16 (d, 1H), 7.69 (s, 1H), 7.07 (d, 1H), 6.91 (d, 1H), 6.50 (d, 1H), 4.99 (t, 1H), 2.73 (m, 3H), 2.55 (m, 2H), 2.43 (t, 3H), 2.13 (t, 3H), 1.99 (bs, 2H), 1.90 (bs, 6H), 1.76 (t, 2H), 1.61 (bs, 6H), 1.20 (m, 2H)

### EXAMPLE 42

### N-(3,5-Dichlorobenzyl-3-(4-{[(2R)-2-hydroxy-2-(8-hydroxy-2-oxo-1,2-dihydroquinolin-5-yl)ethyl]amino}piperidin-1-yl)propanamide

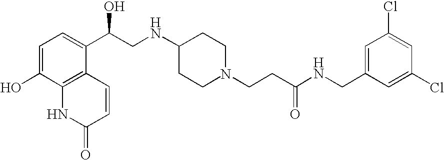

The title compound was prepared from the product of Example 6 step (iii) (3-[4-({[(2R)-2-{[ter-t-butyl(dimethyl)silyl]oxy}-2-(8-hydroxy-2-oxo-1,2-dihydroquinolin-5-yl)ethyl]amino}piperidin-1-yl]propanoic acid) (0.18 g), using HATU (0.25 g), triethylamine (0.066 mL) and 3,5-dichlorobenzylamine (0.13 g) in dry DMF (3.6 mL) with stirring over 18 h according to the procedure described in Example 27. Purification was by reverse phase HPLC using an Xterra® C8 5 micron 19×50 mm column eluting with a gradient of 95% (0.2% aq.NH₃) in acetonitrile to 50% (0.2% aq.NH₃) in acetonitrile give the title compound as a green gum. Yield: 110 mg

MS APCI+ 533/535/537 [M+H]⁺

¹H NMR (300 MHz, d₆-DMSO) δ 10.5 (s, 2H), 8.15 (d, 1H), 7.50 (s, 1H), 7.32 (s, 2H), 7.17 (d, 1H), 6.99 (d, 1H), 6.58 (d, 1H), 5.32 (bm, 1H), 4.30 (d, 2H), 3.54-3.30 (bm, 4H), 3.12 (bm, 2H), 2.96 (bm, 2H), 2.69 (bm, 2H), 2.27 (bm, 2H), 1.80 (bm, 2H). (4H obscured, by the solvent peaks).

### EXAMPLE 43

### 8-Hydroxy-5-{(1R)-1-hydroxy-2-[({4-(hydroxymethyl)-1-[2-(2-phenylethoxy)ethyl]piperidin-4-yl}methyl)amino]ethyl}quinolin-2(1H)-one

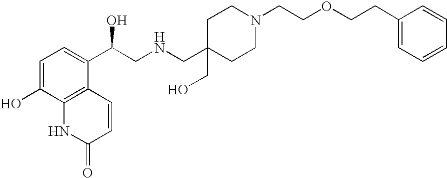

### i) 1-Tert-butyl 4-methyl 4-(iodomethyl)piperidine-1,4-dicarboxylate

n-Butyllithium (33.5 mL of 1.6 M solution in hexanes) was added dropwise to a cooled solution of diisopropylamine (8.2 mL) in THF (250 mL) maintaining the internal temperature between 1.0 and 2.5° C. The reaction mixture was stirred at 0° C. for 10 min then cooled to −78° C. and a solution of 1-tert-butyl 4-methyl piperidine-1,4-dicarboxylate (Eur. J. Org. Chem. 2005, 673) (12.5 g) in THF (150 mL) added dropwise keeping the internal temperature around −72° C. The reaction mixture was stirred at −78° C. for 45 min then diiodomethane (7.9 mL) was added. The reaction mixture was stirred at −78° C. to room temperature overnight then quenched with water and concentrated to a quarter of the volume. EtOAc was added and the layers separated. The aqueous layer was extracted with further ethyl acetate (×2) and the combined organic extracts washed with water, saturated aqueous NaCl, dried (Na₂SO₄) and the volatiles evaporated. Purification was by flash chromatography eluting with 1:9 EtOAc:isohexane to afford the subtitle compound as a colourless oil. Yield: 20.2 g

¹H NMR (300 MHz, CDCl₃) δ 3.87-3.80 (m, 2H), 3.76 (s, 3H), 3.29 (s, 2H), 3.03-2.95 (m, 2H), 2.20-2.16 (m, 2H), 1.52-1.41 (m, 2H), 1.45 (s, 9H).

### ii) 1-Tert-butyl 4-methyl 4-(azidomethyl)piperidine-1,4-dicarboxylate

Sodium azide (4.00 g) was added to a solution of the product of step (i) (1-tert-butyl 4-methyl 4-(iodomethyl)piperidine-1,4-dicarboxylate) (6.04 g) in dry DMSO (60 mL) and the reaction mixture heated at 95° C. for 4 h. After cooling the mixture was diluted with EtOAc and water, the layers separated and the aqueous layer extracted with further EtOAc (×5). The combined organic extracts were washed with water, saturated aqueous NaCl, dried (Na₂SO₄) and the volatiles evaporated to afford the subtitle compound as a pale yellow oil. Yield: 4.35 g

¹H NMR (300 MHz, CDCl₃) δ 3.88-3.80 (m, 2H), 3.77 (s, 3H), 3.42 (s, 2H), 3.04-2.96 (m, 2H), 2.09 (d, 2H), 1.45 (s, 9H), 1.50-1.43 (m, 2H).

### iii) Methyl 4-(azidomethyl)piperidine-4-carboxylate

4M HCl in dioxane (16 mL) was added to the product of step (ii) (1-tert-butyl 4-methyl 4-(azidomethyl)piperidine-1,4-dicarboxylate) (1.90 g). After 2 h at room temperature the volatiles were removed to afford the subtitle compound as a pale yellow solid.

Yield: 2.15 g

¹H NMR (300 MHz, d₆-DMSO) δ 9.13-9.05 (m, 2H), 3.71 (s, 3H), 3.72-3.65 (m, 2H), 3.22-3.17 (m, 2H), 2.94-2.82 (m, 2H), 2.12-2.06 (m, 2H), 1.79-1.69 (m, 2H).

### iv) 2-(2-Phenylethoxy)ethyl trifluoromethanesulfonate

2-(2-Phenylethoxy)ethanol (J. Med. Chem. 1983, 26, 1570) (1.00 g) was dissolved in DCM (20 mL) and cooled to −10° C. Pyridine (0.6 mL) then trifluoromethanesulphonic anhydride (1.2 mL) were added dropwise and the reaction mixture stirred for 1 h before warming to room temperature and stirring overnight. The volatiles were evaporated, then the residue triturated with isohexane, filtered, and washed with further iso-hexane. The solid was discarded and the residue concentrated to afford the subtitle compound as a colourless oil.

Yield: 1.48 g

¹H NMR (300 MHz, CDCl₃) δ 7.33-7.19 (m, 5H), 4.61-4.58 (m, 2H), 3.76-3.69 (m, 4H), 2.90 (t, 2H).

### v) Methyl 4-(azidomethyl)-1-[2-(2-phenylethoxy)ethyl]piperidine-4-carboxylate

The product from step (iii) (methyl 4-(azidomethyl)piperidine-4-carboxylate) (1.06 g) was added to a solution of the product from step (iv) 2-(2-phenylethoxy)ethyl trifluoromethanesulfonate (1.48 g) in DCM (55 mL). Hünig's base (1.7 mL) was added and the reaction mixture was stirred at room temperature overnight. The mixture was diluted with EtOAc and washed with saturated aqueous NaHCO₃, water, saturated aqueous NaCl, dried (Na₂SO₄) and the volatiles evaporated. Purification was by Biotage chromatography eluting with 1:1 to 1:0 EtOAc:isohexane then 1% 7M NH₃/MeOH in EtOAc to afford the subtitle compound as a colourless oil. Yield: 0.94 g

¹H NMR (300 MHz, CDCl₃) δ 7.31-7.20 (m, 5H), 3.75 (s, 3H), 3.65 (t, 2H), 3.56 (t, 2H), 3.40 (s, 2H), 2.88 (t, 2H), 2.74-2.69 (m, 2H), 2.55 (t, 2H), 2.23-2.10 (m, 4H), 1.61-1.51 (m, 2H).

### vi) {4-(Aminomethyl)-1-[2-(2-phenylethoxy)ethyl]piperidin-4-yl}methanol

Lithium aluminium hydride (15 mL, 1M in THF) was added slowly to a cooled (−78° C.) solution of the product from step (v) methyl 4-(azidomethyl)-1-[2-(2-phenylethoxy)ethyl]piperidine-4-carboxylate (0.69 g) in dry THF (10 mL). The reaction mixture was allowed to warm to room temperature gradually and stirred overnight. It was cooled in ice then a mixture of Celite (3.5 g) and sodium sulphate decahydrate (3.5 g) added in portions, keeping the temperature below 10° C. Additional THF was added, followed by 0.4 mL of 10% aqueous NaOH. The solids were removed by filtration and the volatiles evaporated to afford the subtitle compound as a pale yellow oil. Yield: 0.56 g

¹H NMR (400 MHz, CDCl₃) δ 7.30-7.18 (m, 5H), 3.67-3.63 (m, 4H), 3.58 (t, 2H), 2.90-2.81 (m, 4H), 2.58 (t, 2H), 2.54-2.49 (m, 2H), 2.38-2.32 (m, 2H), 1.61-1.55 (m, 2H), 1.46-1.39 (m, 2H).

### vii) 8-(Benzyloxy)-5-{(1R)-1-{[tert-butyl(dimethyl)silyl]oxy}-2-[({4-(hydroxymethyl)-1-[2-(2-phenylethoxy)ethyl]piperidin-4-yl}methyl)amino]ethyl}quinolin-2(1H)-one

A mixture of the product from example 2 step (iii) (8-(benzyloxy)-5-((1R)-2-bromo-1-{[tert-butyl(dimethyl)silyl]oxy}ethyl)quinolin-2(1H)-one) (0.85 g), the product from step (vi) ({4-(aminomethyl)-1-[2-(2-phenylethoxy)ethyl]piperidin-4-yl}methanol) (0.51 g), potassium iodide (0.58 g) and sodium bicarbonate (0.58 g) in DMSO (4.5 mL) was heated at 100° C. for 12 h. It was allowed to cool and diluted with EtOAc and water, and the aqueous material extracted with further EtOAc (×3). The combined organic extracts were washed with water, saturated aqueous NaCl, dried (Na₂SO₄) and concentrated. Purification was by reverse phase HPLC using a Symmetry® column eluting with a gradient of 5-95% acetonitrile in 0.2% aqueous TFA to afford the subtitle compound di-trifluoroacetate salt as a yellow oil. Yield: 0.172 g

MS APCI+ 700 [M+H]⁺

### viii) 8-(Benzyloxy)-5-{(1R)-1-hydroxy-2-[({4-(hydroxymethyl)-1-[2-(2-phenylethoxy)ethyl]piperidin-4-yl}methyl)amino]ethyl}quinolin-2(1H)-one

The product from step (vii) (8-(benzyloxy)-5-{(1R)-1-{[tert-butyl(dimethyl)silyl]oxy}-2-[({4-(hydroxymethyl)-1-[2-(2-phenylethoxy)ethyl]piperidin-4-yl}methyl)amino]ethyl}-quinolin-2(1H)-one) (0.17 g) was dissolved in THF (3 mL) and to it was added triethylamine trihydrofluoride (0.15 mL). The reaction mixture was stirred at room temperature for 2.5 h after which time further triethylamine trihydrofluoride (0.15 mL) was added. The mixture was stirred overnight then the volatiles evaporated to afford the subtitle compound as a yellow oil. Yield: 0.14 g

MS APCI+ 586 [M+H]⁺

### ix) 8-Hydroxy-5-{(1R)-1-hydroxy-2-[({4-(hydroxymethyl)-1-[2-(2-phenylethoxy)-ethyl]piperidin-4-yl}methyl)amino]ethyl}quinolin-2(1H)-one

A suspension of palladium on carbon (10%, 37 mg) in 1:1 DCM:MeOH (2 mL) was added to a solution of the product from step (viii) (8-(benzyloxy)-5-{(1R)-1-hydroxy-2-[({4-(hydroxymethyl)-1-[2-(2-phenylethoxy)ethyl]piperidin-4-yl}methyl)amino]ethyl}quinolin-2(1H)-one) (0.14 g) in 1:1 DCM:MeOH (4 mL) and hydrogenated at 4 bar for 3.5 h. The catalyst was removed by filtration and the solvent evaporated. Purification was by reverse phase HPLC using an XBridge® column eluting with a gradient of 10-40% acetonitrile in 0.2% aqueous TFA) to afford the title compound di-trifluoroacetate salt as a white solid.

Yield: 35 mg

MS APCI+ 496 [M+H]⁺

¹H NMR (300 MHz, d₆-DMSO) δ 10.58 (s, 1H), 10.51 (s, 1H), 8.20 (d, 1H), 7.32-7.20 (m, 6H), 7.00 (d, 1H), 6.57 (d, 1H), 6.29 (s, 1H), 5.43-5.36 (m, 1H), 3.75-3.65 (m, 6H), 3.30-3.24 (m, 2H), 3.14-3.06 (m, 6H), 2.84 (t, 2H), 1.70-1.16 (m, 6H)

### EXAMPLE 44

### 2,6-Dichloro-N-[2-(4-{[(2R)-2-hydroxy-2-(8-hydroxy-2-oxo-1,2-dihydroquinolin-5-yl)ethyl]amino}piperidin-1-yl)ethyl]benzamide

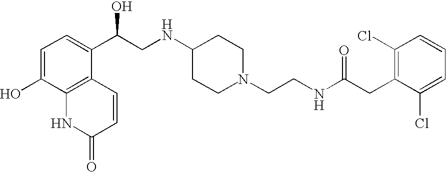

### i) 2,6-Dichloro-N-[2-(4-hydroxypiperidin-1-yl)ethyl]benzamide

A solution of 1-(2-aminoethyl)piperidin-4-ol (0.312 g), 2,6-dichlorobenzoic acid (0.826 g), and triethylamine (0.61 mL) in DMF (10 mL) was treated with PyBROP (1.21 g) at ambient temperature. After stirring for 14 h the mixture was loaded onto a SCX cartridge and eluted with acetonitrile followed by methanol. The product was then eluted off with ammonia/methanol solutions to give the subtitle compound, after solvent evaporation, as a yellow oil. Yield: 0.7 g

MS APCI+ 317/319/321 [M+H]⁺

### ii) 2,6-Dichloro-N-[2-(4-oxopiperidin-1-yl)ethyl]benzamide

A solution of the product of step (i) (2,6-dichloro-N-[2-(4-hydroxypiperidin-1-yl)ethyl]benzamide) (0.1 g) in acetone (2 mL) and water (2 mL) was treated with Jones reagent (0.48 mL) (made by the following procedure: sodium dichromate dihydrate (11.0 g in water (3 mL) and cooled in an ice bath, concentrated sulphuric acid (0.74 mL) was added dropwise. The resulting solution of Jones reagent was then diluted to 5 mL total volume). The mixture was stirred at ambient temperature for 14 h, then loaded onto an SCX cartridge and eluted with water followed by methanol. The product was then eluted off with ammonia/methanol solutions to give the subtitle compound, after solvent evaporation, as a orange oil. Yield: 0.1 g

MS APCI+ 315/317/319 [M+H]⁺

### iii) 2,6-Dichloro-N-[2-(4-{[(2R)-2-hydroxy-2-(8-hydroxy-2-oxo-1,2 dihydroquinolin-5-yl)ethyl]amino}piperidin-1-yl)ethyl]benzamide

A solution of the product of step (ii) (2,6-dichloro-N-[2-(4-oxopiperidin-1-yl)ethyl]benzamide) (0.1 g) and the product of Example 5 step (ii) (5-((1R)-2-amino-1-{[tert-butyl(dimethyl)silyl]oxy}ethyl)-8-hydroxyquinolin-2(1H)-one) (0.053 g) in NMP (3 mL) was treated with sodium triacetoxyborohydride (0.068 g) and acetic acid (0.01 mL) at ambient temperature. After stirring for 14 h the mixture was loaded onto an SCX cartridge and eluted with isopropanol/acetonitrile (1:1). The product was then eluted off with ammonia/methanol solutions and solvent evaporated in vacuo to leave a yellow foam. This was then dissolved in THF (3 mL) and treated with triethylamine trihydrofluoride (0.1 mL) and the mixture stirred at ambient temperature for 14 h. The volatiles evaporated in vacuo to give the crude product. This was purified by reverse phase HPLC using an Xterra® C8 5 micron 19×50 mm column eluting with a gradient of 95% (0.2% aq.NH₃) in acetonitrile to 60% (0.2% aq.NH₃) in acetonitrile give the title compound as a yellow foam. Yield: 20 mg

MS APCI+ 519 [M+H]⁺

¹H NMR (300 MHz, d₆-DMSO) δ 8.88 (t, 1H), 8.17 (d, 1H), 7.50-7.39 (m, 3H), 7.07 (d, 1H), 6.91 (d, 1H), 6.50 (d, 1H), 4.98 (t, 1H), 2.84-2.76 (m, 2H), 2.74-2.65 (m, 2H), 2.57-2.30 (m, 6H), 2.43 (t, 2H), 2.02-1.92 (m, 2H), 1.80-1.68 (m, 2H), 1.30-1.17 (m, 2H)

### EXAMPLE 45

### 8-Hydroxy-5-[(1R)-1-hydroxy-2-({[1-(2-{[(2S)-2-phenylpropyl]oxy}ethyl)piperidin-4-yl]methyl}amino)ethyl]quinolin-2(1H)-one

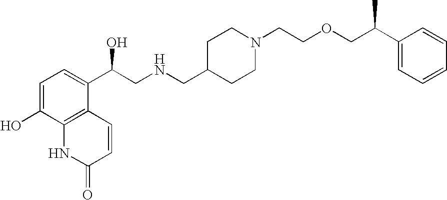

### i) 1-(4-Hydroxymethylpiperidin-1-yl)-2-((S)-2-phenylpropoxy)ethanone

A solution of ((S)-2-phenylpropoxy)acetic acid (0.7 g) (WO97/10227) in dry DMF (30 mL) was treated with HATU (2.5 g), triethylamine (0.037 mL) and 4-piperidinemethanol (0.85 g) at ambient temperature. After 3 days the reaction mixture was partitioned between ethyl acetate and water. The organic layer was washed with 2M HCl, saturated aqueous sodium bicarbonate, water, brine and dried over anhydrous magnesium sulphate. The organic solution was filtered and concentrated under reduced pressure to give the subtitle compound (0.97 g), which was used in the next step without further purification.

MS APCI+ 292 [M+H]⁺

### ii) [1-[2-((S)-2-Phenylpropoxy)ethyl]piperidin-4-yl]methanol

A solution of lithium aluminium hydride (1.8 mL, 1.0M in THF) was added dropwise to a stirred solution of the product from step (i) (1-(4-hydroxymethylpiperidin-1-yl)-2-((S)-2-phenylpropoxy)ethanone) (0.43 g) in dry THF (5 mL) under nitrogen. After stirring for 18 h a mixture of Celite (2.9 g) and sodium sulphate decahydrate (2.9 g) was added batchwise.

A solution of 1M aqueous sodium hydroxide (0.5 mL) was added and the resulting mixture stirred for 30 min. (Tetrahedron, 1996., 52, 8517). The solids were filtered and washed with THF. The combined filtrates were concentrated to give a colourless oil. The crude product was loaded onto an SCX cartridge and eluted with 1:1 isopropanol/acetonitrile. The product was eluted off with 20% 0.880 NH₃ in 1:1 isopropanol/acetonitrile to give the subtitle product as a colourless gum (0.31 g).

MS APCI+ 278 [M+H]⁺

### iii) 1-[2-(S)-2-Phenylpropoxy)ethyl]piperidine-4-carbaldehyde

4 A Molecular sieves (0.05 mg) followed by N-methylmorpholine N-oxide (0.05 g) and TPAP (0.01 g) were added to a solution of the product of step (ii) ([1-[2-((S)-2-phenylpropoxy)ethyl]piperidin-4-yl]methanol) (0.10 g) in DCM (3 mL). After 1.5 h, additional DCM (15 mL), NMO (0.05 g) and TPAP (0.01 g) were added and the mixture was stirred for a further 18 h. The reaction mixture was filtered through Celite and the filtrate concentrated in vacuo. The black gum was purified by column chromatography on deactivated alumina (neutral, Brockman 1) eluting with Et₂O and DCM (1:1) to give the subtitle compound as clear oil. Yield: 0.04 g

MS APCI+ 294 [M+H₂O+H]⁺

### iv) 8-Hydroxy-5-[(R)-1-hydroxy-2-({1-[2-((S)-2-phenylpropoxy)ethyl]piperidin-4-ylmethyl}amino)ethyl]-1H-quinolin-2-one

A solution of the product of example 5 step (ii) (5-((1R)-2-amino-1-{[tert-butyl(dimethyl)silyl]oxy}ethyl)-8-hydroxy quinolin-2(1H)-one) (0.05 g) and the product of step (iii) (1-[2-(S)-2-phenylpropoxy)ethyl]piperidine-4-carbaldehyde) (0.04 g) in methanol (1.6 L) was treated with acetic acid (0.018 mL). Sodium cyanoborohydride (9.8 mg) was added and stirring continued for 18 h. The solvent was removed under reduced pressure and the residue was partitioned between ethyl acetate (50 mL) and water (50 mL) containing concentrated aqueous ammonia (1 mL). The organic layer was dried (MgSO₄), filtered and concentrated under reduced pressure. The residue dissolved in THF (4.0 mL) was treated with triethylamine trihydrofluoride (0.14 mL) with further stirring for 5.5 h. The volatiles were evaporated in vacuo and the residue purified by reverse phase HPLC using Xterra® C8 5 micron 19×50 mm column eluting with a gradient of 95% (0.2% aq.NH₃) in acetonitrile to 70% (0.2% aq.NH₃) in acetonitrile give the title compound as a pale green solid. Yield: 27 mg

MS APCI+ 480 [M+H]⁺

¹H NMR (300 MHz, d₆-DMSO) δ 8.16 (d, 1H), 7.26 (m, 4H), 7.17 (m, 1H), 6.99 (d, 1H), 6.81 (d, 1H), 6.46 (d, 1H), 4.96 (m, 1H), 3.39-3.49 (m, 4H), 2.95 (m, 1H), 2.77 (bd, 2H), 2.65 (m, 2H), 2.38 (m, 4H), 1.84 (t, 2H), 1.56 (d, 2H), 1.18 (d, 3H), 1.04 (m, 2H). (1×H obscured, by the solvent peak).

### EXAMPLE 46

### 5-((1R)-2-{[(1-{2-[2-(2-chlorophenyl)ethoxy]ethyl}-4-hydroxypiperidin-4-yl)methyl]amino}-1-hydroxyethyl)-8-hydroxyquinolin-2(1H)-one

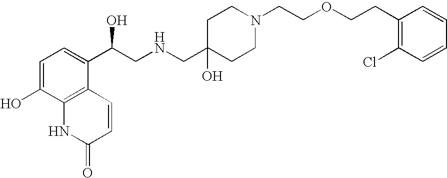

### i) [2-(2-Chloro-phenyl)-ethoxy]-acetic acid

2-(2-Chlorophenyl)ethanol (10 g) was dissolved in DMF (125 mL) and cooled in a ice bath. Sodium hydride (1.687 g, 60% in mineral oil) was added batchwise over 15 min. The reaction mixture was stirred at room temperature for 1 h 45 min then sodium chloroacetate (7.44 g) was added. The mixture was stirred at room temperature for 15 min, heated to 60° C. for 7 h, then left stirring at room temperature for 50 h. The reaction mixture was poured into a large volume of water and thoroughly washed with ether. The aqueous solution was acidified (conc HCl) and was extracted with ether, which was then washed with water and brine. Drying (Na₂SO₄) and evaporation gave the subtitle compound as an amber oil. Yield: 6.9 g

¹H NMR δ (CDCl₃) 3.09 (2H, t), 3.81 (2H, t), 4.13 (2H, s), 7.23-7.14 (2H, m), 7.35 (1H, dd), 7.29 (1H, dd)

### ii) 2-[2-(2-Chloro-phenyl)-ethoxy]-1-(4-hydroxy-piperidin-1-yl)-ethanone

The product of step (i) ([2-(2-chloro-phenyl)-ethoxy]-acetic acid) (6.9 g) was dissolved in DCM (100 mL) and treated with thionyl chloride (5 mL) with dry DMF (1 drop) at reflux.

After 1 h at reflux all volatiles were evaporated and the residue was azeotroped with toluene. A solution of this acid chloride dissolved in DCM (50 mL) was then added to a solution of 4-hydroxy-piperidine (3.25 g) dissolved in DCM (50 mL) and triethylamine (3.24 g). After 2 h stirring at room temperature the reaction mixture was washed with water and sat. aq. sodium bicarbonate solution and the organic solution was evaporated leaving an orange oil. Purification by silica gel column chromatography eluting with ethyl acetate affords the subtitle compound as an oil. Yield: 6.6 g

¹H NMR δ (CDCl₃) 7.34 (1H, dd), 7.30-7.25 (1H, m), 7.22-7.12 (2H, m), 4.15 (2H, s), 4.06-3.96 (1H, m), 3.93-3.85 (1H, m), 3.75 (2H, t), 3.78-3.63 (1H, m), 3.24-3.10 (1H, m), 3.06 (2H, t), 2.04-1.72 (4H, m), 1.55-1.34 (2H, m)

### iii) 1-{2-[2-(2-Chloro-phenyl)-ethoxy]-ethyl}-piperidin-4-ol

The product of step (ii) (2-[2-(2-Chloro-phenyl)-ethoxy]-1-(4-hydroxy-piperidin-1-yl)-ethanone) (6.6 g) in THF (100 mL) under nitrogen was treated with lithium aluminium hydride (50 mL, 1M in THF,) and then set at reflux for 4 h, then left at room temperature for 24 h. Excess EtOAc was cautiously added and the reaction was stirred 2 h. 1M NaOH solution (25 mL) was added and the whole was stirred until the solid mass broke up. Filtration through Celite afforded a two phase mixture which was thoroughly extracted into EtOAc and evaporated. Purification by silica gel column chromatography eluting with EtOAc and then MeOH+3% triethylamine affords the subtitle compound as an oil.

Yield: 3.5 g

¹H NMR δ (CDCl₃) 7.34 (1H, dd), 7.28-7.24 (1H, m), 7.20-7.14 (2H, m), 3.70 (2H, t), 3.62 (2H, t), 3.04 (2H, t), 2.78 (4H, t), 2.70 (2H, t), 2.43 (4H, t)

### iv) 1-{2-[2-(2-Chloro-phenyl)-ethoxy]-ethyl}-piperidin-4-one

The product of step (iii) (1-{2-[2-(2-chloro-phenyl)-ethoxy]-ethyl}-piperidin-4-ol) (710 mg) was dissolved in dry DCM (6 mL) and N-methyl morpholine-N-oxide (360 mg, 3.12 mmol) and terapropylammonium perruthenate (7.5 mol %, 66 mg) were added. The reaction was stirred at room temperature. After 1 h the reaction mixture was diluted with ether and filtered through a bed of alumina deactivated with 6% water. The bed was eluted with ether. Evaporation of fractions gave an oil which was purified by silica gel column chromatography eluting with EtOAc then EtOAc-0.5% triethylamine to afford the subtitle compound as an oil. Yield: 330 mg

¹H NMR δ (CDCl₃) 7.34 (1H, dd), 7.28-7.24 (1H, m), 7.20-7.14 (2H, m), 3.70 (2H, t), 3.62 (2H, t), 3.04 (2H, t), 2.78 (4H, t), 2.70 (2H, t), 2.43 (4H, t)

### v) 5-((1R)-1-{[tert-butyl(dimethyl)silyl]oxy}-2-{[(1-{2-[2-(2-chlorophenyl)ethoxy]ethyl}-4-hydroxypiperidin-4-yl)methyl]amino}ethyl)-8-hydroxyquinolin-2(1H)-one

Sodium hydride (56.6 mg, 60% in mineral oil) was added to dry DMSO (2.9 mL) and trimethyl sulphoxonium iodide (390 mg) was added. The mixture was stirred under nitrogen for 1.25 h at room temperature and then a solution of the product of step (iv) (1-{2-[2-(2-chloro-phenyl)-ethoxy]-ethyl}-piperidin-4-one) (330 mg) in dry THF (2.9 mL) was added. The reaction mixture was stirred under nitrogen for 16 h and then was poured into water. Extraction into EtOAc, drying (Na₂SO₄) and evaporation gave an oil. The oil was dissolved in methanol (0.3 mL) and was treated with the product of Example 5 step (ii) (5-((1R)-2-amino-1-{[tert-butyl(dimethyl)silyl]oxy}ethyl)-8-hydroxyquinolin-2(1H)-one) (99 mg). The solution was heated at 65° C. for 4 h then left at room temperature for 16 h. The mixture was purified by adsorption onto silica gel followed by silica gel chromatography eluting with 7.5% MeOH in DCM then 10% MeOH in DCM and finally with 100% MeOH to afford the subtitle compound. Further purification was by reverse phase HPLC to afford the subtitle compound as a yellow glass. Yield: 39 mg

¹H NMR δ (DMSO) 8.43 (1H, s), 7.57 (2H, s), 7.42 (2H, s), 7.19 (1H, s), 7.07 (1H, s), 6.66 (1H, s), 5.28 (1H, s), 3.24-3.85 (1H, m), 2.92-3.24 (1H, m), 2.34-2.90 (52H, m), 1.59 (3H, s), 1.00 (9H, s), 0.22 (3H, s), 0.00 (3H, s)

### vi) 5-((1R)-2-{[(1-{2-[2-(2-chlorophenyl)ethoxy]ethyl}-4-hydroxypiperidin-4-yl)methyl]amino}-1-hydroxyethyl)-8-hydroxyquinolin-2(1H)-one

The product of step (v) (5-{1-(tert-butyl-dimethyl-silanyloxy)-2-[(1-{2-[2-(2-chloro-phenyl)-ethoxy]-ethyl}-4-hydroxy-piperidin-4-ylmethyl)-amino]-ethyl}-8-hydroxy-1H-quinolin-2-one) (35 mg) was dissolved in dry THF (1 mL) and treated with triethylamine trihydrofluoride (0.2 mL) at room temperature for 16 h. All volatiles were removed in vacuo and the residue was azeotroped with acetonitrile (×2). Purification was by reverse phase HPLC using an Xterra® C8 5 micron 19×50 mm column eluting with a gradient of 95% (0.2% aq.NH₃) in acetonitrile to 50% (0.2% aq.NH₃) in acetonitrile give the title compound as a yellow powder. Yield: 9 mg

MS APCI+ 516 [M+H]⁺

¹H NMR δ (DMSO) 8.19 (1H, d), 7.43-7.34 (2H, m), 7.29-7.20 (2H, m), 7.06 (1H, d), 6.89 (1H, d), 6.49 (1H, d), 5.03-4.98 (1H, m), 3.59 (2H, t), 3.48 (2H, t), 2.92 (2H, t), 2.72-2.64 (3H, m), 2.60-2.36 (258H, m signal under DMSO peak), 2.35-2.23 (6H, m), 1.48-1.36 (4H, m)

### EXAMPLE 47

### N-(2,5-Dimethylbenzyl-3-(4-{[(2R)-2-hydroxy-2-(8-hydroxy-2-oxo-1,2-dihydroquinolin-5-yl)ethyl]amino}piperidin-1-yl)propanamide

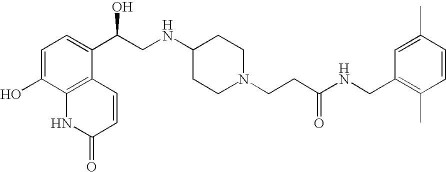

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.15 g), using HATU (0.22 g), triethylamine (0.11 mL) and 2,5-dimethylbenzylamine (0.074 g) in dry DMF (3.0 mL) with stirring over 18 h according to the procedure described in Example 27. Purification was by reverse phase HPLC using an Xterra® C8 5 micron 19×50 mm column eluting with a gradient of 95% (0.2% aq.NH₃) in acetonitrile to 70% (0.2% aq.NH₃) in acetonitrile give the title compound as a pale green gum. Yield: 36 mg

MS APCI+ 493 [M+H]⁺

¹H NMR (300 MHz, d₆-DMSO) δ 8.27 (t, 1H), 8.16 (d, 1H), 7.04 (m, 3H), 6.91 (m, 2H), 6.50 (d, 1H), 4.95 (t, 1H), 4.18 (d, 2H), 2.76 (bm, 2H), 2.68 (d, 2H), 2.37 (m, 2H), 2.27 (t, 2H), 2.23 (s, 3H), 2.19 (s, 3H), 1.90 (bm, 2H), 1.71 (bm, 2H), 1.16 (bm, 2H). (1×H obscured, by the solvent peak).

### EXAMPLE 48

### N-(Adamant-1-ylmethyl)-3-(4-{[(2R)-2-hydroxy-2-(8-hydroxy-2-oxo-1,2-dihydroquinolin-5-yl)ethyl]amino}piperidin-1-yl)propanamide

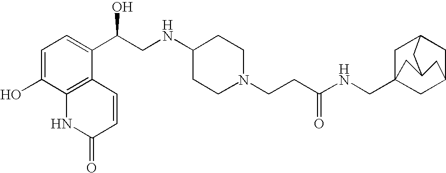

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.15 g), using HATU (0.22 g), triethylamine (0.11 mL) and 1-adamantanylmethylamine (0.1 g) in dry DMF (3.0 mL) with stirring over 18 h according to the procedure described in Example 27. Purification was by mass directed purification to give the title compound as a gum. Yield: 8 mg

MS APCI+ 523 [M+H]⁺

¹H NMR (300 MHz, d₆-DMSO) δ 8.17 (d, 1H), 7.90 (t, 1H), 7.05 (d, 1H), 6.89 (d, 1H), 6.48 (d, 1H), 4.94 (t, 1H), 2.77 (m, 4H), 2.69 (m, 3H), 2.22 (t, 2H), 1.89 (bs, 4H), 1.74 (bt, 2H), 1.64 (d, 4H), 1.56 (d, 4H), 1.41 (bs, 4H), 1.22 (bm, 2H). (4H obscured, by the solvent peal).

### EXAMPLE 49

### N-(3-Chloro-2-methylbenzyl)-3-(4-{[(2R)-2-hydroxy-2-(8-hydroxy-2-oxo-1,2-dihydroquinolin-5-yl)ethyl]amino}piperidin-1-yl)propanamide

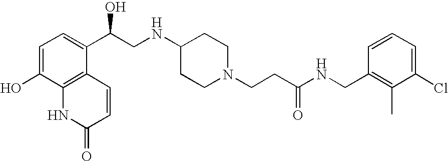

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.15 g), using HATU (0.22 g), triethylamine (0.11 mL) and 3-chloro-2-methylbenzylamine (0.085 g) in dry DMF (3.0 mL) with stirring over 18 h according to the procedure described in Example 27. Purification was by reverse phase HPLC using an Xterra® C8 5 micron 19×50 mm column eluting with a gradient of 95% 0.880 NH₃ in acetonitrile to 70% 0.880 NH₃ in acetonitrile give the title compound as a green gum. The gum was further purified by reverse phase HPLC using an Xterra® C8 5 micron 19×50 mm column eluting with a gradient of 95% TFA in acetonitrile to 50% TFA in acetonitrile and then loaded onto an SCX cartridge using acetonitrile then 20% 0.880 ammonia in acetonitrile as eluent to give the title compound bis trifluoroacetate salt as a pale yellow solid. Yield: 50 mg

MS APCI+ 513 [M+H]⁺

¹H NMR (400 MHz, d₆-DMSO/ND4OD) δ 8.26 (d, 1H), 7.29 (d, 1H), 7.19 (m, 3H), 6.99 (d, 1H, 6.62 (d, 1H), 5.12 (t, 1H), 4.29 (s, 2H), 2.81 (bm, 4H), 2.34 (bt, 2H), 2.29 (s, 3H), 1.97 (bt, 2H), 1.79 (bm, 2H), 1.29 (bm, 2H). (3×H obscured by the solvent peak).

### EXAMPLE 50

### 3-(4-{[(2R)-2-Hydroxy-2-(8-hydroxy-2-oxo-1,2-dihydroquinolin-5-yl)ethyl]amino}piperidin-1-yl)-N-(2-trifluoromethoxybenzyl)-propanamide

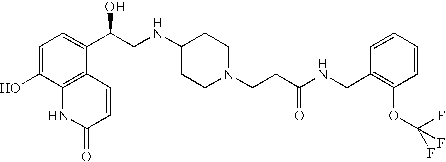

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.15 g), using HATU (0.22 g), triethylamine (0.11 mL) and 2-(trifluoromethoxy)benzylamine (0.12 g) in dry DMF (3.0 mL) with stirring over 18 h according to the procedure described in Example 27. Purification was by reverse phase HPLC using an Xterra® C8 5 micron 19×50 mm column eluting with a gradient of 95% (0.2% aq.NH₃) in acetonitrile to 80% (0.2% aq.NH₃) in acetonitrile give the title compound as a cream solid. Yield: 15 mg

MS APCI+ 549 [M+H]⁺

¹H NMR (300 MHz, d₆-DMSO) δ 8.46 (m, 1H), 8.17 (d, 1H), 7.50 (d, 1H), 7.36 (m, 3H), 7.06 (d, 1H), 6.90 (d, 1H), 6.49 (d, 1H), 4.96 (t, 1H), 4.32 (d, 2H), 2.77 (m, 2H), 2.70 (d, 2H), 2.39 (m, 1H), 2.30 (t, 2H), 1.90 (m, 2H), 1.73 (m, 2H), 1.21 (m, 2H). (2×H obscured, by the solvent peak).

### EXAMPLE 51

### N-((3-Fluoro-5-trifluoromethyl)benzyl)-3-(4-{[(2R)-2-hydroxy-2-(8-hydroxy-2-oxo-1,2-dihydroquinolin-5-yl)ethyl]amino}piperidin-1-yl)propanamide

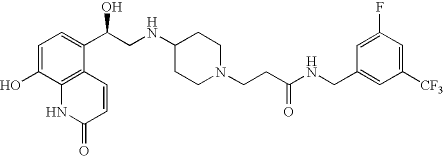

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.15 g), using HATU (0.22 g), triethylamine (0.11 mL) and (3-fluoro-5-trifluoro)benzylamine (0.12 g) in dry DMF (3.0 mL) with stirring over 18 h according to the procedure described in Example 27. Purification was by reverse phase HPLC using an Xterra® C8 5 micron 19×50 mm column eluting with a gradient of 95% (0.2% aq.NH₃) in acetonitrile to 50% (0.2% aq.NH₃) in acetonitrile give the title compound as a beige solid. Yield: 17 mg

MS APCI+ 551 [M+H]⁺

¹H NMR (300 MHz, d₆-DMSO) δ 8.54 (t, 1H), 8.17 (d, 1H), 7.53-7.43 (m, 3H), 7.04 (d, 1H), 6.88 (d, 1H), 6.49 (d, 1H), 4.95 (t, 1H), 4.37 (d, 2H), 2.76 (m, 2H), 2.69 (d, 2H), 2.39 (m, 1H), 2.30 (t, 2H), 1.93 (m, 2H), 1.76 (bt, 2H), 1.21 (bt, 2H). (2×H obscured, by the solvent peak).

### EXAMPLE 52

### N-[2-Fluoro-3-(trifluoromethyl)benzyl]-3-(4-{[(2R)-2-hydroxy-2-(8-hydroxy-2-oxo-1,2-dihydroquinolin-5-yl)ethyl]amino}piperidin-1-yl)propanamide

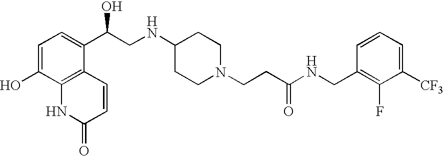

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.15 g), using HATU (0.22 g), triethylamine (0.11 mL) and (2-fluoro-3-trifluoro)benzylamine (0.12 g) in dry DMF (3.0 mL) with stirring over 18 h according to the procedure described in Example 27. Purification was by reverse phase HPLC using an Xterra® C8 5 micron 19×50 mm column eluting with a gradient of 95% (0.2% aq.NH₃) in acetonitrile to 70% (0.2% aq.NH₃) in acetonitrile give the title compound as a cream solid. Yield: 23 mg

MS APCI+ 551 [M+H]⁺

¹H NMR (300 MHz, d₆-DMSO) δ 8.53 (t, 1H), 8.17 (d, 1H), 7.68 (m, 2H), 7.35 (t, 1H), 7.06 (d, 1H), 6.91 (d, 1H), 6.50 (d, 1H), 4.97 (t, 1H), 4.36 (d, 2H), 2.73 (m, 4H), 2.50 (m, 1H), 2.29 (t, 2H), 1.91 (bm, 2H), 1.76 (bm, 2H), 1.20 (bt, 2H). (2×H obscured, by the solvent peak).

### EXAMPLE 53

### N-((2-Chloro-5-trifluoromethyl)benzyl)-3-(4-{[(2R)-2-hydroxy-2-(8-hydroxy-2-oxo-1,2-dihydroquinolin-5-yl)ethyl]amino}piperidin-1-yl)propanamide

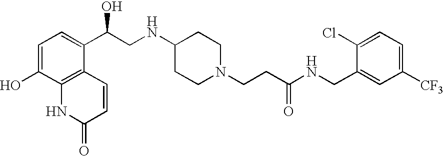

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.15 g), using HATU (0.22 g), triethylamine (0.11 mL) and (2-chloro-5-trifluoromethyl)benzylamine (0.13 g) in dry DMF (3.0 mL) with stirring over 18 h according to the procedure described in Example 27. Purification was by reverse phase HPLC using an Xterra® C8 5 micron 19×50 mm column eluting with a gradient of 95% (0.2% aq.NH₃) in acetonitrile to 70% (0.2% aq.NH₃) in acetonitrile give the title compound as a cream solid. Yield: 22 mg

MS APCI+ 567/569 [M+H]⁺

¹H NMR (300 MHz, d₆-DMSO) δ 8.60 (t, 1H), 8.17 (d, 1H), 7.68 (m, 3H), 7.06 (d, 1H), 6.91 (d, 1H), 6.50 (d, 1H), 4.96 (t, 1H), 4.39 (d, 2H), 2.77-2.69 (m, 4H), 2.34 (m, 2H), 1.93 (m, 2H), 1.72 (m, 2H), 1.20 (bt, 2H). (3×H obscured, by the solvent peak).

### EXAMPLE 54

### N-((5-Fluoro-2-trifluoromethyl)benzyl)-3-(4-{[(2R)-2-hydroxy-2-(8-hydroxy-2-oxo-1,2-dihydroquinolin-5-yl)ethyl]amino}piperidin-1-yl)propanamide

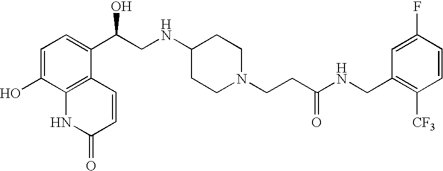

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.15 g), using HATU (0.22 g), triethylamine (0.11 mL) and (5-fluoro-2-trifluoromethyl)benzylamine (0.12 g) in dry DMF (3.0 mL) with stirring over 18 h according to the procedure described in Example 27. Purification was by reverse phase HPLC using an Xterra® C8 5 micron 19×50 mm column eluting with a gradient of 95% (0.2% aq.NH₃) in acetonitrile to 70% (0.2% aq.NH₃) in acetonitrile give the title compound as a cream solid. Yield: 23 mg

MS APCI+ 551 [M+H]⁺

¹H NMR (300 MHz, d₆-DMSO) δ 8.56 (t, 1H), 8.18 (d, 1H), 7.78 (m, 1H), 7.39 (d, 1H), 7.29 (t, 1H), 7.06 (d, 1H), 6.91 (d, 1H), 6.50 (d, 1H), 4.96 (t, 1H), 4.43 (d, 2H), 2.80 (bd, 2H), 2.71 (d, 2H), 2.32 (m, 2H), 1.93 (bm, 2H), 1.74 (bm, 2H), 1.23 (bm, 2H). (3×H obscured, by the solvent peak).

### EXAMPLE 55

### 3-(4-{[(2R)-2-Hydroxy-2-(8-hydroxy-2-oxo-1,2-dihydroquinolin-5-yl)ethyl]amino}piperidin-1-yl)-N-[(2-trifluoromethyl)benzyl]propanamide

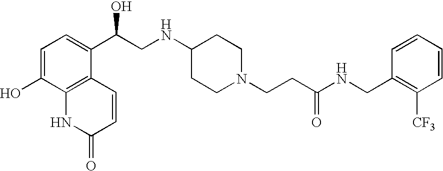

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.15 g), using HATU (0.22 g), triethylamine (0.11 mL) and (2-trifluoromethyl)benzylamine (0.11 g) in dry DMF (3.0 mL) with stirring over 18 h according to the procedure described in Example 27. Purification was by reverse phase HPLC using an Xterra® C8 5 micron 19×50 mm column eluting with a gradient of 95% (0.2% aq.NH₃) in acetonitrile to 70% (0.2% aq.NH₃) in acetonitrile give the title compound as a cream solid. Yield: 27 mg

MS APCI+ 533 [M+H]⁺

¹H NMR (300 MHz, d₆-DMSO) δ 8.53 (t, 1H), 8.18 (d, 2H), 7.70 (d, 1H), 7.65 (s, 1H), 7.46 (m, 1H), 7.07 (d, 1H), 6.91 (d, 1H), 6.50 (d, 1H), 4.97 (t, 1H), 4.44 (d, 2H), 2.80 (bd, 2H), 2.71 (d, 2H), 2.40 (m, 1H), 2.33 (t, 2H), 1.93 (bm, 2H), 1.73 (bt, 2H), 1.20 (bm, 2H). (2×H obscured, by the solvent peak).

### EXAMPLE 56

### N-(5-Chloro-2-methylbenzyl)-3-(4-{[(2R)-2-hydroxy-2-(8-hydroxy-2-oxo-1,2-dihydroquinolin-5-yl)ethyl]amino}piperidin-1-yl)propanamide

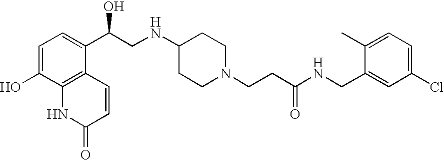

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.15 g), using HATU (0.22 g), triethylamine (0.11 mL) and (5-chloro-2-methyl)benzylamine (0.098 g) in dry DMF (3.0 mL) with stirring over 18 h according to the procedure described in Example 27. Purification was by reverse phase HPLC using an Xterra® C8 5 micron 19×50 mm column eluting with a gradient of 95% (0.2% aq.NH₃) in acetonitrile to 70% (0.2% aq.NH₃) in acetonitrile give the title compound as a cream solid. Yield: 23 mg MS APCI+ 513/515 [M+H]⁺

¹H NMR (300 MHz, d₆-DMSO) δ 8.36 (t, 1H), 8.17 (d, 1H), 7.21 (s, 1H), 7.17 (s, 2H), 7.07 (d, 1H), 6.91 (d, 1H), 6.50 (d, 1H), 4.96 (t, 1H), 4.22 (d, 2H), 2.76 (bd, 2H), 2.70 (d, 2H), 2.38 (m, 1H), 2.29 (t, 2H), 2.23 (s, 3H), 1.89 (bm, 2H), 1.73 (bm, 2H), 1.20 (bm, 2H). (2×H obscured, by the solvent peak).

### EXAMPLE 57

### N-(3,5-Dimethylbenzyl)-3-(4-{[(2R)-2-hydroxy-2-(8-hydroxy-2-oxo-1,2-dihydroquinolin-5-yl)ethyl]amino}piperidin-1-yl)propanamide

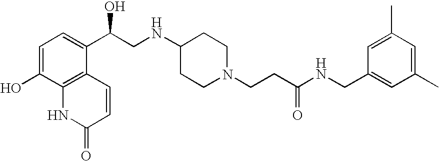

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.15 g), using HATU (0.22 g), triethylamine (0.11 mL) and (3,5-dimethyl)benzylamine (0.085 g) in dry DMF (3.0 mL) with stirring over 18 h according to the procedure described in Example 27. Purification was by reverse phase HPLC using an Xterra® C8 5 micron 19×50 mm column eluting with a gradient of 95% (0.2% aq.NH₃) in acetonitrile to 70% (0.2% aq.NH₃) in acetonitrile give the title compound as a cream solid. Yield: 12 mg

MS APCI+ 493 [M+H]⁺

¹H NMR (300 MHz, d₆-DMSO) δ 8.37 (t, 1H), 8.17 (d, 1H), 7.06 (d, 1H), 6.91 (d, 1H), 6.84 (s, 3H), 6.50 (d, 1H), 4.97 (t, 1H), 4.18 (d, 2H), 2.71 (bm, 4H), 2.39 (m, 1H), 2.23 (s+m, 8H), 1.91 (bm, 2H), 1.72 (bm, 2H), 1.19 (bm, 2H).

### EXAMPLE 58

### 3-(4-{[(2R)-2-hydroxy-2-(8-hydroxy-2-oxo-1,2-dihydroquinolin-5-yl)ethyl]amino}piperidin-1-yl)-N-(3-trifluoromethoxybenzyl)propanamide

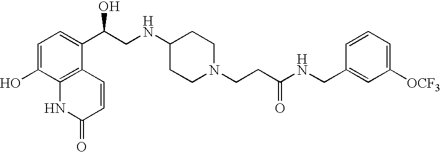

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.15 g), using HATU (0.22 g), triethylamine (0.11 mL) and 3-trifluoromethoxybenzylamine (0.12 g) in dry DMF (3.0 mL) with stirring over 18 h according to the procedure described in Example 27. Purification was by reverse phase HPLC using an Xterra® C8 5 micron 19×50 mm column eluting with a gradient of 95% (0.2% aq.NH₃) in acetonitrile to 70% (0.2% aq.NH₃) in acetonitrile give the title compound as a cream solid. Yield: 16 mg

MS APCI+ 549 [M+H]⁺

¹H NMR (300 MHz, d₆-DMSO) δ 8.49 (t, 1H), 8.17 (d, 1H), 7.44 (t, 1H), 7.31 (d, 1H), 7.22 (s, 2H), 7.06 (d, 1H), 6.90 (d, 1H), 6.49 (d, 1H), 4.96 (t, 1H), 4.31 (d, 2H), 2.71 (m, 4H), 2.29 (m, 2H), 1.92 (bm, 2H), 1.72 (bm, 2H), 1.21 (bt, 2H). (3×H obscured, by the solvent peak).

### EXAMPLE 59

### N-(3-Chloro-2-fluorobenzyl)-3-(4-{[(2R)-2-hydroxy-2-(8-hydroxy-2-oxo-1,2-dihydroquinolin-5-yl)ethyl]amino}piperidin-1-yl)propanamide

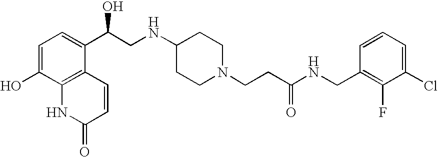

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.15 g), using HATU (0.22 g), triethylamine (0.11 mL) and (3-chloro-2-fluoro)benzylamine (0.10 g) in dry DMF (3.0 mL) with stirring over 18 h according to the procedure described in Example 27. Purification was by reverse phase HPLC using an Xterra® C8 5 micron 19×50 mm column eluting with a gradient of 95% (0.2% aq.NH₃) in acetonitrile to 70% (0.2% aq.NH₃) in acetonitrile give the title compound as a cream solid. Yield: 24 mg

MS APCI+ 517/519 [M+H]⁺

¹H NMR (300 MHz, d₆-DMSO) δ 8.49 (bm, 1H), 8.17 (d, 1H), 7.45 (t, 1H), 7.35 (t, 1H), 7.16 (t, 1H), 7.07 (d, 1H), 6.91 (d, 1H), 6.50 (d, 1H), 4.96 (t, 1H), 4.32 (d, 2H), 2.71 (m, 4H), 2.30 (m, 2H), 1.91 (bm, 2H), 1.76 (bt, 2H), 1.19 (bm, 2H). (3×H obscured, by the solvent peak).

### EXAMPLE 60

### N-[(2-Fluoro-5-trifluoromethyl)benzyl]-3-(4-{[(2R)-2-hydroxy-2-(8-hydroxy-2-oxo-1,2-dihydroquinolin-5-yl)ethyl]amino}piperidin-1-yl)propanamide

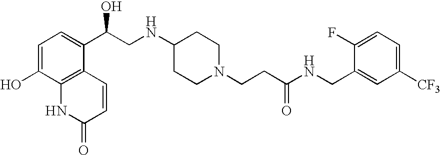

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.15 g), using HATU (0.22 g), triethylamine (0.11 mL) and (2-fluoro-5-trifluoromethyl)benzylamine (0.12 g) in dry DMF (3.0 mL) with stirring over 18 h according to the procedure described in Example 27. Purification was by reverse phase HPLC using an Xterra® C8 5 micron 19×50 mm column eluting with a gradient of 95% (0.2% aq.NH₃) in acetonitrile to 70% (0.2% aq.NH₃) in acetonitrile give the title compound as a white solid. Yield: 19 mg

MS APCI+ 551 [M+H]⁺

¹H NMR (300 MHz, d₆-DMSO) δ 8.54 (m, 1H), 8.17 (d, 1H), 7.66 (m, 2H), 7.42 (t, 1H), 7.06 (d, 1H), 6.90 (d, 1H), 6.50 (d, 1H), 4.96 (t, 1H), 2.70 (m, 4H), 2.31 (m, 2H), 1.91 (bm, 2H), 1.71 (bm, 2H), 1.18 (bm, 2H). (3×H obscured, by the solvent peak).

### EXAMPLE 61

### N-[(5-Chloro-2-fluoro)benzyl]-3-(4-{[(2R)-2-hydroxy-2-(8-hydroxy-2-oxo-1,2-dihydroquinolin-5-yl)ethyl]amino}piperidin-1-yl)propanamide

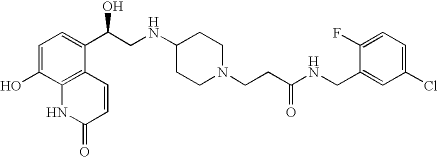

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.15 g), using HATU (0.22 g), triethylamine (0.11 mL) and (5-chloro-2-fluoro)benzylamine (0.10 g) in dry DMF (3.0 mL) with stirring over 18 h according to the procedure described in Example 27. Purification was by reverse phase HPLC using an Xterra® C8 5 micron 19×50 mm column eluting with a gradient of 95% (0.2% aq.NH₃) in acetonitrile to 70% (0.2% aq.NH₃) in acetonitrile give the title compound as a cream solid. Yield: 28 mg

MS APCI+ 517/519 [M+H]⁺

¹H NMR (300 MHz, d₆-DMSO) δ 8.46 (t, 1H), 8.17 (d, 1H), 7.33 (d, 2H), 7.22 (t, 1H), 7.07 (d, 1H), 6.92 (d, 1H), 6.50 (d, 1H), 4.97 (t, 1H), 4.28 (t, 2H), 2.72 (m, 1H), 2.41 (m, 1H), 2.29 (t, 2H), 1.91 (bt, 2H), 1.74 (bt, 2H), 1.24 (bm, 2H). (2×H obscured, by the solvent peak).

### EXAMPLE 62

### 3-(4-{[(2R)-2-Hydroxy-2-(8-hydroxy-2-oxo-1,2-dihydroquinolin-5-yl)ethyl]amino}piperidin-1-yl)-N-(3-trifluoromethyl)benzylpropanamide

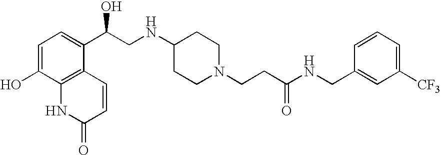

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.15 g), using HATU (0.22 g), triethylamine (0.11 mL) and (3-trifluoromethyl)benzylamine (0.11 g) in dry DMF (3.0 mL) with stirring over 18 h according to the procedure described in Example 27. Purification was by reverse phase HPLC using an Xterra® C8 5 micron 19×50 mm column eluting with a gradient of 95% (0.2% aq.NH₃) in acetonitrile to 70% (0.2% aq.NH₃) in acetonitrile give the title compound as a cream solid. Yield: 24 mg

MS APCI+ 533 [M+H]⁺

¹H NMR (300 MHz, d₆-DMSO) δ 8.51 (t, 1H), 8.18 (d, 1H), 7.56 (m, 4H), 7.07 (d, 1H), 6.92 (d, 1H), 6.50 (d, 1H), 4.98 (t, 1H), 4.35 (d, 2H), 2.72 (m, 4H), 2.41 (m, 1H), 2.29 (t, 2H), 1.91 (bm, 2H), 1.73 (bt, 2H), 1.21 (bm, 2H). (2×H obscured, by the solvent peak).

### EXAMPLE 63

### N-Benzhydryl-3-(4-{[(2R)-2-hydroxy-2-(8-hydroxy-2-oxo-1,2-dihydroquinolin-5-yl)ethyl]amino}piperidin-1-yl)propanamide

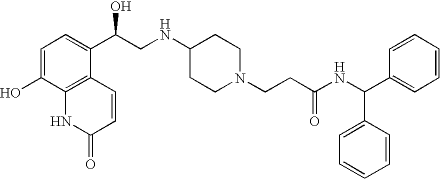

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.13 g), using HATU (0.20 g), triethylamine (0.07 mL) and benzhydrylamine (0.10 g) in dry DMF (3.0 mL) with stirring over 18 h according to the procedure described in Example 27. Purification was by reverse phase HPLC using an Xterra® C8 5 micron 19×50 mm column eluting with a gradient of 95% (0.2% aq.NH₃) in acetonitrile to 80% (0.2% aq.NH₃) in acetonitrile give the title compound as a yellow foam. Yield: 77 mg

MS APCI+ 541 [M+H]⁺

¹H NMR (300 MHz, d₆-DMSO) δ 8.97 (d, 1H), 8.18 (d, 1H), 7.27 (m, 10H), 7.07 (d, 1H), 6.91 (d, 1H), 6.50 (d, 1H), 6.08 (d, 1H), 4.98 (t, 1H), 4.35 (d, 2H), 2.72 (m, 4H), 2.41 (m, 1H), 2.29 (t, 2H), 1.91 (bm, 2H), 1.73 (bt, 2H), 1.21 (bm, 2H). (2×H obscured, by the solvent peak).

### EXAMPLE 64

### N,N-Dibenzyl-3-(4-{[(2R)-2-hydroxy-2-(8-hydroxy-2-oxo-1,2-dihydroquinolin-5-yl)ethyl]amino}piperidin-1-yl)propanamide

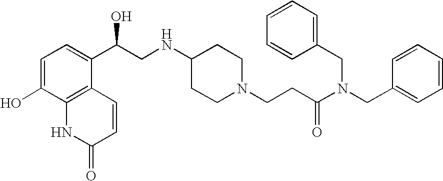

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.13 g), using HATU (0.20 g), triethylamine (0.07 mL) and dibenzylamine (0.11 g) in dry DMF (3.0 mL) with stirring over 18 h according to the procedure described in Example 27. Purification was by reverse phase HPLC using an Xterra® C8 5 micron 19×50 mm column eluting with a gradient of 95% (0.2% aq.NH₃) in acetonitrile to 50% (0.2% aq.NH₃) in acetonitrile give the title compound as a yellow gum. Yield: 70 mg

MS APCI+ 555 [M+H]⁺

¹H NMR (300 MHz, d₆-DMSO) δ 8.16 (d, 1H), 7.29 (m, 10H), 7.05 (d, 1H), 6.90 (d, 1H), 6.49 (d, 1H), 4.95 (t, 1H), 4.53 (s, 2H), 4.50 (s, 2H), 2.68 (d, 4H), 2.34 (m, 1H), 1.88 (m, 2H), 1.69 (bt, 2H), 1.16 (m, 2H). (4×H obscured, by the solvent peak).

### EXAMPLE 65

### N-[(3,5-Bistrifluoromethyl)benzyl]-3-(4-{[(2R)-2-hydroxy-2-(8-hydroxy-2-oxo-1,2-dihydroquinolin-5-yl)ethyl]amino}piperidin-1-yl)propanamide

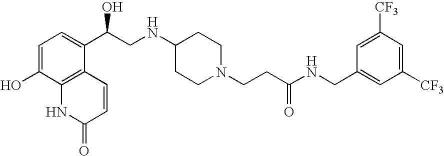

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.13 g), using HATU (0.20 g), triethylamine (0.07 mL) and (3,5-bistrifluoromethyl)benzylamine (0.13 g) in dry DMF (3.0 mL) with stirring over 18 h according to the procedure described in Example 27. Purification was by reverse phase HPLC using an Xterra® C8 5 micron 19×50 mm column eluting with a gradient of 95% (0.2% aq.NH₃) in acetonitrile to 50% (0.2% aq.NH₃) in acetonitrile give the title compound as a yellow foam. Yield: 65 mg

MS APCI+ 601 [M+H]⁺

¹H NMR (300 MHz, d₆-DMSO) δ 8.59 (t, 1H), 8.16 (d, 1H), 7.97 (s, 1H), 7.93 (s, 2H), 7.06 (d, 1H), 6.90 (d, 1H), 6.49 (d, 1H), 4.95 (t, 1H) 4.44 (d, 2H), 2.74 (m, 4H), 2.37 (m, 1H), 2.31 (t, 2H), 1.91 (m, 2H), 1.70 (bt, 2H), 1.16 (m, 2H). (2×H obscured, by the solvent peak).

### EXAMPLE 66

### N-[(Biphenyl-3-yl)methyl]-3-(4-{[(2R)-2-hydroxy-2-(8-hydroxy-2-oxo-1,2-dihydroquinolin-5-yl)ethyl]amino}piperidin-1-yl)propanamide

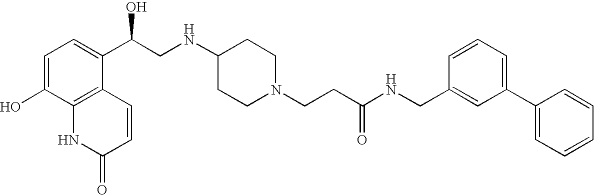

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.15 g), using HATU (0.23 g), triethylamine (0.11 mL) and (biphenyl-3-y)methylamine (0.12 g) in dry DMF (3.0 mL) with stirring over 18 h according to the procedure described in Example 27. Purification was by reverse phase HPLC using an Xterra® C8 5 micron 19×50 mm column eluting with a gradient of 95% (0.2% aq.NH₃) in acetonitrile to 50% (0.2% aq.NH₃) in acetonitrile give the title compound as a yellow solid. Yield: 40 mg

MS APCI+ 541 [M+H]⁺

¹H NMR (300 MHz, d₆-DMSO) δ 8.46 (t, 1H), 8.17 (d, 1H), 7.62 (d, 2H), 7.39 (m, 7H), 7.06 (d, 1H), 6.91 (d, 1H), 6.49 (d, 1H), 4.95 (t, 1H) 4.34 (d, 2H), 2.75 (m, 2H), 2.68 (d, 2H), 2.31 (m, 4H), 1.89 (m, 2H), 1.68 (bt, 2H), 1.15 (m, 2H). (1×H obscured, by the solvent peak).

### EXAMPLE 67

### 3-(4-{[(2R)-2-hydroxy-2-(8-hydroxy-2-oxo-1,2-dihydroquinolin-5-yl)ethyl]amino}piperidin-1-yl)-N-[(5,6,7,8-tetrahydronaphthalen-1-yl)methyl]propanamide

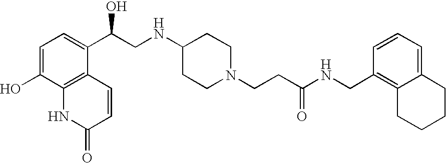

The title compound was prepared from the product of Example 6 step (iii) (3-[4-({[(2R)-2-{[tert-butyl(dimethyl)silyl]oxy}-2-(8-hydroxy-2-oxo-1,2-dihydroquinolin-5-yl)ethyl]amino}piperidin-1-yl]propanoic acid) (0.15 g), using HATU (0.23 g), triethylamine (0.11 mL) and (5,6,7,8-tetrahydronaphthalen-1-yl)methylamine (0.12 g) (Chem. Ber., 1922, 55 1705) in dry DMF (3.0 mL) with stirring over 18 h according to the procedure described in Example 27. Purification was by reverse phase HPLC using an Xterra® C8 5 micron 19×50 mm column eluting with a gradient of 95% (0.2% aq.NH₃) in acetonitrile to 50% (0.2% aq.NH₃) in acetonitrile give the title compound as a yellow foam. Yield: 45 mg

MS APCI+ 519 [M+H]⁺

¹H NMR (300 MHz, d₆-DMSO) δ 8.26 (m, 1H), 8.17 (d, 1H), 7.02 (m, 3H), 6.91 (m, 2H), 6.49 (m, 1H), 4.95 (t, 1H), 4.16 (d, 2H), 2.72 (m, 6H), 2.62 (t, 2H), 2.33 (m, 2H), 2.27 (t, 2H) 1.90 (bm, 2H), 1.72 (m, 6H), 1.14 (bm, 2H). (1×H obscured, by the solvent peak)

### EXAMPLE 68

### 5-((1R)-2-{[(1-{2-[2-(2,6-Dichlorophenyl)ethoxy]ethyl}-4-hydroxypiperidin-4-yl)methyl]amino}-1-hydroxyethyl)-8-hydroxyquinolin-2(1H)-one

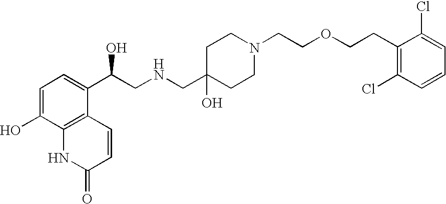

### i) [2-(2,6-Dichlorophenyl)ethoxy]acetic acid

2-(2,6-Dichlorophenyl)ethanol (4.70 g) was dissolved in DMF (53 mL) and cooled in a ice bath and then Sodium hydride (1.08 g, 60% in mineral oil) was added in portions. The reaction mixture was stirred at room temperature for 2 h then sodium chloroacetate (2.63 g) was added. The mixture was then heated to 60° C. for 6 h, then poured onto water and extracted with ether (×6). The aqueous material was acidified with conc. HCl from pH 9 to pH 2 then extracted with EtOAc (×7). The combined EtOAc extracts were washed with water, saturated aqueous NaCl, dried (Na₂SO₄) and concentrated to afford the subtitle compound as a pale yellow solid. Yield: 3.36 g

¹H NMR (300 MHz, CDCl₃) δ 7.30 (d, 2H), 7.12 (dd, 1H), 4.14 (s, 2H), 3.80 (t, 2H), 3.33 (t, 2H).

### ii) 1-{[2-(2,6-Dichlorophenyl)ethoxy]acetyl}piperidin-4-ol

The product from step (i) ([2-(2,6-dichlorophenyl)ethoxy]acetic acid) (2.06 g) was dissolved in DMF (35 mL) and to it were added HATU (3.92 g), triethylamine (2.9 mL) and piperidin-4-ol (3.47 g). The reaction mixture was stirred at room temperature overnight then DMF partially evaporated and the residue diluted with EtOAc. The mixture then washed with 2M aqueous HCl, saturated aqueous NaHCO₃, water, saturated aqueous NaCl, and the organic layer dried (Na₂SO₄) and evaporated. Purification was by Biotage, eluting with EtOAc to afford the subtitle compound as a pale yellow oil. Yield: 2.44 g

MS APCI+ 332/334/336 [M+H]⁺

### iii) 1-{2-[2-(2,6-Dichlorophenyl)ethoxy]ethyl}piperidin-4-ol

Lithium aluminium hydride (14 mL, 1M in THF) was added slowly to a cooled (0° C.) solution of the product from step (ii) (1-{[2-(2,6-dichlorophenyl)ethoxy]acetyl}piperidin-4-ol) (2.28 g) in dry THF (35 mL). The reaction mixture was allowed to warm to room temperature gradually and stirred for 2 days. It was cooled in ice then a mixture of Celite (5 g) and sodium sulphate decahydrate (5 g) added in portions. Additional THF was added, followed by 1 mL of 10% aqueous NaOH. The solids were removed by filtration and the volatiles evaporated. The residue was dissolved in EtOAc, dried (Na₂SO₄) and the volatiles evaporated to afford the subtitle compound as a yellow oil. Yield: 2.05 g

MS APCI+ 318/320/322 [M+H]⁺

### iv) 1-{2-[2-(2,6-Dichlorophenyl)ethoxy]ethyl}piperidin-4-one

The product from step (iii) (1-{2-[2-(2,6-dichlorophenyl)ethoxy]ethyl}piperidin-4-ol) (0.75 g) was dissolved in dry DCM (10 mL) and to it were added powdered 4 Å molecular sieves, TPAP (83 mg) and NMO (0.46 g). The reaction mixture was stirred for 1 h at room temperature. Ether (10 mL) was added and the mixture filtered. The filter pad was washed with EtOAc and the filtrate concentrated in vacuo. Purification was by Biotage column eluting with 1:3 hexane:EtOAc then EtOAc then 0.5% Et₃N in EtOAc to afford the subtitle compound as a colourless oil. Yield: 0.22 g

MS APCI+ 316/318/320 [M+H]⁺

### v) 6-{2-[2-(2,6-Dichlorophenyl)ethoxy]ethyl}-1-oxa-6-azaspiro[2.5]octane

Sodium hydride (54 mg, 60% in mineral oil) was added to dry DMSO (3.5 mL) and the mixture stirred for 15 min, then trimethylsulphoxonium iodide (0.17 g) added, and the mixture stirred for 2.25 h. A solution of the product from step (iv) (1-{2-[2-(2,6-dichlorophenyl)ethoxy]ethyl}piperidin-4-one) (0.22 g) in dry THF (3.5 mL) was added and the reaction mixture stirred overnight then diluted with EtOAc and water. The layers were separated and the aqueous layer extracted with further EtOAc (×3). The combined organic extracts were washed with water, saturated aqueous NaCl, dried (Na₂SO₄) and the volatiles evaporated to afford the subtitle compound as a yellow oil. Yield: 0.23 g

MS APCI+ 330/332/334 [M+H]⁺

### vi) 5-((1R)-1-{[tert-Butyl(dimethyl)silyl]oxy}-2-{[(1-{2-[2-(2,6-dichlorophenyl)ethoxy]ethyl}-4-hydroxypiperidin-4-yl)methyl]amino}ethyl)-8-hydroxyquinolin-2(1H)-one

A solution of the product from example 5 step (ii) (5-((1R)-2-amino-1-{[tert-butyl(dimethyl)silyl]oxy}ethyl)-8-hydroxyquinolin-2(1H)-one (0.27 g) and the product from step (v) 6-{2-[2-(2,6-dichlorophenyl)ethoxy]ethyl}-1-oxa-6-azaspiro[2.5]octane) (0.23 g) in methanol (5 mL) was heated at reflux for 20 h. The solvent was evaporated in vacuo and purification was by flash chromatography eluting with 0.7M NH₃ in MeOH (2.5 to 15%) in DCM to afford the subtitle compound as a yellow gum. Yield: 0.39 g

MS APCI+ 664/666/668 [M+H]⁺

### vii) 5-((1R)-2-{1-[(1-{2-[2-(2,6-Dichlorophenyl)ethoxy]ethyl}-4-hydroxypiperidin-4-yl)methyl]amino}-1-hydroxyethyl)-8-hydroxyquinolin-2(1H)-one

The product from step (vi) (5-((1R)-1-{[tert-butyl(dimethyl)silyl]oxy}-2-{[(1-{2-[2-(2,6-dichlorophenyl)ethoxy]ethyl}-4-hydroxypiperidin-4-yl)methyl]amino}ethyl)-8-hydroxyquinolin-2(1H)-one) (0.39 g) was dissolved in THF (9 mL) and to it was added triethylamine trihydrofluoride (0.3 mL). The reaction mixture was stirred overnight then the volatiles evaporated. Purification was by reverse phase HPLC using an XBridge® column eluting with a gradient of 5-75% acetonitrile in 0.2% aqueous TFA) to afford the titled compound ditrifluoroacetate salt as a yellow solid. Yield: 0.20 g

MS APCI+ 550/552/554 [M+H]⁺

¹H NMR (400 MHz, d₆-DMSO) δ 10.51 (s, 2H), 10.06 (s, 1H), 8.68 (s, 1H), 8.42 (s, 1H), 8.20 (d, 1H), 7.48 (d, 2H), 7.31 (t, 1H), 7.15 (d, 1H), 6.99 (d, 1H), 6.59 (d, 1H), 6.20 (s, 1H), 5.60 (s, 1H), 5.45-5.42 (m, 1H), 3.81-3.07 (m, 16H), 1.88-1.74 (m, 4H).

### EXAMPLE 69

### 8-Hydroxy-5-{(1R)-1-hydroxy-2-[({4-hydroxy-1-[2-(2-methyl-2-phenylpropoxy)ethyl]piperidin-4-yl}methyl)amino]ethyl}quinolin-2(1H)-one

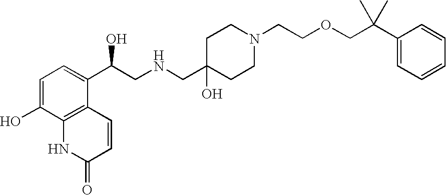

### i) (2-methyl-2-phenylpropoxy)acetic acid

The subtitle compound was prepared from 2-phenyl-2-methyl-propanol (4.05 g) by the method of Example 46 step (i) as a clear oil. Yield: 4.2 g

¹H NMR δ (CDCl₃) 1.38 (6H, s), 3.59 (2H, s), 4.03 (2H, s), 7.18-7.27 (2H, m), 7.19-7.24 (1H, m), 7.30-7.35 (2H, m), 7.38-7.42 (2H, m)

### ii) 1-(4-Hydroxy-piperidin-1-yl)-2-(2-methyl-2-phenyl-propoxy)-ethanone

The subtitle compound was prepared from the product of step (i) ((2-methyl-2-phenylpropoxy)acetic acid) (2.44 g) by the method of example 46 step (ii) as a clear oil.

Yield: 2.5 g

¹H NMR δ (CDCl₃) 7.40-7.35 (2H, m), 7.33-7.27 (2H, m), 7.22-7.16 (1H, m), 4.06 (2H, s), 4.03-3.94 (1H, m), 3.88-3.78 (1H, m), 3.64-3.54 (1H, m), 3.52 (2H, s), 3.12 (1H, ddd), 2.99 (1H, ddd), 1.89-1.79 (1H, m), 1.78-1.74 (1H, m), 1.72-1.62 (1H, m), 1.50-1.38 (1H, m), 1.34 (7H, s)

### iii) 1-[2-(2-Methyl-2-phenyl-propoxy)-ethyl]-piperidin-4-ol

The subtitle compound was prepared from the product of step (ii) (1-(4-hydroxy-piperidin-1-yl)-2-(2-methyl-2-phenyl-propoxy)-ethanone) (2.5 g) by the method of example 46 step (iii) as a clear gum. Yield: 1.76 g

¹H NMR δ (CDCl₃) 1.32 (6H, s), 1.59-1.47 (3H, m), 1.88-1.78 (2H, m), 2.18-2.09 (2H, m), 2.53 (2H, t), 2.77-2.68 (2H, m), 3.43 (2H, s), 3.50 (2H, t), 3.68-3.58 (1H, m), 7.18 (1H, t), 7.29 (2H, t), 7.38 (2H, d)

### iv) 1-[2-(2-Methyl-2-phenyl-propoxy)-ethyl]-piperidin-4-one

The subtitle compound was prepared from the product of step (iii) (1-[2-(2-methyl-2-phenyl-propoxy)-ethyl]-piperidin-4-ol) (0.9 g) by the method of example 46 step (iv) as a Clear gum. Yield: 0.57 g

¹H NMR δ (CDCl₃) 1.33 (6H, s), 2.37 (4H, t), 2.65 (2H, t), 2.71 (4H, t), 3.45 (2H, s), 3.54 (2H, t), 7.20-7.15 (1H, m), 7.32-7.26 (2H, m), 7.39-7.35 (2H, m)

### v) 8-Hydroxy-5-{(1R)-1-hydroxy-2-[({4-hydroxy-1-[2-(2-methyl-2-phenylpropoxy)ethyl]piperidin-4-yl}methyl)amino]ethyl}quinolin-2(1H)-one

The title compound was prepared from the product of step (iv) (1-[2-(2-Methyl-2-phenyl-propoxy)-ethyl]-piperidin-4-one) (150 mg) by the methods of example 46 step (v) and example 46 step (vi) without purification of the intermediates as a beige solid. Yield: 58 mg

MS: APCI+ 510 [M+H]⁺

¹H NMR δ (DMSO) 8.19 (1H, d), 7.38 (2H, d), 7.28 (2H, t), 7.16 (1H, t), 7.07 (1H, d), 6.91 (1H, d), 6.50 (1H, d), 5.06-5.01 (1H, m), 3.44 (2H, t), 3.41 (2H, s), 2.76-2.65 (2H, m), 2.47-2.39 (6H, m), 2.35-2.26 (2H, m), 1.49-1.37 (4H, m), 1.24 (6H, s)

### EXAMPLE 70

### 5-{(1R)-2-[({1-[2-(1,1-Dimethyl-2-phenylethoxy)ethyl]-4-hydroxypiperidin-4-yl}methyl)amino]-1-hydroxyethyl}-8-hydroxyquinolin-2(1H)-one

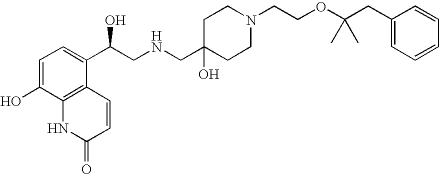

### i) (1,1-Dimethyl-2-phenyl-ethoxy)-acetic acid ethyl ester

Ethyl diazoacetate (9.13 g) was added slowly dropwise over 4 h to 2-methyl-1-phenyl-propan-2-ol (24.35 g) containing Rh(OAc)₂ dimer (0.7 mol %) at RT. The reaction was stirred at room temperature 16 h. Vacuum distillation (at bp 110-120° C./5 mm) afforded the subtitle compound as a clear liquid. Yield: 12.5 g

¹H NMR δ (CDCl₃) 1.15 (6H, s), 1.26 (3H, t), 2.80 (2H, s), 4.05 (2H, s), 4.19 (2H, q), 7.15-7.28 (5H, m)

### ii) (1,1-Dimethyl-2-phenyl-ethoxy)-acetic acid

The product of step (i) ((1,1-dimethyl-2-phenyl-ethoxy)-acetic acid ethyl ester) (11.82 g) in ethanol (100 mL) plus water (50 mL) and NaOH (4 g) were refluxed together for 6 h, then left at room temperature for 16 h. The ethanol was evaporated in vacuo and the aqueous residue was washed well with EtOAc. The aqueous solution was acidified (conc HCl) and extracted into EtOAc. Drying and evaporation gave an oil which was purified by silica gel column chromatography eluting with 9:1 isohexane-EtOAc to afford the subtitle compound as a clear liquid. Yield: 7 g

¹H NMR δ (CDCl₃) 1.22 (6H, s), 2.84 (2H, s), 4.07 (2H, s), 7.16-7.35 (5H, m), 9.22-9.65 (1H, m)

### iii) 2-(1,1-Dimethyl-2-phenyl-ethoxy)-1-(4-hydroxy-piperidin-1-yl)-ethanone

The product of step (ii) ((1,1-dimethyl-2-phenyl-ethoxy)-acetic acid) (3.124 g), 4-hydroxy-piperidine (4.55 g) and HATU (6.84 g) were dissolved in dry DMF (50 mL) and triethylamine (3.03 g) was added. The reaction was stirred at RT for 60 h The reaction was poured into water (600 mL) and acidified (conc HCl). The aqueous solution was ether extracted and the organic phase was washed with satd aq. sodium bicarbonate solution, dried and evaporated to afford the subtitle compound as a clear liquid. Yield: 1.6 g

¹H NMR δ (CDCl₃) 7.17-7.31 (5H, m), 4.11 (2H, s), 4.00-4.09 (1H, m), 3.79-3.95 (2H, m), 3.12-3.27 (2H, m), 2.81 (2H, s), 2.80 (1H, s), 1.74-1.94 (2H, m), 1.42-1.56 (2H, m), 1.19 (6H, s)

### iv) 1-[2-(1,1-Dimethyl-2-phenyl-ethoxy)-ethyl]-piperidin-4-ol

The subtitle compound was prepared from the product of step (iii) (2-(1,1-dimethyl-2-phenyl-ethoxy)-1-(4-hydroxy-piperidin-1-yl)-ethanone) (1.6 g) by the method of example 46 step (iii) as gum. Yield: 0.9 g

¹H NMR δ (CDCl₃) 7.32-7.16 (5H, m), 3.73-3.63 (1H, m), 3.56 (2H, t), 2.86-2.78 (2H, m), 2.78-2.75 (2H, m), 2.57 (2H, t), 2.27-2.17 (2H, m), 1.94-1.84 (2H, m), 1.72-1.66 (1H, m), 1.65-1.52 (2H, m), 1.14 (6H, s)

### v) 1-[2-(1,1-Dimethyl-2-phenyl-ethoxy)-ethyl]-piperidin-4-one

The subtitle compound was prepared from the product of step (iv) (1-[2-(1,1-dimethyl-2-phenyl-ethoxy)-ethyl]-piperidin-4-ol) (0.9 g) by the method of example 46 step (iv) as a gum. Yield: 0.58 g

¹H NMR δ (CDCl₃) 1.17 (6H, s), 2.45 (4H, t), 2.72 (2H, t), 2.79 (2H, s), 2.84 (4H, t), 3.61 (2H, t), 7.30-7.17 (5H, m)

### vi) 5-{(1R)-2-[({1-[2-(1,1-Dimethyl-2-phenylethoxy)ethyl]-4-hydroxypiperidin-4-yl}methyl)amino]-1-hydroxyethyl}-8-hydroxyquinolin-2(1H)-one

The title compound was prepared from the product of step (v) (1-[2-(1,1-dimethyl-2-phenyl-ethoxy)-ethyl]-piperidin-4-one) (220 mg) by the method of example 46 step (v) and example 46 step (vi) without purification of the intermediate as a beige solid. Yield: 58 mg

MS APCI+ 510 [M+H]⁺

¹H NMR δ (DMSO) 8.19 (1H, d), 7.29-7.14 (5H, m), 7.08 (1H, d), 6.92 (1H, d), 6.50 (1H, d), 5.06 (1H, t), 3.49 (2H, t), 2.77-2.67 (4H, m), 2.48-2.31 (4H, m), 1.53-1.41 (4H, m), 1.06 (6H, s)

### EXAMPLE 71

### 5-((1R)-2-{[(1-{2-[2-(2,3-Dichlorophenyl)ethoxy]ethyl}-4-hydroxypiperidin-4-yl)methyl]amino}-1-hydroxyethyl)-8-hydroxyquinolin-2(1H)-one

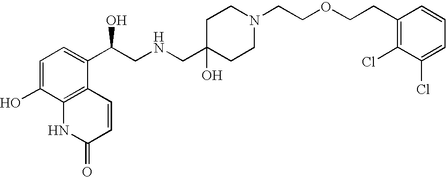

### i) [2-(2,3-Dichlorophenyl)ethoxy]acetic acid

2-(2,3-Dichlorophenyl)ethanol (5.03 g) was dissolved in DMF (53 mL) and cooled with a ice bath. Sodium hydride (1.15 g, 60% in mineral oil) was added in portions. The reaction mixture was stirred at room temperature for 1.75 h then sodium chloroacetate (2.80 g) was added. The mixture was then heated to 60° C. for 6 h, then poured onto water and extracted with ether (×6). The aqueous material was acidified with conc. HCl from pH 9 to pH 2 then extracted with EtOAc (×5). The combined EtOAc extracts were washed with water, saturated aqueous NaCl, dried (Na₂SO₄) and concentrated to afford the subtitle compound as a pale yellow oil. Yield: 1.57 g

¹H NMR (400 MHz, CDCl₃) δ 7.35 (dd, 1H), 7.24 (dd, 1H), 7.14 (t, 1H), 4.11 (s, 2H), 3.82 (t, 2H), 3.13 (t, 2H).

### ii) 1-{[2-(2,3-Dichlorophenyl)ethoxy]acetyl}piperidin-4-ol

The product from step (i) ([2-(2,3-dichlorophenyl)ethoxy]acetic acid) (2.41 g) was dissolved in DMF (45 mL) and to it were added HATU (4.42 g), triethylamine (4.1 mL) and piperidin-4-ol (2.94 g). The reaction mixture was stirred at room temperature for 28 h then the DMF partially evaporated and the residue diluted with EtOAc. It was washed with 2M aqueous HCl, saturated aqueous NaHCO₃, water, saturated aqueous NaCl, The organic layer collected, dried (Na₂SO₄) and the volatiles evaporated to afford the subtitle compound as a yellow oil. Yield: 2.75 g

MS APCI+ 332/334/336 [M+H]⁺

### iii) 1-{2-[2-(2,3-Dichlorophenyl)ethoxy]ethyl}piperidin-4-ol

Lithium aluminium hydride (16 mL, 1M in THF) was added slowly to a cooled (0° C.) solution of the product from step (ii) (1-{[2-(2,3-dichlorophenyl)ethoxy]acetyl}piperidin-4-ol) (2.59 g) in dry THF (40 mL). The reaction mixture was allowed to warm to room temperature gradually and stirred for 3 h. It was cooled in ice then a mixture of Celite (5 g) and sodium sulphate decahydrate (5 g) added in portions. Additional THF was added, followed by 1 mL of 10% aqueous NaOH. The solids were removed by filtration and the volatiles evaporated to afford the subtitle compound as a yellow oil. Yield: 1.98 g

MS APCI+ 318/320/322 [M+H]⁺

### iv) 1-{2-[2-(2,3-Dichlorophenyl)ethoxy]ethyl}piperidin-4-one

The product from step (iii) (1-{2-[2-(2,3-dichlorophenyl)ethoxy]ethyl}piperidin-4-ol) (0.39 g) was dissolved in DCM (8 mL) and to it were added NMO (0.18 g) and powdered 4 Å molecular sieves (0.5 g). The reaction mixture was stirred for 30 min then TPAP (83 mg) was added. The reaction mixture was stirred for 30 min at room temperature then further NMO (0.18 g), TPAP (32 mg) and molecular sieves (0.5 g) were added. After a further 40 min, ether was added and the mixture filtered. The filter pad was washed with EtOAc and the filtrate concentrated. Purification was by flash chromatography, eluting with 10% MeOH in DCM, to afford the subtitle compound as a black oil. Yield: 0.23 g

MS APCI+ 316/318/320 [M+H]⁺

### v) 6-{2-[2-(2,3-Dichlorophenyl)ethoxy]ethyl}-1-oxa-6-azaspiro[2.5]octane

Sodium hydride (80 mg, 60% in mineral oil) was added to dry DMSO (4 mL), followed by trimethylsulphoxonium iodide (0.24 g), and the mixture stirred for 2 h. A solution of the product from step (iv) (1-{2-[2-(2,3-dichlorophenyl)ethoxy]ethyl}piperidin-4-one) (0.23 g) in dry THF (5 mL) was added and the reaction mixture stirred for 3 h then diluted with EtOAc and water. The layers were separated and the aqueous layer extracted with further EtOAc (×3). The combined organic extracts were washed with water, saturated aqueous NaCl, dried (MgSO₄) and the volatiles evaporated to afford the subtitle compound as a colourless oil. Yield: 0.25 g

MS APCI+ 330/332/334 [M+H]⁺

### vi) 5-((1R)-1-{[tert-Butyl(dimethyl)silyl]oxy}-2-{[(1-{2-[2-(2,3-dichlorophenyl)ethoxy]ethyl}-4-hydroxypiperidin-4-yl)methyl]amino}ethyl)-8-hydroxyquinolin-2 (1H)-one

A solution of the product from Example 5 step (ii) (5-((1R)-2-amino-1-{[tert-butyl(dimethyl)silyl]oxy}ethyl)-8-hydroxyquinolin-2(1H)-one) (0.30 g) and the product from step (v) (6-{2-[2-(2,3-dichlorophenyl)ethoxy]ethyl}-1-oxa-6-azaspiro[2.5]octane) (0.24 g) in methanol (5 mL) was heated at reflux for 20 h. The solvent was evaporated and purification was by flash chromatography eluting with 1.4M NH₃ in MeOH (2 to 25%) in DCM to afford the product as a yellow foam. Yield: 0.26 g

MS APCI+ 664/666/668 [M+H]⁺

### vii) 5-((1R)-2-{[(1-{2-[2-(2,3-Dichlorophenyl)ethoxy]ethyl}-4-hydroxypiperidin-4-yl)methyl]amino)}-1-hydroxyethyl)-8-hydroxyquinolin-2(1H)-one

The product from step (vi) (5-((1R)-1-{[tert-butyl(dimethyl)silyl]oxy}-2-{[(1-{2-[2-(2,3-dichlorophenyl)ethoxy]ethyl}-4-hydroxypiperidin-4-yl)methyl]amino}ethyl)-8-hydroxyquinolin-2(1H)-one) (0.26 g) was dissolved in THF (9 mL) and to it was added triethylamine trihydrofluoride (0.4 mL). The reaction mixture was stirred at room temperature for 3 d then the volatiles evaporated. Purification was by reverse phase HPLC using an Ace® column eluting with a gradient of 5-50% acetonitrile in 0.2% aqueous TFA) to afford the titled compound ditrifluoroacetate salt as a cream solid. Yield: 90 mg

MS APCI+ 550/552/554 [M+H]⁺

¹H NMR (400 MHz, d₆-DMSO) δ 10.58 (s, 1H), 10.50 (s, 1H), 9.65 (s, 1H), 8.78 (s, 1H), 8.45 (s, 1H), 8.22 (d, 1H), 7.52 (d, 1H), 7.40 (d, 1H), 7.31 (t, 1H), 7.16 (d, 1H), 7.00 (d, 1H), 6.58 (d, 1H), 5.45-5.43 (m, 1H), 3.79-3.76 (m, 2H), 3.71 (t, 2H), 3.50-3.10 (m, 12H), 3.05 (t, 2H), 1.88-1.78 (m, 4H)

### EXAMPLE 72

### 5-{(1R)-2-[({1-[2-(1,1-Dimethyl-2-phenylethoxy)ethyl]piperidin-4-yl}methyl)amino]-1-hydroxyethyl}-8-hydroxyquinolin-2(1H)-one

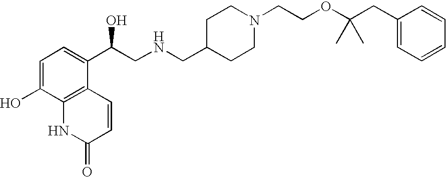

### i) 1-[2-(1,1-Dimethyl-2-phenyl-ethoxy)-ethyl]-piperidine-4-carbaldehyde

Trimethylsilyl-diazomethane (179 mg) was added to a solution of lithium diisopropylamide (prepared from n-BuLi (0.627 mL, 2.5M in hexanes) and diisopropylamine (1.315 g) in THF (3 mL)) at −78° C., and stirred for 30 min at −78° C. The product of Example 70 step (v) (1-[2-(1,1-dimethyl-2-phenyl-ethoxy)-ethyl]-piperidin-4-one) (360 mg) in dry THF (3 mL) was added dropwise and the reaction was further stirred for 1 h at −78° C. then refluxed for 3 h. After quenching with ice cold water the reaction was extracted into ether, the ether extract was dried (Na₂SO₄) and evaporated, affording an orange oil. The was dissolved in EtOAc (25 mL) containing silica gel (4 g) and was stirred for 16 h under nitrogen. The silica gel was filtered off and further washed with EtOAc, and the combined organic solutions were evaporated affording an orange oil. Yield: 230 mg

¹H NMR δ (CDCl₃) 9.64 (1H, s), 7.29-7.23 (2H, m), 7.23-7.17 (3H, m), 3.56 (2H, t), 2.92-2.84 (2H, m), 2.77 (2H, s), 2.57 (2H, t), 2.27-2.14 (3H, m), 1.93-1.84 (2H, m), 1.74-1.62 (2H, m), 1.14 (6H, s)

### ii) 5-{(1R)-2-[({1-[2-(1,1-Dimethyl-2-phenylethoxy)ethyl]piperidin-4-yl}methyl)amino]-1-hydroxyethyl}-8-hydroxyquinolin-2(1H)-one

The product of step (i) (1-[2-(1,1-dimethyl-2-phenyl-ethoxy)-ethyl]-piperidine-4-carbaldehyde) (986 mg) and the product of example 5 step (ii) (5-((1R)-2-amino-1-{[tert-butyl(dimethyl)silyl]oxy}ethyl)-8-hydroxyquinolin-2(1H)-one) (100 mg) were dissolved in MeOH (3.2 mL) and treated with water (36 uL) and AcOH (36 mg) and then sodium cyanoborohydride (19 mg) was added. The reaction was stirred at room temperature for 16 h. The solvent was evaporated and the residue was partitioned between water and EtOAc. Evaporation of the organic solution gave a pink solid which was dissolved in dry THF (3 mL). The solution was treated with triethylamine trihydrofluoride (161 mg) at room temperature and stirred for 16 h. The reaction mixture was diluted with toluene and evaporated to dryness. Purification was by reverse phase HPLC using an Xterra® C8 5 micron 19×50 mm column eluting with a gradient of 95% (0.2% aq.NH₃) in acetonitrile to 50% (0.2% aq.NH₃) in acetonitrile give the title compound as a gum. Yield: 62 mg

MS: APCI+ 494 [M+H]⁺

¹H NMR δ (DMSO) 8.18 (1H, d), 7.14-7.29 (6H, m), 7.06 (1H, d), 6.92 (1H, d), 6.50 (1H, d), 5.04 (1H, dd), 3.47 (2H, t), 2.85 (2H, d), 2.63-2.76 (6H, m), 2.36-2.47 (4H, m), 1.89-1.96 (2H, m), 1.87 (6H, s), 1.57-1.67 (2H, m), 1.29-1.42 (1H, m), 1.01-1.15 (9H, m)

### EXAMPLE 73

### 8-Hydroxy-5-{(1R)-1-hydroxy-2-[({1-[2-(2-methyl-2-phenylpropoxy)ethyl]piperidin-4-yl}methyl)amino]ethyl}quinolin-2(1H)-one

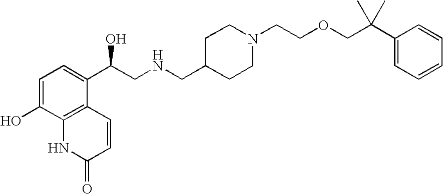

### i) 1-[2-(2-Methyl-2-phenyl-propoxy)-ethyl]-piperidine-4-carbaldehyde

The subtitle compound was prepared from the product of Example 69 step (iv) (1-[2-(2-methyl-2-phenyl-propoxy)-ethyl]-piperidin-4-one) (360 mg) by the method of Example 72 step (i) as an oil. Yield: 230 mg

¹H NMR δ (CDCl₃) 9.62 (1H, s), 7.38 (2H, d), 7.29 (2H, t), 7.18 (1H, t), 3.50 (2H, t), 3.42 (2H, s), 2.82-2.75 (2H, m), 2.53 (2H, t), 2.22-2.06 (3H, m), 1.87-1.78 (2H, m), 1.68-1.56 (2H, m), 1.32 (6H, s)

### ii) 8-Hydroxy-5-{(1R)-1-hydroxy-2-[({1-[2-(2-methyl-2-phenylpropoxy)ethyl]piperidin-4-yl}methyl)amino]ethyl}quinolin-2(1H)-one

The title compound was prepared from the product of step (i) (1-[2-(2-Methyl-2-phenyl-propoxy)-ethyl]-piperidine-4-carbaldehyde) (85.6 mg) by the method of Example 72 step (ii) as a glass. Yield: 44 mg

MS: APCI+ 494 [M+H]⁺

¹H NMR δ (DMSO) 8.17 (1H, d), 7.37 (2H, d), 7.27 (2H, t), 7.16 (1H, t), 7.06 (1H, d), 6.92 (1H, d), 6.49 (1H, d), 5.04 (1H, dd), 3.43 (2H, t), 3.40 (2H, s), 2.63-2.82 (4H, m), 2.36-2.47 (4H, m), 1.78-1.93 (1H, m), 1.53-1.62 (2H, m), 1.27-1.37 (1H, m), 1.24 (6H, s), 0.99-1.11 (2H, m)

### EXAMPLE 74

### 8-Hydroxy-5-{(2R)-1-hydroxy-2-[(4-hydroxy-1-{2-[2-(5,6,7,8-tetrahydronaphthalen-1-yl)ethoxy]ethyl}piperidin-4-ylmethyl)amino]ethyl}-1H-quinolin-2-one

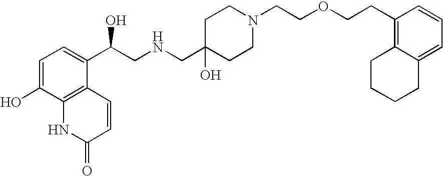

### i) 2-(5,6,7,8-Tetrahydronaphthalen-1-yl)ethanol

Platinum oxide (0.1 g) was added to a solution of 2-(naphth-1-yl)ethanol (1.0 g) in acetic acid. The reaction mixture was hydrogenated at 2 bar pressure for 2 d. An aliquot of platinum oxide (0.10 g) was added and the mixture further hydrogenated at 5 bar pressure for 18 h then filtered through Celite. The filtrate was partitioned between water and ethyl acetate. The organic layer was washed with saturated aqueous sodium bicarbonate, saturated sodium chloride, dried and the solvent evaporated in vacuo. The residue was purified by silica gel chromatography eluting with 2:8 ethyl acetate: isohexane to afford the subtitle compound as a clear oil. Yield: 0.7 g

¹H NMR (CDCl₃) δ 7.05 (m, 3H), 3.82 (m, 3H), 2.87 (t, 2H), 2.76 (m, 4H), 1.78 (m, 4H), 1.40 (t, 1H).

### ii) [2-(5,6,7,8-Tetrahydronaphthalen-1-yl)ethoxy]acetic acid

The subtitle compound was prepared from the product of step (i) ([2-(5,6,7,8-tetrahydronaphthalen-1-yl)ethoxy]acetic acid) (0.65 g), using sodium hydride (0.15 g, 60% in mineral oil) in anhydrous DMF (4.7 mL) according to the procedure described in Example 38, step (i) to afford a pale yellow oil. Yield: 0.74 g

¹H NMR (CDCl₃) δ 7.06 (m, 1H), 6.98 (m, 2H), 4.13 (s, 2H), 3.76 (t, 2H), 2.93 (t, 2H), 2.78 (t, 2H), 2.72 (t, 2H), 1.80 (m, 4H).

### iii) 1-(4-hydroxypiperidin-1-yl)-2-[2-(5,6,7,8-tetrahydronaphthalen-1-yl)ethoxy]ethanone

To a solution of the product of step (ii) ([2-(5,6,7,8-tetrahydronaphthalen-1-yl)ethoxy]acetic acid) (0.74 g) in DCM (126 mL) was added thionyl chloride (0.63 mL) followed by 3 drops of DMF and the whole stirred at room temperature for 2 h. The solution was azeotroped twice from toluene. DCM (16 mL) was added to the residue and this suspension then added dropwise to a solution of 4-piperidinol (0.64 g) in triethylamine (0.44 mL) in DCM (16 mL). The mixture was stirred for a further 2 days. The mixture was partioned between EtOAc and water. The organic phase was washed with 2M HCl, saturated sodium bicarbonate solution, then saturated aqueous NaCl. The organic layer was collected, dried (MgSO₄) and the solvent evaporated to leave a colourless gum. Purification was by silica gel chromatography eluting with EtOAc/isohexane (8:2 to 10:0 to give the subtitle compound as a clear oil. Yield: 0.60 g

MS APCI+ 318 [M+H]⁺

### iv) 1-{2-[2-(5,6,7,8-Tetrahydronaphthalen-1-yl)ethoxy]ethyl}piperidin-4-ol

The subtitle compound was prepared from the product of step (iii) (1-(4-hydroxypiperidin-1-yl)-2-[2-(5,6,7,8-tetrahydronaphthalen-1-yl)ethoxy]ethanone) (0.62 g), using lithium aluminium hydride (3.8 mL, 1M solution in THF) in anhydrous THF (5.7 mL) according to the procedure described in Example 45, step (ii) as a gum. Yield: 0.57 g

MS APCI+ 304 [M+H]⁺

### v) 1-{2-[2-(5,6,7,8-Tetrahydronaphthalen-1-yl)ethoxy]ethyl}piperidin-4-one hydrate

The subtitle compound was prepared from the product of step (iv) (1-{2-[2-(5,6,7,8-tetrahydronaphthalen-1-yl)ethoxy]ethyl}piperidin-4-ol) (0.62 g), using 4 A molecular sieves (0.26 g), NMO (0.13 g) and TPAP (0.023 g) in DCM (4.7 mL) and further quantities of 4 A molecular sieves (0.13 g), NMO (0.06 g) and TPAP (0.012 g) were added after 4 h according to the procedure described in Example 45, step (iii) as a clear oil. Yield: 0.526 g

MS APCI+ 302 [M+H]⁺ and 320 [M+18+H]⁺

### vi) 6-{2-[2-(5,6,7,8-Tetrahydronaphthalen-1-yl)ethoxy]ethyl}-1-oxa-6-azaspiro[2.5]octane

The subtitle compound was prepared according to the procedure outlined in Example 5 step (iii) using a solution of the product from step (v) (1-{2-[2-(5,6,7,8-tetrahydronaphthalen-1-yl)ethoxy]ethyl}piperidin-4-one hydrate) (0.26 g), trimethylsulphoxonium iodide (0.28 g), sodium hydride, (0.06 g, 60% in mineral oil) and dry DMSO (2.75 mL) as a yellow oil. Yield: 0.26 g

MS APCI+ 316 [M+H]⁺

### vii) 8-Hydroxy-5-{(2R)-1-hydroxy-2-[(4-hydroxy-1-{2-[2-(5,6,7,8-tetrahydronaphthalen-1-yl)ethoxy]ethyl}piperidin-4-ylmethyl)amino]ethyl}-1H-quinolin-2-one

A solution of the product of step (vi) (6-{2-[2-(5,6,7,8-tetrahydronaphthalen-1-yl)ethoxy]ethyl}-1-oxa-6-azaspiro[2.5]octane) (0.12 g) and the product of Example 5 step (ii) (0.13 g) in methanol (0.32 mL) was heated under reflux for 4 h and the solvent evaporated under reduced pressure. A solution of the residue in THF (10 mL) was treated with triethylamine trihydroflouride (0.35 mL). After stirring for 18 h at ambient temperature the volatiles were evaporated in vacuo and the residue azeotroped with toluene (×2). The residue was purified by reverse phase using an Xterra® C8 5 micron 19×50 mm column eluting with a gradient of 95% (0.2% aq.NH₃) in acetonitrile to 50% (0.2% aq.NH₃) in acetonitrile give the title compound as a yellow foam. Yield: 56 mg

MS APCI+ 536 [M+H]⁺

¹H NMR (300 MHz, d6-DMSO) δ 8.19 (d, 1H), 7.07 (d, 1H), 6.98 (m, 2H), 6.90 (m, 2H), 6.49 (d, 1H), 5.01 (m, 1H), 3.52 (t, 2H), 3.47 (t, 2H), 2.70 (m, 8H), 2.43 (m, 6H), 2.31 (m, 2H), 1.71 (m, 4H), 1.43 (bm, 4H)

### EXAMPLE 75

### 8-Hydroxy-5-{(1R)-1-hydroxy-2-[({(2S)-1-[3-(2-phenylethoxy)propyl]pyrrolidin-2-yl}methyl)amino]ethyl}quinolin-2(1H)-one

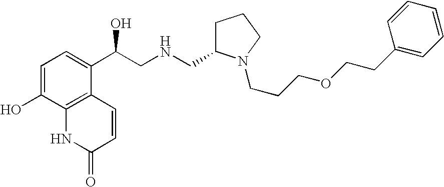

### i) 1-[3-(2-Phenylethoxy)propanoyl]-L-prolinamide

A mixture of L-prolinamide (2.0 g) in DCM (200 mL) and Hunig's base (0.40 mL), and 3-(phenylethoxy)propanoic acid (3.4 g), was treated with HATU (0.84 g). After stirring for 4 h at room temperature, the mixture was poured into water and the organic layer washed with water (×3). The organic layer was collected, dried (Na₂SO₄), and evaporated. This residue was purified by silica gel chromatography eluting initially with ethyl acetate and then obtaining the pure product by ethyl acetate/methanol (4:1) to give the subtitle compound as an oil. Yield: 3.7 g

MS APCI+ 291 [M+H]⁺

### ii) 1-{(2S)-1-[3-(2-Phenylethoxy)propyl]pyrrolidin-2-yl}methanamine

A solution of the product of step (i) (1-[3-(2-phenylethoxy)propanoyl]-L-prolinamide) (3.5 g) in THF (30 mL) was treated portionwise with stirring borane-THF complex (140 ml, 11.0M in THF). The mixture was heated to reflux for 12 h. The solvents were removed by reduced pressure and the residue was treated with dropwise addition of methanol followed by addition of 12M HCl (4 mL). The mixture was heated to reflux for 2 h and then concentrated in vacuo to leave a viscous oil. This was dissolved in acetonitrile/isopropanol mixture (1:1) and loaded onto a 70 g SCX cartridge. After elution of this mixture, the product was eluted with acetonitrile/isopropanol/0.880 ammonia (2:2:1) to give, after evaporation of the solvent, the subtitle compound as a colourless oil.

Yield: 2.1 g

¹H NMR (DMSO) δ 7.18-7.29 (m, 5H), 3.55 (t, 2H), 3.40 (t, 2H), 3.00 (t, 1H), 2.77 (t, 2H), 2.70-2.73 (m, 1H), 2.48-2.53 (m, 2H), 2.25-2.37 (m, 1H), 2.06-2.12 (m, 2H), 1.74 (m, 1H), 1.56-1.66 (m, 5H)

### iii) 8-(Benzyloxy)-5-{(1R)-1-{[tert-butyl(dimethyl)silyl]oxy}-2-[({(2S)-1-[3-(2-phenylethoxy)propyl]pyrrolidin-2-yl}methyl)amino]ethyl}quinolin-2(1H)-one

A solution of the product of example 2 step (iii) (8-(benzyloxy)-5-((1R)-2-bromo-1-{[tert-butyl(dimethyl)silyl]oxy}ethyl)quinolin-2(1H)-one) (310 mg) potassium iodide (210 mg) and the product of step (ii) (1-{(2S)-1-[3-(2-phenylethoxy)propyl]pyrrolidin-2-yl}methanamine) (281 mg) in dry DMSO (3 mL) were heated at 70° C. under nitrogen for 12 h. The mixture diluted with water and extracted with EtOAc (×2) and the combined organics shaken with water, dried (sodium sulfate) to give a brown gum. Purification was by silica gel chromatography eluting with 3-5% methanol in DCM to give the subtitle compound as a orange gum. Yield: 260 mg

MS APCI+ 670 [M+H]⁺

### iv) 8-(Benzyloxy)-5-{(1R)-1-hydroxy-2-[({(2S)-1-[3-(2-phenylethoxy)propyl]pyrrolidin-2-yl}methyl)amino]ethyl}quinolin-2(1H)-one

A solution of the product of step (iii) (8-(benzyloxy)-5-{(1R)-1-{[tert-butyl(dimethyl)silyl]oxy}-2-[({(2S)-1-[3-(2-phenylethoxy)propyl]pyrrolidin-2-yl}methyl)amino]ethyl}quinolin-2(1H)-one) (310 mg) in THF (6 mL) was treated with triethylamine trihydrofluoride (0.6 mL) and the mixture stirred overnight. The volatiles were evaporated under reduced pressure and the residue azeotroped with toluene. Purification was by reverse phase HPLC using an Xterra® C8 5 micron 19×50 mm column eluting with a gradient of 95% 0.880 NH₃ in acetonitrile to 5% 0.880 NH₃ in acetonitrile to give the subtitle compound as beige gum. Yield: 86 mg

MS APCI+ 555 [M+H]⁺

### v) 8-Hydroxy-5-{(1R)-1-hydroxy-2-[({(2S)-1-[3-(2-phenylethoxy)propyl]pyrrolidin-2-yl}methyl)amino]ethyl}quinolin-2(1H)-one

A mixture of the product of step (iv) (8-(benzyloxy)-5-f{(R)-1-hydroxy-2-[({(2S)-1-[3-(2-phenylethoxy)propyl]pyrrolidin-2-yl}methyl)amino]ethyl}quinolin-2(1H)-one) (75 mg), 2M HCl (2 drops), 10% palladium on charcoal (10 mg) in EtOH (3 mL) was hydrogenated at 5 bar for 6 days. Filtration of the catalyst and evaporation of the solvent under reduced pressure gave a brown gum which was purified by reverse phase HPLC using an Xterra® C8 5 micron 19×50 mm column eluting with a gradient of 95% (0.2% aq.NH₃) in acetonitrile to 50% (0.2% aq.NH₃) in acetonitrile give the title compound as a glass. Yield: 19 mg

MS APCI+ 466 [M+H]⁺

¹H NMR (DMSO) δ 8.20 (d, 1H), 7.14-7.27 (m, 5H), 7.06 (d, 1H), 6.93 (d, 1H), 6.47 (d, 1H), 5.08 (t, 1H), 3.53 (t, 2H), 3.36 (t, 2H)

### EXAMPLE 76

### N-[2-(4-{[(2R)-2-hydroxy-2-(8-hydroxy-2-oxo-1,2-dihydroquinolin-5-yl)ethyl]amino}piperidin-1-yl)ethyl]benzenesulfonamide

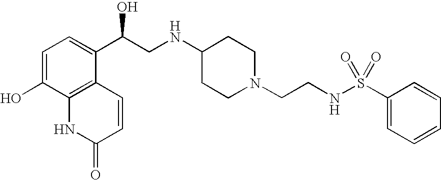

### i) (4-hydroxypiperidin-1-yl)acetonitrile

Bromoacetonitrile (5.7 g) was added dropwise to a solution of 4-hydroxypiperidine (5.0 g) in dry THF (20 mL) while the temperature was maintained between 45-50° C. Thereafter, the mixture was heated to reflux for 30 min before allowing to cool to room temperature. The solvents evaporated in vacuo and the residue purified by silica gel chromatography eluting with 19:1 then 10:1 ether/methanol to give the subtitle compound as a white solid.

Yield: 3.2 g

¹H NMR (DMSO) δ 4.59 (d, 1H), 3.66 (s, 2H), 3.66-3.32 (m, 1H), 2.71-2.64 (m, 2H), 2.26-2.18 (t, 2H), 1.77-1.70 (m, 2H), 1.47-1.35 (m, 2H)

### ii) 1-(2-Aminoethyl)piperidin-4-ol

Lithium aluminium hydride in THF (43 ml, 1.0M in THF) was diluted in dry THF (13 mL) and cooled to 0° C. under nitrogen. The product from step (i) ((4-hydroxypiperidin-1-yl)acetonitrile) (2.0 g) in THF (5 mL) was added slowly via syringe. The reaction mixture was heated at reflux for 5 h then cooled to room temperature. Excess hydride was destroyed by dropwise addition of 1.6 ml of water and 1.6 ml of 15% NaOH, and finally EtOAc was added dropwise until no effervescence was observed. The granular precipitate formed was filtered and washed several times with DCM and EtOAc. The organic layer was dried (MgSO4) and the solvent was evaporated in vacuo to give the subtitle compound as a yellow oil. Yield: 2.0 g

This was used in the next step without purification.

¹H NMR (DMSO) δ 3.44-3.37 (m, 1H), 2.64 (t, 2H), 2.57 (t, 2H), 2.25-2.21 (t, 2H), 1.99-1.94 (m, 2H), 1.70-1.65 (m, 2H), 1.40-1.31 (m, 2H).

### iii) N-[2-(4-hydroxypiperidin-1-yl)ethyl]benzenesulfonamide

The product from step (ii) (1-(2-aminoethyl)piperidin-4-ol) (0.43 g) was suspended in DCM (40 mL) followed by addition of triethylamine (0.84 ml) and benzenesulfonyl chloride (0.53 g). The mixture was stirred for 4 h at room temperature and then partitioned between DCM and water, organics collected, dried (Na₂SO₄) and solvent evaporated to a leave beige solid. Purification was by silica gel chromatography eluting with 20:1 to 10:1 DCM/MeOH to give the subtitle compound as a colourless solid. Yield: 0.43 g

MS APCI+ 285 [M+H]⁺

¹H NMR (DMSO) δ 7.82-7.79 (m, 2H), 7.66-7.57 (m, 3H), 7.49 (s, 1H), 4.49 (s, 1H), 3.45-3.37 (m, 1H), 2.80 (t, 2H), 2.26 (t, 2H), 1.93 (t, 2H), 1.63-1.60 (m, 2H), 1.34-1.25 (m, 2H).

### iv) N-[2-(4-Oxopiperidin-1-yl)ethyl]benzenesulfonamide

The product from step (iii) (N-[2-(4-Hydroxypiperidin-1-yl)ethyl]benzenesulfonamide) (0.50 g) was dissolved in DCM (200 mL) and pyridinium dichromate (3.9 g) added portionwise. The mixture was stirred at room temperature overnight. The mixture was partitioned between DCM and water with the aqueous layer basified to pH8 with aqueous sodium bicarbonate and extracted with DCM (×3). The organics collected, dried (MgSO₄) and evaporated to give the subtitle compound as a yellow oil. Yield: 0.21 g

MS APCI+ 283 [M+H]⁺

¹H NMR (DMSO) δ 7.85-7.79 (m, 2H), 7.66-7.57 (m, 3H), 2.94 (q, 2H), 2.59 (t, 4H), 2.43 (t, 2H), 2.26 (t, 4H).

### v) N-[2-(4-{[(2R)-2-{[tert-butyl(dimethyl)silyl]oxy}-2-(8-hydroxy-2-oxo-1,2-dihydroquinolin-5-yl)ethyl]amino}piperidin-1-yl)ethyl]benzenesulfonamide

The product from example 5 step (ii) (5-((1R)-2-Amino-1-{[tert-butyl(dimethyl)silyl]oxy}ethyl)-8-hydroxyquinolin-2(1H)-one) (140 mg), acetic acid (0.027 mL), activated molecular sieves (8 beads) and the product from step (iv) (N-[2-(4-oxopiperidin-1-yl)ethyl]benzenesulfonamide) (180 mg) in NMP (3 mL) were stirred at room temperature overnight. Sodium triacetoxyborohydride (133 mg) was added, and stirring continued for 2 h. The reaction mixture was loaded onto SCX resin cartridge and eluted with isopropyl alcohol/acetonitrile (4:1). The product was eluted off with isopropyl alcohol/acetonitrile/0.880 ammonia (2:2:1). After evaporation of the volatiles the residue was purified by silica gel chromatography eluting with 20:1 then 10:1 DCM/MeOH to give subtitle compound as a brown gum. Yield: 100 mg

MS APCI+ 601 [M+H]⁺

### vi) N-[2-(4-{[(2R)-2-hydroxy-2-(8-hydroxy-2-oxo-1,2-dihydroquinolin-5-yl)ethyl]amino}piperidin-1-yl)ethyl]benzenesulfonamide

The product from step (v) (N-[2-(4-{[(2R)-2-{[tert-butyl(dimethyl)silyl]oxy}-2-(8-hydroxy-2-oxo-1,2-dihydroquinolin-5-yl)ethyl]amino}piperidin-1-yl)ethyl]benzenesulfonamide) (100 mg) was dissolved in dry THF (4 mL) and treated with triethylamine trihydrofluoride (0.2 mL) then stirred under nitrogen for 3 h at room temperature. The mixture was evaporated and azeotroped with toluene in vacuo. The residue purified by reverse phase HPLC using an Xterra® C8 5 micron 19×50 mm column eluting with a gradient of 95 to 50% (0.1% ammonium acetate/acetonitrile) to the title compound as a beige solid. Yield: 23 mg

MS APCI+ 501 [M+H]⁺

¹H NMR (DMSO) δ 8.16 (d, 1H), 7.38-7.34 (m, 5H), 7.07 (d, 1H), 6.91 (d, 1H), 6.50 (d, 1H), 5.02 (t, 1H), 4.37 (s, 2H), 2.99 (t, 2H), 2.70 (t, 4H) 2.32 (t, 1H), 1.92 (t, 2H), 1.78 (t, 2H), 1.27 (t, 2H).

### Biological Assays

### Experimental Procedures

### Cell Preparation

H292 cells were grown in RPMI (Roswell Park Memorial Institute) medium containing, 10% (v/v) FBS (foetal bovine serum) and 2 mM L-glutamine. Cells were grown in 225 cm2 flasks containing 25 mL media in a humidified incubator at 37° C., 5% CO₂. Cells were harvested from the flask and passaged at a 1 in 10 dilution once per week.

### Experimental Method

The media from flasks containing H292 cells was removed, rinsed with 10 mL PBS (phosphate buffered saline) and replaced with 10 mL Accutase™ cell detachment solution. Flasks were incubated for 15 minutes in a humidified incubator at 37° C., 5% CO₂. The cell suspension was counted and the cells re-suspended in RPMI media (containing 10% (v/v) FBS and 2 mM L-glutamine) at 0.05×10⁶ cells per mL. 5000 cells in 100 μL were added to each well of a tissue-culture-treated 96-well plate and the cells incubated overnight in a humidified incubator at 37° C., 5% CO₂. The culture media was removed, washed twice with 100 μL assay buffer and replaced with 50 μL assay buffer. Cells were rested at room temperature for 20 minutes after which time 25 μL of rolipram (1.2 mM made up in assay buffer containing 2.4% (v/v) dimethylsulphoxide) was added. Cells were incubated with rolipram for 10 minutes after which time test compounds (made up as ×4 concentrated stocks in assay buffer containing 4% (v/v) dimethylsulphoxide) were added and the cells were incubated for 10 minutes at room temperature. Final rolipram concentration in the assay was 300 μM and final vehicle concentration was 1.6% (v/v) dimethylsulphoxide. The reaction was stopped by removing supernatants, washing once with 100 μL assay buffer and replacing with 50 μL lysis buffer. The cell monolayer was frozen at −80° C. for 30 minutes (or overnight).

AlphaScreen™ cAMP Detection

The concentration of cAMP (cyclic adenosine monophosphate) in the cell lysate was determined using the AlphaScreen™ methodology. The frozen cell plate was thawed for 20 minutes on a plate shaker then 10 μL of the cell lysate was transferred to a 96-well white plate. 40 μL of mixed AlphaScreen™ detection beads (containing equal volumes of donor beads (pre-incubated with biotinylated cAMP in the dark for 30 minutes) and acceptor beads), was added to each well and the plate incubated at room temperature for 10 hours in the dark. The AlphaScreen™ signal was measured using an EnVision spectrophotometer (Perkin-Elmer Inc.) with the recommended manufacturer's settings. cAMP concentrations were determined by reference to a calibration curve determined in the same experiment using standard cAMP concentrations (made up in lysis buffer in a 96-well tissue-culture-treated plate and frozen/thawed alongside the test samples) and detected using the same protocol. Concentration response curves for agonists were constructed to determine both the pEC₅₀ and Intrinsic Activity. Intrinsic Activity was expressed as a fraction relative to the maximum activity determined for formoterol in each experiment. The results obtained for the compounds of the Examples are shown in Table 1 below.

TABLE 1

| Compound of | pEC₅₀ | Intrinsic Activity |
|---|---|---|
| Example 2 | 7.7 | 0.9 |
| Example 3 | 7.5 | 0.8 |
| Example 5 | 8.6 | 0.8 |
| Example 6 | 8.1 | 0.8 |
| Example 14 | 8.3 | 1.0 |

Alternative Adrenergic β2 Mediated cAMP Production

### Cell Preparation

H292 cells were grown in 225 cm2 flasks incubator at 37° C., 5% CO₂ in RPMI medium containing, 10% (v/v) FBS (foetal bovine serum) and 2 mM L-glutamine.

### Experimental Method

Adherent H292 cells were removed from tissue culture flasks by treatment with Accutase™ cell detachment solution for 15 minutes. Flasks were incubated for 15 minutes in a humidified incubator at 37° C., 5% CO₂. Detached cells were re-suspended in RPMI media (containing 10% (v/v) FBS and 2 mM L-glutamine) at 0.05×10⁶ cells per mL. 5000 cells in 100 μL were added to each well of a tissue-culture-treated 96-well plate and the cells incubated overnight in a humidified incubator at 37° C., 5% CO₂. The culture media was removed and cells were washed twice with 100 μL assay buffer and replaced with 50 μL assay buffer (HBSS solution containing 10 mM HEPES pH7.4 and 5 mM glucose). Cells were rested at room temperature for 20 minutes after which time 25 μL of rolipram (1.2 mM made up in assay buffer containing 2.4% (v/v) dimethylsulphoxide) was added. Cells were incubated with rolipram for 10 minutes after which time test compounds were added and the cells were incubated for 60 minutes at room temperature. The final rolipram concentration in the assay was 300 μM and final vehicle concentration was 1.6% (v/v) dimethylsulphoxide. The reaction was stopped by removing supernatants, washing once with 100 μL assay buffer and replacing with 50 μL lysis buffer. The cell monolayer was frozen at −80° C. for 30 minutes (or overnight).

AlphaScreen™ cAMP Detection

The concentration of cAMP (cyclic adenosine monophosphate) in the cell lysate was determined using AlphaScreen™ methodology. The frozen cell plate was thawed for 20 minutes on a plate shaker then 10 μL of the cell lysate was transferred to a 96-well white plate. 40 μL of mixed AlphaScreen™ detection beads pre-incubated with biotinylated cAMP, was added to each well and the plate incubated at room temperature for 10 hours in the dark. The AlphaScreen™ signal was measured using an EnVision spectrophotometer (Perkin-Elmer Inc.) with the recommended manufacturer's settings. cAMP concentrations were determined by reference to a calibration curve determined in the same experiment using standard cAMP concentrations. Concentration response curves for agonists were constructed and data was fitted to a four parameter logistic equation to determine both the pEC₅₀ and Intrinsic Activity. Intrinsic Activity was expressed as a fraction relative to the maximum activity determined for formoterol in each experiment.

The results obtained for the compounds of the Examples are shown in Table 2 below.

TABLE 2

| Compound of | pEC₅₀ | Intrinsic Activity |
|---|---|---|
| Example 20 | 9.5 | 1 |
| Example 31 | 8.3 | 0.8 |
| Example 36 | 8.5 | 0.8 |
| Example 41 | 8.4 | 0.8 |
| Example 74 | 9.1 | 0.9 |

### Selectivity Assays

### Adrenergic α1D

### Membrane Preparation

Membranes were prepared from human embryonic kidney 293 (HEK293) cells expressing recombinant human α1_{D} receptor. These were diluted in Assay Buffer (50 mM HEPES, 1 mM EDTA, 0.1% gelatin, pH 7.4) to provide a final concentration of membranes that gave a clear window between maximum and minimum specific binding.

### Experimental Method

Assays were performed in U-bottomed 96-well polypropylene plates. 10 μL [³H]-prazosin (0.3 nM final concentration) and 10 μL of test compound (10× final concentration) were added to each test well. For each assay plate 8 replicates were obtained for [³H]-prazosin binding in the presence of 10 μL vehicle (10% (v/v) DMSO in Assay Buffer; defining maximum binding) or 10 μL BMY7378 (10 μM final concentration; defining non-specific binding (NSB)). Membranes were then added to achieve a final volume of 100 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [³H]-prazosin binding) were determined using serial dilutions typically in the range 0.1 nM to 10 μM. Data was fitted to a four parameter logistic equation to determine the compound potency, which was expressed as pIC50 (negative log molar concentration inducing 50% inhibition of [³H]-prazosin binding).

### Adrenergic β1

### Membrane Preparation

Membranes containing recombinant human adrenergic beta 1 receptors were obtained from Euroscreen. These were diluted in Assay Buffer (50 mM HEPES, 1 mM EDTA, 120 mM NaCl, 0.1% gelatin, pH 7.4) to provide a final concentration of membranes that gave a clear window between maximum and minimum specific binding.

### Experimental Method

Assays were performed in U-bottomed 96-well polypropylene plates. 10 μL [¹²⁵I]-Iodocyanopindolol (0.036 nM final concentration) and 10 μL of test compound (10× final concentration) were added to each test well. For each assay plate 8 replicates were obtained for [¹²⁵I]-Iodocyanopindolol binding in the presence of 10 μL vehicle (10% (v/v) DMSO in Assay Buffer; defining maximum binding) or 10 μL Propranolol (10 μM final concentration; defining non-specific binding (NSB)). Membranes were then added to achieve a final volume of 100 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, 120 mM NaCl, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [¹²⁵I]-Iodocyanopindolol binding) were determined using serial dilutions typically in the range 0.1 nM to 10 μM. Data was fitted to a four parameter logistic equation to determine the compound potency, which was expressed as pIC₅₀ (negative log molar concentration inducing 50% inhibition of [¹²⁵I]-Iodocyanopindolol binding).

### Dopamine D2

### Membrane Preparation

Membranes containing recombinant human Dopamine Subtype D2s receptors were obtained from Perkin Elmer. These were diluted in Assay Buffer (50 mM HEPES, 1 mM EDTA, 120 mM NaCl, 0.1% gelatin, pH 7.4) to provide a final concentration of membranes that gave a clear window between maximum and minimum specific binding.

### Experimental Method

Assays were performed in U-bottomed 96-well polypropylene plates. 30 μL [³H]-spiperone (0.16 nM final concentration) and 30 μL of test compound (10× final concentration) were added to each test well. For each assay plate 8 replicates were obtained for [³H]-spiperone binding in the presence of 30 μL vehicle (10% (v/v) DMSO in Assay Buffer; defining maximum binding) or 30 μL Haloperidol (10 μM final concentration; defining non-specific binding (NSB)). Membranes were then added to achieve a final volume of 300 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, 120 mM NaCl, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [³H]-spiperone binding) were determined using serial dilutions typically in the range 0.1 nM to 10 μM. Data was fitted to a four parameter logistic equation to determine the compound potency, which was expressed as pIC₅₀ (negative log molar concentration inducing 50% inhibition of [³H]-spiperone binding).

### Onset Assay

Dunkin-Hartley guinea-pigs (between 200 g and 300 g on delivery) were supplied by a designated breeding establishment. The guinea-pigs were killed by cervical dislocation and the trachea removed. The adherent connective tissue was removed and each trachea cut into four rings. The tissue rings were then attached to an isometric transducer. The tissues were washed and a force of 1 g was applied to each ring. In all experiments a paired curve design was used. A priming dose of 1 μM methacholine was applied to the tissues. The tissues were then washed (three times, one minute between washes), the resting tension of 1 g was reapplied and the tissues were allowed to rest for 1 hour to equilibrate. Tissues were then contracted with 1 μM methacholine and once a steady response was obtained a cumulative concentration response curve to isoprenaline (10⁻⁹ M-10⁻⁵ M) was constructed. The tissues were then washed (three times, one minute between washes) and left to rest for an hour. At the end of the resting period the tissues were contracted with 1 μM methacholine and a p[A]₅₀ concentration of test compound added. Once the tissue had reached maximum relaxation, a 30×p[A]₅₀ concentration of test compound was added. Once the tissue response had reached a plateau, 10 μM sotalol was added to the bath to confirm that the relaxation was β₂ mediated

Data were collected using the ADInstruments chart4forwindows software, which measured the maximum tension generated at each concentration of agonist.

For each concentration of the isoprenaline cumulative concentration curve, the response was calculated as % relaxation of the methacholine-induced contraction. A curve was plotted of log₁₀[agonist] (M) versus percentage inhibition of the methacholine-induced contraction. These data were then fitted to a non-linear regression curve fit. For each experiment, E/[A] curve data were fitted using a 4-parameter logistic function of the form:

$$E = β + \frac{( β - α ) \cdot {[ A ]}^{m}}{{[ A ]}^{m} + {[ A ]}_{50}^{m}}$$

E and [A] are the pharmacological effect (% relaxation) and concentration of the agonist respectively; α, β, [A]₅₀ and m are the asymptote, baseline, location and slope parameters, respectively. The p[A]₅₀ and IA of each isoprenaline curve was determined from this fit, to determine if the tissue was viable for generating an onset time for the test compounds.

For each p[A]₅₀ concentration of the test compound, the response was calculated as % relaxation of the methacholine-induced contraction. The results were plotted % relaxation against time and the time taken to reach a 90% relaxation value was calculated and recorded.

The addition of a 30×p[A]so concentration enabled determination of the maximum compound effect within the individual tissue. Hence, the % of the maximum compound effect at the p[A]₅₀ concentration was calculated and recorded.

### Pharmacokinetics in the Rat

A dose solution of the test compound was prepared using a suitable dose vehicle. The concentration of the compound in the dose solution was assayed by diluting an aliquot to a nominal concentration of 50 μg·ml⁻¹ and calibrating against duplicate injections of a standard solution and a QC standard at this concentration. Compounds were administered intravenously as a bolus into a caudal vein to groups of three 250-350 g rats (approximately 1 ml·kg⁻¹). For the oral dose, a separate group of 2 or 3 animals were dosed by oral gavage (3 ml·kg⁻¹). Delivered doses were estimated by weight loss. Food was not usually withdrawn from animals prior to dosing, although this effect was investigated if necessary.

Blood samples (0.25 ml) were taken into I'ml syringes from the caudal vein, transferred to EDTA tubes and plasma was prepared by centrifugation (5 min at 13000 rpm) soon after sample collection, before storage at −20° C. Typical sampling times were 2, 4, 8, 15, 30, 60, 120, 180, 240, 300 (min) or until the terminal t1/2 was accurately described.

The concentration of the analyte(s) were determined in plasma by quantitative mass spectrometry. Standard and quality control stock solutions were prepared at a concentration 1 mg/ml in methanol. A range of standard and QC stocks produced by serial dilution were added to control rat plasma (50 μl). The range of concentrations covered the range of levels of analyte present in the rat samples. Standards, QCs and samples underwent liquid extraction using 50 μl of organic solvent and 100 μl of organic solvent containing an internal standard, chosen to closely resemble the analyte. The samples were then mixed by repeated inversion, stored at −20° C. for at least 1 h, and centrifuged at 3500 rpm in a centrifuge for 20 minutes. Aliquots (120 μl) of each sample were transferred for analysis using LC-MSMS. Standard and quality control samples covering the range of concentrations found in the test samples were within 25% of the nominal concentration.

Pharmacokinetic data analysis was achieved using WinNonlin. A standard non-compartmental analysis was used to estimate the parameters such as Tmax, Cmax, Lambda_z, t1/2-Lambda_z, AUCall, AUCINF(observed), Cl(observed), Vss(observed).

1. A compound of formula (I): 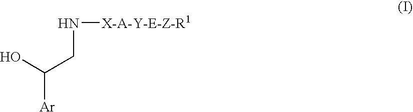
wherein
Ar is 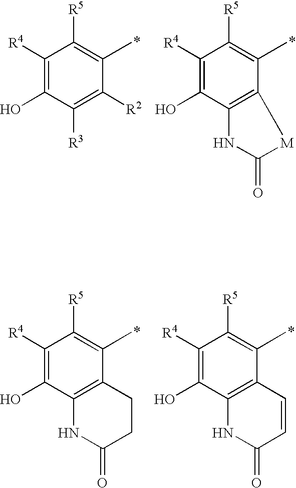
M is C(O), NR⁶, S or CR⁷R⁸;
R², R³, R⁴ and R⁵ are, independently, hydrogen, halogen, trifluoromethyl, cyano, carboxy, hydroxy, nitro, S(O)₂R⁹, NR¹⁰S(O)₂R¹¹, C(O)NR¹²R¹³, NR¹⁴C(O)R¹⁵, C₁₋₆ alkyl, C₁₋₆ alkoxy, C(O)(C₁₋₆ alkyl) or C(O)₂(C₁₋₆ alkyl);
R³ can also be CH₂OH or NHS(O)₂NR¹⁷R¹⁸;
X is a bond, CR²⁷R²⁸ or CR²⁹R³⁰CR³¹R³²;
Y is CR³³R³⁴CR³⁵R³⁶, CR³⁷R³⁸CR³⁹R⁴⁰CR⁴¹R⁴² or CR⁴³R⁴⁴CR⁴⁵R⁴⁶CR⁴⁷R⁴⁸CR⁴⁹R⁵⁰;
or Y is CR⁵¹R⁵² provided that E is C(O)O—;
Z is a bond, CR⁵¹R⁵², CR⁵³R⁵⁴CR⁵⁵R⁵⁶, CR⁵⁷R⁵⁸CR⁵⁹R⁶⁰R⁶¹R⁶² or CR⁶³R⁶⁴CR⁶⁵R⁶⁶CR⁶⁷R⁶⁸CR⁶⁹R⁷⁰;
A is a cycloalkyl-amino group selected from 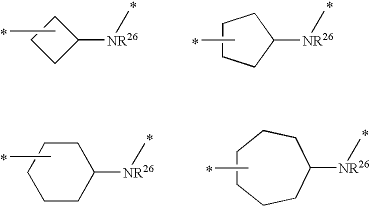
wherein said cycloalkyl ring is unsubstituted or substituted by 1 or 2 substituents independently selected from halogen, C₁₋₄ alkyl (optionally substituted by OR¹¹⁶, NR¹¹⁷R¹¹⁸ or NR¹¹⁹C(O)R¹²⁰), OR¹⁹, NR²⁰R²¹, C(O)NR²²R²³, NR²⁴C(O)R²⁵, CN, S(O)₂R¹⁶, or S(O)₂NR¹¹⁴R¹¹⁵;
when A is a cycloalkyl-amino group A is linked to X through a ring carbon atom and to Y through NR²⁶; or
when A is a cycloalkyl-amino group and X is CR²⁹R³⁰CR³¹R³² A can be linked to X through NR²⁶ and to Y through a ring carbon atom;
when X is a bond A is not connected to X through the ring-carbon atom carrying NR²⁶;
OR A is a heterocyclyl ring selected from 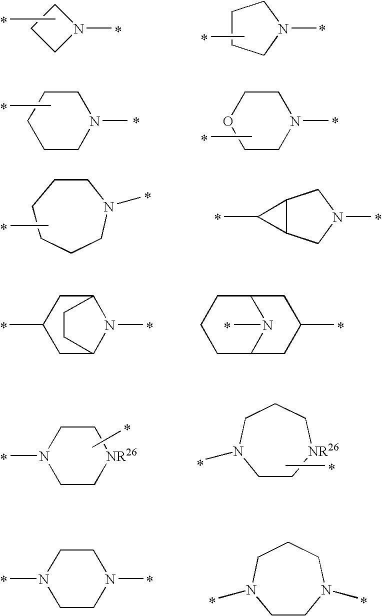
wherein the heterocyclyl ring is unsubstituted or substituted by 1 or 2 substituents independently selected from halogen, C₁₋₄ alkyl (optionally substituted by OR¹²¹, NR¹²²R¹²³ or NR¹²⁴C(O)R¹²⁵), OR¹⁹, NR²⁰R²¹, C(O)NR²²R²³, NR²⁴C(O)R²⁵, CN, S(O)₂R¹²⁶ or S(O)₂NR¹¹⁴R¹¹⁵;
when A is a heterocyclyl ring A is linked to Y through a ring nitrogen atom;
when A is a heterocyclyl ring A can be linked to X through a ring carbon atom; or, when A is heterocyclyl having 2 ring-nitrogen atoms and X is CR²⁹R³⁰CR³¹R³², A can be linked to X through the second ring nitrogen atom;
E is O, S, S(O)₂, NR⁷¹, C(O)NR⁷², NR⁷³C(O), C(O)O, S(O)₂NR⁷⁴ or NR⁷⁵S(O)₂;
R¹ is aryl, aryloxy, NR⁷⁶aryl, S(O)₂aryl, heteroaryl or C₃₋₁₀ cycloalkyl (optionally substituted by C₁₋₆ alkyl, halogen or phenyl); wherein the aryl and heteroaryl rings are optionally substituted by halogen, cyano, trifluoromethyl, phenyl, OCF₃, O(CF₂)ₙO, O(CH₂)ₘO, OR⁷⁸, SR⁷⁹, NR⁸⁰R⁸¹, C(O)NR⁸²R⁸³, NR⁸⁴S(O)₂R⁸⁵, C(O)R⁸⁶, S(O)₂R⁸⁷, S(O)₂NR⁸⁸R⁸⁹, NR⁹⁰C(O)R⁹¹, C(O)OR⁹², C₁₋₆ alkyl (optionally substituted by fluoro, trifluoromethyl, phenyl, heteroaryl, OR⁹³, NR⁹⁴R⁹⁵, C(O)NR⁹⁶R⁹⁷, NR⁹⁸S(O)₂R⁹⁹, S(O)₂R¹⁰⁰ or S(O)₂NR¹⁰¹R¹⁰²) or C₁₋₆ alkoxy (optionally substituted by fluoro, trifluoromethyl, phenyl, heteroaryl, OR¹⁰³, NR¹⁰⁴R¹⁰⁵, C(O)NR¹⁰⁶R¹⁰⁷, NR¹⁰⁸S(O)₂R¹⁰⁹, S(O)₂R¹¹⁰ or S(O)₂NR¹¹¹R¹¹²); wherein 2 substituents on the aryl or heteroaryl ring which is R¹ can join together to form a 4- to 8-membered ring which is carbocyclic or heterocyclics said 4- to 8-membered ring is fused and is optionally substituted by halogen, C₁₋₄ alkyl, CF₃ or C₁₋₄ alkoxy;
when Z is a bond E can also be C(O) provided R¹ is a group selected from: 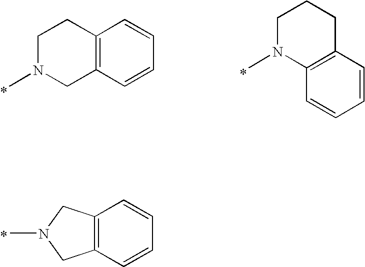
that is optionally substituted as for R¹ above;
n and m are, independently, 1 or 2;
R⁶, R⁷, R⁸, R¹⁰, R¹², R¹³, R¹⁴, R¹⁵, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, R⁷⁴, R⁷⁵, R⁷⁶, R⁷⁷, R⁷⁸, R⁷⁹, R⁸⁰, R⁸¹, R⁸², R⁸³, R⁸⁴, R⁸⁵, R⁸⁶, R⁸⁷, R⁸⁸, R⁸⁹, R⁹⁰, R⁹¹, R⁹², R⁹³, R⁹⁴, R⁹⁵, R⁹⁶, R⁹⁷, R⁹⁸, R¹⁰¹, R¹⁰², R¹⁰³, R¹⁰⁴, R¹⁰⁵, R¹⁰⁶, R¹⁰⁷, R¹⁰⁸, R¹¹¹, R¹¹², R¹¹³, R¹¹⁴, R¹¹⁵, R¹¹⁶, R¹¹⁷, R¹¹⁸, R¹¹⁹, R¹²⁰, R¹²¹, R¹²², R¹²³, R¹²⁴ and R¹²⁵ are, independently, hydrogen or C₁₋₆ alkyl;
R⁵² can also be phenyl;
R⁷² can also be phenyl(C₁₋₄ alkyl);
R⁹, R¹¹, R¹⁶, R⁸⁵, R⁸⁷, R⁹⁹, R¹⁰⁰, R¹⁰⁹, R¹¹⁰ and R¹²⁶ are, independently, C₁₋₆ alkyl;
provided that when R¹ is aryloxy, NR⁷⁶aryl or S(O)₂aryl; and E is O, S, S(O)₂, NR⁷¹, C(O)NR⁷², S(O)₂NR⁷⁴ or NR⁷⁵S(O)₂, then Z is CR⁵³R⁵⁴CR⁵⁵R⁵⁶, CR⁵⁷R⁵⁸CR⁵⁹R⁶⁰CR⁶¹R⁶² or CR⁶³R⁶⁴CR⁶⁵R⁶⁶CR⁶⁷R⁶⁸CR⁶⁹R⁷⁰;
or a pharmaceutically acceptable salt thereof.

2. The compound of formula (I) as claimed in claim 1 wherein Ar is:

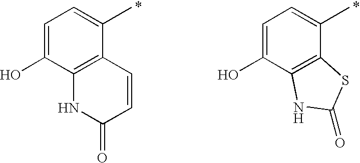

3. The compound of formula (I) as claimed in claim 1 wherein X is a bond, CH₂ or C(CH₃)₂ or (CH₂)₂.

4. The compound of formula (I) as claimed in claim 1 wherein Y is (CH₂)₂, (CH₂)₃ or CH₂C(CH₃)₂CH₂.

5. The compound of formula (I) as claimed in claim 1 wherein E is O or C(O)NR⁷²; and R⁷² is hydrogen or C₁₋₄ alkyl.

6. The compound of formula (I) as claimed in claim 1 of S wherein Z is CH₂ or (CH₂)₂.

7. The compound of formula (I) as claimed in claim 1 wherein A is: 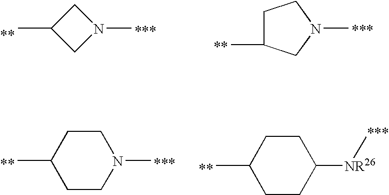
where ** is linked to X and *** is linked to Y; and A is optionally substituted as recited in claim 1; and R²⁶ is as defined in claim 1.

8. The compound of formula (I) as claimed claim 1 wherein R¹ is unsubstituted phenyl or phenyl substituted by the same or different: halogen, C₁₋₄ alkyl, C₁₋₄ alkoxy, cyano, OH, CF₃, OCF₃ or phenyl.

9. The compound of formula (I) as claimed in claim 1 selected from:
8-Hydroxy-5-{(1R)-1-hydroxy-2-[({1-[2-(2-phenylethoxy)ethyl]piperidin-4-yl}methyl)amino]ethyl}quinolin-2(1H)-one;
8-Hydroxy-5-{(1R)-1-hydroxy-2-[(trans-4-{[2-(2-phenylethoxy)ethyl]amino}cyclohexyl)amino]ethyl}quinolin-2(1H)-one;
8-Hydroxy-4-[(1R)-1-hydroxy-2-({1-[3-(2-phenylethoxy)propyl]piperidin-4-yl}amino)ethyl]quinolin-2(1H)-one;
8-Hydroxy-4-[(1R)-1-hydroxy-2-({1-[2-(2-phenylethoxy)ethyl]piperidin-4-yl}amino)ethyl]quinolin-2(1H)-one;
8-Hydroxy-5-{(1R)-1-hydroxy-2-[({4-hydroxy-1-[2-(2-phenylethoxy)ethyl]piperidin-4-yl}methyl)amino]ethyl}quinolin-2(1H)-one;
N-Benzyl-3-(4-{[(2R)-2-hydroxy-2-(8-hydroxy-2-oxo-1,2-dihydroquinolin-5-yl)ethyl]amino}piperidin-1-yl)propanamide;
N-Benzyl-3-[4-({[(2R)-2-hydroxy-2-(8-hydroxy-2-oxo-1,2-dihydroquinolin-5-yl)ethyl]amino}methyl)piperidin-1-yl]propanamide;
5-[(1R)-2-({1-[3-(3,4-Dihydroisoquinolin-2(1H)-yl)-3-oxopropyl]piperidin-4-yl}amino)-1-hydroxyethyl]-8-hydroxyquinolin-2(1H)-one;
3-(4-{[(2R)-2-Hydroxy-2-(8-hydroxy-2-oxo-1,2-dihydroquinolin-5-yl)ethyl]amino}piperidin-1-yl)-N-(2-phenylethyl)propanamide;
N-(2-Chlorobenzyl)-3-(4-{[(2R)-2-hydroxy-2-(8-hydroxy-2-oxo-1,2-dihydroquinolin-5-yl)ethyl]amino}piperidin-1-yl)propanamide;
3-(4-{[(2R)-2-Hydroxy-2-(8-hydroxy-2-oxo-1,2-dihydroquinolin-5-yl)ethyl]amino}piperidin-1-yl)-N-(2-methoxybenzyl)propanamide;
N-(4-Cyanobenzyl)-3-(4-{[(2R)-2-hydroxy-2-(8-hydroxy-2-oxo-1,2-dihydroquinolin-5-yl)ethyl]amino}piperidin-1-yl)propanamide;
N-(2-Hydroxybenzyl)-3-(4-{[(2R)-2-hydroxy-2-(8-hydroxy-2-oxo-1,2-dihydroquinolin-5-yl)ethyl]amino}piperidin-1-yl)propanamide;
8-Hydroxy-5-{(1R)-1-hydroxy-2-[({4-hydroxy-1-[3-(2-phenylethoxy)propyl]piperidin-4-yl}methyl)amino]ethyl}quinolin-2(1H)-one;
4-Hydroxy-7-{(1R)-1-hydroxy-2-[({1-[2-(2-phenylethoxy)ethyl]piperidin-4-yl}methyl)amino]ethyl}-1,3-benzothiazol-2(3H)-one;
4-Hydroxy-7-{(1R)-1-hydroxy-2-[({1-[3-(2-phenylethoxy)propyl]azetidin-3-yl}methyl)amino]ethyl}-1,3-benzothiazol-2(3H)-one;
4-HYDROXY-7-{(1R)-1-HYDROXY-2-[(2-{1-[2-(2-PHENYLETHOXY)ETHYL]PIPERIDIN-4-YL}ETHYL)AMINO]ETHYL}-1,3-BENZOTHIAZOL-2(3H)-ONE;
4-Hydroxy-7-{1-hydroxy-2-[1-(2-phenethyloxy-ethyl)-piperidin-4-ylamino]-ethyl}-3H-benzothiazol-2-one;
4-Hydroxy-7-((1R)-1-hydroxy-2-{[(3R)-1-(2-phenethyloxy-ethyl)-piperidin-3-ylmethyl]-amino}-ethyl)-3H-benzothiazol-2-one;
4-Hydroxy-7-((1R)-1-hydroxy-2-{[(3R)-1-(2-phenethyloxy-ethyl)-piperidin-3-ylmethyl]-amino}-ethyl)-3H-benzothiazol-2-one;
5-{(1R)-2-[({1-[3-(benzyloxy)propyl]-4-hydroxypiperidin-4-yl}methyl)amino]-1-hydroxyethyl}-8-hydroxyquinolin-2(1H)-one;
5-{(1R)-2-[({1-[2-(enzyloxy)ethyl]-4-hydroxypiperidin-4-yl}methyl)amino]-1-hydroxyethyl}-8-hydroxyquinolin-2(1H)-one;
N-(2,5-dichlorobenzyl)-3-(4-{[(2R)-2-hydroxy-2-(8-hydroxy-2-oxo-1,2-dihydroquinolin-5-yl)ethyl]amino}piperidin-1-yl)propanamide;
N-(biphenyl-2-ylmethyl)-3-(4-{[(2R)-2-hydroxy-2-(8-hydroxy-2-oxo-1,2-dihydroquinolin-5-yl)ethyl]amino}piperidin-1-yl)propanamide;
N-(2,6-dichlorobenzyl)-3-(4-{[(2R)-2-hydroxy-2-(8-hydroxy-2-oxo-1,2-dihydroquinolin-5-yl)ethyl]amino}piperidin-1-yl)propanamide;
N-(cyclohexylmethyl)-3-(4-{[(2R)-2-hydroxy-2-(8-hydroxy-2-oxo-1,2-dihydroquinolin-5-yl)ethyl]amino}piperidin-1-yl)propanamide;
N-(2-chloro-6-methylbenzyl)-3-(4-{[(2R)-2-hydroxy-2-(8-hydroxy-2-oxo-1,2-dihydroquinolin-5-yl)ethyl]amino}piperidin-1-yl)propanamide;
3-(4-{[(2R)-2-hydroxy-2-(8-hydroxy-2-oxo-1,2-dihydroquinolin-5-yl)ethyl]amino}piperidin-1-yl)-N-[(1R,2S)-2-phenylcyclopropyl]propanamide;
N-(4-chlorobenzyl)-3-(4-{[(2R)-2-hydroxy-2-(8-hydroxy-2-oxo-1,2-dihydroquinolin-5-yl)ethyl]amino}piperidin-1-yl)propanamide;
N-(3-chlorobenzyl)-3-(4-{[(2R)-2-hydroxy-2-(8-hydroxy-2-oxo-1,2-dihydroquinolin-5-yl)ethyl]amino}piperidin-1-yl)propanamide;
N-(2-chloro-6-fluorobenzyl)-3-(4-{[(2R)-2-hydroxy-2-(8-hydroxy-2-oxo-1,2-dihydroquinolin-5-yl)ethyl]amino}piperidin-1-yl)propanamide;
N-(2,3-dichlorobenzyl)-3-(4-{[(2R)-2-hydroxy-2-(8-hydroxy-2-oxo-1,2-dihydroquinolin-5-yl)ethyl]amino}piperidin-1-yl)propanamide;
N-(2-chlorobenzyl)-3-(4-{[(2R)-2-hydroxy-2-(8-hydroxy-2-oxo-1,2-dihydroquinolin-5-yl)ethyl]amino}piperidin-1-yl)-N-methylpropanamide;
5-((1R)-2-{[(1-{2-[2-(3-chlorophenyl)ethoxy]ethyl}-4-hydroxypiperidin-4-yl)methyl]amino}-1-hydroxyethyl)-8-hydroxyquinolin-2(1H)-one;
benzyl (4-{[(2R)-2-hydroxy-2-(8-hydroxy-2-oxo-1,2-dihydroquinolin-5-yl)ethyl]amino}piperidin-1-yl)acetate;
8-Hydroxy-5-[(1R)-1-hydroxy-2-({[4-hydroxy-1-(4-phenoxybutyl)piperidin-4-yl]methyl}amino)ethyl]quinolin-2(1H)-one;
N-1-Adamantyl-3-(4-{[(2R)-2-hydroxy-2-(8-hydroxy-2-oxo-1,2-dihydroquinolin-5-yl)ethyl]amino}piperidin-1-yl)propanamide;
N-(3,5-Dichlorobenzyl-3-(4-{[(2R)-2-hydroxy-2-(8-hydroxy-2-oxo-1,2-dihydroquinolin-5-yl)ethyl]amino}piperidin-1-yl)propanamide;
8-Hydroxy-5-{(1R)-1-hydroxy-2-[({4-(hydroxymethyl)-1-[2-(2-phenylethoxy)ethyl]piperidin-4-yl}methyl)amino]ethyl}quinolin-2(1H)-one;
2,6-Dichloro-N-[2-(4-{[(2R)-2-hydroxy-2-(8-hydroxy-2-oxo-1,2-dihydroquinolin-5-yl)ethyl]amino}piperidin-1-yl)ethyl]benzamide;
8-Hydroxy-5-[(1R)-1-hydroxy-2-({[1-(2-{[(2S)-2-phenylpropyl]oxy}ethyl)piperidin-4-yl]methyl}amino)ethyl]quinolin-2(1H)-one;
5-((1R)-2-{[(1-{2-[2-(2-chlorophenyl)ethoxy]ethyl}-4-hydroxypiperidin-4-yl)methyl]amino}-1-hydroxyethyl)-8-hydroxyquinolin-2(1H)-one;
N-(2,5-Dimethylbenzyl-3-(4-{[(2R)-2-hydroxy-2-(8-hydroxy-2-oxo-1,2-dihydroquinolin-5-yl)ethyl]amino}piperidin-1-yl)propanamide;
N-(Adamant-1-ylmethyl)-3-(4-{[(2R)-2-hydroxy-2-(8-hydroxy-2-oxo-1,2-dihydroquinolin-5-yl)ethyl]amino}piperidin-1-yl)propanamide;
N-(3-Chloro-2-methylbenzyl)-3-(4-{[(2R)-2-hydroxy-2-(8-hydroxy-2-oxo-1,2-dihydroquinolin-5-yl)ethyl]amino}piperidin-1-yl)propanamide;
3-(4-{[(2R)-2-Hydroxy-2-(8-hydroxy-2-oxo-1,2-dihydroquinolin-5-yl)ethyl]amino}piperidin-1-yl)-N-(2-trifluoromethoxybenzyl)-propanamide;
N-((3-Fluoro-5-trifluoromethyl)benzyl)-3-(4-{[(2R)-2-hydroxy-2-(8-hydroxy-2-oxo-1,2-dihydroquinolin-5-yl)ethyl]amino}piperidin-1-yl)propanamide;
N-[2-Fluoro-3-(trifluoromethyl)benzyl]-3-(4-{[(2R)-2-hydroxy-2-(8-hydroxy-2-oxo-1,2-dihydroquinolin-5-yl)ethyl]amino}piperidin-1-yl)propanamide;
N-((2-Chloro-5-trifluoromethyl)benzyl)-3-(4-{[(2R)-2-hydroxy-2-(8-hydroxy-2-oxo-1,2-dihydroquinolin-5-yl)ethyl]amino}piperidin-1-yl)propanamide;
N-((5-Fluoro-2-trifluoromethyl)benzyl)-3-(4-{[(2R)-2-hydroxy-2-(8-hydroxy-2-oxo-1,2-dihydroquinolin-5-yl)ethyl]amino}piperidin-1-yl)propanamide;
3-(4-{[(2R)-2-Hydroxy-2-(8-hydroxy-2-oxo-1,2-dihydroquinolin-5-yl)ethyl]amino}piperidin-1-yl)-N-[(2-trifluoromethyl)benzyl]propanamide;
N-(5-Chloro-2-methylbenzyl)-3-(4-{[(2R)-2-hydroxy-2-(8-hydroxy-2-oxo-1,2-dihydroquinolin-5-yl)ethyl]amino}piperidin-1-yl)propanamide;
N-(3,5-Dimethylbenzyl)-3-(4-{[(2R)-2-hydroxy-2-(8-hydroxy-2-oxo-1,2-dihydroquinolin-5-yl)ethyl]amino}piperidin-1-yl)propanamide;
3-(4-{[(2R)-2-hydroxy-2-(8-hydroxy-2-oxo-1,2-dihydroquinolin-5-yl)ethyl]amino}piperidin-1-yl)-N-(3-trifluoromethoxybenzyl)propanamide;
N-(3-Chloro-2-fluorobenzyl)-3-(4-{[(2R)-2-hydroxy-2-(8-hydroxy-2-oxo-1,2-dihydroquinolin-5-yl)ethyl]amino}piperidin-1-yl)propanamide;
N-[(2-Fluoro-5-trifluoromethyl)benzyl]-3-(4-{[(2R)-2-hydroxy-2-(8-hydroxy-2-oxo-1,2-dihydroquinolin-5-yl)ethyl]amino}piperidin-1-yl)propanamide;
N-[(5-Chloro-2-fluoro)benzyl]-3-(4-{[(2R)-2-hydroxy-2-(8-hydroxy-2-oxo-1,2-dihydroquinolin-5-yl)ethyl]amino}piperidin-1-yl)propanamide;
3-(4-{[(2R)-2-Hydroxy-2-(8-hydroxy-2-oxo-1,2-dihydroquinolin-5-yl)ethyl]amino}piperidin-1-yl)-N-(3-trifluoromethyl)benzylpropanamide;
N-Benzhydryl-3-(4-{[(2R)-2-hydroxy-2-(8-hydroxy-2-oxo-1,2-dihydroquinolin-5-yl)ethyl]amino}piperidin-1-yl)propanamide;
N,N-Dibenzyl-3-(4-{[(2R)-2-hydroxy-2-(8-hydroxy-2-oxo-1,2-dihydroquinolin-5-yl)ethyl]amino}piperidin-1-yl)propanamide;
N-[(3,5-Bistrifluoromethyl)benzyl]-3-(4-{[(2R)-2-hydroxy-2-(8-hydroxy-2-oxo-1,2-dihydroquinolin-5-yl)ethyl]amino}piperidin-1-yl)propanamide;
N-[(Biphenyl-3-yl)methyl]-3-(4-{[(2R)-2-hydroxy-2-(8-hydroxy-2-oxo-1,2-dihydroquinolin-5-yl)ethyl]amino}piperidin-1-yl)propanamide;
3-(4-{[(2R)-2-hydroxy-2-(8-hydroxy-2-oxo-1,2-dihydroquinolin-5-yl)ethyl]amino}piperidin-1-yl)-N-[(5,6,7,8-tetrahydronaphthalen-1-yl)methyl]propanamide;
5-((1R)-2-{[(1-{2-[2-(2,6-Dichlorophenyl)ethoxy]ethyl}-4-hydroxypiperidin-4-yl)methyl]amino}-1-hydroxyethyl)-8-hydroxyquinolin-2(1H)-one;
8-Hydroxy-5-{(1R)-1-hydroxy-2-[({4-hydroxy-1-[2-(2-methyl-2-phenylpropoxy)ethyl]piperidin-4-yl}methyl)amino]ethyl}quinolin-2(1H)-one;
5-{(1R)-2-[({1-[2-(1,1-Dimethyl-2-phenylethoxy)ethyl]-4-hydroxypiperidin-4-yl}methyl)amino]-1-hydroxyethyl}-8-hydroxyquinolin-2(1H)-one;
5-((1R)-2-{[(1-{2-[2-(2,3-Dichlorophenyl)ethoxy]ethyl}-4-hydroxypiperidin-4-yl)methyl]amino}-1-hydroxyethyl)-8-hydroxyquinolin-2(1H)-one;
5-{(1R)-2-[({1-[2-(1,1-Dimethyl-2-phenylethoxy)ethyl]piperidin-4-yl}methyl)amino]-1-hydroxyethyl}-8-hydroxyquinolin-2(1H)-one;
8-Hydroxy-5-{(1R)-1-hydroxy-2-[({1-[2-(2-methyl-2-phenylpropoxy)ethyl]piperidin-4-yl}methyl)amino]ethyl}quinolin-2(1H)-one;
8-Hydroxy-5-{(2R)-1-hydroxy-2-[(4-hydroxy-1-{2-[2-(5,6,7,8-tetrahydronaphthalen-1-yl)ethoxy]ethyl}piperidin-4-ylmethyl)amino]ethyl}-1H-quinolin-2-one;
8-Hydroxy-5-{(1R)-1-hydroxy-2-[({(2S)-1-[3-(2-phenylethoxy)propyl]pyrrolidin-2-yl}methyl)amino]ethyl}quinolin-2(1H)-one; and
N-[2-(4-{[(2R)-2-hydroxy-2-(8-hydroxy-2-oxo-1,2-dihydroquinolin-5-yl)ethyl]amino}piperidin-1-yl)ethyl]benzenesulfonamide;
and pharmaceutically acceptable salts thereof.

10. A process for preparing a compound of formula (I) as claimed in claim 1, the process comprising:
a. reacting a compound of formula (II) or (IV): 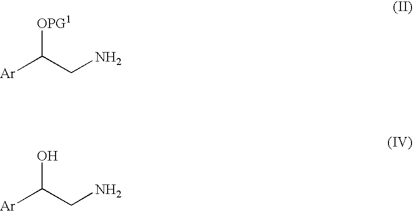
wherein Ar is as defined in claim 1, with or without a suitable protecting group on the phenolic group and wherein PG¹ is a suitable protecting group which may be the same or different to the phenolic protecting group, with a compound of formula (III):
O═X-A-Y-E-Z-R¹ (III)
in the presence of a suitable reducing agent, organic acid and solvent;
b. reacting a compound of formula (V): 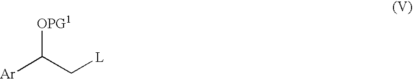
having a suitable protecting group (PG²) on the phenolic group, and wherein PG¹ is a suitable protecting group which may be the same or different to PG² and L is a halogen, with a compound of formula (VI):
H₂N—X-A-Y-E-Z-R¹ (VI)
in the presence of a suitable base and solvent (optionally in the presence of a catalyst);
c. reacting a compound of formula (VII): 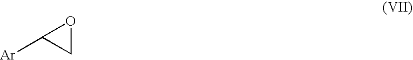
having a suitable protecting group (PG²) on the phenolic group, with a compound of formula (VI) in the presence of a suitable base and suitable solvent;
d. when E is C(O)NR⁷², reacting a compound of formula (XVII): 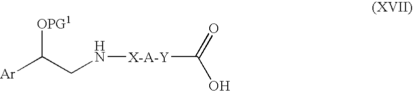
wherein Ar is with or without a suitable protecting group (PG²) on the phenolic group, and wherein PG¹ is hydrogen or a suitable protecting group which is the same or different to PG², with a compound of formula (XVIII): 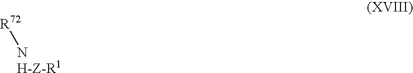
in the presence of a suitable activating coupling agent, a suitable base and a suitable solvent;
e. when Z is a bond, E is C(O) and R¹ is a group selected from: 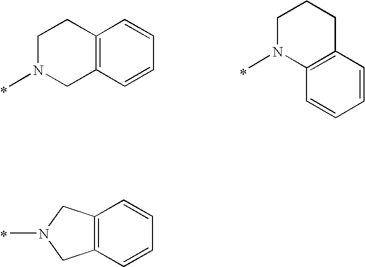
reacting a compound of formula (XVII) wherein Ar is with or without a suitable protecting group (PG²) on the phenolic group, with a compound of formula (XIX):
H—R¹ (XIX)
in the presence of a suitable activating coupling agent, a suitable base and a suitable solvent.

11. A pharmaceutical composition comprising a compound of formula (I) or a pharmaceutically acceptable salt thereof as claimed in claim 1 in association with a pharmaceutically acceptable adjuvant, diluent or carrier.

12-14. (canceled)

15. A method of treating, or reducing the risk of, a disease or condition in which modulation of β2 adrenoreceptor activity is beneficial which comprises administering to a patient in need thereof a therapeutically effective amount of a compound of formula (I) or a pharmaceutically acceptable salt thereof as claimed in claim 1.

16. A method of treating, or reducing the risk of, an inflammatory disease or condition which comprises administering to a patient in need thereof a therapeutically effective amount of a compound of formula (I) or a pharmaceutically acceptable salt thereof as claimed in claim 1.

17. A method according to claim 15 or claim 16, wherein the disease or condition is adult respiratory distress syndrome (ARDS), pulmonary emphysema, bronchitis, bronchiectasis, chronic obstructive pulmonary disease (COPD), asthma or rhinitis.

18. A combination comprising a compound of formula (I) as claimed in claim 1 and one or more agents selected from the list comprising:
a non-steroidal glucocorticoid receptor (GR-receptor) agonist;
a steriod (such as budesonide or fluticasone);
a PDE4 inhibitor;
a muscarinic receptor antagonist;
a modulator of chemokine receptor function; or,
an inhibitor of p38 kinase function.

19. A chiral intermediate of formula (IV): 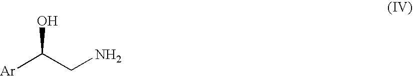
wherein
Ar is: 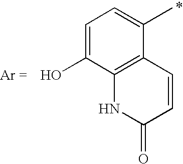
that is, the compound 5-[(1R)-2-amino-1-hydroxyethyl]-8-hydroxyquinolin-2(1H)-one.

20. An intermediate compound of formula (XX): 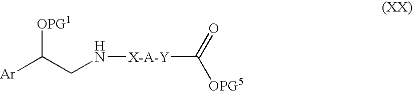
wherein:
Ar is: 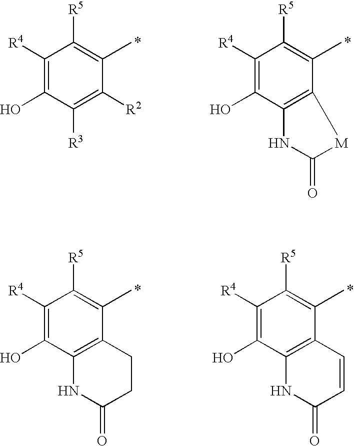
M is C(O), NR⁶, S or CR⁷R⁸;
R², R³, R⁴ and R⁵ are, independently, hydrogen, halogen, trifluoromethyl, cyano, carboxy, hydroxy, nitro, S(O)R⁹, NR¹⁰S(O)₂R¹¹, C(O)NR¹²R¹³, NR¹⁴C(O)R¹⁵, C₁₋₆ alkyl, C₁₋₆ alkoxy, C(O)(C₁₋₆ alkyl) or C(O)₂(C₁₋₆ alkyl);
R³ can also be CH₂OH or NHS(O)ₙNR¹⁷R¹⁸;
PG¹ is suitable protecting group;
X is a bond;
A is piperidinyl linked to X through the 4-position and N-linked to Y;
Y is (CH₂)₂; and
PG⁵ is either hydrogen or a suitable protecting group.
